(19) United States
(12) Patent Application Publication
(10) Pub. No.: US 2025/0062040 A1
(43) Pub. Date: Feb. 20, 2025

(54) COMPUTER-BASED SYSTEMS FOR ACQUIRING AND ANALYZING OBSERVATIONAL SUBJECT DATA

(71) Applicant: PGI DRUG DISCOVERY LLC, Jacksonville, FL (US)

(72) Inventors: Daniela BRUNNER, Bronx, NY (US); Alberto AMBESI-IMPIOMBATO, New York, NY (US); Jean-Sebastien VALOIS, Pittsburgh, PA (US); Hasan ALMAWI, Hackensack, NJ (US); Mukesh BANSAL, San Diego, CA (US); Andriy YELISYEYEV, New York, NY (US); Sung-Cheol KIM, Ridgewood, NJ (US); Ian RUSSELL, Nyack, NY (US); Mitchell SILVERSTEIN, Upper Nyack, NY (US); David High, Plymouth, MN (US); Steven LEISER, Valley Cottage, NY (US); Maxim DOLGUIKH, Naples, FL (US)

(21) Appl. No.: 18/720,603

(22) PCT Filed: Dec. 16, 2022
(86) PCT No.: PCT/US2022/081831

§ 371 Date: Jun. 14, 2024

Related U.S. Application Data
(60) Provisional application No. 63/295,057, filed on Dec. 30, 2021.
(60) Provisional application No. 63/295,085, filed on Dec. 30, 2021.
(60) Provisional application No. 63/295,105, filed on Dec. 30, 2021.
(60) Provisional application No. 63/295,124, filed on Dec. 30, 2021.
(60) Provisional application No. 63/295,164, filed on Dec. 30, 2021.
(60) Provisional application No. 63/295,184, filed on Dec. 30, 2021.
(60) Provisional application No. 63/295,208, filed on Dec. 30, 2021.
(60) Provisional application No. 63/295,232, filed on Dec. 30, 2021.
(60) Provisional application No. 63/295,242, filed on Dec. 30, 2021.
(60) Provisional application No. 63/295,260, filed on Dec. 30, 2021.
(60) Provisional application No. 63/295,298, filed on Dec. 30, 2021.
(60) Provisional application No. 63/295,391, filed on Dec. 30, 2021.
(60) Provisional application No. 63/295,421, filed on Dec. 30, 2021.
(60) Provisional application No. 63/290,208, filed on Dec. 16, 2021.

(51) Int. Cl. G16H 70/40; A61B 5/00; G06V 10/26; G06V 10/62; G06V 10/764; G06V 10/82; G06V 20/52; G06V 40/20; G16H 40/63; H04N 13/236; H04N 13/243; H04N 25/21; H04N 25/47

(52) U.S. Cl. CPC G16H 70/40; A61B 5/4848; G06V 10/26; G06V 10/62; G06V 10/764; G06V 10/82; G06V 20/52; G06V 40/25; G16H 40/63; G06V 2201/03; H04N 13/236; H04N 13/243; H04N 25/21; H04N 25/47

(57) ABSTRACT

The present disclosure provides devices, systems, and methods for capturing and analyzing behavioral and physiological data of subjects for treatment, modification, and manipulation discovery. In some embodiments, a method for classifying a drug is provided. The method includes obtaining observational data concerning an animal subject to which the drug is administered, the observational data acquired using an enclosure for the animal subject, the enclosure instrumented with at least one sensing device. The method also includes extracting features by applying the observational data to a machine-learning feature-extraction component. The method further includes predicting a class label of the drug by applying the features to a machine-learning classifier component, the machine-learning classifier component trained to predict the class label of the drug from, at least in part, the features. The method further includes providing an indication of the class label.

### CROSS-REFERENCE TO RELATED APPLICATIONS

This application claims the benefit of priority of U.S. Provisional Patent Application No. 63/290,208, filed Dec. 16, 2021, U.S. Provisional Patent Application No. 63/295,085, filed Dec. 30, 2021, U.S. Provisional Patent Application No. 63/295,242, filed Dec. 30, 2021, U.S. Provisional Patent Application No. 63/295,298, filed Dec. 30, 2021, U.S. Provisional Patent Application No. 63/295,184, filed Dec. 30, 2021, U.S. Provisional Patent Application No. 63/295,057, filed Dec. 30, 2021, U.S. Provisional Patent Application No. 63/295,391, filed Dec. 30, 2021, U.S. Provisional Patent Application No. 63/295,124, filed Dec. 30, 2021, U.S. Provisional Patent Application No. 63/295,164, filed Dec. 30, 2021, U.S. Provisional Patent Application No. 63/295,232, filed Dec. 30, 2021, U.S. Provisional Patent Application No. 63/295,208, filed Dec. 30, 2021, U.S. Provisional Patent Application No. 63/295,105, filed Dec. 30, 2021, U.S. Provisional Patent Application No. 63/295,421, filed Dec. 30, 2021, all of which are incorporated herein by reference in their entireties.

### TECHNICAL FIELD

The present disclosure relates to devices, systems, and methods for capturing and analyzing behavioral and physiological data of subjects under modification.

### BACKGROUND

The development of new drugs and compounds involves the study of their effects on various animals. Animal tests provide the safety and efficacy data necessary to support subsequent human trials.

Behavioral assessments are an important component of such animal tests. The effects of experimental substances on animal behavior can provide information about the potential clinical effects of the experimental substances on human subjects. For example, the discovery that chlorpromazine produces differential effects on avoidance and escape behavior in animals encouraged the evaluation of the behavioral effects of other experimental antipsychotic drugs.

A behavioral assessment can include comparing the behavior of an animal treated with an experimental substance to the normal behavior of the animal (or another animal). A behavioral phenotype can be generated from a set of monitored behaviors. The behavioral phenotype can be used to correlate treatment with the experimental substances and the behavior or physiology of the animal. Behavioral platforms can automatically collect observational data for the generation of such behavioral phenotypes. As compared to manual collection of observational data automatic data collection can be more complete, repeatable, reliable, and accurate.

### SUMMARY

The present disclosure describes devices, systems, and methods for capturing and analyzing behavioral, physiological data of subjects for treatment, modification, and manipulation discovery.

According to an exemplary aspect of the present disclosure, a computer-implemented method of classifying a drug is provided. The method includes obtaining observational data concerning an animal subject to which the drug is administered. The observational data is acquired using an enclosure for the animal subject. The enclosure is instrumented with at least one sensing device. The method includes extracting features by applying the observational data to a machine-learning feature-extraction component. The method also include predicting a class label of the drug by applying the features to a machine-learning classifier component. The machine-learning classifier component has been trained to predict the class label of the drug from, at least in part, the features. The method includes providing an indication of the class label.

According to an exemplary aspect of the present disclosure, a computer-implemented method of classifying a drug is provided. The method includes obtaining observational data concerning an animal subject to which the drug is administered. The observational data includes at least one of thermal data or respirational data. The observation data can be acquired using an enclosure for the animal subject. The enclosure can be instrumented with at least one sensing device. The method includes extracting features by applying the observational data to a machine-learning feature-extraction component. The method includes predicting a class label of the drug by applying the features to a machine-learning classifier component trained to predict the class label of the drug from, at least in part, the features. The method includes providing an indication of the class label.

According to an exemplary aspect of the present disclosure, a computer-implemented method of classifying a psychedelic drug is provided. The method includes obtaining observational data concerning an animal subject to which a predetermined dose of the psychedelic drug is administered. The observation data is acquired using an enclosure for the animal subject. The enclosure is instrumented with at least one sensing device. The method includes extracting features by applying the observational data to a machine-learning feature-extraction component. The features include at least one of head twitch, nose scratch, ear scratch, head shake, body elongation, or elongation-contraction. The method includes predicting a class label of the psychedelic drug at the predetermined dose by applying the features to a machine-learning classifier component trained to predict the class label of the psychedelic drug from, at least in part, the features. The method includes providing an indication of the class label.

According to an exemplary aspect of the present disclosure, a computer-implemented drug screening method is provided. The method includes obtaining observational data concerning an animal subject, the observational data acquired using an enclosure for the animal subject. The enclosure is instrumented with at least one sensing device. The method includes extracting instant behavioral features from the observational data. The method includes creating a treatment signature. The treatment signature includes higher-order features derived from the instant behavioral features using a first machine-learning component. The higher-order features include at least one of a state feature, a motif feature, or a domain feature. The method includes generating a target signature difference between the treatment signature and a baseline signature. The method includes identifying at least one reference drug or condition based on the target signature difference. The identification includes generating an upregulation enrichment score and a downregulation enrichment score for the at least one reference drug or condition using the target signature difference and a reference signature difference corresponding to the one of the at least one reference drug or condition; generating a combined enrichment score for the at least one reference drug or condition using the target signature difference and a reference signature difference corresponding to the one of the at least one reference drug or condition; or generating a similarity value for the at least one reference drug or condition using the target signature difference and a reference signature difference corresponding to the one of the at least one reference drug or condition. The method includes providing an indication of the similarity value, combined enrichment score, or upregulation and downregulation enrichment scores for the at least one reference drug or condition.

According to an exemplary aspect of the present disclosure, a computer-implemented drug screening method. The method includes, in a training phase: obtaining, for each first animal subject in three of more sets of first animal subjects, each of the first sets corresponding to a combination of values of two or more characteristics of the first animals, a first value for each behavioral feature in a set of features; and determining, using a second machine-learning component and the first values, a mapping between at least two dimensions and corresponding functions of the feature. The dimensions include a treatment dimension and a secondary dimension. The features include instant behavioral features extracted from observational data acquired for the first animal subjects using an enclosure instrumented with at least one sensing device; and higher-order features derived from the instant behavioral features using a first machine-learning component. The method includes, in a screening phase: obtaining a second value for each behavioral feature in the set of the features for a second animal subject to which a compound is administered; determining, using the mapping and the second values, a treatment effect of the compound; and providing an indication of the treatment effect.

According to an exemplary aspect of the present disclosure, a computer-implemented method of classifying a drug is provided. The method includes obtaining EEG data from a plurality of electrodes positioned on an animal subject to which the drug is administered at a first dose. The method includes obtaining acceleration data from one or more accelerometers positioned on the animal subject to which the drug is administered. The method includes predicting a class label for the drug by applying the EEG data and the acceleration data to a machine-learning classifier component trained to predict the class label using the EEG data and the acceleration data. The method includes providing an indication of the class label.

According to an exemplary aspect of the present disclosure, a computer-implemented method of extracting gait features of a rodent is provided. The method includes obtaining video data illustrating a rodent, to which a drug is administered, over a predetermined period. The video data is acquired using an enclosure for the rodent. The enclosure can be instrumented with an illuminated track for the rodent. The enclosure can be instrumented with at least one imaging device positioned to image the underside of the illuminated track. The at least one imaging device may include a video recording device configured to obtain video data. The method includes annotating frames in the video data with labels using two machine-learning components. The method includes generating segmented images using the annotating frames, the segmented images divided into segments corresponding to third object classes including hind left, hind right, front left, and front right paws. The method includes extracting gait features of the rodent from the segmented images. The method includes extracting the silhouette of the rodent from the segmented images. The labels include a first one of the two machine-learning components configured to divide a frame in video data into segments corresponding to first object classes, the first object classes comprising a paw class. The labels include a second one of the two machine-learning components configured to detect bounding boxes corresponding to second object classes, the second object classes including hind left, hind right, front left, and front right paws.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 illustrates an exemplary data processing flow diagram for a behavioral treatment discovery, consistent with disclosed embodiments.

FIG. 2 illustrates an exemplary data processing flow diagram for a behavioral treatment discovery, consistent with disclosed embodiments.

FIG. 3A illustrates an exemplary computer-based behavioral treatment discovery system, consistent with disclosed embodiments.

FIG. 3B illustrates an exemplary computer-based behavioral treatment discovery system, consistent with disclosed embodiments.

FIG. 4 illustrates an exemplary feature engineering data flow diagram for a behavioral treatment discovery, consistent with disclosed embodiments.

FIG. 5 illustrates an exemplary computer-based behavioral treatment discovery system, consistent with disclosed embodiments.

FIG. 6 illustrates an exemplary enclosure with a sensor's field-of-view, location, and minimum distance, consistent with disclosed embodiments.

FIG. 7 illustrates an exemplary nested U-net model, consistent with disclosed embodiments.

FIG. 8 illustrates an example of a real model segmentation result by mapping the changes in pixel distance of the center of the mouse over time, consistent with disclosed embodiments.

FIG. 9 illustrates an exemplary ellipse model used to geometrically fit an ellipse around the mouse, consistent with disclosed embodiments.

FIG. 10 illustrates an exemplary animal body segmentation where the body is also segmented in sub-parts, consistent with disclosed embodiments.

FIG. 11 illustrates exemplary animal body segmentation where the body is also segmented in sub-parts, consistent with disclosed embodiments.

FIG. 12 illustrates exemplary animal body segmentation where the body is also segmented in sub-parts, consistent with disclosed embodiments.

FIG. 13 illustrates exemplary segmentation and resulting optical flow after combining the optical flow output with the segmentation, consistent with disclosed embodiments.

FIG. 14 illustrates exemplary segmentation and resulting optical flow after combining the optical flow output with the segmentation, consistent with disclosed embodiments.

FIG. 15 illustrates exemplary Gaussian sampling points on each end of the ellipse model, consistent with disclosed embodiments.

FIG. 16 illustrates an exemplary thermal image of a mouse injected with a drug by a thermal camera, consistent with disclosed embodiments.

FIG. 17 illustrates an example of the ocular temperature of the mouse as a function of observation period separated by observations of the subject's fur after the end of the observation period, consistent with disclosed embodiments.

FIG. 18 illustrates an example of differing thermal fur texture between mice injected with vehicle and those injected with a compound, consistent with disclosed embodiments.

FIG. 19 illustrates an exemplary set of measured features, consistent with disclosed embodiments.

FIG. 20 illustrates a series of exemplary signals from two accelerometer channels (A-B) and 4 EEG signal channels, consistent with disclosed embodiments.

FIG. 21 illustrates examples of the pEEG spectral power signature of two different drugs, consistent with disclosed embodiments.

FIG. 22 illustrates an exemplary Recurrent Neural Network (RNN) model architecture for a pEEG drug class classifier), consistent with disclosed embodiments.

FIG. 23 illustrates an exemplary RNN, consistent with disclosed embodiments.

FIG. 24 illustrates an exemplary RNN, consistent with disclosed embodiments.

FIG. 25 illustrates an exemplary output of a trained classifier, consistent with disclosed embodiments.

FIG. 26 illustrates an exemplary segmentation pipeline flow diagram for gait feature extraction, consistent with disclosed embodiments.

FIG. 27 is a graph of coordinates of the body center and HL paw center as a function of frame, consistent with disclosed embodiments.

FIG. 28 illustrates an exemplary silhouette of the subject emphasizing the amplified movements from frames of a corresponding video clip for measuring murine respiration, consistent with disclosed embodiments.

FIG. 29 illustrates a subject's respiration rates as defined by the maximum of the remaining frequencies, consistent with disclosed embodiments.

FIG. 30 is an infrared camera image of the laboratory cage scene in the infrared spectrum including the test subject and the low and high temperature Peltier elements of a Run-Time Thermal Measurement System (RTTMS), consistent with disclosed embodiments.

FIG. 31A illustrates an exemplary thermal assembly attached to the side of a cage, consistent with disclosed embodiments.

FIG. 31B illustrates an exemplary electronic schematic diagram of a RTTMS, consistent with disclosed embodiments.

FIG. 32 illustrates an exemplary fixture of an Infrared Camera Calibration System, consistent with disclosed embodiments.

FIG. 33 illustrates an exemplary Infrared Camera Calibration Software Application graphic user interface (GUI), consistent with disclosed embodiments.

FIG. 34 illustrates an exemplary thermal camera view of water and a thermocouple sensor measuring the water temperature in real time, consistent with disclosed embodiments.

FIG. 35 illustrates an example of calibration testing, consistent with disclosed embodiments.

FIG. 36 illustrates exemplary eye segmentation image processing, consistent with disclosed embodiments.

FIG. 37 illustrates an exemplary measurement of a decrease of estimated eye temperature, consistent with disclosed embodiments.

FIG. 38 is an exemplary graph showing an average eye temperature for different classes of psychoactive compounds, measured consistent with disclosed embodiments.

FIG. 39 is an exemplary graph showing dose-dependent increase of eye temperature as measured by an automated thermographic system of a behavioral platform, consistent with disclosed embodiments.

FIG. 40 illustrates a block diagram of an exemplary computer-based system for dynamically modifying a database schema to create a mapping of a class label and/or sub-class label to a manipulation within a database of data sets, consistent with disclosed embodiments.

FIG. 41 illustrates a flowchart for dynamically modifying a database schema to create a mapping from a class label and/or sub-class label to a manipulation within a database of data sets, consistent with disclosed embodiments.

FIG. 41A illustrates distribution of exemplary features before normalization, consistent with disclosed embodiments.

FIG. 42B illustrates distribution of exemplary features after normalization, consistent with disclosed embodiments.

FIG. 43 illustrates an exemplary fully connected model architecture for outlier removal, consistent with disclosed embodiments.

FIG. 44 illustrates an exemplary fully connected model architecture for a hybrid hierarchical classifier, consistent with disclosed embodiments.

FIG. 45 illustrates an exemplary RNN model architecture for binary drug activity classifier, consistent with disclosed embodiments.

FIG. 46 illustrates an exemplary visualization output of binary discrimination in the ranked de-correlated feature space, consistent with disclosed embodiments.

FIG. 47 illustrates an exemplary representation of feature values and ranking, consistent with disclosed embodiments.

FIG. 48 illustrates an exemplary de-correlated feature space coordinate system for assessing the recovery of different features that separate the Control and the Model groups, consistent with disclosed embodiments.

FIG. 49 illustrates an overlap of relative fold changes between Control versus Model (black bars) and Control vs Treated Model group (white bars) showing recovery of various features following drug treatment, consistent with disclosed embodiments.

FIG. 50 illustrates assessing statistical significance of variability between control and disease groups in the de-correlated feature space, consistent with disclosed embodiments.

FIG. 51 illustrates exemplary DRFA analysis of the TG4510 model of Alzheimer's Disease, consistent with disclosed embodiments.

FIG. 52 illustrates exemplary DRFA analysis of the TSC1 mouse Model of Tuberous Sclerosis Complex, consistent with disclosed embodiments.

FIG. 53 illustrates exemplary DRFA analysis of the Mecp2 model of Rett syndrome, consistent with disclosed embodiments.

FIGS. 54A-54D illustrate Cube phenotypic signature in the LacQ140I(*) mouse model, consistent with disclosed embodiments.

FIG. 55 illustrates an exemplary overall process for using subject feature profiles as phenotypes for drug discovery, consistent with disclosed embodiments.

FIG. 56 illustrates an example of a target signature for the TG4510 transgenic model of Alzheimer's Disease at 6 months of age, consistent with disclosed embodiments.

FIGS. 57A-57C illustrate exemplary determination of an ES score using a target signature and a list signature in three different scenarios, consistent with disclosed embodiments.

FIG. 58 is an exemplary GSEA plot illustrating the leading edge of the positive enrichment, consistent with disclosed embodiments.

FIG. 59 illustrates exemplary signatures of top compounds similar to Scopolamine, consistent with disclosed embodiments.

FIG. 60 illustrates exemplary signatures of top compounds with reverse list signature to Scopolamine, consistent with disclosed embodiments.

FIG. 61 illustrates Recognition Index for saline, galantamine, and 3 novel compounds after 3 minutes of exploration of a novel and a familiar object, consistent with disclosed embodiments.

FIG. 62 illustrates effects of Tianeptine treatment on Q175 HET mice treated acutely or chronically with Tianeptine or saline, consistent with disclosed embodiments.

FIG. 63 illustrates an overview of an exemplary Drug-induced Behavioral Signature Recovery (DBSA) method, consistent with disclosed embodiments.

FIG. 64 illustrates a total activity as a function of dose for the psychedelic drug lysergic acid diethylamide (LSD), consistent with disclosed embodiments.

FIG. 65 illustrates a n×m similarity data matrix, consistent with disclosed embodiments.

FIG. 66 illustrates a histogram of DDTT diagonal indices as a function of their ranks, consistent with disclosed embodiments.

FIG. 67 illustrates eight different compounds from a library of novel compounds and their “psychedelic” profile using spider graphs to represent all the diagonal indexes DDTR obtained comparing the test drug (T) against the psychedelic drugs (R), consistent with disclosed embodiments.

FIG. 68 illustrates the Class signatures of the Racemate Compound I and its two enantiomers, consistent with disclosed embodiments.

FIG. 69 illustrates long lasting antidepressant-like effects of ketamine and Enantiomer I2 of Racemate Compound I, consistent with disclosed embodiments.

FIG. 70 illustrates an exemplary network visualization, consistent with disclosed embodiments.

FIG. 71 is a response curve for R-(−)-4-iodo-2,5-dimethoxyamphetamine (DOI), consistent with disclosed embodiments.

FIG. 72A shows frames of a video clip showing a DOI-induced head twitch response (HTR), consistent with disclosed embodiments.

FIG. 72B illustrates combined mouse silhouettes from the FIG. 72A image frames, consistent with disclosed embodiments.

FIG. 73A shows an image frame sequence from a captured video illustrating ear scratch behavior with forepaw (ESF), consistent with disclosed embodiments.

FIG. 73B shows combined silhouettes from the image frame sequence depicted in FIG. 73A, consistent with disclosed embodiments.

FIG. 74A shows an image frame sequence from a captured video illustrating ear scratch behavior with hindpaw (ESH), consistent with disclosed embodiments.

FIG. 74B shows combined silhouettes from the image frame sequence depicted in FIG. 74A, consistent with disclosed embodiments.

FIG. 75A shows an image frame sequence from a captured video illustrating rapid elongation-contraction of body (ELO) behavior associated with lysergic acid diethylamide (LSD) treatment, consistent with disclosed embodiments.

FIG. 75B shows combined silhouettes from the image frame sequence depicted in FIG. 75A, consistent with disclosed embodiments.

FIG. 76A illustrates a quantification of psychedelic-related behaviors detected in behavioral platform videos for LSD and DOI at a first dose, consistent with disclosed embodiments.

FIG. 76B illustrates a quantification of psychedelic-related behaviors detected in behavioral platform videos for LSD and DOI at a second dose, consistent with disclosed embodiments.

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that some embodiments may be utilized and that changes may be made without departing from the scope of the disclosed embodiments. Thus, the materials, methods, and examples are illustrative only and are not intended to be necessarily limiting.

In some embodiments, illustrative computing devices of present disclosure provide methods to gather information regarding the amount and type of movement of a simple or complex object using a device such as a camera to infer health status, physiological processes, behavioral process, including drug effects, compound effects, genetic manipulation effects, optogenetic manipulation effects and recovery thereof. The embodiments described herein can include methods to capture instant features. The disclosed embodiments can also infer higher order functional features such as states, motifs, and domains. In some embodiments, the higher-order features can be inferred using a hierarchical processing system, where lower-level features are used to generate higher level features.

In some embodiments, illustrative computing devices of present disclosure provide robust methods to process and analyze the features to form phenotypes of behavioral signatures for comparing and discovering new manipulations and/or combinations of manipulations. Embodiments of the present disclosure provide machine learning models, statistical analyses, similarity analyses, among other analyses and devices configured therefore to use the features of the device to improve manipulation discovery and assessment.

Advances in technology and artificial intelligence provide an opportunity to capture objective subtle behavioral and other effects. Artificial intelligence can be used to understand the effect of drugs in healthy animals and animal models of disease. The embodiments herein describe systems and methods that can provide an objective, detailed, and comprehensive analysis of animal behavior for the development of therapies for a myriad of health conditions.

In some embodiments, the system can be used for the development of drugs using a phenotypic approach that enables discovery of novel compounds with novel polypharmacological action. Medicinal and computational chemistry structure-activity-relationship (SAR) techniques can then be applied to these novel compounds. Analogs of these novel compounds can be designed and tested as part of a development process that may culminate in a novel lead compound.

In some embodiments, the system can be used to find novel compounds that mimic the pharmacological effect of a marketed drug for a particular indication. The system can include integration of behavioral with other data, such as electroencephalographic (EEG) or physiological data, and the use of healthy subjects such as wild type mice, or animal models of disease.

In some embodiments, the systems and methods can be used to identify a mechanism of action of a novel molecule using a phenotypic comparison against reference drugs characterized in the system.

In some embodiments, the systems can also be used to design drugs de novo. For example, the system can be used to design drugs through generative models that can include varying aspects of compounds, including SMILES strings to molecular graphs, 2D and 3D molecular conformation, structural descriptors, physicochemical properties and in vitro, and ex vivo drug effects.

In some embodiments, the system can predict that the action of certain combinations of compounds can be superior to the action of each compound in isolation (e.g., in general or for a particular disease, disorder, syndrome, or symptom, or combination thereof).

In some embodiments, the system can predict that a particular compound, novel or already in the market, can be desirable in general or for a particular disease, disorder, syndrome, or symptom, or combination thereof.

In some embodiments, the system can predict that a particular compound, novel or already in the market, will prove to have a particular pharmacological action at a particular receptor or other target, including whether the action is activating or blocking such target, its potency, and potential biomarkers that can track target engagement.

In some embodiments, the system can predict that a particular compound, novel or already in the market, will have a particular action in a particular neural circuit.

In some embodiments, the system can be used to identify a compound of interest for a particular therapeutic action and at the same time that can quantify an undesirable characteristic, such as the potential for both antidepressant action and hallucinogenic potential.

In some embodiments, the system can be used to identify a compound of interest as an initial hit for a novel chemical series as part of an SAR. In such endeavor the system can be used to ensure that new analogs in the chemical series maintain the desired in vivo profile. As an example, the pharmacokinetics (PK) of a compound can be optimized by synthesis of novel compounds that are filtered out if they produce a different signature. In a different example, the hallucinogenic potential of a compound can be removed by SAR while maintaining the antidepressant signature in the system.

In some embodiments, the system can predict that a particular compound, novel or already in the market, will have a particular action in a particular follow up preclinical test.

In some embodiments, the system can be used to assess, describe, and quantify the phenotype of an animal model of disease, wherein the model is created by genetic, optogenetic, pharmacological, electroconvulsive, or any other manipulation and combination thereof.

In some embodiments, the system can be used to investigate if a compound, such as an antagonist at a receptor, can block the effects of another compound, such as an agonist at such receptor. The system can also identify synergism and potentiation of compound combinations.

In some embodiments, the system can detect observational features characterizing the effect of a drug on a subject (e.g., a rodent), in a way that enables detection of subtle features of importance for disease and drug effects, in addition to other measurable features. The detected features can be integrated into a hierarchy, with lower-level features being used to generate higher-level features that capture more complex behaviors or physiological effects, or behaviors.

In some embodiments, illustrative computing devices of present disclosure can be programmed with one or more techniques of the present disclosure and can be utilized in conjunction with various behavioral platforms utilizing one or more sensors, alone or in combination, to detect observational features of in vivo subjects. In some embodiments, such behavioral platforms can include, e.g., SmartCube® (SC) (PsychoGenics Inc., NJ), NeuroCube® (NC)(PsychoGenics Inc., NJ), eCube (EC)(PsychoGenics Inc., NJ), PhenoCube (PC)(PsychoGenics Inc., NJ). For example, a behavioral platform can employ computer vision and sensors to detect spontaneous and evoked or elicited behavior responses. In some embodiments, a behavioral platform can detect spontaneous and evoked behavior eliciting responses through anxiogenic, startling and other types of stimuli without limitation. In some embodiments, a behavioral platform can be configured to acquire 100, 500, 1000, 2000, or more high-level features, and over about 1 million, such as over about 2 million or about 10 million, instant features. In some embodiments, a behavioral platform can obtain behavioral data concerning locomotion, trajectory complexity, body posture and shape, simple behaviors, behavioral sequences, or any combination thereof. As an additional example a behavioral platform can employ computer vision techniques to extract behavioral features concerning gait (e.g., gait geometry and gait dynamics such as stance, swing, propulsion, or the like) from image data. Such behavioral features can be used, for example, in rodent models of neurological disorders, pain and neuropathies. As described herein, such computer vision techniques can also be used to extract features representing non-gait behaviors.

Consistent with disclosed embodiments, a behavioral platform can acquire observational data concerning a subject. The observational data can include subject data that concerns the subject directly, such as behavioral data or physiological data, and external data that concerns the environment in which the subject exists (e.g., whether a stimulus has been applied to the subject). The behavioral platform can extract observational features from the observational data. These observational features can include subject features extracted from subject data (e.g., behavioral features extracted from behavioral data, physiological features extracted from physiological data, or the like), or external features extracted from external data (e.g., a Boolean variable indicating application of an electric shock or flashing of a strobe light). Higher-level features can be generated from lower-level features (of the same type, e.g., a set or sequence of multiple behavioral features having a particular temporal relationship) or differing types (e.g., a combination of behavioral and physiological features indicating a state, like fear or cold). High-level features can also be generated from lower-level features in combination with observational data. Consistent with disclosed embodiments, the observational features can be used for various analyses as detailed herein. While described with regards to a particular behavioral platform, the disclosed analyses are not so limited. Other devices, systems, and/or platforms for generating suitable observational features can be used.

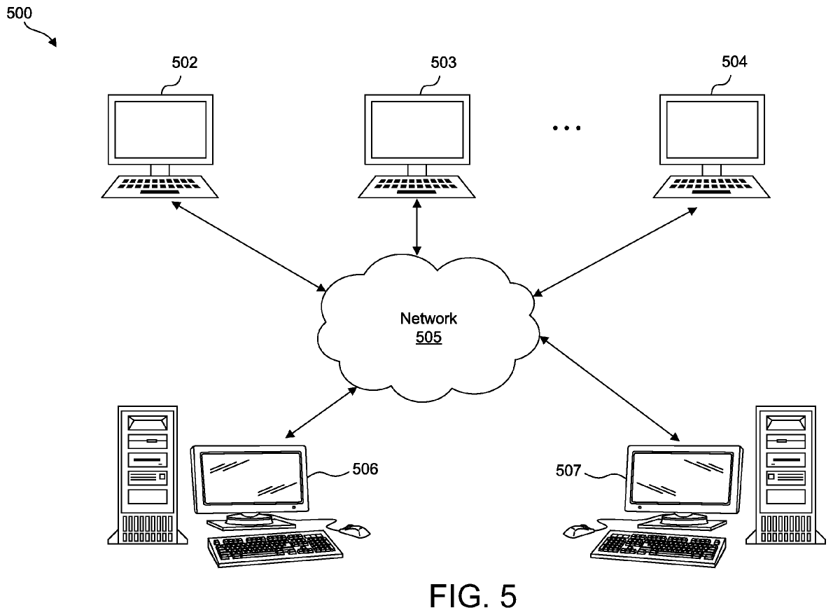

Consistent with disclosed embodiments, observational data acquired (and/or observational features generated) by a behavioral platform can be analyzed by one or more other computing systems. Such computing systems could include a tablet, laptop, desktop, mainframe, cluster, or cloud computing platform. Such computing systems can be communicatively connected to the behavioral platform. Such computing systems may perform data acquisition and/or analysis automatically. The system could be remote from the behavioral platform (e.g., connected over a network link to the behavioral platform). Alternatively, a user of the behavioral platform could transfer observational data (and/or observational features) from the behavioral platform to the computing device manually (e.g., using a non-transitory computer readable medium, such as a USB drive, optical disk, magnetic disk, or by data entry through a user interface). Such a computing system can be implemented using the platform described below with respect to FIG. 5. For clarity of discussion, the analysis of observational data or observational features is described herein with reference to the behavioral platform. However, consistent with disclosed embodiments, some or all of that analysis can be performed by the one or more other computing systems. Accordingly, the disclosed embodiments are not limited to embodiments that perform observational data acquisition, observational feature generation, and observational feature analysis on a behavioral platform.

The disclosed embodiments address multiple technical problems that arise in the context of behavioral analysis (e.g., for drug development or discovery, or other suitable use). For example, independent analysis of observational features acquired using a behavioral platform (e.g., comparison of the behavior features across phenotypes using differential statistics, or the like) may not reveal the overall underlying phenotype or phenotypic relational properties (e.g., observational features differences, similarities, or synergies). Disclosed embodiments can address this technical problem by providing computational methods for in vivo drug discovery using large-scale datasets. These computational methods can use correlative analysis to perform drug screening. The correlative analysis can use a wide collection of observational data following treatment with a drug or compound together with machine-learning models to discover functional connections between the drug or compound and diseases, disorders, or dysfunctions.

In some embodiments, illustrative computing devices of present disclosure can be programmed with one or more techniques of the present disclosure to produce, process and utilize observational data derived from one or more sensors and/or other suitable measurement devices. Observational data can be high-content such that the behavioral data includes, e.g., 300 or more features, 400 or more features, 500 or more features, 600 or more features, 700 or more features, 800 or more features, 900 or more features, 1000 or more features, or other suitable number of features. The term modification and manipulation are used herein to refer to a treatment; administration of a drug or compound; a disease, disorder, or dysfunction, or injury; a genetic difference, a surgical treatment, or the like. In some embodiments, the number of observational features derived from the observational data can be selected to be sufficient to capture the effects of a modification (e.g., to predict functional connections among drugs or between drugs and diseases).

Consistent with disclosed embodiments, phenotypes can be formed from one or more extracted observational features. The observational features can be extracted (e.g., using one or more feature extractors) from sensor or measurement data, or the like. Consistent with disclosed embodiments, feature extractors can include at least one instant features extractor, at least one state features extractor, at least one motif features extractor and/or at least one domain extractor.

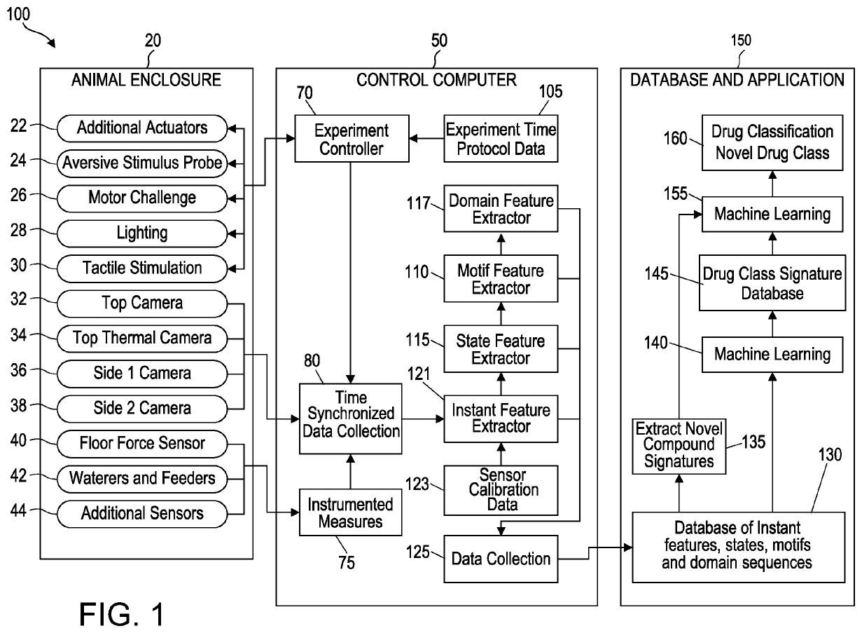

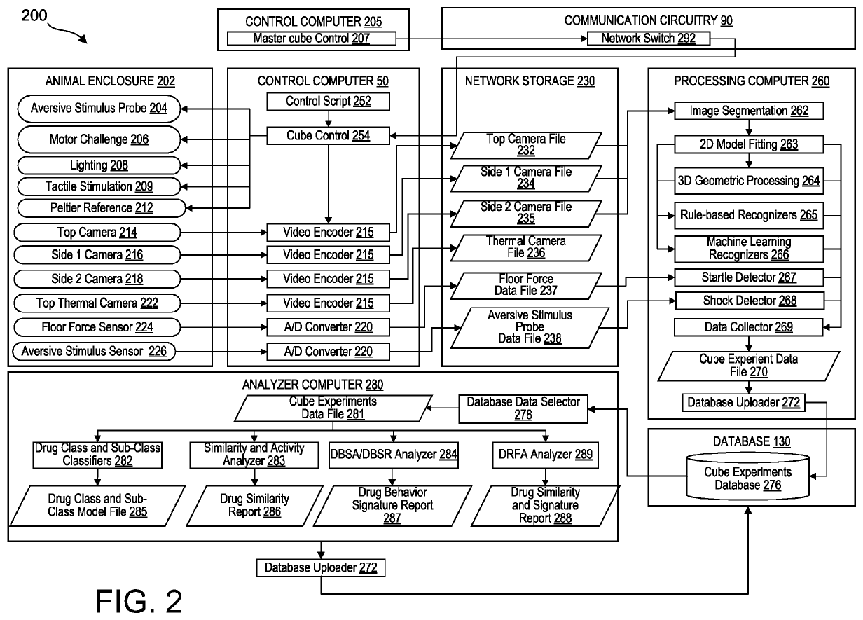

FIGS. 1 and 2 illustrate exemplary data processing flow diagrams for behavioral treatment discovery systems, consistent with disclosed embodiments. Flow diagram 200 illustrates a front end and a back end of a behavioral platform. In the exemplary embodiment shown in FIG. 2, a computer 205 can execute a cube control 207 module. The instructions from cube control 207 module can be relayed using communication circuitry 90 to enclosure control software 254 executed by computer 250. Enclosure control software 254 can control a plurality of actuator devices in animal enclosure 202. The actuator devices can provide stimuli to a subject in accordance with the experimental plan. Actuator devices can include, for example, an aversive stimulus probe 204, a motor challenge 206, lighting 208 and tactile stimulation 209.

In some embodiments, the plurality of sensors can include sensors associated with actuators. These sensors can be configured to capture specific responses to the associated actuators. In some embodiments, the plurality of sensors can be combined with multiple actuators to challenge the test subject to react to various events which are recorded and analyzed by the system. The plurality of sensors can monitor the subject and/or measurement parameters related to the enclosure 20. Exemplary sensors include, for example, a top 2D or 3D camera 214, a 2D or 3D side 1 camera 216, a 2D or 3D side 2 camera 218, a floor force sensor 224, an aversive stimulus sensor 226, a top thermal camera 222, and any combination thereof without limitation. Other arrangements are also contemplated. Exemplary sensor arrangements are described herein with regards to Sections A, B, and C.

The output signal data from these sensors can then be relayed to at least one video encoder 215 or at least one analog-to-digital (A/D) converter 220 in computer 250 as shown in FIG. 2. In some embodiments, the output data from the at least one video encoder 215 and the at least one A/D converter 220 can be stored in a network storage 230, for example, as a top camera file 232, a Side 1 camera file 234, a Side 2 camera file 235, a floor force data file 237, and an aversive stimulus probe data file 238. A may be appreciated, camera files 232, 234 and 235 can be MPEG files, or any other suitable file format for storing image data (or video data).

In some embodiments, the top camera file 232, the Side 1 camera file 234, and the Side 2 camera file 235 can be relayed to an image segmentation module 262 in a processing computer 260 in FIG. 2. The image segmentation module 262 can analyze the files by applying the data from the image segmentation module 262 to a 2D Model Fitting module 263 and a 3D Geometric processing module 264. In some instances, data output from the modules can include instant features that can be applied to a hardcoded, or rule-based recognizers module 265 and/or a machine-learning recognizers module 266 to extract observational features. As an additional example, the floor force data file can be relayed to a startle detector, and the aversive stimulus probe data file can be relayed to an aversive stimulus detector.

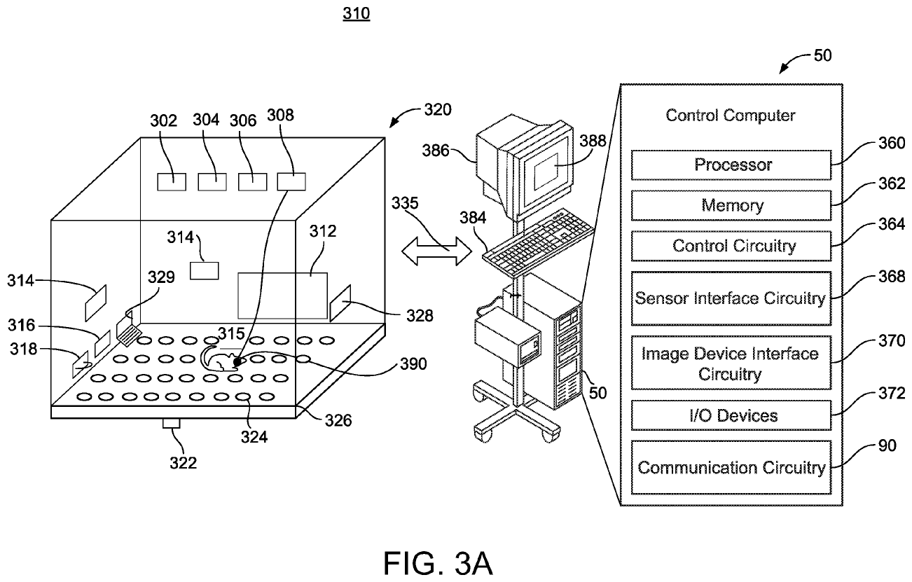

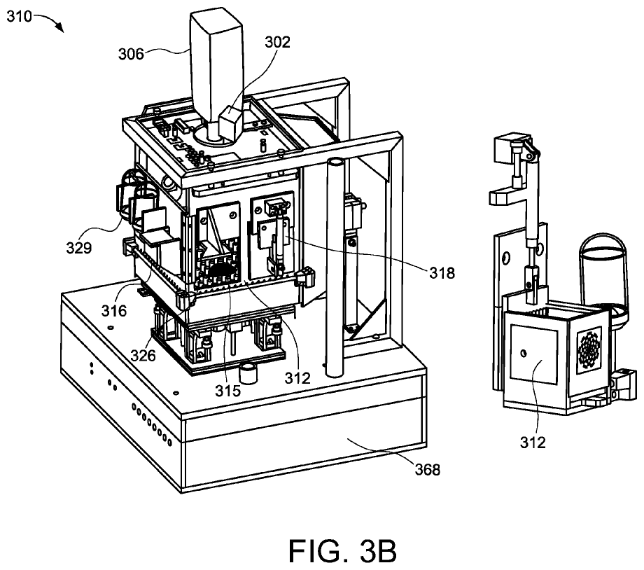

Consistent with disclosed embodiments, the at least one processor 360 in FIG. 3A or processing computer 260 in FIG. 2 can use one or more of the image segmentation module 262, the rule-based recognizers module 265, or the machine-learning recognizers module 266 to detect respiration and/or respiration cycles using image data. For example, image data from the top camera file 232, the Side 1 camera file 234, and the Side 2 camera file 235, among other data, can be used to detect respiration and/or respiration cycles, as described in Section D.

Consistent with disclosed embodiments, the processing computer 260 can use one or more of the image segmentation module 262, the rule-based recognizers module 265, or the machine-learning recognizers module 266 to detect subject temperature using thermal image data from a top thermal camera 222, as described Section E.

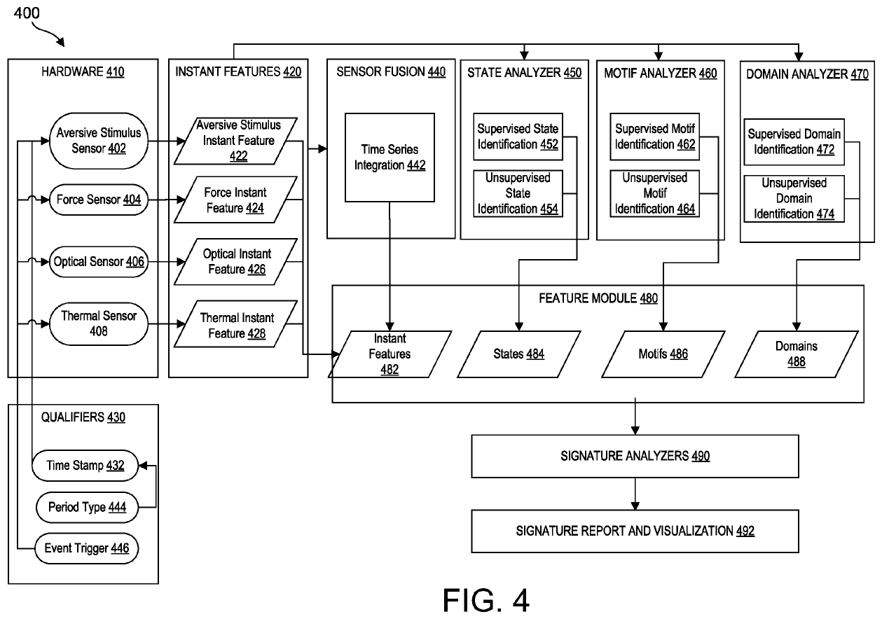

As illustrated in FIGS. 1-2, observational data can be input to a data collection module 125 or data collector 269 and subsequently stored as an experiment data file which can be loaded into database 130. In some embodiments, the database 130 can be communicatively coupled to an analyzer computer 280. A database data selector 278 can receive experimental data file(s), which can be subsequently applied to signature analyzers 490 to generate data (e.g., a subject feature profile, a class or subclass label, similarity score, or the like). This data can be input to signature report and visualization module 492 as shown in FIG. 4. For example, the signature analyzers 490 can include a drug class and subclass classifiers module 282, a similarity and activity analyzer module 283, a drug-induced behavioral signature analysis (DBSA)/drug-induced behavioral signature recovery (DBSR) module 284, and a decorrelated ranked feature analysis (DRFA) module 289, as shown in FIG. 2.

In some embodiments, the drug class and subclass classifiers module 282 can include a suitable machine learning classifier for classifying drugs (or optionally other modifications, such as genetic manipulations, diseases, injuries, or the like) based on observational features. The observational features can be extracted from the observational data described above. In some embodiments, the datasets used to train the machine learning classifiers can differ in the statistical distribution of one or more observational features. Consistent with disclosed embodiments, the datasets can be normalized prior to model training. In some instances, the drug class and subclass classifiers module can be configured to utilize a hybrid classifier as described in Section F.

In some embodiments, the DRFA module 289 can include a suitable analytical and/or machine learning model for scoring and ranking observational features. Observational features can be scored and ranked according to relevance, including ranking the extracted data described above for a particular experimental session. Such an experimental session can be designed to investigate the effect of a subject having particular modification (e.g., administration of a particular dose of a drug to the subject). In some embodiments, the DRFA module 289 can be configured to utilize a support vector machine learning method (e.g., a linear SVM, a nonlinear SVM) to rank each de-correlated feature based on its discrimination power (for example, ability to separate the two groups, e.g., Control and Target) as described in Section G.

In some embodiments, the DBSA/DBSR module 284 can include a suitable analytical and/or machine learning model for screening list signatures stored in database 130, based on subject feature profiles generated from observational data. In some embodiments, the DBSA/DBSR module 284 can be configured to utilize an enrichment technique to compare subject feature profiles obtained during a particular experiment (e.g., involving a particular modification) to stored list signatures (or stored information usable to derive such list signatures) for a library of modifications. The comparison can be based on or use phenotypes constructed from the obtained subject feature profiles. In some embodiments, the DBSA/DBSR module 284 can utilize an enrichment methodology as described in Section H.

In some embodiments, the DBSA/DBSR module 284 can include a suitable analytical and/or machine learning model for screening list signatures stored in database 130. The stored list signatures can be screened based on subject feature profiles generated from observational data. In some embodiments, the DBSA/DBSR module 284 can be configured to use a phenotypic recovery technique to compare subject feature profiles obtained during a particular experiment (e.g., involving a particular modification) to stored list signatures (or stored information usable to derive such list signatures) for a library of modifications. The comparison can be based on or use phenotypes constructed from the obtained subject feature profiles. In some embodiments, the DBSA/DBSR module 284 can utilize a phenotypic recovery methodology as described in Section I.

In some embodiments, the similarity and activity analyzer module 283 can include an analytical and/or machine learning model configured to identify pharmacologically similar modifications. This identification can be based on behavioral effects of each modification as represented by list signatures stored in database 130. In some embodiments, the similarity and activity analyzer module 283 can include a metric (e.g., a distance metric or the like) based on the similarity between two or more sets of observational data. In some embodiments, the metric can be used to find drugs or compounds with pharmacological profiles similar to a target, desired compound or drug. In some embodiments, the metric can be used to rank compounds in a library of compounds according to a similarity between a compound and different reference modifications (e.g., different reference drugs or compounds; or different reference animal models of a disease, disorder, or dysfunction). In some instances, the metric can be used to compare the dose responses of two or more drugs or compounds. In some embodiments, the similarity and activity analyzer module 283 can be configured to use a similarity analysis as described in Section J.

In some embodiments, the drug class and subclass classifiers module 282 can output a drug class and subclass model file 285. The similarity and activity analyzer module 283 can output a drug similarity report 286. The DBSA/DBSR module 284 can output a drug behavior signature report 287. The DRFA module 289 can output a drug similarity and signature report 288.

In some embodiments, the drug class and subclass model file 285, the drug similarity report 286, the drug behavior signature report 287, and the drug similarity and signature report 288 can be uploaded by a database uploader 272 to the database 130.

In some embodiments, each of computer 205, control computer 50, the processing computer 260, and analyzer computer 280 can include at least one processor, at least one memory and/or storage device, I/O devices, and communication circuitry.

FIGS. 3A-3B illustrates an exemplary computer-based system 310 for analyzing movements of an object 315, in accordance with one or more embodiments of the present disclosure. FIG. 4 illustrates a feature engineering data flow diagram in accordance with disclosed embodiments. The feature engineering flow diagram illustrates how the features are obtained in system 310. The at least one processor 360 can process the data generated by the hardware of the enclosure 320 by applying qualifiers (e.g., a time stamp, a period type, an event trigger, or the like) to data from, for example, an aversive stimulus sensor, a force sensor, an optical sensor, or a thermal sensor. The at least one processor 360 can use the output data from these sensors in extract algorithms to extract instant features (e.g., aversive stimulus instant features, force instant features, optical instant features, thermal instant features, or the like). These extracted instant features can be relayed to a sensor fusion module 440, and machine-learning recognizer modules (a state analyzer module 450, a motif analyzer module 460, and a domain analyzer module 470) (See FIG. 4). Examples of suitable sensor fusion modules, state analyzer modules, motif analyzer modules, and domain analyzer modules are presented in Sections A, B, and C, though other analyzers and modules can be employed in any combination.

In some embodiments, behavioral data can be used in the extraction of “Instant Features” as represented by sets of values taken by the measured variables. Observational data can be obtained from an experimental session for data acquisition and/or analysis performed over a predefined duration, analyses of videos can be done frame by frame, or of other time series done at the highest resolution possible. For example, a set of instant behavioral features for a mouse at a given point of time within an experimental session can include the set of x, y, z coordinates and time derivatives (e.g., velocity and acceleration), for its head, body center, paw positions, heart rate and eye temperature.

Consistent with disclosed embodiments, analyses can accept as input instant features and/or higher-order features or modified features generated using instant features. Such higher-order features can include states generated using multiple instant features, motifs generated using states (and optionally instant features), and domains generated using motifs (and optionally states and/or instant features). Accordingly, complex phenotypic models can be generated from the instant features. Consistent with disclosed embodiments, such models can support machine-learning-based investigations into the effects of modifications.

In some embodiments, instant features can be collected and related to object characteristics. A state can be a certain combination of variables that occur with a certain probability. States can be predefined by the user or the literature or discovered by analysis of the probabilities of occurrence of certain variables' values and their combinations. Automated patterns or state discovery may be performed using machine learning models such as hidden Markov Models (HMM), for example. An HMM can evaluate the transition probability between a number of states. The HMM can identify a combination of variables that can be observed, and such combination of variables can be attributed to a state which can be influenced by a Markov process.

Machine-learning models can be trained in a supervised manner to identify predetermined states (e.g., rearing, grooming, locomotion, immobility, freezing, or the like). The confidence of a model can be expressed in terms of a score. Such models can use sensor fusion to identify states. For example, visual data can be combined with EEG or temperature data to identify when a subject is in a sleep state. Machine learning models can also be trained to identify states in a non-supervised manner. For example, increased circling behavior and high temperature can be caused by a drug such as MK801. Such unusual behavior can be discovered with anomaly detection algorithms or other techniques. Such states are not predetermined and can lack predetermined labels or names.

In some embodiments, a motif can be a particular set or sequence of values (e.g., at least two time samples) in a time series stream that occurs with a probability higher than chance. Values can represent instant features and/or states. Transitions between every pair of discrete states can be from one instant feature to another, or also to the same feature. This can be a “first order” motif. In some instances, a set or sequence of several states can occur with a certain probability. Such a set or sequence of “n” states can be labeled an “n-order” motif.

In some embodiments, a motif can be defined apriori and identified in a time series, or a motif can be discovered using unsupervised machine learning methods. As described herein, the time series can include observational data. This observational data can include subject data (e.g., behavioral data, physiological data, or the like) and/or external data (e.g., environmental data, indications of stimuli or rewards presented to the subject, or the like). In some embodiments, a times series can include multiple types of data (e.g., different types of subject data, or a combination of subject data and external data, or the like). In various embodiments, a time series can be specific to a particular type of subject data (e.g., a single kind of behavioral data or physiological data, or the like) or external data.

Consistent with disclosed embodiments, a time series of observational features can be generated. In some embodiments, natural language processing (NLP) can be used for analysis and modeling of such time series data to identify or describe normal, abnormal, and drug-induced changes. In some embodiments, NLP can be used to build a lexicogrammar where the unit of measurement (e.g., features, such as instant features, states, motifs, or domains, or the like) can be understood in the context of a general model that incorporates behavior and drug action occurring at multiple temporal scales and affecting multiple features or combinations of features.

In some embodiments, sensor fusion can be used to identify motifs (e.g., such as the head twitch response), using optical cameras and floor force sensors. For this case, the motif can involve a type of head movement, involving a set or sequence of instant features. A response to an aversive stimulus can trigger a motif that can include an approach to the stimulus, exploration, avoidance, defensive burying response. This can be expected and identified using hardcoded algorithms or supervised machine learning. A motif can be the second highest feature hierarchy below domain.

In some embodiments, a domain can be a particular scenario, area, or type of collected data that can be related to behavioral manifestation, physiological manifestation, or both. A domain can be the highest feature hierarchy. For example, a collection of features representing physiology, such as temperature, can represent a domain. Other domains can be measured by behavioral platforms that can include gait geometry, motor coordination, paw position and paw pressure. Other domains can be exploratory behavior versus consummatory behavior. Domains can be defined using features from the same or across modality such as optical information for overt behavior or thermal information for temperature.

The above hierarchy of features are simply illustrative. Any suitable feature extractor or combination of feature extractors can be employed to form a subject feature profile for a subject (e.g., a test animal) or a group of subjects (e.g., a test animal group). Such a subject or group of subjects can be subject to a modification or can constitute a relevant control group for such a modification. Consistent with disclosed embodiments, the subject feature profile can include any selection or combination of observational features (e.g., instant features, states, motifs, domains, or any combinations thereof). In some embodiments, the subject feature profile can include observational data.

As shown in FIG. 4, in some embodiments, feature module 480 can receive instant features 482, state features 484, motif features 486, and domain features 486. The instant feature module 420 can relay the extracted features to the instant features 482. The sensor fusion module 440 can execute a time series integration module 442 that generates additional instant features that can be relayed to the instant features 482 in feature module 480. The state analyzer module 450 can apply the instant features to a supervised state identification module 452 and/or an unsupervised state identification module 454 to generate the state features that can be relayed to the states features 484 in the feature module 480. The motif analyzer module 460 can apply the instant features to a supervised motif identification module 462 and/or an unsupervised motif identification module 464 to generate the motif features that can be relayed to the motif features 486 in the feature module 480. The domain analyzer module 470 can apply the instant features to a supervised domain identification module 472 and/or an unsupervised domain identification module 474 to generate the domain features that can be relayed to the domain features 488 in feature module 480.

In some embodiments, the instant features 482, state features 484, motif features 486 and domain features 488 in the feature module 480 can be applied to signature analyzers 490. Signature analyzers 490 can generate a subject feature profile based on the experimental plan. The subject feature profile can be input to a signature report and visualization module 492 that can output a signature report and/or visualization on a GUI of a display.

In some embodiments, the signature report and visualization module 492 can be configured to perform one or more statistical methods to quantify and visualize a subject feature profile, by, for example, using network analysis methods, such as Markov Chains, to illustrate the temporal dynamics of identified instant features, states, motifs, and domains. Such visualization can be used to illustrate, for example, the significant changes in network dynamics between two cohorts, such as an animal model of disease and its wild-type control, based on the instant features, states, motifs, and/or domains that differ between the groups of interest. This network visualization can help in understanding the overall feature changes specific to a given cohort relative to a control group. Comparison of these networks in an animal model between treatment to the given cohort and vehicle administered to the control group, for example, can also help in the understanding of the features rectified by, for example, a modification or drug treatment. An example of a suitable statistical model for creating a network representation to visualize the signification changes is described in Section K.

In some embodiments, the signature analyzers 490 can be configured to evaluate behaviors induced by modifications having a particular neurological effect, such as, e.g., seizures, head twitches, straub tail, among others. Such behaviors can be known to be mediated by particular receptors and/or modulated by particular receptors. Thus, the signature analyzers 490 can use the subject feature profile for a library/compound screen to identify modifications having a particular neurological effect based on the subject feature profile. The subject feature profile can include instant features, states, motifs and/or domains. Machine learning models can be trained to automatically identify and add observational features having predictive or discriminative power to a subject feature profile.

FIG. 4 illustrates an example of a drug development method using subject feature profiles, in accordance with disclosed embodiments. In this example, the method is being used to develop effective and long-lasting antidepressant drugs. In this example, the method uses signature analyzers 490 to generate the subject feature profiles and uses the subject feature profiles to identify and/or screen compounds with psychedelic-like signatures.

In some embodiments, the signature analyzers 490 can receive a database data selector 278, e.g., from the database 130 as described above and illustrated in FIG. 2. The database data selector 278 is used to select a dataset representing a compound or set of compounds to be submitted to a “similarity” analysis if the signature, according to the signature analyzers 490, shows the potential for a desired target therapeutic signature.

In some embodiments, such desired target therapeutic signature can be identified by the signature analyzers 490, drug class and subclass classifiers module 282, the DRFA module 289, and/or any other analyzer for evaluating a relationship between the library/compound screen and the desired target therapeutic signature, without limitation. The drug class and sub-class classifiers can include, for example, the hybrid classifier Section F as described herein. The similarity and activity analyzer can, for example, use the similarity model Section J as described herein. The DRFA analyzer can include, for example, the feature ranking model as described in Section G. In some embodiments, the signature analyzers 490 can produce a psychedelic-like signature for a compound. For example, the drug class and sub-class classifiers can produce a psychedelic class and/or subclass based on the classification of the compound's features. The similarity and activity analyzer can produce a “psychemimic” psychedelic profile by the quantification of the similarity between the compound and a panel of psychedelic reference compounds of interest. The DRFA analyzer can determine that the compound belongs to a psychedelic and/or antidepressant drug group based on decorrelated and/or ranked features.

In a representative drug discovery process, the psychedelic signature selection by the system can trigger resynthesis of the compound and reanalysis in the system for confirmation of the signature. In some instances, involving chiral compounds, an additional step of enantiomer separation can support identification of a specific psychedelic-like chemistry. This enantiomer separation can involve the use of specific chemical synthesis routes.

In some embodiments, each modification in the screened library can additionally or alternatively be analyzed with a suitable analytical and/or machine learning model to evaluate behaviors induced by psychedelics (or PDIB, Psychedelic Drug Induced Behaviors), such as head twitch responses (HTRs) and ear scratches (ERs). Such behaviors can be mediated by serotonin 5HT2A receptors and modulated by 5HT2C in animal models. Accordingly, such behaviors can be good predictors of drug-induced hallucinations in healthy human subjects. In some embodiments, a behavioral platform can use a head twitch algorithm that includes one or more analytical and/or machine learning models to automatically detect HTRs and other PDIBs as described in Section L. These detected behavioral features can be used in the analyses as described herein.

Accordingly, the psychedelic-like antidepressant signature identified by the signature analyzers 490 can be complemented by the quantification of PDIBs by the head twitch algorithm. Such complementary characterizations of a compound can result in a compound being marked as a lead for a drug development project. For example, an antidepressant profile devoid of PBIBs, at least for a range of doses, can be a lead hit. Alternatively, there can be no separation between the two assessments, with high PDIBs at various doses tested with an antidepressant signature, which can make this compound suitable for further SAR development, binding assay characterization for assessment for example of 5HT2A activity, or abandonment.

In some embodiments, the psychedelic-like antidepressant compound can be investigated by confirmation assays to determine that the psychedelic-like antidepressant from the manipulations of the screened library shows efficacy in other traditional pre-clinical assays. In some embodiments, the confirmation assays can include, e.g., an antidepressant action, a plasticity assay, biomarkers, a binding profile, among other assays or any suitable combination thereof.

The aforementioned examples are illustrative and not restrictive.

FIG. 5 illustrates a block diagram of an exemplary computer-based system 500 in accordance with disclosed embodiments. As may be appreciated, some embodiments may not include all the illustrated components. Variations in the arrangement and type of the components can be made without departing from the spirit or scope of the disclosed embodiments. In some embodiments, system 500 (or components thereof) can be configured to dynamically modify a database schema to create a mapping of at least one outlier data point to at least one label of a plurality of labels associated with a plurality of chemical compounds, as detailed herein. In some embodiments, the system 500 can be based on a scalable computer and/or network architecture that incorporates varies strategies for assessing the data, caching, searching, and/or database connection pooling. Such a scalable architecture can be capable of operating multiple servers and adding or removing servers in response to demand. In some embodiments, the exemplary inventive computing devices and/or the exemplary inventive computing components of the system 500 can be configured to manage various modules, for example, without limitation, identified in FIGS. 1-4.

In some embodiments, referring to FIG. 5, devices 502-504 (e.g., clients) of the system 500 can include suitable computing devices. Such computing devices can be capable of simultaneously launching a plurality of software applications via a network (e.g., cloud network), such as network 505, to and from another computing device, such as servers 506 and 507, each other, and the like. In some embodiments, the devices 502-504 can be personal computers, multiprocessor systems, microprocessor-based or programmable consumer electronics, network PCs, and the like. In some embodiments, one or more member devices within devices 502-504 can be devices that are capable of connecting using a wired or wireless communication medium such as a laptop, tablet, desktop computer, a netbook, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.).

In some embodiments, one or more member devices within devices 502-504 can be configured for launching one or more applications, such as Internet browsers and mobile applications. In some embodiments, one or more member devices within devices 502-504 can be configured to receive and to send web pages, and the like. In some embodiments, various modules of FIGS. 1-4 can be configured to receive and display graphics, text, multimedia, and the like, employing virtually any web-based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within devices 502-504 can be specifically programmed by either Java, .Net, QT, C, C++ and/or other suitable programming language. In some embodiments, one or more member devices within devices 502-504 can be specifically programmed to include or execute an application to perform a variety of possible tasks, such as, without limitation, messaging functionality, browsing, searching, or displaying various forms of content, including locally stored or uploaded messages, images and/or videos.

In some embodiments, the exemplary network 505 can provide network access, data transport and/or other services to any computing device coupled to it. In some embodiments and, optionally, in combination of any embodiment described above or below, the exemplary network 505 can also include, for instance, at least one of a local area network (LAN), a wide area network (WAN), the Internet, a virtual LAN (VLAN), an enterprise LAN, a layer 3 virtual private network (VPN), an enterprise IP network, or any combination thereof.

In some embodiments and, optionally, in combination of any embodiment described above or below, at least one computer network communication over the exemplary network 505 can be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 505 can also include mass storage, such as network attached storage (NAS), a storage area network (SAN), a content delivery network (CDN) or other forms of computer or machine-readable media.

In some embodiments, the server 506 or the server 507 can be a web server (or a series of servers) running a network operating system, examples of which can include but are not limited to Microsoft Windows Server, Novell NetWare, and/or Linux. In some embodiments, the server 506 or the server 507 can be used for and/or provide cloud and/or network computing.

In some embodiments and, optionally, in combination of any embodiment described above or below, for example, one or more exemplary computing devices 502-504, the server 506, and/or the server 507 can include a specifically programmed module that can be configured to dynamically modify the database schema to create a mapping of the at least one outlier data point to the at least one label of the plurality of labels within the database of data sets associated with the plurality of chemical compounds.

### Section A

In some embodiments, tools to diagnose and monitor disease progression and possible treatment effects in preclinical models and in the clinic can include behavior, movement, activity, posture, and gait analysis and quantification. Optical cameras may be used to capture a 2D or 3D view of the animal from the top, side, or bottom with respect to the subject's body. The use of more than one camera can enable the behavioral platform to gather depth information (e.g., in addition to the 2D view obtained from a single camera). Camera arrangements can be used as disclosed in the embodiments herein as 2D/3D sensor examples, which may leverage systems using other data generation methods that use active electronics illumination devices (such as lamp and laser projectors), or event triggered cameras, also known as a neuromorphic cameras, which can respond to local changes in brightness. As with camera arrangements, the optical information and relative position of those instruments may be used to produce 3D depth information.

In some embodiments, the disclosed embodiments can determine that the subject may be in a particular location, such as in proximity to a particular sensor such as a high-speed high-resolution camera, and such information may be used to enable processing of such sensor's data for identification of particular body, body segments or other subject features.

In some embodiments, stereovision may be used to create 2D or 3D models. The system may use one or more of a number of technologies available to acquire 3D images of a scene. These may include sonar, millimeter wave radar, scanning lasers, structured light, and stereovision. A number of novel technologies that can be available for the embodiments disclosed herein are shown in Table I. As described herein, sensors, and sensor configurations capable of generating 3D data, may be referred to as 3D-sensors. An example of the relative performance of these technologies is summarized in Table I.

TABLE I
Comparison of 3D-sensor technologies

| | | | | | Depth | Detectable by the |
|---|---|---|---|---|---|---|
| Technology | Resolution | Accuracy | Speed | Cost | Accuracy | Experimental Subject |
| Sonar | Very Low | Very | Low | Low | Low | No |
| | | Low | | | | |
| Radar | Very Low | Low | High | High | Low | No |
| Scanning | High | High | Low | High | Very | Sometimes |
| Laser | | | | | High | |
| Structured | Medium | High | Medium | Low | Medium | Sometimes |
| Light | | | | | | |
| Stereovision | High | High | High | Low | Medium | No |
| Time-of- | High | High | High | High | Medium | No, more suitable for |
| Flight | | | | | | long-range |
| Event | High | High | Highest | High | Medium | No |
| Sensors | | | | | | |

The disclosed embodiments are not limited to any particular one, or combination, of the technologies disclosed in Table I. Despite the relative performance of 3D-sensors, continued development for commercial applications of the technologies shown in Table I is constantly shifting the relative performance and cost of different approaches. Machine learning and artificial intelligence methods may use the information generated by various technologies to detect the effect of drugs on animal behaviors as described in the embodiments disclosed herein.

Consistent with disclosed embodiments, sonar and radar may have resolution limitations that hinder use in the envisioned embodiments. Scanning laser range finders can be expensive and/or fragile devices. Systems that use structured light protection may experience difficulty capturing fast motions. Furthermore, the projected pattern of light may be visible (and distracting) to the test subjects. Stereovision based approaches offer multiple benefits. Stereovision based-approaches have a long history of successful use, available 3D-image processing algorithms, low-cost, low-power data acquisition technology, mechanical reliability, and lack of projected light pattern. Furthermore, stereovision-based approaches are flexible in that most of the work of producing a stereo range image is performed by reconfigurable software. Furthermore, stereovision can use images from closely spaced cameras that are typically arranged along a horizontal “baseline.” Images (or full-speed video) are taken simultaneously from all of the cameras. Once a time-synchronized set of images has been taken, it can be converted into a 3D-range image.

Depth information may also be obtained by methods using RGB-D cameras and 3D depth sensors such as the MICROSOFT KINECT, ASUS XTION, and INTEL REALSENSE cameras. Referring to Table I, those sensors can fall into the Structured Light category. The MICROSOFT KINECT, for example, utilizes a combination of an IR projector and two cameras: one IR and one RGB camera. RGB stands for a system that acquires images in three basic colors: red, green, and blue, which can be later combined into an image. In some embodiments, two elements (one passive and one active, or two passive) can be used to triangulate the information to obtain 3D data. As a result, these stereo systems can be made up of two cameras or lenses and one projector, or of one camera that takes several images after changing position. In the KINECT camera, for example, the depth information comes from analysis of the refracted infrared beams, and final image comes from the integration of the data from the two cameras. 3D depth sensors used in some smartphones use time-of-flight (ToF) 3D camera sensors which also use the projection of a patterned light beam array and sense the differentially reflected light by surfaces at different distances. 3D cameras can include simple 3D photos with realistic depth to very accurate 3D depth sensors used in phones, 3D scanners, drones, and tablets.

Consistent with disclosed embodiments, 3D data can be obtained using triangulation of data from two or more Event Sensors. Event Sensors can be designed to capture rapid changes in scene illumination. Rather than being triggered at a constant and fixed rate, event sensors may transmit pixel-level changes at an equivalent of thousands of frames per second. In a 3D configuration, an Event Sensor can first match events locations in 2D images from two or more cameras, then triangulating those between different cameras to obtain the 3D information.

Consistent with disclosed embodiments, information from other sensors can be combined with 3D sensor configuration information. These additional sensors include force sensors such as piezoelectric, infrared detectors, radiofrequency detectors, and the like. In certain applications of the embodiments disclosed herein, a sensor can include both a source of emission such as an infrared beam, a radiofrequency, a source of heat, a source of optical signals, or other such source. The use of two optical cameras can provide depth or 3D information. The particular configuration of sensors can be adapted for optimal capture of information regarding an object (e.g., an animal). Such information can include an instant assessment (namely features, variables, or primitives) of the object's state or a sequence of such assessments in a time series.

As described above, FIGS. 3A-3B illustrates a system 310 for analyzing movements of an object 315, in accordance with one or more embodiments of the present disclosure. System 310 may include a control computer 50 communicating 335 with a computer-controlled enclosure 320 to which a plurality of sensors and/or actuators may be coupled thereon to execute an experiment on object 315, such as a rodent. The rodent may be administered, for example, with a compound in accordance with an experimental plan and observed with the plurality of sensors such that the system 310 may determine the behavioral and/or physiological effects of the administered compound on the object over a predetermined time period (e.g., the experimental session).

In some embodiments, the plurality of sensors and/or actuators coupled to the enclosure may include, but are not limited to, sensors in accordance with the experimental plan for determining the behavioral and/or physiological effects of the administered compound on the object 315 over the predetermined time period. For example, sensors and/or actuators may include an aversive stimulus probe 318 that may be deployed and retracted, a motor challenge 326 that may be deployed or retracted so as to force the object 315 to walk on a plurality of physical obstacles 324 arranged in an array with a predefined pitch to provide a motor challenge, lighting 304 that may be configured to change illumination intensity levels and/or light wavelengths as applied to the object 315, a tactile stimulator 316 to administer tactile stimuli to the object 315, a top camera 306, a top thermal camera 302, a first side camera 328 a second side camera 314, a floor force sensor 322, waterers and feeders 329, and as additional actuators 22 for applying any additional suitable stimuli to the object 315 and/or any additional sensors 44 (see FIG. 1). The enclosure may include social pod 312, only shown as an opening in enclosure 320 in FIGS. 3A-3B to allow the social pod 312 to be affixed to enclosure 320 via the opening. This permits a second subject in the social pod 312 to interact with object 315.

Note that the top camera 306, the first side camera 328, and the second side camera 314 may be a 2D camera, a 3D camera, or any combination thereof. A 3D camera for acquiring 3D image data may include multiple 2D cameras arranged at various positions and angles around enclosure 320 to obtain the 3D image data.

In some embodiments, the first side camera 328 and the second side camera 314 may be oriented to capture images in the computer-controlled enclosure 320 from different perspectives. An angle between the orientation of the first side camera 328 and the second side camera 314 may be any suitable angle to capture movement in all directions throughout the computer-controlled enclosure 320. For example, the angle may be 90 degrees, though other angles may be used, such as, e.g., any angle from about 1 degree to about 179 degrees, such that imagery from both the first side camera 328 and the second side camera 314 may be processed to determine movement in all directions within the computer-controlled enclosure 320.

In some embodiments, the plurality of sensors may include sensors associated with some actuators to capture specific responses to the actuators. In some embodiments, the plurality of sensors may be combined with multiple actuators to challenge the test subject to react to various events, which are recorded and analyzed by the system. The resulting ethophysiogram, or collection of physiological and behavioral responses, may create a dataset (e.g., a content-rich dataset) suitable for use with the disclosed systems and methods.

As used herein, object can be a subject may be used interchangeably herein. In the context of the present disclosure, the object or subject may refer to a rodent, for example.

The control computer 50 may include at least one processor 360 for executing any suitable computer applications as described later herein for performing the analysis of the movements of the object 315, at least one memory and/or suitable storage device denoted by memory 362 for storing the computer code and any databases used in the analyses of the acquired data over the predetermined time period, control circuitry 364 for controlling the plurality of actuators in accordance with the experimental plan, sensor interface circuitry 368 for outputting data from the plurality of sensors, image device interface circuitry 370 for receiving the output data from any or all of the cameras and/or thermal cameras, input and output (I/O) devices 85, and/or communication circuitry 90 to enable the control computer 50 to communicate over any suitable communication network.

In some embodiments, the I/O devices 372 may include, for example, a display 386 and/or a keyboard 384. Keyboard 384 may allow a user or operator of system 310 to input data to the control computer 50. The at least one processor 360 may control a graphic user interface (GUI) 388 displayed on the display 386. The GUI 388 may display any suitable parameters and/or data visualizations related to the experimental session and/or results of analyses of the data acquired by the plurality of sensors coupled to enclosure 320 in accordance with the experimental plan.

In some embodiments, experimental sessions for the same dose of the same drug may be repeated to provide confirmation, replace old data, replace outliers, or add more data for other purposes without limitation. A particular experimental session may be applied to a few subjects such as 5-6 mice, or 8 mice, and may be applied to as many as 100 mice per dose tested, for example.

Regarding 3D sensors selection criteria, the selection of sensors, with and without active illumination, may be driven by design criteria including 3D sensing range and view angle. The size and shape of the test subjects, together with the dimensions of the test enclosures, may be considered when designing and selecting the 3D sensors.

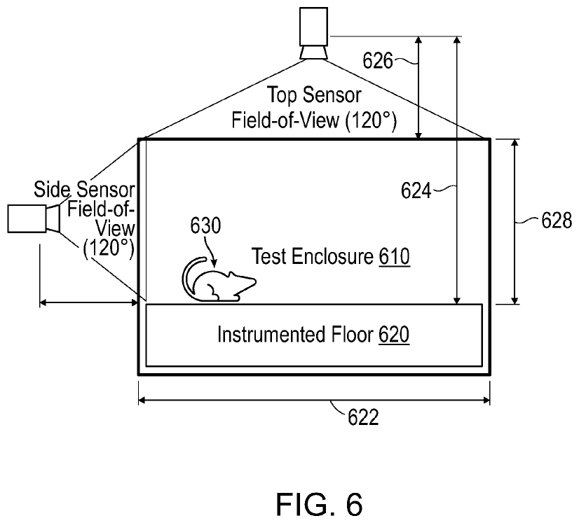

FIG. 6 illustrates an exemplary enclosure with a sensor's field-of-view, location, and minimum distance, in accordance with one or more embodiments of the present disclosure. FIG. 6 illustrates the balance between sensor location, the minimum sensor range and field-of-view. Small rodent applications as shown in FIG. 6 may need sensing ranges between 5 and 30 cm, but for larger animals such as for a rat, may need sensing ranges between 50 to 200 cm. In other cases, large animal pens for dogs may need ranges between 2 to 20 m. The individual and aggregate view angles provided by all sensors may be selected to provide full coverage of the enclosure from the view angles that are critical to detect the animal or animals' instant features, states, actions or behaviors.

In some embodiments, for small rodents, an enclosure 610 may be a square box with length 622 and height 628. For example, enclosure 610 may have dimensions of approximately 30 centimeters by 30 centimeters by 15 centimeters high. A top 3D sensor with a field of view of 120 degrees may be located at height 626 above the enclosure floor. For example, height 626 may be approximately 23.7 centimeters above the enclosure floor. Side 3D sensors of the same field of view may be located a distance 629 from the side glass wall. For example, side 3D sensors can be 4.3 centimeters away from a side glass wall, allowing 3D measurements from the side of the enclosure. The minimum sensing range for such an arrangement may be about 1 cm, about 2 cm, about 3 cm, about 4.33 cm, e.g., the closest distance between the animal 630 and any of the 3D sensors (here the side view sensor), with a maximum of no less than 34.33 cm.

Another design criteria can be 2D and 3D data resolution. For 3D sensors, a volumetric pixel may be used as one unique unit of 3D data. The volumetric pixel is the smallest piece of data that may be changed or edited in 3D. Resolution may be based on three factors: the total number of pixels in each the x, y, and the z direction. More resolution (more pixels) may produce more image detail in the 3D volume of data. Also, higher resolution 3D data may improve the ability of the imaging device to resolve two points that are close together in distance. The higher the resolution, the smaller the detail that may be resolved from an object. Note that the pixel resolution may not be equal in all directions, and the range (z-direction) may be typically less as compared to the x and y directions. For the applications disclosed herein, the 3D data resolution measure may dictate how fine or coarse body parts of the animal may be observed. For example, the general 3D contour of an adult small rodent may involve a pixel resolution of approximately 5 pixels per centimeter (body length 10 cm), as compared to 33 pixels per centimeter for its tail (with diameter ranging from 4-6 mm at the tail base to from 1-1.5 mm at the tip in a mouse, much larger in a rat), and a much higher pixel density of 125 pixels per centimeter to fully detect its whiskers (30-180 μm diameter in rats and less than 60 μm in mice).

In some embodiments as disclosed herein, the sampling rate of a sensor may be set to 1000 Hz or more, so as to capture features of very short duration (e.g., short-duration instant features, states, motifs, or domains), as exemplified below using an EEG signal. Recording behavior and physiology with a high sampling rate can increase statistical power and can capture many aspects of behavior in standard behavioral tests.

The sensor frame rate, expressed as frames per second (fps), can be the number of 3D frames the sensor may acquire per second. Lower frame rates may result in choppy or broken movement but may be less critical for temporal computer vision 3D features that occur at slower rate, such as animal locomotion or rearing. Fast moving actions, such as animal digging and grooming, may need 30 fps or more in order to capture instant features, such as paw location.

The sensor integration time and exposure may be factors limiting the maximum frames per second that a 3D sensor may provide. Integration can define the interval during which the sensor's clocks are set to capture and retain charge. The integration can be delimited by internal electronics. The exposure can be the interval during which the detector may be exposed to incident light by the shutter. The effective exposure can be the interval in which the 3D sensor is both exposing and integrating. For multi-modality sensors, the final frame rate can be determined by the longest effective exposure of the sensing modalities.

While capturing fast motions, long exposure may allow the subject to move through the frame and to blur the 2D/3D pixels. Blurred 3D pixels can reduce the effective data resolution and may add uncertainty to the extraction of instant features. Adjustments in the intensity of the background illumination may be performed to reduce exposure and therefore increase the overall frame rate. Grooming and other behaviors in small rodents can be detected at 30 Hz frame rate, but the tracking of specific fast mouse paw or head movements may need high frame rates

The term “multispectral data” can refer to observational data obtained by sensors capable of capturing light in the visible to infrared spectral bands. The wavelength range, or spectral sensitivity, may be optimized for 400-1100 nm (310 nm UV, and 1100 nm NIR). Sensors operating in the visible range can be used in most commercial cameras. Visible sensors may be well-suited for segmenting rodent images within a test enclosure. In some embodiments, there should be sufficient color contrast between the animal's coat and the enclosure. The required contrast value may be dictated by the signal-to-noise ratio (SNR) for the pixel intensity. The required SNR level for segmentation may be driven by factors such as the feature size, motion pattern, and industry standards such as a SNR thresholds of 40:1 or 10:1.

In some embodiments, thermal data may be acquired. Rodents and humans have a similar warm core body temperature (median of 37.0° C. in humans and 36.6° C. in mice). Most of the radiation emitted by the (human) body is in the infrared region, mainly at the wavelength of 12 micron. The wavelength of infrared radiation may be between 0.75 to 1000 micron. Multispectral or specifically IR sensors therefore may be used to measure body temperature, which may be processed together with other optical 3D sensor data or may be processed separately.

In some embodiments, the location of the 3D-sensor can be selected to minimize the potential for disruptive occlusion of body parts or portions thereof during experiments. Missing 3D data concerning the occluded body parts or portions thereof may potentially limit the ability to accurately detect certain behavior. For example, frontal grooming may be identified when an animal uses its front paws to rub its face. This action may be easily observed from systems with one or more sensors located at the floor level. However, an overhead sensor may have a partially occluded view of the front paws by the animal's head, preventing accurate detection of this behavior.

Exploration in rodents is an important aspect of their normal behavior. Exploration may be expressed by not only patterns of locomotion and rearing, which may bring the subject in proximity to different areas of their habitat, but also by the animals sensing the environment through visual, tactile, and olfactory methods. Movements of the head in the vertical direction (“sniffing”) and horizontal (“scanning”) may both be indicative of exploratory behavior in response to a novel environment, spontaneous behavior, or behavior related to the sensing of olfactory stimuli. An optimal placement and configuration of the 3D-sensors can assist in capturing the movement of the head accurately and equally within the test enclosure.

As described above, FIG. 1 illustrates a data flow pipeline 100 suitable for system 310, in accordance with disclosed embodiment. This pipeline can be executed, for example, by the at least one processor 360 of the control computer 50 (FIG. 3A). System 310 may use computer vision using deep learning for generating observational features (e.g., subject features) as shown in the exemplary data flow pipeline 100 of FIG. 1. Control computer 50 may include an experiment controller 70 (e.g., the control circuitry 364 of FIG. 3A) that may receive commands from experiment time protocol data 105 to control the plurality of actuators as shown in FIG. 3A. The plurality of sensors (e.g., 2D cameras and/or 3D cameras and/or thermal cameras) may send output data to the image device interface circuitry 370 configured to perform time synchronized data collection. Additional sensors may output data to the sensor interface circuitry 368 also referred to instrumented measurers.

In some embodiments, the information from the output data of the sensors may be inputted to at least one extractor machine learning model. These may include at least one instant feature extractor 121, at least one state feature extractor 115, at least one motif feature extractor 110, and/or at least one domain feature extractor 117. Sensor calibration data 123 may be used by the algorithms and/or machine learning models to extract the instant features, state features and/or motif features.

In some embodiments, at least some of the extracted observational features can be applied to a data collection module 125 to query a database 130 of features to identify a target signature associated with a target condition where the subject may exhibit a plurality of target features representative of the target condition. The outputted target signature can be inputted to a first machine learning model 140 whose output may be used to query a drug class signature database 145. In parallel, the at least one processor 360 may extract novel compound features 135 from the outputted target signature from the database 130. In some embodiments, the output of the drug class signature database 145 and the extracted novel compound features 135 can be inputted to a second machine learning model 155 to output a drug classification 160 for novel drug class.

In some embodiments, the data flow pipeline 100 shown in FIG. 1 may include a segmentation model to identify the object 315 and its parts. In some embodiments, the object 315 may be an animal. In some embodiments, object 315 may be a rodent such as a mouse or rat. In some embodiments, the at least one processor 360 can perform operations to segment body parts (e.g., the head, body, tail, or the like) of an animal, in addition to identifying the entire animal as a single entity.

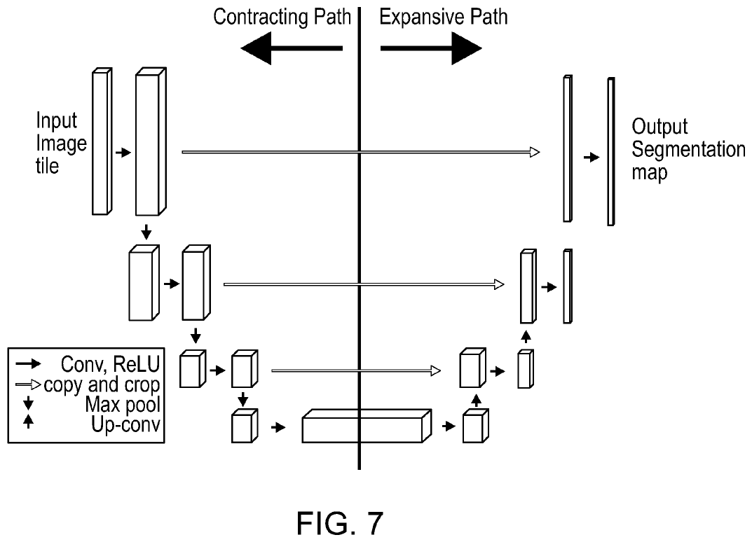

In some embodiments, segmentation models may be used to identify the object and separate it from the background (e.g., a U-net convolutional neural network architecture, or another suitable model). A nested U-net may include several U-net layers as shown in FIG. 7.

In FIG. 7, the contracting path (left) may use convolutions and maximum pooling to reduce the image data into a vector. The expansive path (right) may use convolution and up-convolution, while re-using data from the left path, to obtain a final segmentation image with the same resolution as the input image.

In some embodiments, models for segmentation may be trained using ground truth, which are labeled images, videos, or other data, labeled by a trusted agent. The trusted agent can be a human or a trained model. In some embodiments for video data, an accuracy metric may be used such as the IoU (intersection over union) of the predicted segmented pixels with the ground truth segmented pixels in the validation images. During training, the model may undergo cross fold validation (at least 5 and preferably up to 1000 or more), where a percentage of data (preferably between 5 and 25%) of the data may be randomly sampled evenly amongst each view and set aside as a test set. Once a model achieves at least a level of accuracy, such as about 80% to about 95% accuracy, during cross validation, the final model may be tested with the test set to assess accuracy. The entire dataset may then be used before the model is used in a production pipeline to achieve the best results. Failures of intermediate models may be analyzed and documented to identify the failure cause(s). New training data may be added as needed to improve training accuracy.

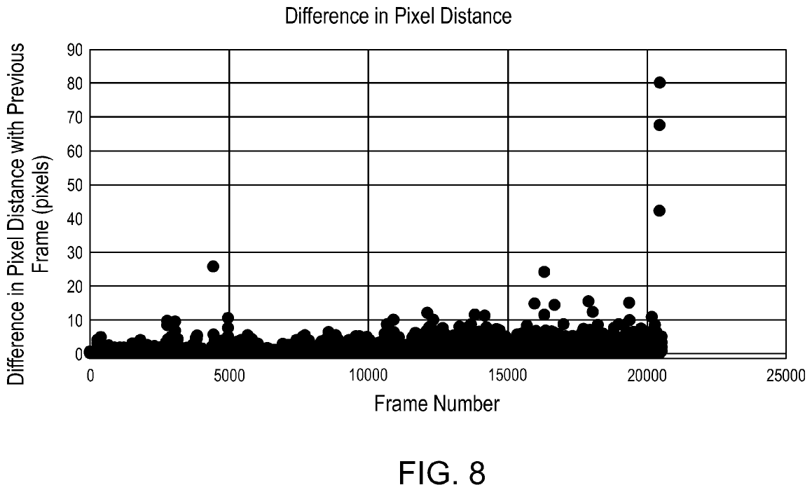

FIG. 8 illustrates an example of a real model segmentation tested on 10 new videos by mapping the changes in pixel distance over time of the center of the mouse (head+body+tail) over time showing very few outliers. A phase of post processing may be used to remove artifacts, or more training data may be used to further optimize the segmentation.

In some embodiments, within an experimental session for data acquisition and/analysis performed over a predefined duration, analyses of videos may be done frame by frame, or of other time series done at the highest resolution possible, so as to be used in the extraction of instant features. Such instant features can then be combined into higher-order features, such as states, motifs, or domains.

In some embodiments, the following parameters may be used to qualify and define features:

Frame Number: The frame number starting at 0 with increments of 1 or n if sampling every nth frame.

Timestamp: The time at which the frame is sampled. A particular signal may be used to synchronize the videos with other time series collected by, for example, an LED flash. The timestamp, for example, may therefore be the top camera (D1), such as top 3D camera 32, time series timestamp after LED syncing with increments of 0.1333 seconds if the sampling is done every 4th frame in a 30 frames per second video.

Additional Cameras: Putative additional cameras (D2, D3, etc) may be set in certain angles to a principal camera (D1) that contain an amount of segmented object pixels (Number of Head Pixels+Number of Body Pixels+Number of Tail Pixels).

As may be appreciated, features may be identified using one or more optical sensors, one or more thermal sensor, one or more pressure sensor, or any combination of the foregoing. The features can include instant features, such as:

Top Body Part Center X: (D1 View) The pixel X-coordinate of the center of the body part (such as the head) segmentation pixels. The pixel locations of all the head segmentation pixels may be averaged to acquire the pixel coordinate of the center.

Top Body part Center Y: (D1 View) The pixel Y-coordinate of the center of the body part segmentation pixels. The pixel locations of all the head segmentation pixels may be averaged to acquire the pixel coordinate of the center.

Top Body Part Num Pixels: (D1 View) The total number of body part segmentation pixels in the frame.

In some embodiments, an ellipse algorithm may be used to geometrically fit an ellipse around the mouse to determine the nose and tail positions, rostral and ventral areas, and the overall shape and orientation of the object. The head and body pixels may be summed together to create one single blob. The algorithm may determine which X and Y directions may include more segmented pixels and may fit an ellipse around the most extreme points along all the possible X and Y directions of the segmented blob.

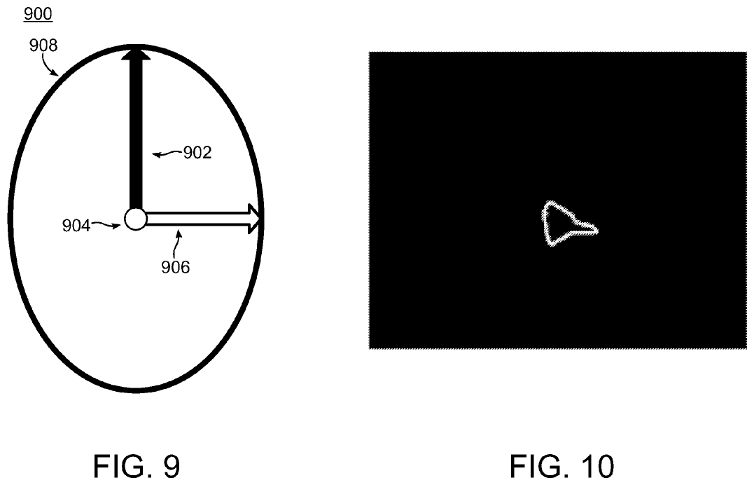

FIG. 9 illustrates the major axis 902 with a black arrow, the center 904 with an open circle, and the minor axis 906 with an open arrow. Instant features captured by this system include, for example, but are not limited to:

Ellipse Center X: The pixel X-coordinate of the center of the ellipse may be drawn around the blob (relative to the top left corner of the image).

Ellipse Center Y: The pixel Y-coordinate of the center of the ellipse may be drawn around the blob (relative to the top left corner of the image).

Ellipse Major or Minor Axis 0: The X distance in pixels from the center coordinate of the ellipse to the longest edge of the ellipse.

Ellipse Major or Minor Axis 1: The Y distance in pixels from the center coordinate of the ellipse to the longest edge of the ellipse.

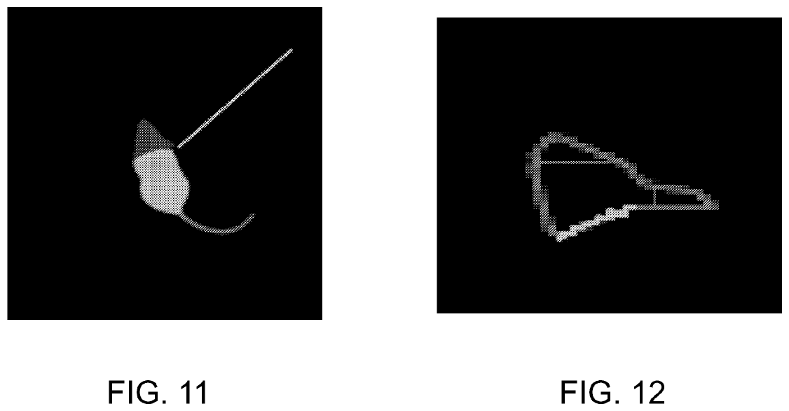

In some embodiments, with the segmentations generated, the nose, shoulder, and rump positions may be extracted from the head and body segmentations. A points-on-the-object algorithm may be used, where the shoulder point, for example, may be selected by first taking the border of the body and head segmentation as shown FIG. 10. This may be performed by using an edge detector, such as a Canny edge detection algorithm or another suitable edge detection technique, which may be applied to the body segmentation. Each “border” point that is on the border of the head and body may be then selected and averaged to get the center of the head and body border pixels. The center of the border between the head and body can be selected as the shoulder point in pixels as marked by the white line in FIG. 11.

In some embodiments, the nose point may be selected by first taking the border of the head segmentation. This may be performed using Canny edge detection applied to the head segmentation. Each “border” point distance from the shoulder point (e.g., the center of the orange line in FIG. 12) may then be calculated. Next, the distance between the points as well as their horizontal and vertical corresponding head segmentation pixel point (Red line in FIG. 12) may be used to determine if the point is a peak. The point with the largest distance from the shoulder point and shortest corresponding distance point in vertical and horizontal may be selected as the nose candidate.

The rump point may be selected by first taking the border of the body segmentation. This may be performed using a Canny edge detection applied to the body segmentation. Each “border” point of the body segmentation may then be used to calculate the distance to the center of the head. The furthest ten-pixel coordinates can be selected and averaged to get the rump point. After each point is selected from D1, D2 and D3 views in pixel coordinates, the 3D points may be calculated to determine the 3D position of the nose, shoulder, and rump. This operation may use known techniques to estimate a location of the point in a 3D environment using the D1 point, the D1 camera matrix, lateral point, and lateral camera matrix. This results in two 3D points for each point, one from the D2 view and the other from the D3 view. Instant features resulting from this modeling include but are not limited to:
Body Part View: (D2 or D3) The view that contained a better view of the body part (such as nose or shoulder) and the correct segmentation needed.
Body Part 3D X: The X coordinate of the body part of the mouse in 3D cube space (0 to 22 cm)
Body Part 3D Y: The Y coordinate of the nose of the mouse in 3D cube space (0 to 22 cm)
Body Part 3D Z: The Z coordinate of the nose of the mouse in 3D cube space (0 to 22 cm)

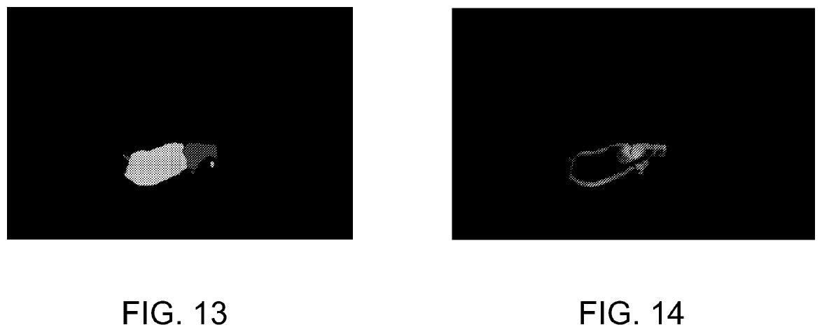

FIGS. 13 and 14 illustrate exemplary segmentation and resulting optical flow after combining the optical flow output with the segmentation in accordance with one or more embodiments of the present invention. Optical flow can be a measure of the pixel movements between two frames in radians and magnitude. In some embodiments, the system algorithm may first produce the optical flow mapping between two frames, which may then be combined with the segmentations produced for the head or the body to get the optical flow angle and magnitudes of each part without the surrounding optical flow noise as shown in FIGS. 13-14.

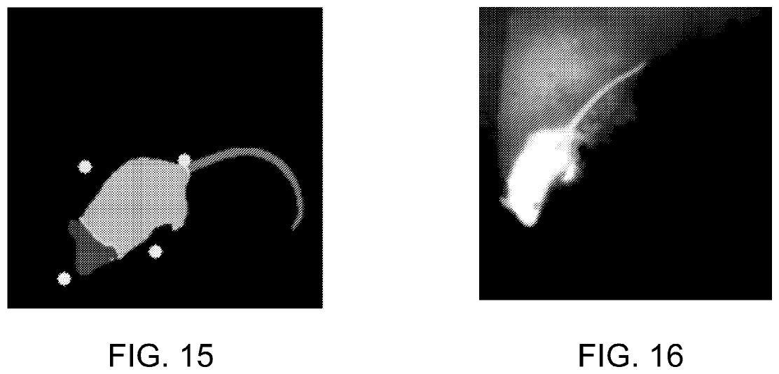

FIG. 15 illustrates the points (in yellow) where the Gaussian sampling may take place in accordance with one or more embodiments of the present disclosure. For Major 0, Major 1, Minor 0, and Minor 1 magnitudes and angles, a Gaussian sampling point may be set on each end of the ellipse parameter, and the optical flow may be multiplied with Gaussian sampled points to determine the movement of the bedding between frames as shown in FIG. 15.

Instant features concerning or derived from optical flow data can include:

Optical Flow Body Part Angle: Angle in radians (up to 2π). The direction in which the body part, such as the head or the body, pixels may be moving on average between frame N and N+1.

Optical Flow Body Part Magnitude: Distance in pixels. The magnitude in which the head pixels may be moving on average between frame N and N+1.

Optical Flow Body Part Angle: Angle in radians (up to 271). The direction in which the body pixels may be moving on average between frame N and N+1.

Instant features concerning or derived from thermal data can include:

Thermal data obtained from the thermal video may include segmentation of the body as described above, segmentation of the eye, temperature of the eye, temperature image of the environment in the arena and around the subject, texture quantification of the fur, segmentation and temperature of paws and limbs.

Instant optical features, as described above with reference to optical cameras, which can be similarly obtained with a thermal camera with a similar view.

Combined optical and thermal features that can be obtained from data obtained from a combination of optical camera(s) and thermal camera(s). For example, if the color of the mouse is very similar to the background color, thermal camera data may be used to improve image segmentation when generating a times series of nominally optical features. In addition, some aspects and parts of the body may be difficult to segment using the optical sensors and may be identified with more ease using differential temperature information between the area of interest, such as the paws, and the background.

Instant features concerning or derived from pressure-based data:

Data obtained with pressure-based data, such as with piezoelectric sensors, may include time series with high sampling rate quantifying the force exerted on each sensor, and features derived herein using signal processing methods and others as described herein.

Additionally, or alternatively, observational features identified using optical and/or thermal cameras can be used with pressure-based data to generate additional features.

In some embodiments, the 3D sensor field-of-view and number of light-capturing-pixels may directly affect the 3D measurement resolution. For accurately mapping fine body details, such as the nose, paws or tail, high resolution 3D data may be involved in obtaining sub-millimeter 3D resolution. Specifically, a subject treated with a toxic dose of a substance such as an opioid that may present with a Straub tail, which can be the tail of an animal (typically a rodent) that is rigidly held upwards in an angle greater than 450 to the body's main axis and a few centimeters higher than the floor surface or limb paw level. The tail of a rodent, being thin and narrow, may need a high degree of pixel resolution to capture fine changes in the depth information.

In some embodiments, the responses in a social situation may be captured when an experimental subject is assessed for their response to a target social subject in tests of sociality. Test of sociality may be important as assessment of phenotypes of models of and recovery after treatment.

Some aspects of social interaction may occur when two subjects are in close proximity, creating occlusion from view from any unique camera setting. Quick movements of the nose may indicate an exploration of the social counterpart. Behavioral platforms consistent with disclosed embodiments can support camera mounting, resolution, and sampling rates that can enable automatic extraction of features corresponding to minute aspects of social behavior. In contrast, data acquired by conventional platforms may lack sufficient detail for automatic extraction and therefore may require manual extraction of such features. In addition, the approach to modelling the temporal aspects of all measured features may capture the complex temporal dynamics of such a social encounter.

Consistent with disclosed embodiments, an analysis considering the temporal aspect of individual behavior and interactions between subjects may be more accurate and/or may provide more information than an analysis that summarizes such behavior or interactions over a period of time, such as the duration of a social encounter. For example, a temporal analysis using autocorrelation and lag analysis may indicate how the autocorrelation structure of natural behavior, or higher-order features of a subject change (e.g., changes in motifs, states, domains, or the like) as a result of the simple presence of a social counterpart.

In some embodiments, the 3D-sensor technology frame rate may directly affect the type of motions and actions that may be detected by the automated system, e.g., the system 310. Hence, faster actions such as head twitch, scratching, shake-offs, sniffing, sneezing, or trembling may need faster frame acquisition rates to capture and decompose them into their core components. In contrast, slower actions such as locomotion, rearing or drinking and eating may also use high frame rate data.

Hardware factors may dictate the maximum frame rate include the minimum light integration time by the sensor, the data transfer rate by the imaging hardware, but also the data bandwidth between the computer and the camera. Higher frame rate equipment tends to cost more, so careful analysis of the required minimum frame rates may be needed to limit the cost of production systems. Specifically, head twitch may be often observed when an animal is injected with a psychedelic drug. The action may last between ¼ second, and up to 3 seconds. At a slow frame rate, the action is perceived as a blurred motion of the head. However, at frames rates of 30 Hz or higher, the 3D lateral and roll motion of the head may be easily observed. The addition of a fast mechanical actuator, such as a piezoelectric may provide additional high sampling rate information to improve the identification of fast movements and the like.

In some embodiments, certain conditions and drug effects may induce a tremor in rodents. A motif may include capturing tremor, tremor intensity, and tremor duration. The tremor may be detected in the head or body parts and the tracking of descriptor of such body parts may determine a 2D or 3D wave of position as a function of time. Tremors in mice may be induced by a drug. For example, by harmaline (15 mg/kg), tremorine (20 to 30 mg/kg) or lysergic acid diethylamide (4 mg/kg), which may give rise to tremors between 14 and 25 cycle/s which are regular, but with varied amplitude. Tremors may be different from movements in the 22-25 cycle/second range shown in control animals, which may be irregular by nature. Tremors may occur during locomotion or rest.

To capture tremors and to differentiate between natural and drug-induced fast movements, it is important to have acuity and accuracy in the estimation of the wave characteristics in order to estimate the frequency, period regularity, and amplitude variability. The addition of a fast mechanical actuator, such as a piezoelectric actuator, may provide additional high sampling rate information to improve the identification of tremor as well. Accurate sampling of the wave may allow the estimation of the autocorrelation. For example, the autocorrelation rₓ(τ)≡∫x(t)×(t+τ)dt, can indicate periodicity, if there is a local maximum when τ>0, and the existence of a T₀ period which may determine that the local maximum will be found at nT₀ (where n is an integer). 1/T₀ is the fundamental or lowest frequency and may determine harmonics if the amplitude of the signal has other maxima at frequency N/T0, where is M is another integer.

If the wave characteristics are constant, then the wave can be a stationary wave. Fourier or wavelet transforms may be used to explore the stationary versus non-stationary characteristics of a putative tremor or oscillatory movement in the extent that the sensor sampling frequency is at least the square of twice the frequency 1/To. If the frequency in which the object wave characteristics are measured is equal or greater than twice its period, and more than double samples are taken, it may be possible to determine if the observed movement if simply noise or a real tremor, indicative of particular internal state.

Moreover, the quantification of the fundamental frequency and its harmonics may allow the description of its “quality”, done similarly in the frequency analysis of sounds waves, enabling complex and granular descriptions of biological signals which may be applicable to behavior, physiology, or electrophysiology. Lag analysis of the autocorrelation may also be used to explore the periodic nature of the signal. If the signal is indeed simply noise, then autocorrelation decays rapidly with increasing lag, whereas if the signal is perfectly periodic, the autocorrelation changes but remains significant as the lag increases.

In some embodiments, rostral digging or pushing bedding or other material in a forward direction may be normal behavior of many animals. For example, rodents may perform these behaviors in a forward direction with the nose, head or front paws or limbs. Without assessment of the substrate surrounding the subject, it may be difficult to capture this behavior since the body and head movement may resemble a simple head extension and retraction, which may be part of very different behavioral motifs.

Ventral digging, on the other hand, may involve moving substrate under the subject's body with the subject's paws or limbs. It may be hard to capture these movements if the body occludes the substrate being moved around. Proper camera positioning to minimize occlusion may alleviate this problem. The addition of different sensors can provide a plethora of information not only about the subject, but also about the surrounding area. A thermal camera, for example, may be used to detect changes in temperature in the bedding underlying a mouse. Such bedding signal will change if the mouse pushes the bedding or digs in the area. In addition, analysis of the pixel noise distribution in the area surrounding the subject may detect changes in the distribution due to the subject's digging behavior.

FIG. 16 illustrates an exemplary thermal image of a mouse injected with the drug MK801 in a behavioral platform as seen through the thermal camera in accordance with one or more embodiments of the present disclosure. Note the heated area on the top left denotes bedding where the subject had been resting and that had not been perturbed through digging.

In some embodiments, a number of mechanisms of action may induce low temperature, which may be accompanied by piloerection. For example, serotonin full and partial agonism at the 1A receptor may result in both hypothermia and piloerection. An agonism at the 5HT2A receptor may also produce both hypothermia and piloerection, although antipsychotics with strong 5HT2 antagonism may seem to be more frequently associated with hypothermia.

Adrenergic α1A and 2A agonism and antagonism, respectively, may induce piloerection. The ability to detect both temperature and piloerection may add sensitivity of the system 310 to various pharmacological effects. For certain compounds, these two features may be correlated. Hypothermia may be caused by inhibition of sympathetic regulatory mechanisms or central mechanisms such as regulation by the preoptic region of the ventral hypothalamus.

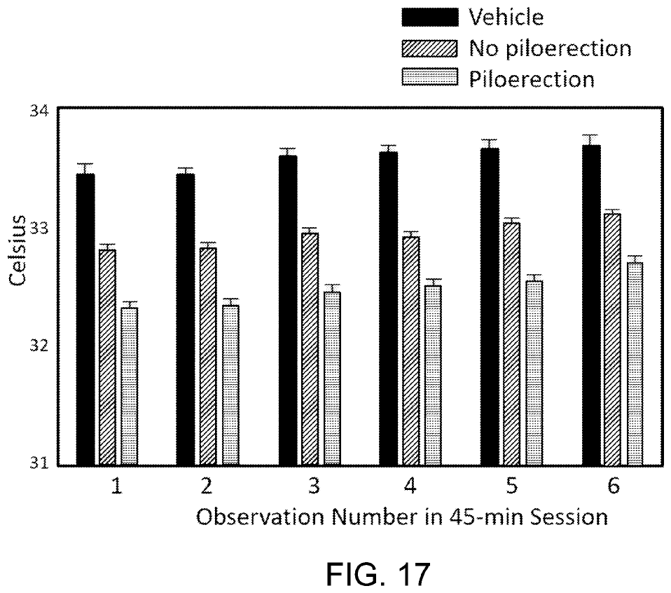

FIG. 17 illustrates an example of the ocular temperature of the mouse as a function of observation period separated by observations of the subject's fur after the end of the observation period, in accordance with disclosed embodiments. Subjects injected with vehicle show normal temperature that increases slightly with session time due to the anxiogenic testing environment, whereas subjects injected with compounds show some low temperature without piloerection and frank hypothermia with piloerection. FIG. 17 shows the mean (−/+ standard error of the mean) temperature as a function of observation time for vehicle injections that produce no piloerection, compounds that induced low temperature without piloerection, and compounds that induced hypothermia with piloerection.

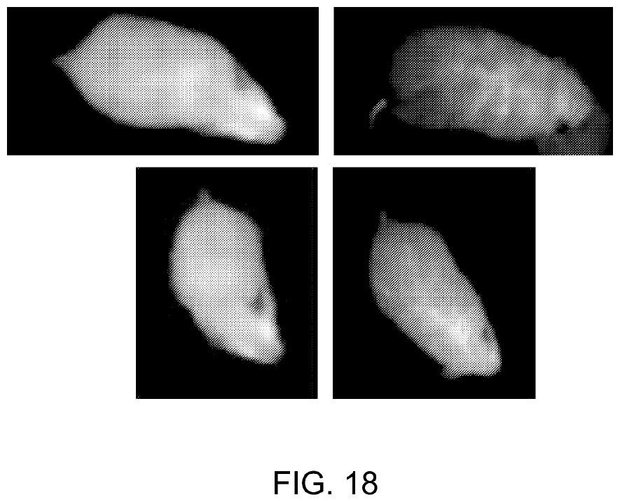

FIG. 18 illustrates an example of differing thermal fur texture between mice injected with vehicle and those injected with a compound, in accordance with disclosed embodiments. The compound caused ocular hypothermia and piloerection in the mice injected with the compound. These effects are illustrated in FIG. 18, demonstrating that the disclosed systems and method can detect eye and body temperature, and adaptation to hypothermia. FIG. 18 shows two mice injected with vehicle (left) and two mice injected with one of the compounds from FIG. 17 that induced both hypothermia and piloerection. In addition to the automatic measurement of temperature, the systems and methods disclosed herein capture the quality of the fur, indicative of general health but also piloerection, which may be harder to assess with an optical camera. FIG. 18 shows that piloerection may be accompanied by different thermal textures of the fur, as an additional example that may be used to poke the potential therapeutic value or deleterious effects of drugs or novel compounds or other treatments.

In some embodiments, a system may include various sensors, actuators, and devices. For example, the system may include memory, a plurality of actuators, an enclosure, a plurality of imaging devices, at least one thermal imaging sensor, at least one floor force sensor, at least one piezoelectric sensor, and at least one processor. The plurality of actuators may include any combination of an aversive stimulus probe, a motor challenge floor, a controllable light source, a tactile stimulator, or waterers and feeders. The enclosure may include the plurality of actuators coupled to at least one side, a floor, or any combination thereof of the enclosure. The plurality of imaging devices may be configured to capture image data of a plurality of images of a predefined region of the enclosure, including a subject, after the subject has been administered with at least one drug. The subject may exhibit a plurality of target features representative of a target condition. The plurality of imaging devices may include a plurality of three-dimensional (3D) sensors, a plurality of 2D sensors, or any combination thereof positioned at predetermined locations relative to the enclosure. The predetermined locations of the plurality of imaging devices may be configured to minimize visual occlusions of portions of a body of the subject. The at least one thermal imaging sensor configured to capture thermal image data of the subject of the predefined region of the enclosure. The at least one floor force sensor may be coupled to the floor of the enclosure. The at least one piezoelectric sensor may be coupled to at least one side of the enclosure. The at least one processor may be configured to execute computer code stored in the memory that causes the at least one processor to continuously receive the image data of the plurality of images of the predefined region comprising the subject over a predefined time interval. The at least one processor may be configured to continuously receive thermal imaging data of the subject from the at least one thermal imaging sensor over the predefined time interval, to continuously receive movement data of the least one side of the enclosure, from the floor of the enclosure, or both respectively from the at least one piezoelectric sensor or the floor force sensor over the predefined time interval. The at least one processor may be configured to input the image data of the plurality of images, the thermal imaging data, the movement data of the least one side of the enclosure, the movement data from the floor of the enclosure, or any combination thereof into at least one feature extractor machine learning model or a rule-based feature extractor to generate a set of measured features. The at least one feature extractor machine learning model may include an instant feature extractor, a state feature extractor, and a motif features extractor, and to query at least one database using the set of measured features to identify a target signature associated with the target condition, where the target signature may include the plurality of target features.

Note that the 2D sensors and/or 3D sensors may also be referred herein to as 2D cameras and/or 3D cameras.

In some embodiments, each 3D sensor from the plurality of 3D sensors or a combination of at least two 3D sensors may provide a minimum and a maximum sensing range sufficient for measuring a size and a shape of the subject, a sufficient amount of 3D volumetric pixel resolution varied based on a particular part of the subject, a sufficient data sampling rate to capture meaningful movement activities made by the subject, and a spectral sensitivity of light in a wavelength range from 400-1100 nm.

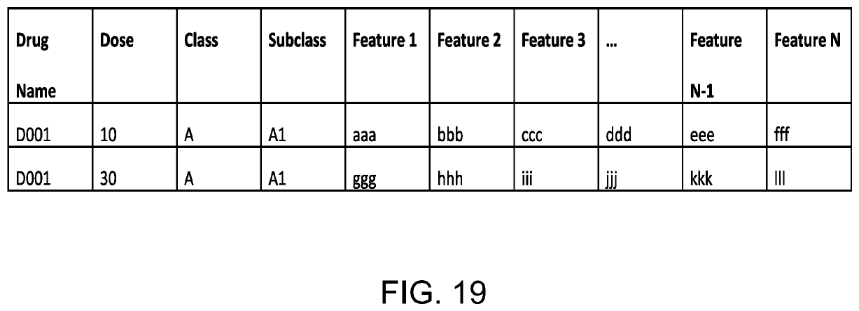

FIG. 19 illustrates an exemplary set of measured features, in accordance with disclosed embodiments. The exemplary set of measured features may be determined by the data flow pipeline 100 shown in FIG. 1 and used to query database 130 of instant features, states, motif sets or sequences, and domains to identify a target signature associated with a target condition. The number of features N could range from a few dozen to more than a million.

As shown in FIG. 2, illustrates an exemplary data processing flow diagram for obtaining target signature from the set of extracted features, in accordance with disclosed embodiments. The exemplary target signature associated with the target condition may be displayed to a user of the system 310 on GUI 388 as shown in FIG. 3A.

As described above, FIG. 4 illustrates a feature engineering data flow diagram 400, in accordance with disclosed embodiments. This data flow is similar to the dataflow illustrated in FIG. 2. With reference to FIGS. 1-4, the feature engineering data flow diagram 400 illustrates how the features are obtained in system 310. The at least one processor 360 may process the data generated by the sensors 410 of the enclosure 320 by applying qualifiers 430 such as a time stamp 432, a period type 444, and an event trigger 446 to the managing the data from an aversive stimulus sensor 402, a force sensor 404, an optical sensor 406, and a thermal sensor 408. The at least one processor 360 may respectively use the output data from these sensors in extract algorithms to extract instant features 420 such as an aversive stimulus instant feature 422, a force instant feature 424, an optical instant feature 426, and a thermal instant feature 428. These extracted instant features may be relayed to a sensor fusion module 440, a state analyzer module 450, a motif analyzer module 460, and a domain analyzer module 470.

In some embodiments, feature module 480 may receive instant features 482, state features 484, motif features 486 and domain features 488 as follows: The instant feature module 420 may directly relay the extracted features to the instant features 482. The sensor fusion module 440 may execute a time series integration module 442 that generates additional instant features that may be relayed to the instant features 482 in feature module 480. The state analyzer module 450 may apply the instant features to a supervised state identification module 452, and an unsupervised state identification module 454 to generate the state features that may be relayed to the states features 484 in the feature module 480 module. The motif analyzer module 460 may apply the instant features to a supervised motif identification module 462, and an unsupervised motif identification module 464 to generate the motif features that may be relayed to the motif features 486 in the feature module 480. The domain analyzer module 470 may apply the instant features to a supervised domain identification module 472, and an unsupervised domain identification module 474 to generate the domain features that may be relayed to the domain features 488 in the feature module 480.

In some embodiments, the instant features 482, state features 484, motif features 486 and domain features 488 in the feature module 480 module may be applied to signature analyzers 490 for generating the target signatures based on the experimental plan. The signature analyzers 490 may generate data that can be input to a signature report and visualization module 492. The signature report and visualization module 492 can generate a report or visualization that may be output on the GUI 388 of the display 386.

The aforementioned examples are illustrative and not restrictive.

### Section B

Pharmaco-EEG (pEEG) was pioneered in the late 19th and early 20th century but was displaced as a methodology of preference by the arrival of imaging and molecular biology techniques. Yet, the power to map brain activity and drug effects using this cheap and robust technique cannot be underestimated.

Embodiments of the present disclosure herein describe systems and methods for using a high throughput behavioral platform to develop an automated machine learning system to use EEG data for identification of potential beneficial potential of existing and novel drugs. The translatability of animal pEEG may be enhanced by using powerful machine learning techniques and their ability to process large pharmacological pEEG data. An important aspect of the disclosed embodiments is the use of machine learning classifiers that use human-based class labels for the drugs used as machine learning training references.

In some embodiments, a behavioral platform can use pEEG to investigate the effects of compounds of different drug classes on electrical brain activity. The behavioral platform can provide a uniform testing platform to record actigraphy and quantitative pEEG from one or more, such as one, two, or four, brain regions of unanesthetized subjects before and after drug administration. As described herein, physiological data, including pEEG other biometrics, and features derived at least in part from the physiological data can be used to train a machine learning classifier. The classifier can then be used to identify novel compounds that have a desired effect on pEEG activity. The behavioral platform can be used to phenotype disease models including autism spectrum disorder, rare genetic epilepsies, Huntington Disease, Alzheimer's Disease, for example. As pEEG yields objective pharmaco-dynamic signatures specific to pharmacological action, it may be used to evaluate translational biomarkers in CNS disorders and rapidly screen compounds for potential activity at specific pharmacological targets to provide valuable information for guiding the early stages of drug development.

Additionally, the embodiments disclosed herein may use a similarity analysis for quantifying the similarity between the pEEG signature for two different compounds. The similarity analysis is described in Section J and incorporated herein by reference. For example, rodent pEEG may translate well to the receptor affinity at the benzodiazepine receptor; thus, a compound that may be similar to a benzodiazepine (e.g., according to features derived from physiological data acquired by the behavioral platform) may be predicted to be as equally potent at the benzodiazepine receptor as measured from in vitro binding experiments.

The disclosed embodiments may incorporate a system for collection of pEEG representing reference drug effects (approved drugs and tool ligands) and the automated analysis of such data for prediction of potential beneficial therapeutic and toxic or other side effects or drugs (for repurposing or combination goals) and novel compounds. A particular aspect of the invention is the assessment of sleep and wake states to avoid confounding aspects of the differential effects of drug on sleep versus awake animals.

In some embodiments, the system may provide a method to automatically separate wake from sleep pEEG. In some embodiments, the automated identification of sleep and wake periods to distinguish the differential drug effects may be done based on pEEG and accelerometer data, using machine learning methods.

Consistent with disclosed embodiments, system 310 of FIGS. 3A-3B can be adapted for challenging a subject with different stimuli and analyzing movements and brain signals in response to the stimuli, in accordance with one or more embodiments of the present disclosure. In some embodiments, the subjects may be rodents. In some applications, the rodents may be mice or rats.

In some embodiments, a head mount 390 may be placed on the subject's head (e.g., skull). The head mount may include at least one electrode to measure brain electrical activity such as electroencephalogram (EEG) signals, for example. In some embodiments, the head mount 390 may also include at least one accelerometer. The signals from the at least one electrode and/or the at least one accelerometer in the head mount 390 may be coupled via wires to circuitry 308 that can relay the signals for processing to the at least one processor 360.

In some embodiments, the accelerometer data, the pEEG data, and other sensor data such as from the optical cameras, may be used to identify behavioral features, such as instant features (e.g., body shape and posture at a given time), states (such as locomotion or immobility), motifs (such as a stereotypic set or sequence of movements), or domains (such as hyperarousal). The ability to construct ethograms and to collect pEEG signatures in an integrated manner allows a deeper understanding of drug effects and the bidirectional interplay of pharmacology and behavior.

In some embodiments, system 310 may use the sensitivity of pEEG as compared to other pharmaco-machine learning content rich approaches such as those simply based on overt behavior. For example, whereas at the behavioral level, both benzodiazepines and 5HT1A agonists show anxiolytic profiles, their corresponding pEEG signature may be fundamentally different. Another example is gaboxadol, a GABA receptor agonist, which like classical benzodiazepines, may cause sedation in both rodents and humans, but has a different profile regarding its effect on beta, delta and theta bands.

In some embodiments, the use of labels referring to human proven efficacy and mechanism of action in supervised machine learning classifiers implicitly incorporates backtranslation from the clinic to the preclinical realm. Thus, despite robust differences in rodents and humans in some respects, the disclosed embodiments may provide a robust translational mechanism to further drug development and translatability to the clinic.

In some embodiments, system 310 shows an exemplary experimental setup to access a pEEG database of compounds with known mechanism and therapeutic value in subjects as stored in the memory and/or storage devices 65. To obtain a pEEG database of compounds with known mechanism and therapeutic value in subjects, one or two surface recording electrodes on the headmount 390 may be used. In some embodiments, a prefabricated electrode headmount with additional one or two indwelling wire electrodes and connectors for the wires connected to cortical screw electrodes may be used. This may enable turnkey implantation with pre-cut electrodes for accurate depth placement for specific brain areas such as the striatum and hippocampus. In some embodiments, a prefabricated electrode headmount with at least 2 electrodes (e.g., 4 electrodes, with 2 surface and 2 deep electrodes or any combination thereof) may be used. In some embodiments, a prefabricated electrode headmount with more than 4 electrodes, with different surface and deep electrodes combinations, may be used. In some embodiments, one of the channels may be used as a reference control electrode. In some embodiments, one of the channels may be used as an intramuscular (EMG) electrode. Groups of up to 128 subjects may be tethered and recorded simultaneously. Subjects may be chronically implanted and reused after washout to reduce animal numbers. A preamplifier may be added to the system as needed. Object 315 may be a rodent, such as a mouse or a rat, for example. The disclosed embodiments may be applied as is to any area of research using systemic administration of compounds to the extent that the drugs are blood brain barrier (BBB) penetrant. The disclosed embodiments may also be used for applications focusing on CNS-acting drugs without limitation. Non-BBB penetrant drugs may be administered intracerebrally using a different mount design as needed.

In some embodiments, several cohorts of subjects may be chronically implanted with the headmounts that may be used for a collection of pEEG for a variety of drugs at multiple doses, including vehicle treatment to create a unified data set of pEEG which may be used to train a classifier. Each drug at a particular dose (drug-dose) may be administered to a group of subjects (n=5-8, preferably n=10-12, or more). Drugs and doses may need to be carefully selected to cover various therapeutic indications and efficacies. A dataset may include pEEG profiles from more than 30 drugs divided into more than 2 and less than 10 classes. In some embodiments, the number of drugs may exceed 80, including control treatment, separated in more than a dozen classes, where each drug may be represented with at least 3 doses. In yet some embodiments, the number of drugs may include a total of more than 2000 samples.

Table II shows examples of the drugs used for a CNS application and Table III shows examples of the therapeutic classes of interest. Specifically, Table II shows examples of drug treatments used for training of the pEEG classifiers for a CNS application. Table III shows examples of therapeutic classes used for supervised training of pEEG classifiers for a CNS application.

TABLE II

Allopregnanolone
Alprazolam
Amitriptyline Hydrochloride
Amisulpride
D-Amphetamine
Aripiprazole
Atomoxetine
Bupropion
Buspirone
Caffeine
Carbamazepine
Chlordiazepoxide

TABLE III
Drug Class

Antidepressant
Anxiolytic
Antipsychotic
Cognitive Enhancer
Hallucinogen
Mood stabilizer
Psychostimulant
Control/Vehicle

In some embodiments, pEEG may be recorded 1 hour before drug administration and for 2 hours after drug administration from all recorded brain regions simultaneously. To maximize the clinical translatability of a pEEG preclinical database, the frontal cortex and parietal cortex can be selected without limitation. ECoG EEG from these electrodes may be shown to offer direct rodent-to-human translation. Additionally, the hippocampus and striatum can be targeted for indwelling local field potential (LFP) recordings to investigate pharmacological responses on brain regions known to be innervated by different neurotransmitter systems.

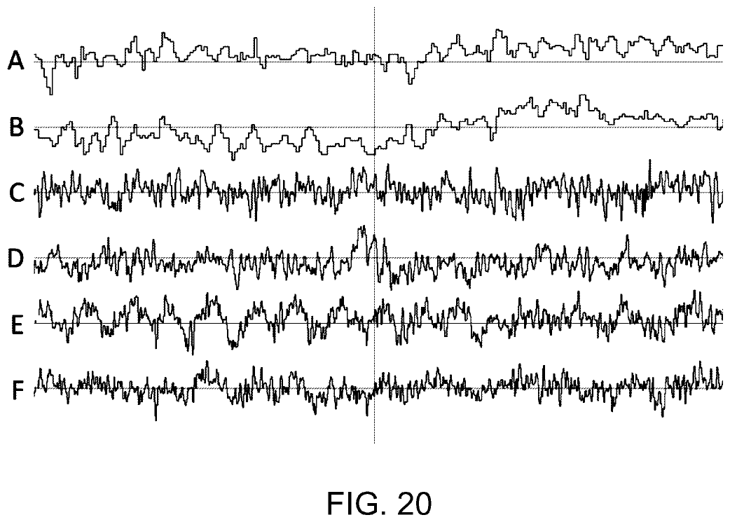

In some embodiments, the ECoG EEG and LFP data as shown below in FIG. 20 can also be accompanied by a 2-dimensional accelerometer which captures up-down and side-side movement of the head. This data can be used as a surrogate to sleep-staging data and can also be used to determine if a drug influences locomotor activity (stimulation or sedation).

FIG. 20 is a series of signals: segment of two accelerometer channels (A-B) and 4 EEG signal channels (C: hippocampus, D: striatum, E: parietal, F: frontal) from a typical recording. Accelerometer data can be sampled at a rate of 30-125 Hz and EEG signals can be sampled at a rate of 500-1000 Hz in accordance with one or more embodiments of the present disclosure.

In some embodiments, pEEG data consumed by a behavioral platform classier or other machine learning model can be processed first to remove artifacts. Such pEEG processing can include the automatic removal of artifacts typically seen from unanesthetized recordings such as movement or electrical artifacts. To determine the threshold for the artifact peaks detection, the signal may be first normalized with a z-score followed by a probability density function of the amplitude using a kernel distribution as a nonparametric representation of the probability density function. Whereas the distribution of the pEEG data can be approximated by a normal distribution, artifacts may form peripheral peaks at extreme positive and negative values, thus allowing identification and removal from subsequent analyses. Next, subject features can be extracted from both accelerometer and pEEG channels. The subject features can include simple standard features obtained through calculation of the power spectrum. In order to extract subject features for pEEG, Z-score normalization may be applied to each pEEG channel to correct for possible between-animal variability of the signal's amplitude. Next, a power spectrum can be computed for each pEEG channel using Matlab or other suitable software or algorithm. Specifically, to compute the time-dependent spectrum of each nonstationary signal, the signal can be divided into overlapping segments, a Kaiser window can be applied, and a short-time Fourier transform can be computed. Finally, the transforms can be concatenated to form a matrix. The spectrum can be computed with frequency limits between 1 and 100 Hz for standard power frequency bands, or between 1 to 250 Hz to capture high frequency ripples. The frequency resolution can be between 1-10 Hz. The average standard frequency bands are shown in Table IV listing the analyzed standard frequency bands. Ripples above 100 Hz may be captured in 10-25 Hz bands, or alternatively in 25-50 Hz bands. The asterisked (*) sub-band in Table IV corresponds to the range [57, 63] Hz that was filtered out using a notch filter to remove 60 Hz line noise. In some embodiments, the raw pEEG signal or derived measures (e.g., coherence), can be used as input to a model. In some cases, the pEEG signal can be first processed with a manifold learning or a strange attractor model for acquisition of robust pEEG features and/or dimensionality analysis and reduction.

TABLE IV

| | Band name | Frequencies, Hz |
|---|---|---|
| 1 | Delta | [1, 4] |
| 2 | Theta | [4, 8] |
| 3 | Alpha1 | [8, 11] |
| 4 | Alpha2 | [11, 14] |
| 5 | Beta1 | [14, 18] |
| 6 | Beta2 | [18, 32] |
| 7 | Gamma1 | [32, 50] |
| 8 | Gamma2 | [50, 100] (*) |

In addition to the pEEG signals, the accelerometer signals can also be processed. Z-score normalization can be applied to each accelerometer channel to homogenize individual subjects' scale. Next, the signal spectrum can be computed similar to pEEG with frequency limits between 1 and 50 Hz, a temporal resolution of 10 seconds, and a frequency resolution of 5 Hz, although other ranges may be used without limitation.

In some embodiments, a normalization against the baseline pre-drug may be convenient, although other types of normalization may be used without limitation. pEEG and accelerometer spectrums can be normalized to the corresponding mean value x_mean during the pre-dosing interval [0, 1 hour](baseline): x_norm=(x−x_mean)/x_mean. For both pEEG and accelerometer data, the post-dosing features (obtained during the 2-hour post-dosing period) can be expressed as percent change from pre-dose baseline.

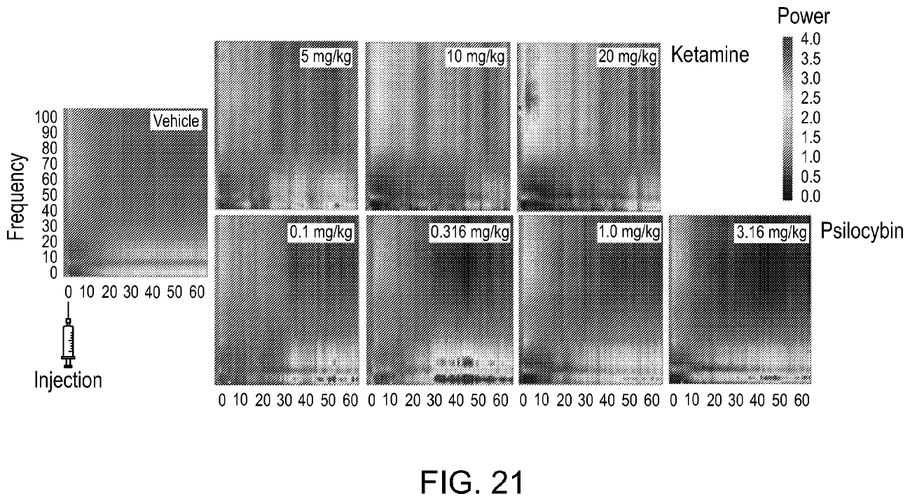

FIG. 21 shows examples of the pEEG spectral power signature of two different drugs in accordance with a particular embodiment of the present disclosure. Subjects were recorded for a 1-h baseline and 2 h post-treatment recording. Example heatmaps such as those shown in FIG. 21, may show the changes in power for all frequencies for the 2-h post-injection period. Power may be normalized against the baseline values for each individual before averaging per dose. Different doses of ketamine (FIG. 21, top) and psilocybin (FIG. 21, bottom) show dose-dependent short-term increase or longer-term inhibition of high frequencies (gamma), respectively.

In some embodiments, the pEEG data may be generated from multiple drug doses that may be used to exemplify the process of classifier training, and to obtain predictions of therapeutic classes such as those shown in Table III. A Recurrent Neural Network (RNN) may be used as a classifier model as shown in FIG. 23. Briefly, the network may be based on a bidirectional Long Short-Term Memory (LSTM, 32 hidden units) layer, two fully connected layers (“elu and “softmax” activations), batch normalization and dropout layers. The model may be trained using cross-entropy loss function and “adam” optimizer, for at least 30-50 epochs. The last layer of the model may be a dense layer, which may provide probabilities for more than a dozen drug classes.

In some embodiments, to decrease the influence of outlier samples on the predictive models, it may be possible to remove samples from the training process. To identify outlier samples, all samples may be first used in the training set. When the same samples are used in the test set, any sample meeting an “outlier” criterion may be removed. In some embodiments, outlier criteria may encompass that a sample is removed when (i) the correct class for this sample is not among the top 3 predicted classes, or (ii) the predicted probability of the correct class is less than 20%, or (iii) the difference between the predicted probability of the correct class and the probability of the top prediction is greater than 20%. This process may exclude about 10% of the data as outliers.

In some embodiments, to improve the robustness of the predictive model, independent implementations of a general model architecture may be built by randomly initializing the weights for the RNN. Final predictions may be derived from the model with highest accuracy, or from the average such as the median or mean results from all trained models. To check the accuracy of the resulting model system, different cross validation approaches may be used such as a n-samples-out, or n-doses-out, and/or a whole-drug-out, where an n-samples subset, an n-doses subset, and/or a whole drug, respectively, may be systematically removed from the training dataset, and the rest of the drugs may be used for training a new model. The excluded data may then be used for model validation as a test set. Model prediction may be done at a sample level, so as to get a prediction for a given drug-dose. The predictions for each sample may be combined by taking the median or other averaging statistic, over each class and normalizing to preserve the unit sum such that the total of probabilities across all classes sum to 100%.

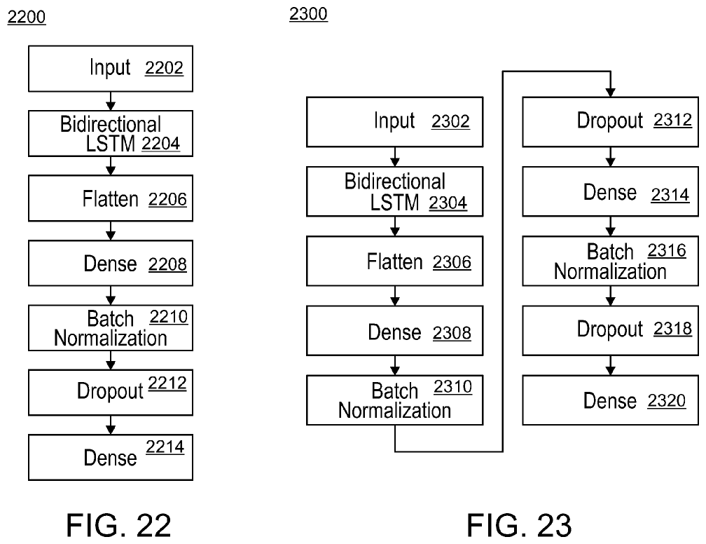

FIG. 22 is an exemplary RNN model architecture 2200 for a pEEG drug class classifier in accordance with one or more embodiments of the present disclosure. The input layer 2202 can receive 4 timestamps (the number of 30-minute bins in 2 post-dosing hours). Each timestamp can be represented by at least 54 subject features: 4 pEEG channel×8 frequency bands+2 accelerometer channels×11 frequencies. The output of input layer 2202 can be passed to a bidirectional Long Short-term Memory Networks (LSTM) (e.g., with 32 hidden units) layer, which can consider the time sequence of the data and produce higher-order features enriched with the time dependency of the sequence passed. The output of the LSTM layer may be flattened in layer 2206 before it is passed to dense layer 2208, batch normalization layer 2210 and dropout layer 2212 with 64 and 32 units consecutively, and “elu” activation. The model may be trained using cross-entropy loss and “adam” optimizer trained for 30-50 epochs. The last layer of the model can be a binary dense model 2214, with sigmoid activation. This layer may return the probability of the drug sample being in the possible therapeutic classes.

In some embodiments, a particular model can be selected after trying several different model architectures, model parameters, and processing of the input data, yielding the best results. To evaluate the overall accuracy of the model, a whole-drug-out assessment can be run as described herein. For each left-out drug at different doses, the class with the highest probability may be considered and called a correct prediction (true positive) if matched with the correct label or a wrong prediction (false positive) if not matched. Thus, the overall precision (where precision measures how many of the samples were classified in a class that are correctly classified in such class) and recall (where recall measures how many of all the samples in a class are correctly classified in such class) of the model for different classes may then be calculated. For the specific example dataset and model architecture represented in FIG. 22, an estimated total accuracy may be around 70%. The 4 classes with the lowest F1 scores (where F1=2*((Precision*Recall)/(Precision+Recall))) had an average precision of about 30% and low recall (5%) indicating that these were under-represented, or highly heterogeneous classes in the model. These classes may be simply removed from the classifier, or improved by including more training drugs, doses, and/or samples. The next 5 classes with medium F1 scores had an average precision of 71% and recall of 54%. The top 4 classes with the highest F1 scores had an average precision of 69% and a recall of 94%.

Alternative embodiments may maximize precision at the expense of recall or vice versa.

In some embodiments, with regard to tradeoff between precision and recall, since the classifier is trained on predefined classes, the classifier may force the signal of a test compound to be classified into one of the trained classes only. However, it may be possible that a compound may produce a response outside the realm of the available classes and therefore, the output of an n-class classifier may not represent the true underlying compound signature. It may be possible that a novel class of compounds exists that has not been included in the set of class labels used for training. To overcome this, an additional class, designed as “unknown class” can be included in the final output of the classifier. This “unknown class” can indicate that the compound is active, but the classification system could not reliably assign certain features or patterns (which may be either novel or may occur from insufficiently strong changes exhibited at lower doses) to any CNS class, although the difference may be detected from the vehicle. The “unknown” signature may be calculated by comparing any compound data with the corresponding vehicle data using an independent binary classifier. This classifier may be used to ascertain the compound's overall activity, which may then be used to update the overall classification output of the “n-class” classifier.

In some embodiments, to calculate the activity of each compound compared to its vehicle, each sample can be pre-processed as described before, for example, in 30-minute bins and different frequency bands. Data presenting artifacts known to an EEG expert can be removed from the sample sets. To utilize the time dependency in the data, an RNN can be used as a classifier model as shown below on FIG. 23.

In some embodiments, the evaluation of a particular drug dose can be done in two steps that can include forming subsamples, randomly selected from the compound and vehicle sample sets, and dividing each subsample into training (75%) and test (25%) subsets. These two processes can be repeated several times and the results can be averaged to define the overall “activity” of the compound (which, in short, is the accuracy of this 2-class classifier). The output of this classifier can be used to rescale the output from the n-class classifier. Scaling can be done by comparing the activity of the compound according to the 2-class classifier (A₂) with the sum of the class predictions of the n-class classifier (Aₙ). If A₂<Aₙ, then Aₙ is set to Aₙ=A₂, and the probability of all classes can be rescaled such that sum of these equals A₂. If A₂>Aₙ then U=A₂−Aₙ can be set as the “unknown class” probability. If A₂=Aₙ, no change may be required.

FIG. 23 shows an RNN model architecture for binary drug activity classifier in accordance with one or more embodiments of the present disclosure. Briefly, the network is based on the bidirectional Long Short-term Memory Networks (LSTM) (e.g., with 32 hidden units) layer, which can consider the time sequence of the data and produce higher-order features enriched with the time dependency of the sequence passed. An input layer 2302 can accept inputs similar to those of input layer 2202, above. For example, the input layer 2302 can receive 4 timestamps (the number of bins in 2 post-dosing hours). Each timestamp may be represented by 54 subject features: 4 pEEG channel×8 frequency bands+2 accelerometer channels×11 frequencies. The output of input layer 2302 can be passed to LSTM layer 2304. The output of LSTM layer 2304 may be flattened in layer 2306 before it is passed to a first set of dense, batch normalization and dropout layers (e.g., layers 2308, 2310, and 2312) with 64 units and a second set of dense, batch normalization and dropout layers (e.g., layers 2314, 2316, and 2318) with 32 units, both sets having “elu” activation. The model may be trained using cross-entropy loss and “adam” optimizer trained for 30-50 epochs. The output layer of the model, dense layer 2318, can have sigmoid activation. The output layer may return the probability of a drug sample being different from vehicle.

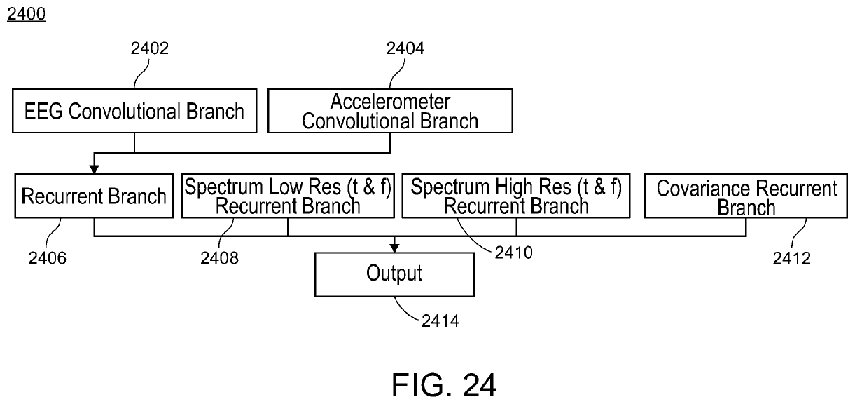

FIG. 24 shows an alternative architecture that may be much deeper and diversified in different branches in accordance with one or more embodiments of the present disclosure. In some embodiments, an alternative classification scheme can be used to define and process more features. The alternative scheme can include more than one branch, such as 4 branches, corresponding to different types of features, as shown above in FIG. 24. The final class membership decision can be made using outputs of all 4 branches (output 2414). The first two branches (EEG Convolutional Branch 2402 and Accelerometer Convolutional Branch 2404) can process raw subject data from both pEEG- and accelerometer channels. A set of convolutional layers may down sample the signals and input the subject data to the recurrent layers (Recurrent Branch 2406) to reveal their temporal dynamics. The second branch (Spectrum Low Res 2408) can use the subject features from the basic classification scheme, namely, robust but low temporal (t) and frequency band (f) resolution power bands. Since the set of subject features used in the basic classification scheme may have low temporal resolution (t=30 minutes) and a limited number of frequency bands (8 bands, see Table IV), the third branch (Spectrum High Res 2410) may analyze the spectrum dynamics in time with high temporal (t=1 minute) and frequency (f=1 Hz) resolution. The last branch (Covariance Recurrent Branch 2412) can process interactions between the pEEG channels, which may be represented by the matrices of covariance between the channels computed for 1-minute bins in 8 frequency bands.

In some embodiments, early layers may separate the data into wake and sleep periods before branching off into specific subnets.

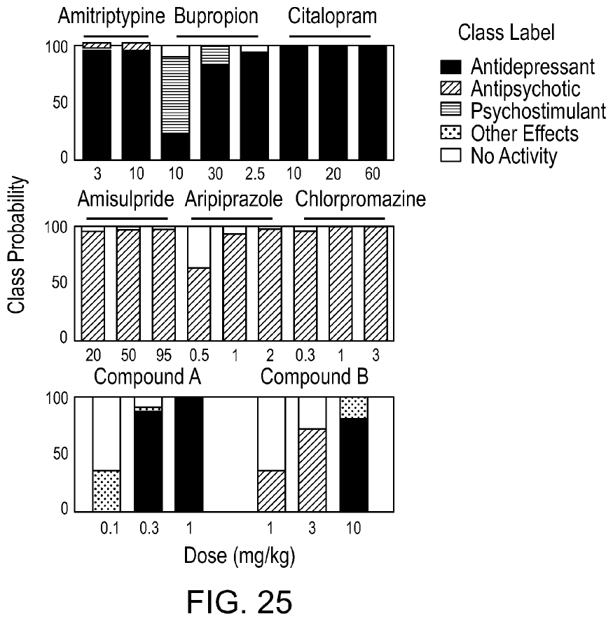

The classification for each drug-dose can be graphed using bar charts with standardized colors or patterned fills. FIG. 25 shows an exemplary output of the classifier using reference antidepressants (Amitriptyline, Bupropion and Citalopram) and antipsychotic compounds (Amisulpiride, Aripiprazole and Chlorpromazine) classified using a classifier trained with a set that did not include those drugs. FIG. 25 shows the therapeutic class signature of the reference drugs obtained using a deep learning classifier trained on all the other available drugs in accordance with one or more embodiments of the present disclosure. More than a dozen classes can be available, but only five are shown for clarity. Amitriptyline and citalopram showed a clean antidepressant signal (black fill) whereas bupropion showed a more stimulant profile (mix of antidepressant, black fill, with psychostimulant, dotted fill) that was consistent with clinical data. All antipsychotics tested showed a clean antipsychotic profile (stripped fill). Two novel compounds were also screened and showed a robust antidepressant signature (compound A; note the low dose had a low, different signal which included an unknown class probability, shown as crosshatched fill), and a mixed antipsychotic (lower doses) and antidepressant (higher dose) signature (compound B). Note that Compound A is very potent compared with the reference antidepressant, possibly making it a good candidate for starting a novel chemical series exemplifying the power of the present invention.

In summary, the disclosed embodiments can include a deep learning classifier that may be trained using the reference drug dataset, divided in n classes plus vehicle. The classifier example as shown in this disclosure had a high precision (percent of correct classifications) for the classes that had ample training data sets (i.e., antipsychotics, antidepressants, and GABAergic anxiolytics) with an overall precision of >70% (note that random choice of class for such drug dataset results in 8% precision).

In some embodiments, a system may include a memory, an enclosure, a plurality of imaging devices, at least one electrode, at least one electrode, and at least one processor. The enclosure may include a plurality of actuators coupled to at least one side, a floor, or any combination thereof of the enclosure. The plurality of imaging devices may be configured to capture image data of a plurality of images of a predefined region of the enclosure, including a subject, after the subject has been administered with at least one drug candidate. The at least one electrode may be coupled to the head of the subject. The at least one accelerometer may be coupled to the head of the subject. The at least one processor may be configured to execute computer code stored in the memory that causes the at least one processor to continuously receive the image data of the plurality of images of the predefined region including the subject over a predefined time interval, to continuously receive EEG signal data from the at least one electrode, to continuously receive accelerometer data from the at least one accelerometer, and to input the image data of the plurality of images, the accelerometer data, the EEG data, or any combination thereof into at least one trained machine learning model to output a classification of the at least one drug candidate, where the classification may (i) a drug class, and (ii) a drug sub-class.

In some embodiments, the class analysis can be done for different periods post-dosing to show the changing nature of the drug effect and its dynamics. In such embodiments, classifiers are trained for the required post-dosing periods.

In some embodiments of the disclosure, the time-course of the drug effect is synchronized in time using time-warping methods as a way to handle the different PK/PD patterns of the reference and testing compounds.

One or more technological problems include efficiently identifying drugs or compounds having similar or reverse effects to a target model, where a target model signature refers to the pEEG signature of a model of disease or symptom as compared with its experimental control. This drug electroencephalographic signature analysis (DESA) can enable efficient comparison of the set of EEG features to those of the drugs or compounds, thus resulting efficient and reliable way to screen drugs looking for therapeutic treatments based on their pEEG signatures. DESA can also be used to find compounds that are good candidates for combinations, due to their complementary, synergistic, or partially opposed pEEG signature.

In some embodiments, pEEG may also be analyzed with DRFA to generate statistically independent combinations of original observational features (“de-correlated features”). Each de-correlated feature may be a statistically independent, weighted combination of all observational features. The de-correlated features may be used for dimensionality reduction without loss of relevant information, which is essential for visualization and data interpretation. The DRFA method may be used with pEEG data to create, for example, a multidimensional space in which a group signature of all antidepressants and psychedelic drugs may be compared against another group of, for example, anxiolytic drugs. In this analysis, the top features driving the separation between the two groups may be queried and analyzed. For example, it may be shown that the main difference between the two groups is the power in the gamma and ripples frequencies or their respective pEEG. Such space may also be used to test if a compound's signature is closer to that of the anxiolytic drug group or not.

Complex pEEG data reflects the recruitment of large sets of neurons in different brain areas. Such complex dynamics may be analyzed using Hidden Markov Models (HMMs). The differential effects of drugs may then be mapped onto such HMM to provide a deeper understanding of the drug effect.

Discretizing pEEG states may allow the use of Markov chains for visualization of the drug effects of states durations and transitions as compared to a control vehicle, capturing in such a way the dynamics of the drug time course.

In some embodiments, results of drug screening may be visualized using topological data analysis, which is used to map the multidimensional pEEG space identifying an intrinsic cluster structure for projection onto a 2D or 3D space.

The aforementioned examples are illustrative and not restrictive.

### Section C

Consistent with disclosed embodiments, behavioral platform can characterize dynamic gait features (e.g., stride duration, step sequences, and the like). Such gait features may be difficult to characterize, even by a trained human observer. Consistent with disclosed embodiments, a behavioral platform can assess motor or locomotor behavior, such as gait behavior, in an unbiased and automated way. Such assessment can include calculating gait features, which may be instant, state, motif, or domain features as described herein (e.g., paw shape, location, and movement). The disclosed embodiments can provide an automatic, objective, fast and consistent assessment of the effects of modifications. For example, the disclosed embodiments can provide an automatic, objective, fast and consistent assessment of the level of injury and course of recovery in animal models of neurodegeneration and other gait and motor coordination disorders. The systems disclosed herein can also be used to assess other types of motor or neurologic dysfunction.

Consistent with disclosed embodiments, a behavioral platform can be configured to assess rodent behavior based on a granular analysis of locomotion at the gait cycle level, providing a way to separate gait features for different types of locomotor patterns. Using a combination of different deep learning algorithms, the disclosed embodiments can enable automated identification of a subject's body and body parts in any type of arena. The observational data obtained can be used to define a phenotype corresponding to a modification. The effectiveness of a treatment can then be assessed in terms of a reduction in pathological aspects of the phenotype resulting from the treatment.

FIGS. 3A-3B illustrate an exemplary system 310 for analyzing dynamic gait features of an object 315 in accordance with one or more embodiments of the present disclosure. System 310 may include a control computer 50 communicating 335 with a computer-controlled enclosure 320 to which a plurality of sensors and/or actuators may be coupled thereon to execute an experiment on object 315, such as a rodent. The enclosure may include a transparent floor (not displayed in FIGS. 3A-3B), made from glass, for example, through which light may illuminate the object 315. Object 315 may be administered, for example, with a compound that may include at least one drug in accordance with an experimental plan and observed with the plurality of sensors. System 310 may be configured to determine the behavioral and/or physiological effects of the administered compound on the gait features of the subject over a predetermined time period (e.g., the experimental session).

In some embodiments, the plurality of sensors and/or actuators coupled to the enclosure may include, but are not limited to, an aversive stimulus probe 318 that may be deployed or retracted, lighting 304 that may be configured to change illumination intensity levels and/or light wavelengths as applied to the object 315, a tactile stimulator 316 to administer tactile stimuli to the object 315, a top camera 306, a top thermal camera 302, a first side camera 328, a second side camera 314, a floor force sensor 322, waterers and feeders 329 as well as additional actuators 22 for applying any additional suitable stimuli to the object 315 and/or any additional sensors 44 in accordance with the experimental plan. In some embodiments, lighting may also be applied through a transparent floor such as a glass floor, for example.

In some embodiments, the processing and storage resources in this disclosure may include various interconnected computers and/or storage devices that support at least some operations related to the capture, processing and/or archival of data as related to various operations associated with the system 310 for analyzing gait features of the object 315. In some embodiments, at least some resources described herein, including, without limitation, some the equipment directly attached to the animal enclosures, may be connected through network connections on a local area network, and/or using cloud services, as detailed herein.

Conventional approaches to rodent behavioral assessment can rely upon manual assessment of behavior, such as gait. Such manual assessment can be time-consuming and subjective. Consistent with disclosed embodiments, machine learning models can be used to assess rodent gait in order to quantify different types of gaits, neurological conditions and neurodegenerative disorders leading to gait dysfunction, and recovery thereof through experimental treatment. The disclosed embodiments can correctly detect paw shape and identify the corresponding limb using video recordings. These video recordings can also capture behaviors other than locomotion, while also filtering out common undesired artifacts such as urine marks or genitals images. The disclosed embodiments provide can provide an improved method for detecting paw segmentation and correctly assigning limb identification. With error correction at multiple levels, the disclosed embodiments can be used to calculate dynamic gait features with high accuracy.

In some embodiments, an apparatus with a transparent glass floor may be used. In some embodiments, for use with mice for example, this arena (e.g., enclosure 320) may be a long rectangular corridor of width between 5 to 25% wider than the subject cross-section. In another embodiment, a square floor may be used. In yet another embodiment, the arena may be a circular corridor that may be used with rats, for example. The glass floor may be illuminated from the side. This arrangement may ensure that light will be diffracted by any object in contact with the glass, thus allowing for improved detection of the paws and other body parts in close contact with the floor surface. In some embodiments, the floor may be illuminated by colored light emitting diodes (LEDs) that may provide a strong contrast with a ceiling color light. The top of the apparatus, which becomes the background of the image, may be of a contrasting color, such that the outline of the animal may be clearly defined by the shadow it casts. A conventional illuminated glass technique can be used to estimate plantar pressure. The vertical component of the force exerted by the limbs estimated through the analysis of illuminated pixels can correspond closely with the forces measured through a classic force transducer. The disclosed embodiments can use the information captured through analysis of illuminated pixels of both hind and forepaws to analyze limb coordination and gait analysis.

Consistent with disclosed embodiments, the behavioral platform can record rodents using an optical camera. In some embodiments, the experimental sessions may be of short time intervals ranging from 4 to 5 minutes, but it can also last for one hour, depending on the experiment protocol used and the size of the enclosure. In some embodiments, an IP-enabled color camera may be used, and the recorded video file format may be “mjpeg,” which is typical for continuous recording over a network. Any digital color camera can be used for this step, but the recorded video should have an accurate timestamp, a sufficient resolution to accurately pick up individual paw shape (e.g., minimum of 5 by 5 or 10 by 10 pixels), and the typical frame per second (fps) is 30. In some embodiments, the whole extent of the experimental arena can be covered by more than one camera and the resulting videos can be concatenated or data jointly post-processed.

In some embodiments, the at least one processor 360 may use image segmentation software to identify each pixel that belongs to a specific area of the rodent body. The at least one processor 360 may distinguish all pixels belonging to one of the following classes: “Background—0”, “Body Positive—1”, “Body Negative—2”, “Hind Left (HL) Paw—6”, “Hind Right (HR) Paw—5”, “Front Left (FL) Paw—4”, “Front Right (FR) Paw—3” and “Unknown—8”. The at least one processor 360 may use a neural network architecture such as u-net, for example, to create the output array that has the same image size of the raw video frame, but where each pixel has the class identification number. In real-life examples, measured artifacts from the rodent genitals may often touch the floor surface, creating an image, and the rodent's urine may create a similar stain to the paw print, for example, may cause some detection errors. In some embodiments, the software executed by the at least one processor 360 can be configured to ignore these real-life artifacts and limit classification only to the above seven classes for object detection. Note that “Body Positive” is the body area when a rodent moves from left to right (clockwise direction in a rat circular arena), and “Body Negative” corresponds to the opposite direction (counterclockwise). In narrow arenas, subjects may move in a rather linear trajectory parallel to the long side such that only one of those two directions is possible.

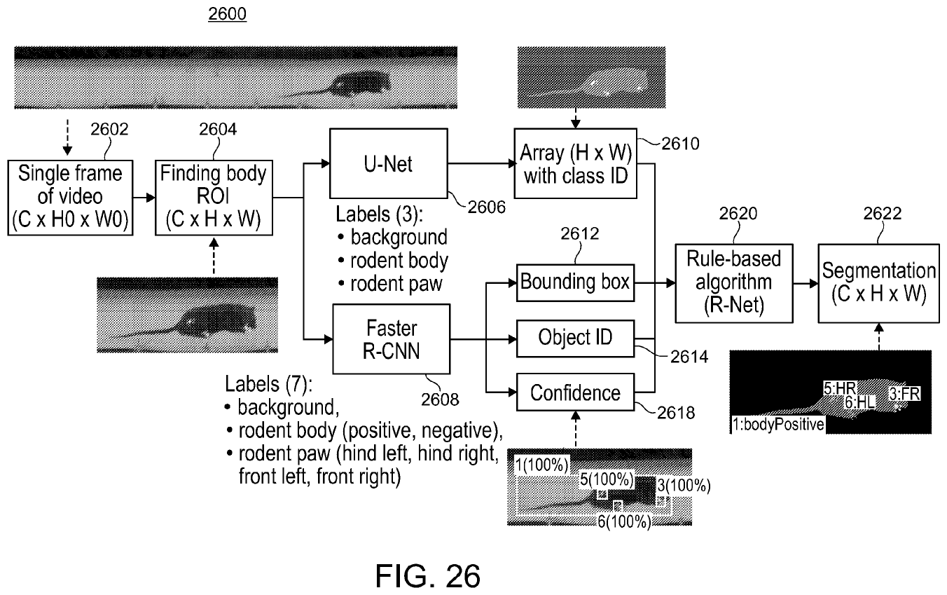

FIG. 26 illustrates a segmentation pipeline flow diagram 2600 in accordance with one or more embodiments of the present disclosure and executed by the at least one processor 360. The analysis may start with a single frame 2602 of the recorded video, and the animal body region 2604 may be selected by threshold method with reference frame for faster R-CNN. The selected region may be input into two machine learning models: a u-net neural network 2606 and a faster R-CNN 2608 that uses different labels than the u-net neural network 2606. Then the at least one processor 360 may combine the outputs from the two networks by application of heuristic rules, and the final segmentation result may be obtained.

Consistent with disclosed embodiments, the u-net neural network 2606 can be configured to accurately detect contours of different body parts. The output of u-net neural network 2606 can be an array 2610 of pixels with class ids for each pixel. However, the computational accuracy of the u-net neural network model may decrease as the number of classes increases, as the network may require more layers for each additional class. Furthermore, the u-net neural network may also require excessive time when using high-resolution cameras. Accordingly, the at least one processor 360 may reduce the input image size by focusing on a smaller image patch around the location of the animal. To get the image patch, the at least one processor 360 can apply two methods depending on the video quality. In one approach, the at least one processor 360 may use a video reference frame where there is no animal. The at least one processor 360 can subtract the video reference frame from a current frame in the video and for detecting the animal silhouette. This method is the fastest with the clean reference image; however, it does not guarantee accurate contours of the animal body. In another approach, the at least one processor 360 may use another neural network (e.g., faster R-CNN) which is fast and efficient for object detection. This second neural network can be used to detect the bounding box 2612 of a specific object in an image. The faster R-CNN 2608 may be trained to detect the previously mentioned classes. For U-Net, the at least one processor 360 may combine Body Positive and Body Negative as “Body” labels and HL, HR, FL and FR paws as “Paw” labels and “Background” for the rest of the image. With these three simplified classes, U-Net can exhibit sufficient performance and speed. Therefore, in RodentNet (which uses this two-stage segmentation algorithm as shown in FIG. 26), the at least one processor 360 may process the raw image with U-Net algorithm first and the Faster R-CNN algorithm next. The pixel-wise class information and bounding box 2612 with complete seven class identification 2614 and confidence scores 2618 may therefore be obtained.

In some embodiments, the at least one processor 360 can apply heuristic rules and filters as described here. To increase the accuracy of each neural network, the at least one processor 360 can use several additional heuristic rules 2620 to correct paw identification and paw segmentation masks. At the frame level, the overlapping bounding boxes 2612 can be first combined with the confidence scores 2618 to select the bounding box with the highest score. Then starting with the HL paw, the detected paws can be matched with the highest score for each paw. After the matching process, the secondary paw identification code can be executed, and the matching process can be repeated if there are remaining paws to be detected. Since there is only one rodent in a frame, possible assignments may have a maximum of one paw in each class. If paws remain unidentified, they may be assigned according to their positional relationship, in that Front Paws are in front of Hind Paws, and Left Paws are to the left of Right Paws, even if the algorithms classify with low confidence scores. The output can be an improved segmentation map 2622.

In some embodiments, the at least one processor 360 may generate a data table as described here. The segmentation and identification information for each video frame can be fundamental data to calculate the gait features. Given that the video image data is a 2D array, it can be summarized and converted into a tabular data format for further machine learning or statistical analysis steps. Tabulated data for each frame with the detected body part can be used as the source of information. For example, the center of the area can give the coordinate of the body or detected paws. The area and perimeter of the body area can provide shape information. In some embodiments, the gait feature extraction can be performed using multiple machine learning models, as described herein. The at least one processor 360 can then create a data table for each paw and body using a publicly available OpenCV contour libraries.

In some embodiments, regarding postprocessing, the paw identification accuracy may be evaluated at multiple frame levels and corrected. The center of mass of each paw and the body may be calculated so that those coordinates are consistent over multiple frames. If the coordinate differences between frames is measured to be less than a predefined threshold, then the paws may be identified as the same class, even if a detection error is reported by the machine learning models used to identify the paws. All paw labels can be checked with these criteria and assigned to a new label if the exchange of paw identification gives fewer discrepancies.

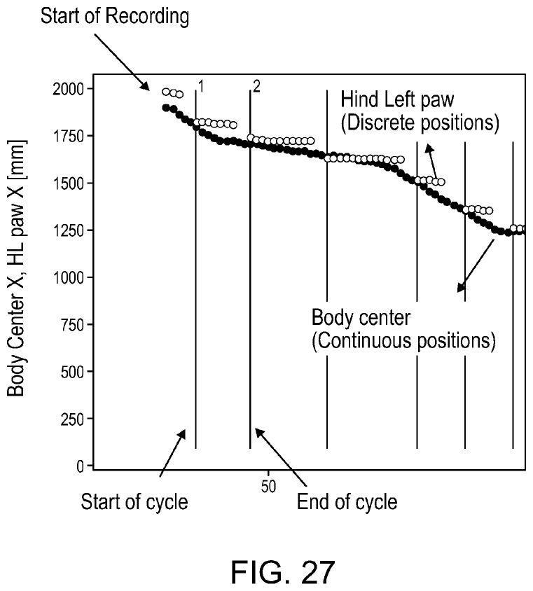

FIG. 27 illustrates a graph showing coordinates of the body center and HL paw center as a function of frame in accordance with one or more embodiments of the present disclosure. Over multiple frames, the at least one processor 360 may calculate the x, y coordinates of the different body parts. The same paw may be placed over the same position to increase accuracy of the paw identification. Rodent movement may be analyzed at the Cycle level, as defined as the sequence of paws until the first paw (the “Anchor”) is detected for a second time (e.g., a behavioral motif). Cycles may be marked with vertical lines. For each cycle, each paw may be shown as a connected line, indicating continuous contact with the floor. FIG. 27 shows 15 Cycles based on HL paw as an anchor.

In some embodiments, with regard to gait analysis and gait cycles, the next step is for the at least one processor 360 to calculate the gait patterns which may require positional information of all paws over multiple frames. The walking process of the rodent may be partitioned into anchor paw cycles (e.g., a behavioral motif). In one cycle, the anchor paw (typically the HL paw) may initiate a “Step” as it touches the ground, remains in contact while the subject's body moves forward, and then “Swings” up in the air. The full cycle may end with the next touchdown of the anchor paw. Other paws may go through the same cycle steps. Normally, each paw's cycle may be synchronized in a specific way; however, it may be assumed the most general configuration in which the cycle of each paw is independent of the others. The at least one processor 360 may calculate the starting time of the periodic cycles for each anchor paw and evaluate the gait features within a single cycle for different anchor paws (HL, HR, FL, and FR). The advantage of this approach is that locomotor behavior may be observed in minute detail. The embodiments disclosed herein may focus on freely moving subjects, therefore, many behavioral patterns may be observed over time, from immobility to slow walking, to fast running. By observing gait at a cyclical level, the aggregate gait features and patterns at different speeds may be aggregated.

In some embodiments, with regard to behavioral categories, the rodent behavior may be categorized in seven simple classes (e.g., behavioral states) mainly based on the body velocity: “walk,” “other movement,” “immobile,” “turn around,” and “backward walk.” in the circular arena, “walk” and “other movement” have two directions: clockwise (CW; left to right on a rectified video) and counterclockwise (CCW; right to left). Walk may include subclasses such as “run” and “trot.” If the rodent exhibits a speed higher than a certain threshold (set at 100 mm/s in a preferred embodiment) and shows a correct paw sequence, the cycle may be assigned a “walk” class. “Other” refers to when the rodent moves while changing direction often, so that the perpendicular displacement is more significant than that of the “walk” activity, also resulting in lower speed. Exploratory behavior and other slow movements may be categorized as “Other”. “Immobile” may be assigned when the velocity is less than a predefined threshold (100 mm/s) and may include the subclasses of “grooming,” and “freeze” behaviors. “Turn around” may be assigned when the rodent changes its head and movement direction. “Backward walk” may require no change of the head direction, but the rodent exhibits a movement backward.

In some embodiments, with regard to gait analysis, the at least one processor 360, having detected the paw position and its identity, defined the cycle and its type, gait analysis pipeline initiates a gait cycle analysis phase. The standard definitions of “stride,” “splay,” “base,” “swing,” and “step” can be used to define slightly over 600 features per subject per session. The following features are calculated: cycle type duration, total distance moved, average speed, body and paw parameters (area, perimeter, horizontal and vertical length, intensity, and others), number of paws detected, movement (forward and sideways), stride (length and duration), step (length and duration), splay (length), swing (duration), stand (duration), base (width), measures of asymmetry, paw position parameters, and cycle sequence type.

In some embodiments, with regard to gait features summary, the at least one processor 360 may provide a summary by summarizing the gait features over multiple cycles. Features can be averaged separately for the different behaviors (all, other, walk), directions (CCW, CW), statistics (average, standard deviation, count), and paw identity (HL, HR, FL, and FR).

In some embodiments, the at least one processor 360 can use the Gait Features for Phenotypic Analysis. In having calculated the Gait features, the at least one processor 360 can use a DRFA tool to define the phenotypic separation of a modification and a corresponding suitable control (e.g., a model of pain in rats, a model of a neurodegenerative disorder in mice, or a model of autism spectrum disorder). In addition, a database of phenotypic data from different types of gaits (such as Parkinsonian, hemiplegic, ataxic, and the like) may be used to train a machine learning classifier for the automated “diagnosis” of gait in a novel modification of an animal.

In some embodiments, a behavioral platform may include a memory, an enclosure, a plurality of actuators, a plurality of imaging devices, and at least one processor. The enclosure may include a glass floor. The plurality of actuators can include a controllable light source configured to illuminate the glass floor. The plurality of imaging devices can be configured to capture image data of a plurality of images of a predefined region of the enclosure, including a subject having a modification (e.g., a subject following administration of at least one drug). The at least one processor can be configured to execute computer code stored in the memory that causes the at least one processor to receive the image data of the plurality of images of the predefined region including the subject over a predefined time interval, where each image in the plurality of images can include a timestamp within the predefined time interval, to detect a contour of at least one segment representation of at least one portion of a body of the subject for each image in the plurality of images by inputting the image data of each image into at least one machine learning model, to identify gait cycles from changes in at least one coordinate of the contour of a specific portion from the at least one portion of the body from each image of the plurality of images and the timestamp of each image, to determine gait features of the subject by inputting data over multiple gait cycles to a gait analysis data pipeline, and to generate a summary of the gait features over the multiple gait cycles.

In some embodiments, the at least one machine learning model may be a u-net model, a faster R-CNN model, or another suitable deep learning model capable of precise image segmentation.

In some embodiments, the system may include a force sensor coupled to the glass floor.

The aforementioned examples are illustrative and not restrictive.

### Section D

Embodiments of the present disclosure herein describe systems and methods for using a behavioral platform for automated computer vision-enabled measurements of murine respiration as a physiological indicator of drug efficacy or toxicity in conjunction with other automated physiological and/or behavioral measures. Rodent respiration frequency can be a key physiological indicator of drug dose response and toxicity. For example, anesthetized, rodents have a slow (<2 Hz), regular breathing pattern, whereas they have a faster and more irregular pattern when they are awake (2-4 Hz), and even higher when they are actively exploring the surrounding (up to 12 Hz) during active odor sampling. Consistent with disclosed embodiments, a behavioral platform can use the disclosed computer vision-based respiration measurement techniques rodent behavioral testing. As described herein, such behavioral testing can be used for both drug screening and phenotypic treatment response analysis. As may be appreciated, the disclosed respiration measurement techniques can be used by any suitably configured behavioral platform to acquire physiological data concerning respiration. Likewise, the physiological data acquired can be used to generate features (e.g., physiological instant features, states, motifs, domains, or the like) suitable for use with the disclosed analysis techniques or other suitable analysis techniques. The disclosed embodiments are not limited an any particular behavioral platform or analysis technique.

As described herein, FIGS. 3A-3B illustrate a system 310 for measuring murine respiration of an object 315 in accordance with one or more embodiments of the present disclosure. System 310 may include a control computer 50 communicating 335 with a computer-controlled enclosure 320 to which a plurality of sensors and/or actuators may be coupled thereon to execute an experiment on object 315, such as a rodent. The rodent may be administered, for example, with a compound in accordance with an experimental plan and observed with the plurality of sensors such that the system 310 may determine the behavioral and/or physiological effects of the administered compound on the respiration of the object 315 over a predetermined time period (e.g., the experimental session).

In some embodiments, the plurality of sensors and/or actuators coupled to the enclosure can include, but are not limited to, an aversive stimulus probe 318 that may be deployed and retracted, a motor challenge 326 that can be deployed or retracted so as to force the object 315 to walk on a plurality of physical obstacles 324 arranged in an array with a predefined pitch to provide a motor challenge, lighting 304 that can be configured to change illumination intensity levels and/or light wavelengths as applied to the object 315, a tactile stimulator 316 to administer tactile stimuli to the object 315, a top camera 306, a top thermal camera 302, a first side camera 328, a second side camera 314, a floor force sensor 322, waterers and feeders 329 as well as additional actuators 22 for applying any additional suitable stimuli to the object 315 and/or any additional sensors 44 in accordance with the experimental plan for determining the behavioral and/or physiological effects of the administered compound on the respiration of the object 315 over the predetermined time period.

The embodiments in FIGS. 3A-3B are shown merely for visual and conceptual clarity and not by way of limitation of the embodiments described herein. Enclosure 320 may be of any suitable shape with the plurality of sensors and/or actuators placed in any suitable location around or within the enclosure. The at least one processor 360 can be located in any suitable cloud computing systems and/or remote computing devices coupled to the computer-controlled enclosure 320 over a communication network. The enclosure 320 as shown in FIGS. 3A-3B may be used in any suitable behavioral platform. Such a system can employ computer vision and mechanical actuators to detect spontaneous and evoked behavior eliciting responses through anxiogenic, startling and other types of stimuli without limitation, resulting in measurements of over two thousand high level composite features, and over about 1 million, such as over about 2 million or about 10 million, raw features.

In some embodiments, the measurement of the rodent's respiration can be made via measurement of the count of segmented pixels corresponding with the rodent for each frame of video capturing the animal. Such measurements can require video capture with sufficient resolution (e.g., minimum 1080 p) and frame rate (minimum 30 frames/second). As may be appreciated the minimum framerate can be arise from the frequency of rodent respiration and the requirement that the sampling frequency sufficiently exceed the Nyquist frequency, to enable accurate detection of respiration.

In some embodiments, if the computer vision pipeline generates segmented subject frames at the minimum frame rate of 30 fps, these original segmented frames may be fed directly into the respiration detection system. However, as many computer vision pipelines use a reduced frame rate to minimize required computational resources, additional re-segmentation of usable periods within the given video may be needed, assuming the base frame rate of the raw video is sufficient.

In some embodiments, the respiration measurement may further require that the subject remain relatively motionless for a sufficient duration and that the motion of the subject from frame to frame be amplified. In some embodiments, the processing and storage resources list in this description can include various interconnected computers and/or storage devices that support at least some operations related to the capture, processing and/or archival of data as related to various operations associated with system 310 for measuring murine respiration of the object 315. In some embodiments, at least some resources described herein, including, without limitation, some the equipment directly attached to the animal enclosures, may be connected through network connections on a local area network, and/or using cloud services, as detailed herein.

In some embodiments, the determination of the subject's activity can be done with an automated activity sensor system and process. The respiration assessment by such activity sensing system can be triggered automatically in real time, or as part of a post processing step.

In some embodiments, the analysis of respiration can be done for the duration of the experimental session and periods data artifacts caused by high activity or other undesired artifacts can be identified during post processing for exclusion of the corresponding respiration data.

In some embodiments, the analysis of respiration rate can indicate particular states such as sedation, inactive wake, sleep, without limitation.

In some embodiments, the behavioral platform can be configured to isolate respiration motions from other types of body motion. Such isolation can be achieved by restricting subject motion. Image data (e.g., sequences of video frames) corresponding to periods in which the subject is expected to remain relatively motionless can be analyzed to determine respiration. When the respiration analysis is performed on such image data, the e amplified motions of the subject can be representative of respiration rather than other types of body motion. In some embodiments, the inactivity of the subject may be automatically detected by the system using a variety of sensors and machine learning applications during the experimental session or during post-processing. In some instances, periods of inactivity may be more frequent during a particular phase of a protocol (e.g., when the subject is presented with an aversive stimulus inducing freezing). The behavioral platform can be configured to attempt to identify prospective periods of sufficient immobility for the purpose of respiration detection during such phases. In some embodiments, a behavioral platform can be configured to isolate respiration motions from other types of body motion using a machine-learning model. For example, deep learning techniques may be used to identify and isolate respiration signals over and above other overt body movements.

In some requirements, motion enhancement may be needed to ensure that there is sufficient periodic variation in the number of segmented body pixels between inhale and exhale portions of the respiration cycle. The disclosed embodiments are not limited to any particular method of motion enhancement. In some embodiments, motion enhancement can be performed using Eulerian Video Magnification, or another suitable technique.

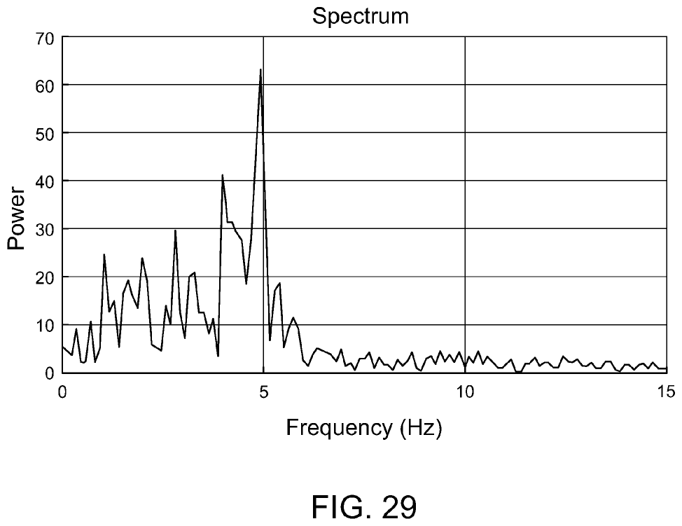

In some embodiments, the respiration rate may be calculated by the at least one processor 360 by performing the steps of:
segmenting the object of interest using computer vision techniques, such as a deep learning segmentation model;
counting the pixels, the largest contiguous segmented subject image for each frame (Pt, where t is the time stamp of a particular frame);
calculating the mean (P_{t.mean}) of the segmented object pixel counts corresponding to the two-second interval surrounding each frame [Pₜ₋₁−Pₜ₊₁];
calculating P_{dif}=Pₜ−Pₜ₋ₘₑₐₙ;
normalizing P_{dif} using a z-score approach;
defining the threshold standard deviation as the maximum of 0.1 or the absolute value of the minimum z-score;
removing frame pixel counts exceeding the threshold standard deviation;
applying a Fast Fourier transform to determine the frequencies inherent in the variations of the pixel counts;
normalizing derived frequencies;
removing frequencies outside the expected range of 1 Hz to 12 Hz; and
returning respiration rate as defined by the maximum of the remaining frequencies (as shown below in FIG. 29).

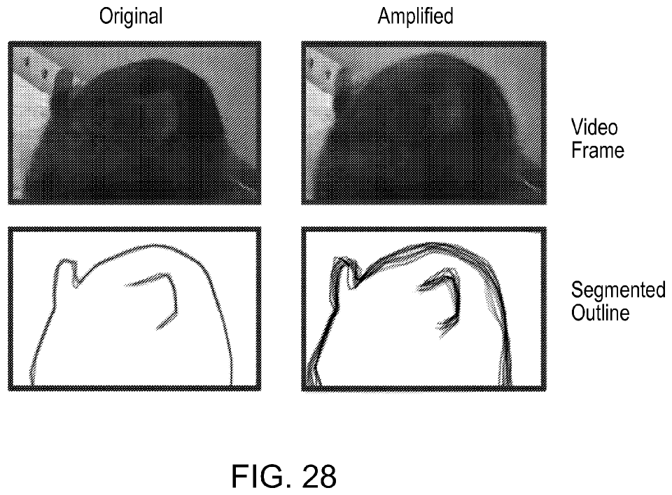

FIG. 28 illustrates a silhouette of the subject emphasizing the amplified movements from frames of a corresponding video clip for measuring murine respiration in accordance with one or more embodiments of the present disclosure.

In some embodiments, the subject's body may be segmented based on the optical density distribution on each frame. The segmented body's contour may be traced and used for respiration analysis by finding the largest 8-connected object in the binarized image, as an example of a method without limitation.

In some embodiments, physiological features can be extracted from the respiration data. These features can be used in the analysis of modifications, as described herein. For example, these physiological features can be used to assess drug effects, genetic manipulations, phenotypic signature using machine learning methods.

In some embodiments, a system may include a memory, a plurality of actuators, an enclosure, a plurality of imaging devices, and at least one processor. The plurality of actuators may include any combination of (i) a sound generator, (ii) a controllable light source, or (iii) a tactile stimulator. The enclosure may include the plurality of actuators coupled to at least one side, a floor, or any combination thereof of the enclosure. The plurality of imaging devices may be configured to capture image data of a plurality of images of a predefined region of the enclosure, including a subject, after the subject has been administered with at least one drug. The at least one processor may be configured to execute computer code stored in the memory that causes the at least one processor to continuously receive the image data of the plurality of images of the predefined region comprising the subject over a predefined time interval, where each image in the plurality of images may include a timestamp within the predefined time interval, to generate a segmentation 2D or 3D model for each image in the plurality of images by imputing the image data of each image into at least one machine learning model, wherein the segmentation 2D or 3D model for each image may include at least one segment representation of a body of the subject, to determine from 2D or 3D coordinates of each pixel in the at least one segment representation in a sequence of images in the plurality of images that the subject is a motionless state over a motionless time period, where the motionless time period is determined from the timestamp of the images in the sequence, to determine a pixel count of pixels in the at least one segment representation of each image in the sequence during the motionless time period, to identify an inhale portion and an exhale portion in sequential respiration cycles of the subject during the motionless time period by analyzing the pixel count of the at least one segment representation of each image in the sequence, and to compute a respiration rate of the subject from the timestamp of each image in the inhale portion and the exhale portion of the sequential respiration cycles in the motionless time period.

In some embodiments, the system may be further configured to induce the motionless state in the subject by applying to the subject any combination of (i) tactile stimuli from a tactile stimulator, (ii) an aversive stimulus from the aversive stimulus probe, or (iii) a sound from the sound generator.

In some embodiments, the system may induce the motionless state in the subject by applying to the subject any combination of (i) tactile stimuli from a tactile stimulator, (ii) an aversive stimulus from the aversive stimulus probe, or (iii) a sound from the sound generator, and a drug that increases the response to the stimuli or induces reduced activity.

The aforementioned examples are illustrative and not restrictive.

### Section E

Embodiments of the present disclosure herein describe systems and methods for using behavioral platforms with thermal cameras. Thermal cameras can be configured to automatically capture thermal data. Physiological features can be extracted from the thermal data. These physiological features can be used, as described herein, in assessing the effect of modifications on animal behavior or physiology using machine learning techniques. Such assessment can be performed as part of a program for developing novel therapeutics.

Preclinical research can require assessment of function in different domains. Physiological measures can open a window into the biological processes underlying health and disease, and recovery thereof. Capturing physiological and other non-behavioral subject features of animal function may provide an advantage over classical systems focused on the analysis of overt behavior. In addition, the behavioral platform may be designed to be integrated with other sensing modes, such as optical, electric, and/or mechanical sensors. Different body parts or areas may be used for the estimation of temperature. Axial and rectal temperature can be preferred for traditional thermometry. The tail, body, or the eye can be preferred for thermographic cameras.

In the embodiments disclosed herein, measuring temperature can be one way to assess physiology, anxiety, stress, and drug effects in the preclinical realm for research and drug discovery. Such measurements can involve the handling of the animal, which can consequently increase temperature due to the stress of being handled or can involve manual selection of an area within the enclosure or cage for the use of thermographic cameras. Disclosed embodiments include integrating thermographic cameras with a method of calibration, and automated extraction of thermal data for conversion to temperature of specific body parts of interest.

As described herein, FIG. 3A depicts a system 310 that can include different sensors in accordance with one or more embodiments of the present disclosure. Consistent with disclosed embodiments, at least one thermal camera can be combined with sensors such as optical, electrical, and/or mechanical sensors in system 310. As described herein a behavioral platform can be designed and configured to use of a thermal assembly coupled to enclosure 320 for observing the object 315.

The embodiments in FIG. 3A are shown merely for visual and conceptual clarity and not by way of limitation of the embodiments described herein. The thermal cameras and associated calibration and/or control circuitry may be placed in any suitable location in or around enclosure 320.

The terms enclosure and cage may be used interchangeably herein.

In some embodiments, the processing and storage resources listed in this description may include various interconnected computers and/or storage devices that support at least some operations related to the capture, processing and/or archival of data as related to various operations associated with system 310. In some embodiments, at least some resources described herein, including, without limitation, some the equipment being directly attached to the animal enclosures, may be connected through network connections on a local area network, and/or using cloud services, as detailed herein.

In some embodiments, a Thermal Measurement System (TMS) can include three systems: an Infrared Camera, an RTTMS, and a novel Infrared Camera Calibration System. The infrared camera can record thermal images of object 315 within the Laboratory Cage (e.g., enclosure 20). In each image frame, the RTTMS temperature elements can be visible to provide reference low and high intensity-to-temperature indicators. The Calibration System can adjust the camera operating parameters to span the range of reference temperatures for the RTTMS.

In some embodiments, the camera used in the infrared (IR) camera system can be a non-radiometric complementary metal-oxide-semiconductor (CMOS) infrared camera positioned atop the ceiling of the Laboratory Cage. From atop (e.g., the top thermal camera 34), the IR camera can image a mouse subject below in the cage in the infrared spectra. The IR camera can image a subject at 3 frames per second. The camera can be a FUR Boson 320 Camera 640×512 resolution (92°(2.3 mm) 30/60 fps w/Hard Carbon Coating—Industrial Grade 40 mK) with its field of view set to capture the interior side walls and floor. 16-bit images can be captured by the camera and transmitted via USB.

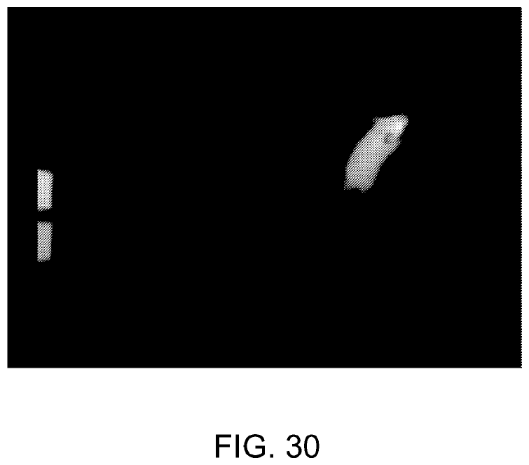

FIG. 30 shows an infrared camera image of the laboratory cage scene in the infrared spectrum including the test subject and the low and high temperature Peltier elements of the RTTMS in accordance with one or more embodiments of the present disclosure.

In some embodiments, the RTTMS may include a dual-Peltier Controller Sub-System, Data Acquisition System, and Video Capture and Imaging Software Services. The Dual-Peltier Controller Sub-System can include a Controller that further includes two ON-OFF current-amplified thermostats, each cycling around a fixed setpoint. One thermostat may control around a low setpoint, the other around a high setpoint. These setpoints can be adjusted to span the range of temperatures expected to span the temperature ranges of the experimental subjects. A negative-temperature coefficient (NTC) thermistor (e.g., a separate high precision PT100 Resistance) can send temperature feedback to the thermostat.

In some embodiments, Temperature Detectors (RTDs) can measure the temperature. A 1-5 volt conversion using a Head Mount RTD Signal Converter can follow, which may then be inputted to a data acquisition hardware. The Data Acquisition analog-to-digital (ADC) counts can then be converted to temperatures and recorded with a timestamp.

In some embodiments, the RTTMS mechanical assembly can include two 15 mm square Peltier elements. Beneath each element may be an NTC thermistor and an RTD sensor that are fixed in place with thermal epoxy. The Peltier elements can act as thermal generators. White thermal conductive tape can be applied to the surface of the elements while standard white tape can be applied to all other parts of the assembly. The assembly can be attached to the side of the Laboratory Cage (e.g., enclosure 20).

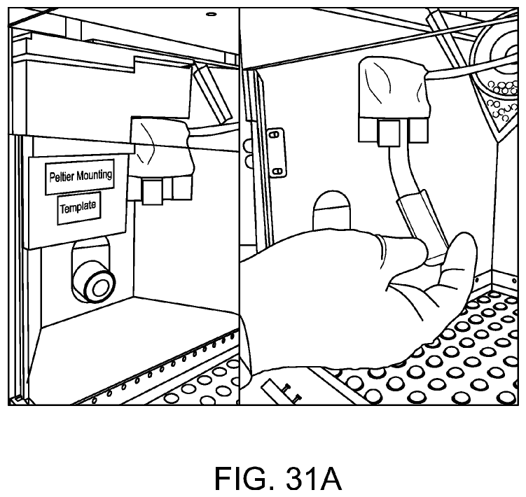

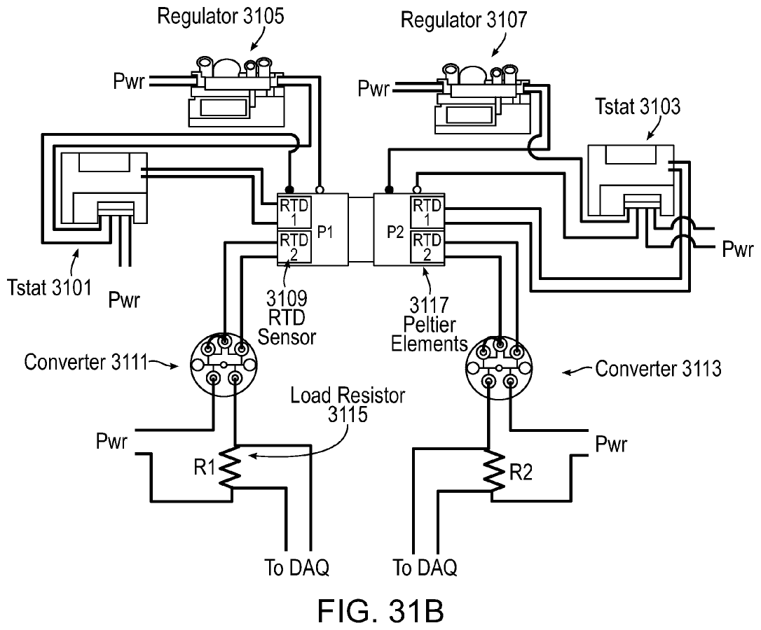

FIG. 31A illustrates a thermal assembly attached to the side of the cage (enclosure) in accordance with one or more embodiments of the present disclosure. A template can be used for aligning the location of the assembly within the Laboratory Cage housing as shown in FIG. 31A.

FIG. 31B is an electronic schematic diagram of the RTTMS in accordance with one or more embodiments of the present disclosure.

In some embodiments, the RTTMS assembly as shown in FIGS. 31A-31B may include two sets of digital thermostats (Tstat 3101 and 3103), current voltage regulators (Regulator 3105, 3107), RTD signal converters (Converters 3111, 3113), and load resistors (e.g., Load Resistor 3115). The thermostats can have a digital LED display and programmable interface for setting the setpoint, hysteresis, and temperature limit parameters. The thermostats can be powered by DC power supply voltage and may utilize relays that cycles on-off. Control signals can then be boosted using a current voltage regulator, powered by a power supply voltage, which output signals to the Peltier elements 3117. The thermistors can provide temperature feedback to the thermostat. The RTD sensors (e.g., RTD sensor 3109) return signal can be first amplified in a signal converter, and then converted to voltages using the load resistors. The Laboratory Cage power bus may provide the power supplies to power the electronics.

In some embodiments, voltage readings from the RTTMS can be received by a Peripheral Component Interconnect Express (PCIe) hardware component. The voltages may be converted to 16-bit counts, which may then be converted by the experimental hardware controller software to a temperature. The temperature can span a maximum of 80 degrees Celsius. These values can then be written to a data file along with a timestamp at one second intervals. The data file, along with the generated video, can be later processed for analysis of the subject temperature as described herein.

In some embodiments two software services may be employed to operate and monitor the infrared camera, including a video capture service and an imaging service. Each service can be deployed in individual machines controlling the individual laboratory cages. These services can start automatically at machine startup and await requests from a client application or service run in production.

In some embodiments, a video capture service can receive requests from the controller computer software application and can execute requests to record videos for the infrared camera. The camera can be pre-configured to record at 3 frames per second. An example of the process is as follows:

A WINDOWS service can receive a command from the controller to establish a connection to the infrared camera.

The service attempts to connect to the camera. If the camera is in a Ready state and indicates that the Calibration Complete, then it can return True.

If True, then the Controller can respond with a request to start recording with a message that includes a unique Test Study-Subject ID, a target output directory in which to store the video, and the video duration in minutes.

Using the Study-Subject ID, the service can generate a unique File Key as the name of the video file.

Using the AFORGE API, the service can initialize a video and begins recording.

The service can then start an internal timer in which to monitor the elapsed time and compares it to the expected time.

Recording proceeds until the service receives a request to stop recording from either the Master Controller or the internal timer.

After which, the video file can be saved on the local machine, timestamped using an FFMPEG process, and then copied to the targeted output directory.

In some embodiments, the Imaging service can receive requests from the Controller Computer. The service can handle requests to retrieve snapshot images from an infrared camera selected by the user using the AFORGE API to service the request. The retrieved image can then be displayed in the application for the user to review prior to conducting a Test Run.

In some embodiments, the Infrared Camera Calibration System can include an electro-mechanical fixture and a software application.

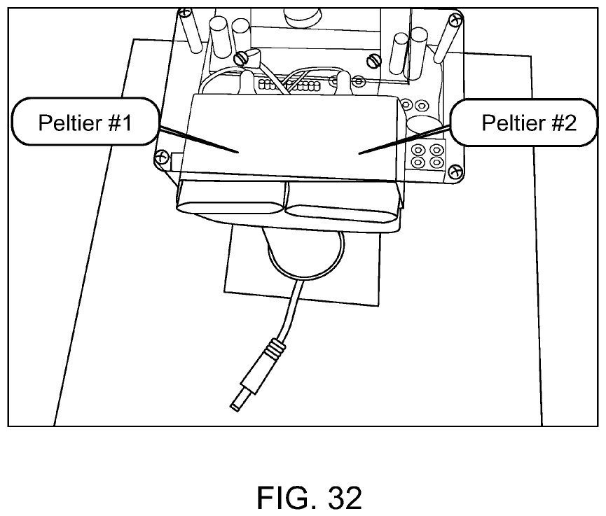

In some embodiments, the Calibration Fixture can include a floor, a pedestal, and a dual-Peltier Electro-Mechanical Sub-System. The Peltier assembly can sit atop the pedestal. The assembly can be the same as used in the RTTMS, as described herein above. The thermal elements can be covered in black thermal conductive tape and are 30 mm square. The pedestal can sit atop a flat white plastic floor. The floor can be sized to fit flat within the Laboratory Cage with the pedestal positioned in such a way as to be directly beneath and centered in view of the Infrared camera positioned above. The electrical assembly can be housed beneath a protective clear plastic cover (see FIGS. 31A-32).

FIG. 32 illustrates a fixture of the Infrared Camera Calibration System in accordance with one or more embodiments of the present disclosure.

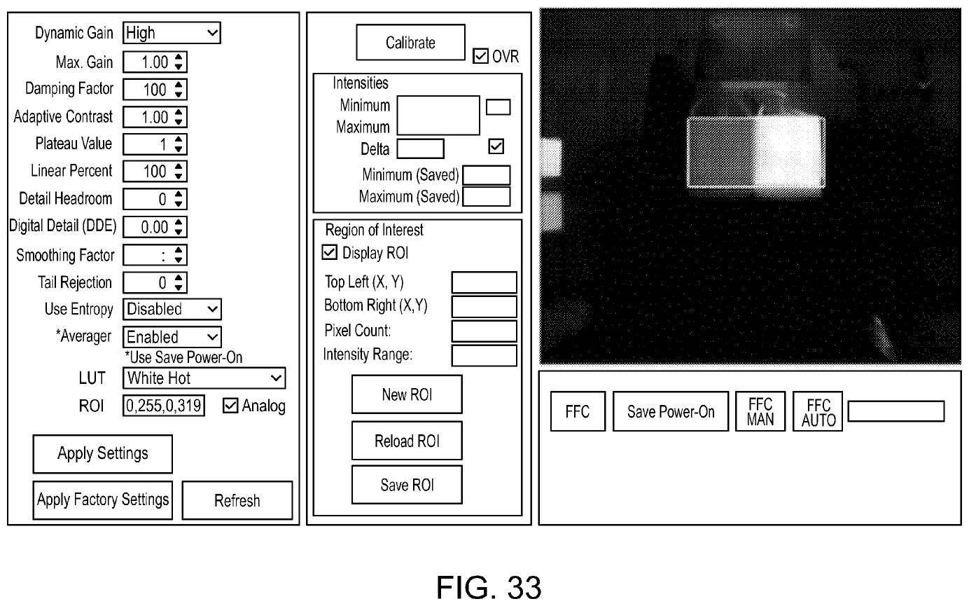

In some embodiments, the Calibration Software application can be a GUI-based application used by lab personnel for setting the camera operational parameters. The calibration settings can be uploaded to the camera using commands sent via the FLTR API and FFMPEG library. Laboratory personnel can use the software to send a fixed set of pre-configured parameters to the cameras and draw a Region-of-Interest (ROI) perimeter around the energized Peltier elements displayed in the GUI. This action can be performed weekly for each system assigned to each Laboratory Cage to reduce or eliminate the effects of sensor thermal drift as shown in FIG. 33.

FIG. 33 illustrates an Infrared Camera Calibration Software Application graphic user interface (GUI), in accordance with disclosed embodiments. This GUI can be displayed for example on GUI 388 of display 386.

In some embodiments, the computer vision software for inferring core body temperature may need to quantify the calibrated optical density of the subject from a thermal video channel. A behavioral platform may be equipped with an infrared thermal camera and two standard reference temperatures maintained by Peltier devices placed on the side of the infrared camera view at the periphery. The Peltier devices may be maintained at constant temperature.

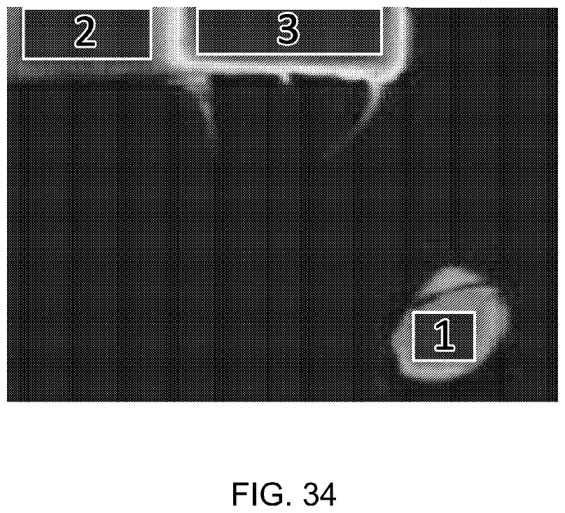

In some embodiments, a calibration test can be carried out by placing a container filled with hot water (e.g., a temperature-reference liquid) in the thermal camera view along with a thermocouple sensor measuring the water temperature in real time as shown in FIG. 34 below. In the example displayed in FIG. 34, the readout from the thermocouple measured the decrease in the water temperature over time and can be used to calibrate the thermal camera's settings to capture the dynamic range of relevant temperatures. As an example, the dynamic range can be between about 24 and 36 degrees Celsius. Temperature calibration can be required for each image as there can be substantial inter-frame variation of Optical Density (OD) and potential drift bias over time. In a preferred behavioral platform, two Peltier devices may be installed and set to reference constant temperatures of 28 and 34 degrees Celsius, around the range expected by the normal (or drug-induced) temperature variation in mice. In another embodiment, the range of values may be wider, ranging from severe hypothermia to severe hyperthermia and considering that the eye temperature may be lower than the core temperature, but higher than the coat temperature.

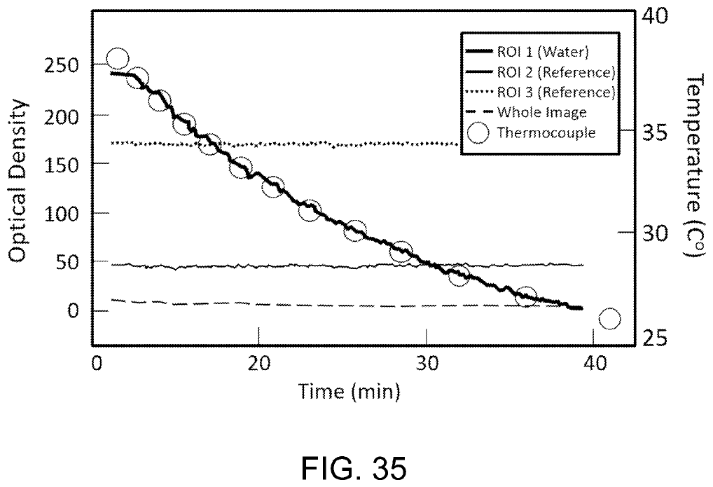

FIG. 35 illustrates an example of calibration testing, in accordance with disclosed embodiments. In this example, the regions of interest (ROI) are numbered as 1 (hot water), 2 (low standard temperature), 3 (high standard temperature) on the top figure. The thermal image shows enhanced pseudo-colors (high temperature in red, low temperature in blue). The bottom figure is a graph of the optical density measurements in this example calibration test over almost 40 minutes (left axis on graph) for the three ROIs and the whole image, and real time temperatures of the thermocouple probe placed in the water cup (right axis on graph). As this example shows, a computer system can derive the temperature data from the optical density to the extent that the standard Peltier devices can be used.

In some embodiments, the thermal video processing may include the steps of image calibration, eye segmentation, feature extraction, and validation.

In some embodiments, with regard to image calibration, in an experimental session, actual Peltier temperatures may be logged and saved along with the thermal camera video. The logged Peltier temperatures in Celsius can be paired to the optical density (OD) levels measured at the two Peltier ROIs in each corresponding frame, so that a linear calibration curve can be used to convert the OD of measured eye ROIs to degrees Celsius.

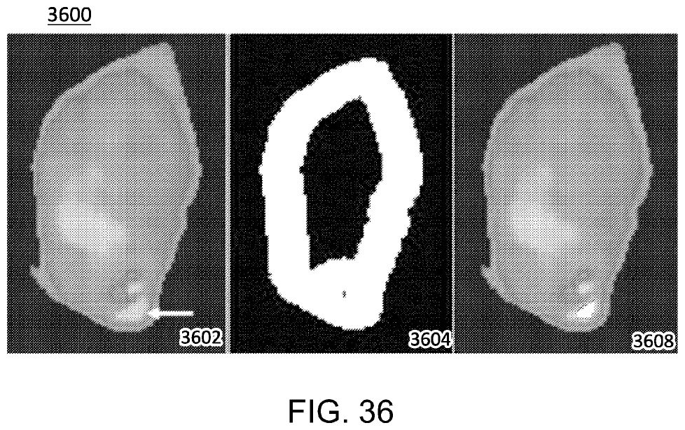

FIG. 36 illustrates eye segmentation image processing, in accordance with disclosed embodiments. From left to right: detail of thermal video frame of a mouse in SmartCube, with white arrow 3602 pointing to the eye; masking of periphery of mouse body to restrict search of eye ROI 3604; image with eye ROI segmented 3608 (white).

In some embodiments, the subject body, such as a mouse, can be segmented based on the optical density distribution on each frame. The contour of the mouse can be masked by finding the largest eight-connected objects in the binarized image as shown in FIG. 36. To identify the eye region, the central area of the mask can be removed when a pixel's distance from the closest edge of the mask is more than half of the maximum distance overall. This may limit the search to the periphery of the mouse. Within this mask, the eye can be selected as the largest object identified by selecting pixels that are more than the top X % of the signal range. In a preferred embodiment, X can equal 4. Finally, the top half of the pixel values can be averaged to estimate the OD of the eye region. The size of the eye region relative to the mask and shape properties such as ‘Eccentricity’ (ratio of the distance between the foci of the ellipse and its major axis length) and ‘Solidity’ (proportion of the pixels in the convex hull that are also in the region, computed as Area/ConvexArea) can be measured to filter out false positives, when those parameters may be outside of the range learned from manually annotated ground truth examples.

In some embodiments, with regard to feature extraction, the average OD of automatically detected eye ROIs can be converted to Celsius based on the calibration curve. The temperature measurements can be averaged across each behavioral platform period. For each period, temperature features (in degrees Celsius) related to the expected value and variability can be extracted. For example, for each period, the mean, median, standard deviation, standard error of the mean (SEM), and other estimates of the expected value and variability, may be extracted.

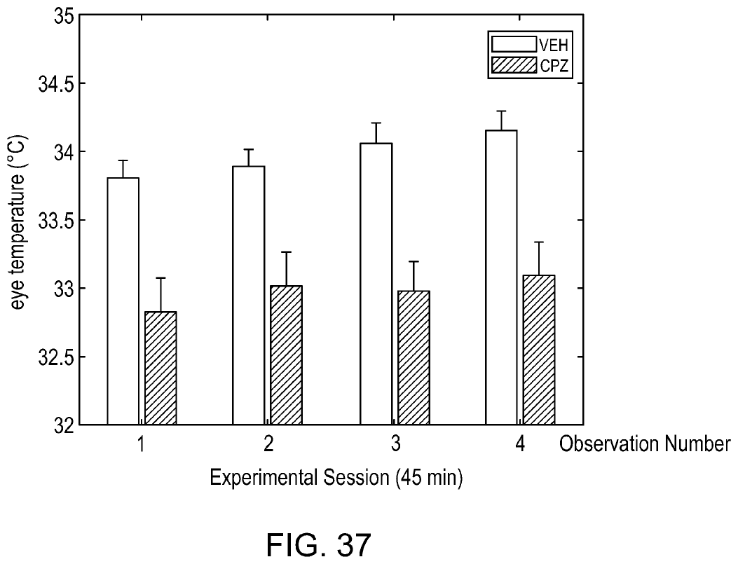

FIG. 37 illustrates an exemplary measurement of a decrease of estimated eye temperature, consistent with disclosed embodiments. In this example, the decrease of estimated eye temperature occurred in behavioral platform observations (over a 45-minute session) in mice treated with 1 mg/kg chlorpromazine (CPZ) or vehicle (VEH). In some embodiments, a validation experiment can test whether this method may effectively detect changes in body temperature induced by drugs from thermal videos. In one such test, two groups of mice can be treated with chlorpromazine 1 mg/kg (n=13) or vehicle (n=14) intraperitoneally, and tested after 15 minutes under the thermal camera system. An average change of 0.9 degree Celsius in core temperature was observed by measuring rectal temperature of animals before injection and just prior to their thermal camera recording.

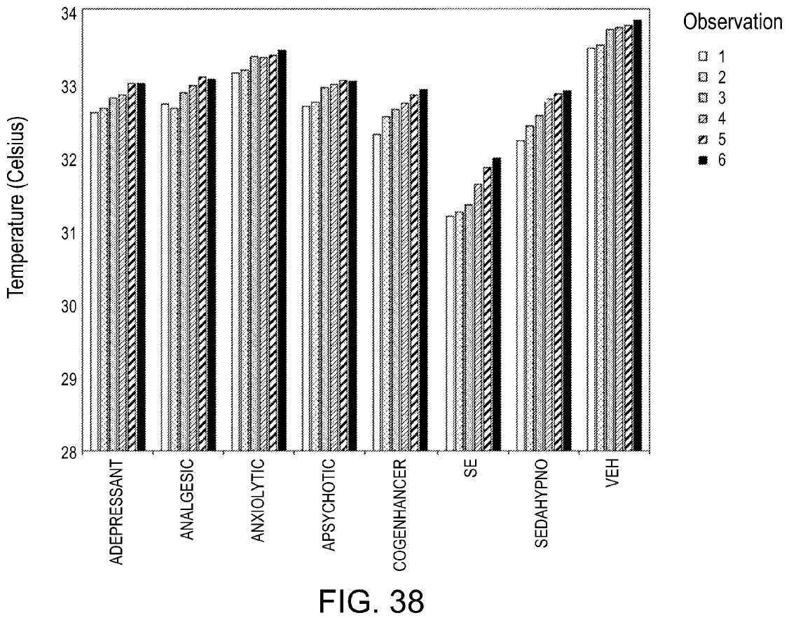

FIG. 38 illustrates an exemplary graph showing an average eye temperature for different classes of psychoactive compounds, measured in accordance with disclosed embodiments. The average eye temperatures were measured using the automated thermographic system across experimental periods of a 45-min session. The analysis of eye temperature extracted from thermal videos detected a significant decrease in estimated eye temperature across the approximately 45 min of the experimental session, confirming that this method may detect changes in body temperatures induced by drugs.

In some embodiments, such as in the example illustrated in FIG. 38, the psychoactive compounds may include: ADEPRESSANT: Antidepressant; ANALGESIC: Analgesic; ANXIOLYTIC: Anxiolytic; APSYCHOTIC: Antipsychotic; COGENHANCER: Cognitive Enhancer; SE: Side effect; SEDAHYPNO: Sedative/Hypnotic; VEH: Vehicle, and other classes without limitation. The average detected eye temperature across 6 different observations of a 45-min behavioral platform measurement session showed a decrease in temperature for most classes, with maximal decreased in the side effect class as shown above in FIG. 38. In addition, an increase of about 0.5 degrees C. was observed over time, across the different behavioral platform periods, with modulation of the increase by the different drug classes.

In some embodiments, the system can be used to analyze the effect on temperature of representative drugs for different drug classes. Drug classes may include different types of antidepressants, psychedelics, hallucinogens, entheogens, antipsychotics, anxiolytic, sedative-hypnotic, mood stabilizers, psychostimulant, anticonvulsants, anti-migraine, drugs of abuse (such as alcohol, nicotine, cocaine, heroin), and the like without limitation.

In some embodiments, the treatment being tested can comprise compounds that do not cross the blood-brain barrier but are still of interest with respect to their effects on temperature. Such drugs can be tested with intracranial administration to compare their peripheral versus central effects.

In some embodiments, the treatments can be non-pharmacological such as manipulation of the environment before or during the experimental session, genetic, optogenetic, or any other manipulation of the subjects.

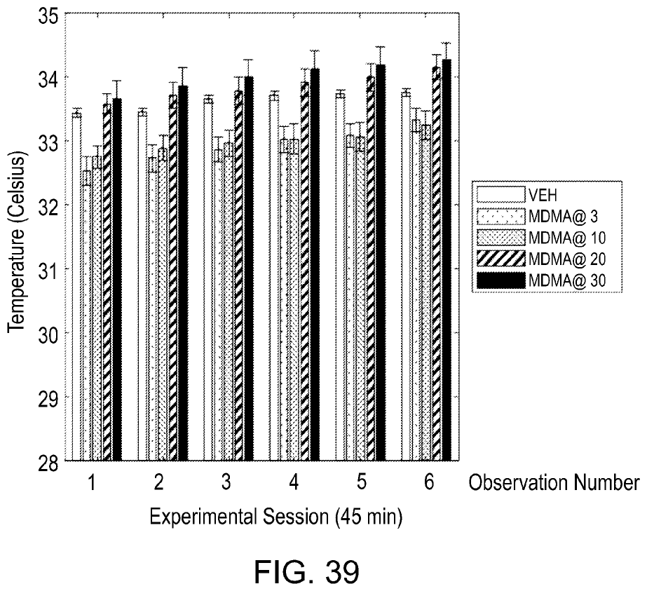

FIG. 39 illustrates an exemplary graph showing dose-dependent increase of eye temperature as measured by an automated thermographic system of a behavioral platform. The eye temperatures were observed after administration of various doses of 3,4-methylenedioxymethamphetamine (MDMA), a compound known to increase body temperature.

In some embodiments, a system comprises a memory, an enclosure, a container, a temperature calibration module, at least one thermal imaging camera, and at least one processor. The temperature calibration module can include at least one first Peltier element and at least one second Peltier element placed on a wall of the enclosure. The temperature calibration method can also include calibration circuitry to maintain the at least one first Peltier element at a first reference temperature, and at least one second Peltier element to maintain the at least one second Peltier element at a second reference temperature. The container can include a temperature-reference liquid placed in the enclosure. The temperature-reference liquid can have an initial temperature at an initial measurement timestamp in a predefined measurement time interval higher that the first reference temperature and the second reference temperature. The predefined measurement time interval can be a time between a final measurement timestamp and the initial measurement timestamp. The at least one thermal imaging camera can be configured to capture thermal image data of a plurality of thermal images of the at least one first Peltier element, the at least one second Peltier element and the temperature-reference liquid in the container. Each thermal image in the plurality of thermal images can include a timestamp within the predefined measurement time interval. The at least one processor can be configured to execute computer code stored in the memory that causes the at least one processor to continuously receive thermal imaging data from the at least one thermal imaging camera over the predefined measurement time interval. The at least one processor can be configured to determine, using the thermal imaging data, an optical density of the at least one first Peltier element, the at least one second Peltier element and the temperature-reference liquid in the container by inputting the thermal image data of each thermal image in the plurality of thermal images into at least one image processing module. The at least one processor can be configured to compute a temperature of the temperature-reference liquid at each timestamp associated with each thermal image in the predefined measurement time interval from the initial temperature at the initial measurement timestamp to a final temperature at the final measurement timestamp, as the temperature-reference liquid cools, based on the first reference temperature of the at least one first Peltier element, the second reference temperature of the at least one second Peltier element, and the optical density of the at least one first Peltier element, the at least one second Peltier element, and the temperature-reference liquid in the container. The at least one processor can be configured to generate thermal camera calibration data using the temperature of the temperature-reference liquid at each timestamp for each thermal image over the predefined measurement time interval.

In some embodiments, the temperature-reference liquid can be water.

In some embodiments, the at least one processor can be configured to upload the thermal camera calibration data to the at least one thermal imaging camera.

In some embodiments, the system can further include a subject in the enclosure. The at least one thermal imaging camera calibrated with the thermal camera calibration data can be configured to receive thermal imaging data of the subject.

In some embodiments, the at least one processor can be configured to compute the body part temperature of the body part of the subject by determining an optical density of the body part of the subject from the thermal imaging data.

The aforementioned examples are illustrative and not restrictive.

### Section F

In at least some cases, observational data can originate from disparate electronic sources (e.g., different databases). For example, data in a first source can be obtained utilizing a first data generating technique and/or can be collected at a first time. In turn, data in a second source can be obtained utilizing a second data generating technique and/or can be collected at a second time. There might be one or more structural differences between the first and second sources. For example, in some cases, data in the first source might be stored differently than data in the second source due to differences in database schemas between them. Typically, a machine-learning classifier can be trained to classify data from a particular data source. Consequently, to at least address the above identified technological problem when dealing with different databases, the present disclosure utilizes, in various embodiments, computer-based systems configured to dynamically normalize datasets. The normalized datasets can then be used for training and applying hybrid classifiers.

In some embodiments, illustrative computing devices of present disclosure may be programmed with one or more techniques of the present disclosure to use content-rich observational data to train a supervised machine learning classifier to map features for a modification to a corresponding clinical-used pharmacological “label” (e.g., CNS Indication or Mechanism of Action). As an example, a classifier of a behavioral platform can be trained using support vector machine trained on profiles of reference modifications, including a class, profiled at multiple doses. Dose responses for the reference manipulations can be generated using multiple doses targeting both efficacious doses as well as doses that exhibit side effect profiles (e.g., in test animals including mice, or other subjects). Each drug dose may be profiled using a suitable number of replicas, such as, e.g., 10, 15, 20 or more independent replicas.

In some embodiments, the output of the resulting classification algorithm can be a probability distribution over the set of labels. The labels can represent a specific biological response and/or therapeutic indication associated with the modification. In some embodiments, the classification can also use the probability distribution to predict quantities such as “unknown activity” (differences from vehicle, e.g., as quantified using an “Activity” algorithm, not attributable to any specific feature patterns in the training set), as well as “total activity” of the drug (e.g., sum of probabilities of all labels, including unknown activity, except for vehicle).

In some embodiments, illustrative computing devices of present disclosure can be programmed with one or more techniques of the present disclosure to use profiles for a selection of drugs from two or more datasets. The datasets can be produced from profiles provided by multiple behavioral platforms, stored in different databases, or other separate technologies and techniques for creating, formatting, and storing the profiles of the datasets. Such a combination of data from multiple sources may improve the robustness of the classifier by increasing the training dataset.

Consistent with disclosed embodiments, a behavioral platform can be configured to normalize each feature of each dataset when combining multiple datasets into a combined dataset. In some embodiments, the behavioral platform can normalize features on a per feature basis, using an independent normalization value for each feature.

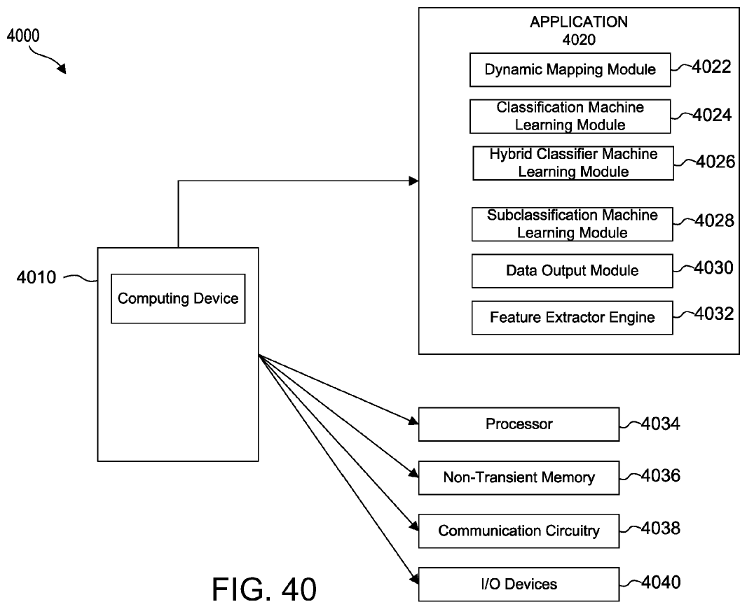

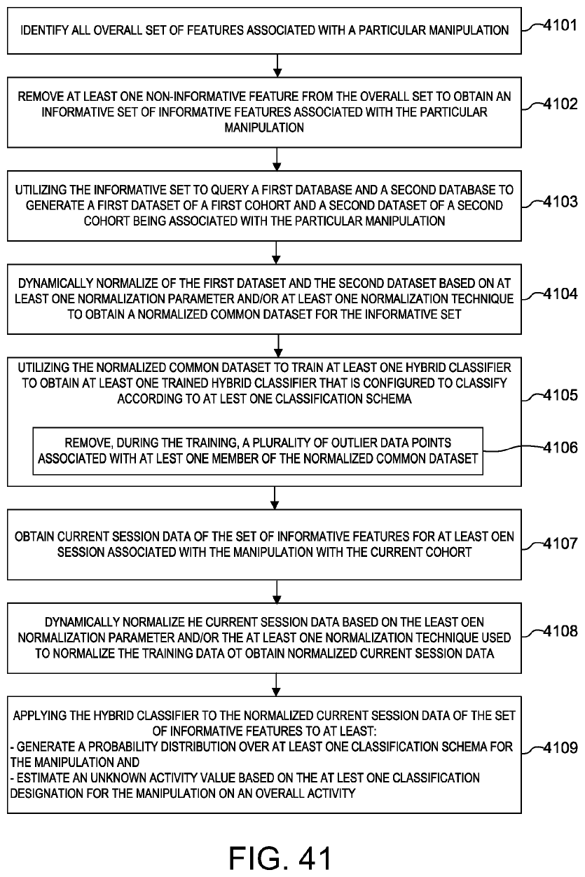

FIG. 40 illustrates a block diagram of an exemplary computer-based system and platform for dynamically modifying a database schema to create a mapping of a class label and/or sub-class label to a manipulation within a database of data sets, in accordance with at least one embodiment. FIG. 41 illustrates a flowchart of an exemplary computer-based method for dynamically modifying a database schema to create a mapping of an outlier data point to a label within a database of data sets, in accordance with at least one embodiment.

In some embodiments, as shown in FIG. 40, an illustrative computing system 4000 of the present disclosure may include a computing device 4010 associated with a user and an application 4020 In some embodiments, application 4020 can be stored on computing device 4010. In some embodiments, application 4020 may reside on a server computing device (not shown). In some embodiments, the computing device 4010 may include a processor 4034, a non-transient memory 4036, a communication circuitry 4038 for communicating over a communication network (not shown), and input and/or output (I/O) devices 4040 such as a keyboard, mouse, a touchscreen, and/or a display, for example.

In some embodiments, application 4020 may can include a dynamic mapping module 4022, a classification machine learning module 4024, executing an exemplary hybrid classifier 4026, a subclassification machine learning module 4028, a data output module 4030, and/or a feature extractor engine 4032.

In some embodiments, feature extractor engine 4032 of the present disclosure can include one or more feature extractors. The feature extractors can be configured to extract observational features from input observational data. These observational data can include “raw” or processed observational data. Exemplary raw observational data can include raw observational data can include, without limitation, sensor data, aversive stimulus data, motor challenge data, lighting data, tactile stimulus data, pseudo-3D map data, two-dimensional (2D) camera data, three-dimensional (3D) camera data, thermal camera data, side 2D camera data, side 3D camera data, floor force sensor data, water and feeder data, and/or additional sensor data. Processed observational data can include raw observational data that has been filtered, deduplicated, de-meaned, reformatted, scaled, binned, or the like.

The feature extractor engine 4032 can store sets or sequences of the features in a database according to a database schema. In some embodiments, the database may store a library of list signatures for corresponding modifications, as described herein.

In some embodiments, the feature extractor engine 4032 may obtain input data associated with the response of at least one subject to a modification. In some embodiments, the feature extractor engine 4032 may utilize multiple feature extractors to generate observational features (e.g., instant features, states, motifs, domains, or the like) from the input data.

In some embodiments, a dataset may be formed from an experimental session for data acquisition and/or analysis performed over a predefined duration, analyses of videos may be done frame by frame, or of other time series done at the highest resolution possible. Such observational data can be used to generate instant features. For example, the instant feature set of a mouse in a given point of time within an experimental session may be the set of x, y, z coordinates for its head, body center, paw positions, heart rate and eye temperature.

Consistent with disclosed embodiments, an instant feature may be analyzed “as is,” or may be summarized and/or integrated into higher order features or modified in any number of other ways. These higher-order features can include states, motifs, or domains. In this manner, the observational data can be used to build complex phenotypic models, as described herein.

For example, a behavioral platform can employ computer vision and mechanical actuators to detect spontaneous and evoked behavior eliciting responses through anxiogenic and startling stimuli. These sensors can acquire over two thousand higher-order features and more than half a million instant features. Consistent with disclosed embodiments, a behavioral platform can be programmed to extract features concerning locomotion, trajectory complexity, body posture and shape, simple behaviors, behavioral sequences, or any combination thereof. For example, a behavioral platform can employ computer vision to detect changes in gait geometry and gait dynamics in rodent models of neurological disorders, pain and neuropathies, and extract gait (gait geometry and gait dynamics such as stance, swing, propulsion, etc.) and non-gait behaviors. Typically, the output of such a behavioral platform can be a large set of observational features that can be used for various analyses as detailed herein.

In some embodiments, the domain feature extractor may refer to a collection of features representing physiology such as temperature. In some instances, the domain feature extractor may be gait geometry, motor coordination, paw position and paw pressure, while other domain feature extractors could be exploratory behavior versus consummatory behavior.

In some embodiments, illustrative computing devices of present disclosure may be programmed with one or more techniques of the present disclosure to, at step 4101, identify an overall set of features associated with a particular manipulation. In some embodiments, the overall set of features may be a compilation of all features formulated for all test subjects associated with a particular manipulation. In some embodiments, each feature in the set of features may include a statistical distribution of feature values extracted from each test subject.

In some embodiments, illustrative computing devices of present disclosure may be programmed with one or more techniques of the present disclosure to, at step 4102, remove at least one non-informative feature from the overall set to obtain an informative set of informative features associated with the particular modification.

In some embodiments, to develop a hybrid classifier, non-informative features in the overall set of features may be removed. In some embodiments, non-informative features may be defined as features having an inter-percentile difference in the associated statistical distributions between two predetermined threshold percentiles that is below an inter-percentile threshold value. In some embodiments, an example of a non-informative feature may be a feature having an inter-profile difference between the 90^{th} percentile and the 10^{th} percentile of a predetermined threshold (e.g., zero or other suitable value). In such an example, each feature in the overall set of features may be tested by calculating inter-percentile difference for each feature by taking the difference of 90 percentile and 10 percentile and removing from the overall set of features those with an inter-percentile difference of a predetermined threshold (e.g., zero, or other suitable value), resulting in selection of an informative set of features for the hybrid classifier model.

In some embodiments, illustrative computing devices of present disclosure may be programmed with one or more techniques of the present disclosure to, at step 4103, utilizing the informative set of features to query a first database and a second database to generate a first dataset of a first cohort and a second dataset of a second cohort being associated with the particular modification.

In some embodiments, using the query of the informative set of features may cause each database to identify the features matching each informative feature in the set of informative features. Each database may then return the matching features. In some embodiments, the features from the first database may be grouped into the first dataset and may be associated with a first cohort associated with the source of the first dataset. Similarly, the features from the second database may be grouped into the second dataset and may be associated with a second cohort associated with the source of the second dataset.

In some embodiments, two cohorts from two sources may have variations in the type, format, calibration, etc. of the data. Accordingly, training a machine learning model from both cohorts can result in inaccurate modelling of the ground truth effects of a manipulation, while training the machine learning model from only one cohort may be insufficient data to achieve accuracy.

In some embodiments, illustrative computing devices of present disclosure may be programmed with one or more techniques of the present disclosure to, at step 4104, dynamically normalize the first dataset and the second dataset based on at least one normalization parameter and/or at least one normalization technique to obtain a normalized common dataset for the informative set.

In some embodiments, to enable the two cohorts to be combined for a single unified dataset of features, each of the first dataset and the second dataset may be normalized. However, different types of features may represent differently and/or have different types of data. Thus, specific normalization techniques and/or independent and separate normalization for each feature type or for each individual feature. In some embodiments, to balance computational resources while ensuring effective normalization, the features may be grouped by type, and the features of each feature type may be normalized together yet independent form the features of each other feature type. Other types of features may be used with each normalized customized particularly for the feature type.

In some embodiments, after normalization, the underlying distribution of features profiled by each source may overlap (FIG. 41) to a greater extent than before normalization (FIG. 40). Thus, the normalization can make features of each type more comparable, increasing the strength of the training dataset to enable training of the hybrid classifier with both cohorts from both sources.

### Classification Machine Learning Module 4024

Consistent with disclosed embodiments, a behavioral platform can, at step 4105, use the normalized common dataset to train one or more hybrid classifiers of the classification machine learning module 4024 to obtain at least one trained hybrid classifier that is configured to classify according to a classification schema.

In some embodiments, before inputting the features for training the hybrid classifier of the classification machine learning module 4024, the data may be filtered. For example, any not-a-number (NAN) or infinite value may be changed to 0. In some embodiments, all the features may be rounded to a particular number of decimal places, such as, e.g., 4 decimal places, or any other suitable number of decimal places and/or significant figures. In some embodiments, to further pre-process the features, each feature may be transformed using a suitable statistical evaluation (e.g., a suitable statistical comparison and/or statistical scoring, such as a z-score or the like). For example, the features may be z-score transformed to a reference mean (e.g., a mean of zero) and a reference standard deviation (e.g., a standard deviation of one).

Consistent with disclosed embodiments, a behavioral platform can, at step 4106 and prior to or during training of the hybrid classifier of the classification machine learning module 4024, remove outliers from the normalized common dataset.

In some embodiments, the behavioral platform can remove a plurality of outlier samples from the input data based on an applied dynamic prediction associated with the chemical compound. In some embodiments, the behavioral platform can use the data set of the plurality of chemical compound signatures at multiple doses to build a classifier to predict the plurality of labels.

Consistent with disclosed embodiments, a hybrid classifier of a behavioral platform can include a suitable neural network. In some embodiments and, optionally, in combination of any embodiment described above or below, an exemplary implementation of neural network may be executed as follows:
define neural network architecture/model,
transfer the input data to the exemplary neural network model,
train the exemplary model incrementally,
determine the accuracy for a specific number of timesteps,
apply the exemplary trained model to process the newly received input data, and
optionally and in parallel, continue to train the exemplary trained model with a predetermined periodicity.

Consistent with disclosed embodiments, the trained neural network model can specify a neural network by at least a neural network topology, a series of activation functions, and connection weights. For example, the topology of a neural network may include a configuration of nodes of the neural network and connections between such nodes. Consistent with disclosed embodiments, a trained neural network model can include other parameters, such as bias values/functions, activation functions, or aggregation functions. An activation function can include a step function, sine function, continuous or piecewise linear function, sigmoid function, hyperbolic tangent function, or other type of mathematical function that represents a threshold at which a node of a neural network is activated. In some embodiments and, optionally, in combination of any embodiment described above or below, an aggregation function can be a function that combines (e.g., sum, product, etc.) input signals to a node of a neural network. Consistent with disclosed embodiments, an aggregation function output can be an input to an activation function for a node of the neural network. Consistent with disclosed embodiments, a bias may be a constant value or function that may be used by the aggregation function and/or the activation function to make the node more or less likely to be activated.

In some embodiments, the hybrid classifier may be the trained classifier. In some embodiments, rather than a single trained classifier, the hybrid classifier can include an ensemble of models. For such an ensemble, a selected number of different models (e.g., 20, 25, 30, 35, 40, 45, 50, 55, 60, 65, 70, 75, 80, 85, 90, 95, 100 or more) may be trained on the same data, with different model weight initializations. In some embodiments, to apply the hybrid classifier, observational features acquired from a subject of a modification (e.g., a subject administered a certain dose of a drug) can be processed into a suitable format (e.g., through normalization as described herein) and input to each model in the ensemble. Each model can predict a probability across all classes for the subject. When observational features acquired from multiple subjects of a modification are provided, each model can predict a probability across all classes for each subject. The median of these probabilities can be taken as a final probability for the model for this modification (e.g., the effect of the administration of the dose of a drug). The output of the hybrid classifier can be a function of the predictions of the individual models in the ensemble. For example, the output of the hybrid classifier can be the median of the probabilities for each class across the individual models in the ensemble. In some embodiments, the final prediction may be scaled to have 100% probability across all classes.

### Sub-Classification Machine Learning Module 4028

Consistent with disclosed embodiments, a behavioral platform can optionally, at step 4105, use the normalized common dataset to train one or more hybrid classifiers of the sub-classification machine learning module 4028 to obtain at least one trained hybrid classifier that is configured to classify according to a sub-classification schema. In some embodiments, the hybrid classifier may include the classifier of the classification machine learning module 4024, the sub-class classifier of the sub-classification machine learning module 4028, or the classifier of the classification machine learning module 4024 may be combined with the sub-class classifier of the sub-classification machine learning module 4028.

Consistent with disclosed embodiments, a behavioral platform can, at step 4106 and prior to or during training, remove outliers from the normalized common dataset.

Consistent with disclosed embodiments, upon training the hybrid classifier, a behavioral platform can, at step 4107, obtain a current session data of the set of informative features for a session associated with the manipulation with a current cohort. In some embodiments, the current session data may be identified according to a time associated with the datasets.

Consistent with disclosed embodiments, a behavioral platform can, at step 4108, dynamically normalize the current session data based on the at least one normalization parameter and/or the at least one normalization technique used to normalize the training data to obtain normalized current session data. In some embodiments, the normalization may occur with respect to the overall set of features, as described above to obtain the normalized current session data.

Consistent with disclosed embodiments, a behavioral platform can, at step 4109, apply the hybrid classifier to the normalized current session data of the set of informative features. In some embodiments, the hybrid classifier can include the classifier of the classification machine learning module 4024, the sub-class classifier of the sub-classification machine learning module 4028, the independent binary classifier, or any suitable combination thereof. As a result, the hybrid classifier can output a probability distribution over the classification schema including the classes and/or subclasses (e.g., hierarchical classifier). Additionally, the independent binary classifier can estimate an unknown activity value based on the classification designation for the manipulation as described above. The unknown activity value can then be used to adjust the classification by the hybrid classifier, e.g., as described above.

In some embodiments, the training of the classifier and/or the sub-class classifier can include a determination of “unknown” signals. Since the classifier is trained on predefined classes or subclasses, the features of a test compound may be forced to be classified as one of the trained classes and/or subclasses only. However, it is possible that a compound produces a response outside the realm of the available domain and therefore the output of the classifier does not represent the true underlying compound signature.

Consistent with disclosed embodiments, a behavioral platform can be configured to address this limitation by including an additional class, called an “unknown class” in the final output of the hybrid classifier. The unknown class indicates that the manipulation is active, but the classification system could not reliably assign certain features or patterns (which may be new or insufficiently strong changes exhibited at lower doses) to any known class and/or sub-class.

Consistent with disclosed embodiments, a behavioral platform can calculate the “unknown” signature by comparing any manipulation data with a corresponding vehicle data using an independent binary classifier. In some embodiments, the independent binary classifier may be used to ascertain the overall activity of the manipulation, which may then be used to update the overall classification output of the classifier.

In some embodiments, the non-transient memory 4036 can store the plurality of classified labels that can be displayed on the computing device 4010 via the at least one GUI having the at least one programmable GUI element. In some embodiments, the non-transient memory 4036 can store the normalized uniform data set associated with the plurality of chemical compound signatures and the input data. In some embodiments, the non-transient memory 4036 can store the identified outlier data point associated with the uniform data set based on the at least one calculated probability distribution value and the plurality of labels. In some embodiment, the non-transient memory 4036 can store output of the exemplary dynamic mapping module 4022. In some embodiments, the non-transient memory 4036 can store output of the plurality of feature extractor engine 4032. In some embodiments, the non-transient memory 4036 can store output of the classification machine learning module 4024. In some embodiments, the non-transient memory 4036 can store output of the subclassification machine learning module 4028. In some embodiments, the non-transient memory 4036 can store output of the data output module 4030.

As a non-limiting example, content-rich observational data was used to train a supervised machine learning classifier to reliably map features for a drug to its corresponding clinical-used pharmacological “label” (e.g., CNS Indication or Mechanism of Action). Specifically, two distinct databases were used, each having subject feature profiles obtained from experiments with drugs (e.g., SC or NC) that might have been performed at different times, under different data collection techniques, etc. From two databases, a selection was made of a group of unique drugs, examples shown in Table V.

TABLE V

Alprazolam
Amitriptyline
Aricept
Aripiprazole
Aspirin
Atomoxetine
Bupropion
Buspirone
Caffeine
Carbamazepine
Chlordiazepoxide
Chlorpromazine
Citalopram

A set of features were obtained for each unique drug in the group. For the normalization, the non-informative features from the entire data were removed. To remove non-informative features, inter-percentile difference for each feature was calculated by taking the difference of 90 percentile and 10 percentile and removed features whose inter-percentile difference was zero, resulting in selection of over 1,034 features for the final model. Next, the data extracted from the two databases for the informative features was normalized so that it could be combined to build a classifier. The normalization was done differently for different sets of features.

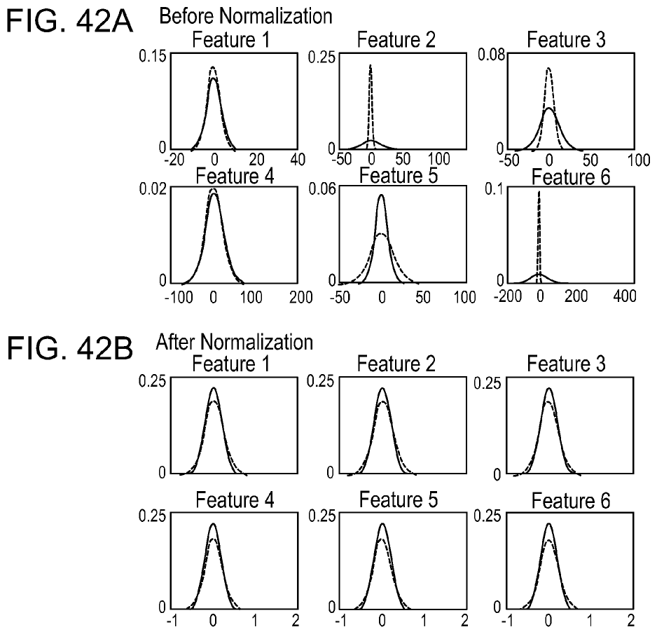

FIGS. 41A-42B illustrate distributions of exemplary features A) before normalization and B) after normalization consistent with embodiments of the present disclosure. Thick and dotted lines indicate features extracted from two databases respectively. After the normalization, as shown in FIGS. 41A-42B, the underlying distribution of features profiled in in two databases overlapped significantly (FIG. 42B) compared to their distribution before the normalization (FIG. 42A).

Next, a classifier was trained for both class and subclass analyses using labels of drugs that are, for example, without limitation, currently prescribed or have been clinically validated for a specific therapeutic indication.

### A Non-Limiting Example of Training a Class Classifier

All the drugs in the selected group of unique drugs, such as some provided, for example, in Table V, may cover different indications/classes, as for example identified in Table VI. Consequently, the dataset of all these drugs profiles at multiple doses was used to build a new so-called “hybrid” classifier to predict, for example, without limitation, labels in Table VI.

TABLE VI
Drug classes in the classifier.
Drug Class

Antidepressant
Analgesic
Anxiolytic
Antipsychotic
Cognitive Enhancer
Psychostimulant
Side effect
Sedative
Control/Vehicle

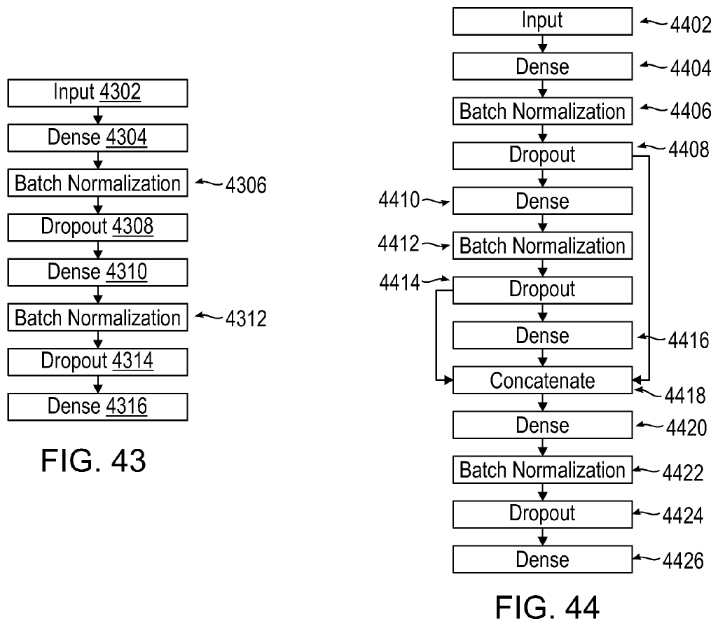

The observational features along with the associated drug class labels were used to build a classifier model using neural networks. Before inputting the data, any not-a-number (NAN) or infinite value was changed to 0 and all the features were rounded off to 4 decimal places and z-score transformed with mean 0 and unit standard deviation. Further, outlier samples were removed from the training. To identify outlier samples, a neural network model, having an exemplary non-limiting architecture as shown in FIG. 43, was trained on all the data and applied for prediction on the same set. The model was trained using multi-class cross-entropy loss and Adam algorithm-based optimizer with a suitable learning rate of 10-4, batch size of 64 and for 30 epochs. The predicted results of the samples which fall into anyone of the following criteria, were removed as outlier and not used for training: 1) the correct drug class for the sample was not among the top 3 predicted classes; 2) predicted correct class had a probability of less than 0.2 and 3) the difference between the probabilities of the predicted correct class and maximum probability of any other class was greater than 0.2.

FIG. 43 illustrate an exemplary fully connected model architecture for outlier removal process. The input layer 4302 receives the data features, and the output layer returns the probability of the drug sample being in the possible 13 classes. The same fully connected model architecture can be used, with different parameters, for the exemplary, non-limiting drug class classifier. In such cases, the input layer receives the data features, and the output layer returns the probability of the drug sample being in the possible 13 classes.

The above detailed exemplary outlier removal process excluded about 10% of the data as outliers. These outlier samples were removed from the final classifier. The data left after the outlier removal process was z-score transformed and used to build the final classifier with an architecture also shown in FIG. 43.

The model architecture was a fully connected model which took an input 4302 of the data features and passed to 2 blocks of dense 4304, 4310, batch normalization 4306, 4312, and dropout 4308, 4314 (p=0.2) layers with ‘elu’ activations. These encoded features were finally passed to classifying dense layer 4316 with ‘softmax’ activation, which provides the probabilities for the 13 or more drug classes. The model was trained using multi-class cross-entropy loss and Adam algorithm-based optimizer with learning rate of 5×10\hat{ }(−4), for 80 epochs. The output of the resulting classification algorithm was a probability distribution over the chosen set of labels.

For the final classifier, 100 different models were trained on the same data, with different model weight initializations. This was done to create an ensemble of models to have a robust prediction. To apply the classifier, all the samples of the test drug dose were first z-score transformed and passed to each of 100 models that predicted the probabilities across all classes for all individual samples for a drug a given dose. For a given model, the median of all these samples was taken and then medians across all models were taken to have a final prediction for a given drug dose. These predictions were finally scaled to have 100% probability across all classes.

In some embodiments, other ways to combined models can be used such as majority voting, or other averaging methods, such as arithmetic or geometric mean, without limitation.

To evaluate the overall accuracy of each model, a whole-drug-out assessment was used where one (1) drug was systematically removed from the dataset, and rest of the drugs were used for training a new model. The excluded data was then used for model validation. For each left-out drug at different doses the class with the highest probability was considered and called a correct prediction (true positive) if it matched with the correct label or a wrong prediction (false positive) if it did not. Thus, the overall precision and accuracy of the model for different classes was calculated. An estimated accuracy of around 75% with class specific accuracy ranging from 40% to 100% for different classes was found. Most of the lower accuracies were for under-represented classes in the model, which can likely be improved in future models by including more training drugs for these classes.

In some embodiments, the accuracy of the model can be assessed building test sets by leaving out a percentage of samples across classes, a percentage of samples for each drug, or for each dose, without limitation.

Another approach was used to look at the result by averaging the predicted probabilities for different classes and representing via, without limitation, a confusion matrix. The confusion matrix shows only the probabilities for each correct class (represented in the diagonal of such matrix) but also shows the biases or systematic mistakes in the models.

### A Non-Limiting Example of Training a Classifier for Predicting Class and Subclass

All the drugs in Table VII were also classified into different subclass labels, illustrative sublabels include, without limitation, the sublabels of Table VII. The dataset of all these drugs profiles at multiple doses, selected as detailed before, were used to build a new “hybrid” hierarchical classifier for predicting both class and subclass labels in Table VII.

TABLE VII
Example of drug sub-classes in the subclass classifier.

| Subclass | Class |
|---|---|
| Non-selective Reversible MAO Inhibitor | Antidepressant |
| NE Reuptake Inhibitor | Antidepressant |
| Selective 5HT Reuptake Inhibitor | Antidepressant |
| Triple Reuptake Inhibitor | Antidepressant |
| Tricyclic | Antidepressant |
| Opiate Agonist | Analgesic |
| Nonsteroidal Anti-inflammatory | Analgesic |
| 5HTIA Agonist | Anxiolytic |
| Benzodiazepine | Anxiolytic |
| Atypical | Antipsychotic |
| Typical | Antipsychotic |

Similar to the above example of training the exemplary, non-limiting Class classifier, the normalized data was pre-processed by changing any not-a-number (NAN) or infinite value to 0 and rounding all the features to 4 decimal places, and finally z-score normalizing with mean 0 and unit standard deviation. Similarly, outlier samples were removed from the training. To identify outlier samples, a neural network model (architecture given in FIG. 44) was trained on all the data and applied for prediction on the same set. The classifier was trained using multi-class cross-entropy loss and Adam algorithm-based optimizer with learning rate of 2×10\hat{ }(−4) for 100 epochs. The predicted results of the samples that satisfied any of the following criteria were removed as outliers and not used for training: 1) the correct drug subclass for the sample was not among the top 3 predicted subclasses; 2) predicted correct subclass had a probability of less than 0.2 and 3) the difference between the probabilities of the predicted correct subclass and maximum probability of any other subclass is greater than 0.2. This process excluded about 10% of the data as outliers. These outlier samples were removed from the final hybrid classifier. The remaining data after the outlier removal process was z-score transformed and used to build the final hybrid classifier shown in FIG. 44. The model architecture was a fully connected multi-task model that took an input 4402 of the data features (1034 features) and generated 13 drug classes and the required subclasses as an output. The data was passed to two blocks of dense 4404, 4410 batch normalization 4406, 4412 and dropout 4408, 4414 (p=0.2) layers with “elu” activations. These encoded features were finally passed to classifying dense layer 4416 with “softmax” activation, which provides the probabilities for the 13 drug classes enumerated in Table VI. The output probabilities of the drug class, produced from this layer along with the encoded features from the first 2 dense layers 4404, 4410 are concatenated 4418 and passed to a block of dense layer 4420 with 64 units, batch normalization 4422 and dropout 4424 (p=0.2) layers with “elu” activations. The encoded features, enriched with class information too, were passed to the final dense layer 4426, trained using multi-class cross-entropy loss for both class and subclass prediction and Adam algorithm-based optimizer with learning rate of 2×10\hat{ }(−5) for 100 epochs. The final dense layer outputs the probabilities for the current 56 drug subclasses enumerated in Table VII.

FIG. 44 illustrates a fully connected model architecture for a hybrid hierarchical classifier. Input layer receives the data features, and the output layer returns the probability of the drug sample being in the possible 56 classes.

For the hybrid hierarchical classifier, 100 different models were trained on the same data, with different model weight initializations to generate an ensemble of models to have a robust prediction. To apply the classifier, all samples of the drug dose were first z-score transformed and passed to each of 100 models that predicted the probabilities across all subclasses for all individual samples for a drug a given dose. For a given model, the median of all these samples was taken and then the median across all models was taken to generate a final prediction for a given drug dose. These predictions were scaled to have 100% probability across all classes. The overall accuracy of the model was assessed using the whole-drug-out process as described herein, for example for the class hybrid classifier. The overall precision and accuracy of the model for different subclasses were calculated, with accuracy overall reaching 70%.

In some embodiments data for subclasses with low accuracy can be reinforced with more data at the dose levels, more doses at the drug level, or more drugs. Subclasses that are confused with each other can be merged, and heterogeneous subclasses can be further divided into more homogeneous subclasses. Subclasses with low accuracy may be defined by an accuracy below 70%, below 65%, below 60%, or below any other suitable accuracy threshold without limitation.

In some embodiments, alternative ways to assess accuracy of a subclass classifier, can be employed, such as those described above, among others or any combination thereof.

### Illustrative Non-Limiting Example of Determination of “Unknown” Signal

Since the above exemplary classifiers were trained on predefined classes/subclasses, it might force the signal of a test compound to be classified as one of the trained classes/subclasses only. In some instances, a candidate compound might produce a response outside the realm of the available domain and therefore the output of the classifier would not represent the true underlying compound signature of the candidate compound. In some instances, an additional class was included, called an “unknown class” in the final output of the hybrid class classifier. The “unknown class” indicated that the candidate compound was active, but the hybrid classifier could not reliably assign certain feature(s) and/or pattern(s) (which may be either novel or just insufficiently strong changes exhibited at lower doses) to any pre-determined class/subclass, although it detected the difference from a vehicle (e.g., a composition that would include the candidate compound during a treatment).

As non-limiting example, the “Unknown” signature was calculated by comparing any compounds data with the corresponding vehicle data using an independent binary classifier (see FIG. 45 below) that was used to ascertain the candidate compound's overall activity that then would be used to update the overall classification output of the hybrid classifier. For example, the classification of the activity with the binary classifier was according to the following steps:
subsampling: k=100 iterations of subsampling of the larger group (between control and drug treated samples) to match sample size n of the smallest group (if the two groups were of equal size, this step was still repeated k times, using all the data (without subsampling));
subsampled dataset was z-normalized;
the dataset was then dimensionality-reduced by Principal Component Analysis (PCA), by selecting the top components covering at least 50% of the variance;
for each k iteration the success rate of a Linear Discriminant Analysis (LDA) binary classifier was computed as follows:
iterate*n*40 times a random partitioning of each group, where ⅓ of the data was left out, and ⅔ were used for training, and compute classification accuracy as (TP+TN)/total;
compute average classification accuracy across random partitioning iterations; and
minimum accuracy capped at 0.5; and
the activity score was computed as the average classification accuracy across the k iterations.

Activity from this classifier was used to rescale the output from the original classifier. Scaling was done by comparing the activity of the candidate compound with the total activity from the original classifier, defined as 1-vehicle activity, where vehicle activity was the probability corresponding to vehicle class/subclass. If the “activity” was less than the total activity, then the probability of the vehicle in the original classifier output was set to the “activity” and probability of all classes were scaled such that sum of these equals 1-activity. If the “activity” was greater than total activity, then the “unknown class” was introduced, and its probability was set to activity minus total activity. The results for the class and subclass analyses could be presented as standardized bar charts with percentages that sum to 100 for each dose.

In some embodiments, the evaluation of a dose associated with the candidate compound can be done in multiple steps. The steps may include two steps, which may include: (1) forming subsamples, randomly selected from the compound and vehicle sample sets, and (2) dividing each subsample into training and test subsets. The proportion of training samples to test sample by be 75% training and 25% test, or any other suitable proportion, such as, e.g., 80% training and 20% test, 85% training and 15% test, 90% training and 10% test, 95% training and 5% test, 99% training and 1% test, or other suitable proportion without limitation.

In some embodiments, the steps for evaluation can be repeated several times and the results can be averaged to define the overall “activity” of the candidate compound (which, in short, is the accuracy of this 2-class classifier). The output of this classifier can be used to rescale the output from the n-class classifier. Scaling may be done by comparing the activity of the candidate compound according to the 2-class classifier (A₂) with the sum of the class predictions of the n-class classifier (Aₙ). If A₂<Aₙ, then Aₙ is set to Aₙ=A₂, and the probability of all classes may be rescaled such that sum of these equals A₂. If A₂>Aₙ then U=A₂−Aₙ may be set as the “unknown class” probability. If A₂=Aₙ, no change may be required.

FIG. 45 illustrates an exemplary RNN model architecture for binary drug activity classifier in accordance with one or more embodiments of the present disclosure. The input layer may receive 4 timestamps (the number of bins in 2 post-dosing hours).

The aforementioned examples are illustrative and not restrictive.

In some embodiments, the present disclosure provides, without limitation, a computer-implemented method that may include steps of:
obtaining, by at least one processor of a computing device, raw test subject data associated with at least one test subject response to a plurality of manifestations;
utilizing, by the at least one processor, a plurality of feature extractor engines to generate input training data associated with a plurality of features based at least in part on the raw test subject data;
generating, by the at least one processor, a database schema including a plurality of data records that store the plurality of features associated with the plurality of manifestations;
generating, by the at least one processor, a uniform data set associated with the input data by normalizing the plurality of chemical compound signatures based on a plurality of data vectors, where at least one data vector of the plurality of data vectors is a latency data vector;
utilizing, by the at least one processor, a classification machine learning algorithm to classify the at least one feature according to at least one label of a plurality of labels based on historical data associated with the plurality of indicative markers associated with the input data and the plurality of data records stored within the database of data sets, where the at least one label of the plurality of labels represent at least one class of chemical compound;
where the historical data is stored according to a pre-determined database schema;
determining, by the at least one processor, at least one calculated probability distribution value associated with each chemical compound signature of the plurality of chemical compounds signatures based on the at least one label of the plurality of labels for the input data;
dynamically predicting, by the at least one processor, a plurality of quantities associated with each chemical compound signature of the plurality of chemical compound signatures based on the at least one calculated probability distribution value, where each quantity in the plurality of quantities is a type of activity associated with each chemical compound of the plurality of chemical compounds;
utilizing, by the at least one processor, a subclassification machine learning algorithm to identify at least one outlier data point associated with the uniform data set based on the at least one calculated probability distribution value and the plurality of labels associated with each chemical compound of the plurality of chemical compounds, where the at least one outlier data point represents at least one sub-class of chemical compound;
dynamically modifying, by the at least one processor, the database schema to create a mapping of the at least one outlier data point to the at least one label of the plurality of labels within the database of data sets; and
verifying, by the at least one processor, the mapping of the plurality of labels and the at least one outlier data point based on a utilization of the plurality of feature extractor engines and the plurality of chemical compound signatures associated with each chemical compound of the plurality of chemical compounds of the input data.

In some embodiments, where the plurality of feature extractor engines includes an instant feature extractor engine that feeds data into a state feature extractor engine that feeds data into a motif feature extractor engine that feeds data into a domain feature extractor engine

### Section G

As may be appreciated, comparisons between values of individual observation features acquired for subjects under different circumstances may not reveal overall or underlying phenotypic differences. Consistent with disclosed embodiments, a behavioral platform can address this technical problem by using decorrelated ranked feature analysis (DRFA) to identify overall or underlying phenotypic differences between sets of observational features. Furthermore, the observational features generated by a behavioral platform can be statistically related to each other. Such relationships can lead to overfitting in downstream analyses and/or erroneous interpretation of highly correlated features. Consistent with disclosed embodiments, a behavioral platform can address this technical problem by generating statistically independent combinations of the original features (further referred to as de-correlated feature(s)). In some embodiments, each de-correlated feature may be a statistically independent, weighted combination of all features. In some embodiments, one or more de-correlated features may be used for dimensionality reduction without loss of relevant information, which may be utilized for visualization and/or data interpretation, as detailed herein.

Consistent with disclosed embodiments, a behavioral platform can generate one or more Gaussian distributions approximating one or more groups of subjects in each given cohort (referred to herein as a “Cloud”) and may use an illustrative embodiment of DRFA to estimate a quantitative measure of separability, defined as discrimination probability, by calculating, for example, without limitation, the overlap between the underlying probability distributions of the two groups. In some embodiments, discrimination probabilities may be rescaled between 50% and 100%, where 50% represents no separation between two groups and 100% represents complete segregation.

Consistent with disclosed embodiments, a behavioral platform can use other suitable discrimination values where, for example, without limitation, the important factor is the associated p-value, where a significant discrimination is given by p<alpha=0.05. In one embodiment a value of alpha=0.01 can be used to minimize false positives (discriminations that are assumed significant when they are not) and in another embodiment the critical value could be alpha=0.1 when minimizing false negatives is more important than false positives. The first can be exemplified when a beneficial effect of a drug needs to be identified for further testing, and the latter, by an example, where missing a negative result (signs of cancer, for example) is problematic.

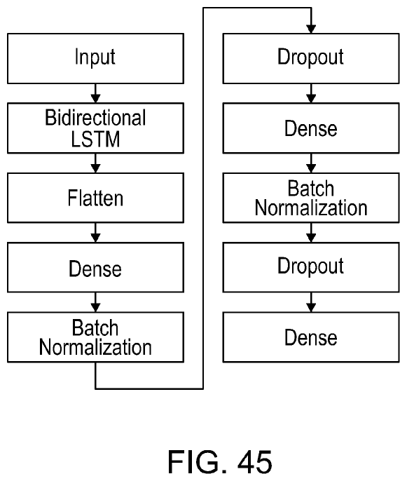

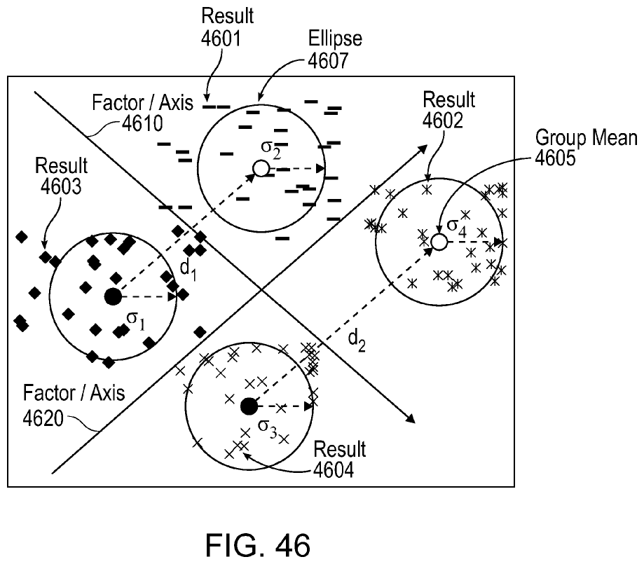

Consistent with disclosed embodiments, a behavioral platform can apply a feature ranking algorithm, derived from an exemplary support vector machine learning method (e.g., a linear SVM, a nonlinear VSM) to rank each de-correlated feature based on its discrimination power (for example, ability to separate the two groups, e.g., Control and Target). In some embodiments, illustrative computing devices of present disclosure may be programmed with one or more techniques of the present disclosure to further visualize sample groups as clouds in the space created by the two highest de-correlated ranked features (DRF1 and DRF2; FIG. 46).

FIG. 46 illustrates an exemplary visualization output of binary discrimination in the ranked de-correlated feature space that illustrative computing devices of present disclosure may be programmed to output. In some embodiments, clouds of experimental results (e.g., results 4601, 4602, 4603, 4604) can correspond to four experimental groups in the 2-dimensional de-correlated features space. In some embodiments, each cloud can be centered on mean for the corresponding experimental group (e.g., group mean 4605). An outer ellipse (e.g., ellipse 4607) can represent one standard deviation from the group mean for the elements in the corresponding experimental group, and/or an inner ellipse represents one standard error of the mean (SEM). The features DRF1 and DRF2 can form axes on the two dimensional space (e.g., factor/axis 4610 and factor/axis 4620). In some embodiments, illustrative computing devices of present disclosure may be programmed with one or more techniques of the present disclosure to further select DRF1 and DRF2 based on a ranking method. In some embodiments, DRF1 and DRF2 may capture the highest variability in the data, thus also provide a good representation of the data in two dimensions. As may be appreciated, this approach can be extended to illustrate the top 3 DRFs, plotted in a three-dimensional space.

### Illustrative Non-Limiting Examples of Feature Ranking

Consistent with disclosed embodiments, a behavioral platform can apply a feature ranking algorithm to estimate the discrimination power (ability to separate the two groups e.g., control and disease) of each feature. In some embodiments, an exemplary feature ranking algorithm can be programmed to weigh each feature change by its relevance: if there is a significant change in some irrelevant feature measured for a particular phenotype the low rank of this feature would automatically reduce the effect of such change in our analyses, so to avoid the so-called conventional “feature selection” approach and discard information buried in the less informative features. In some embodiments, illustrative computing devices of present disclosure can be programmed with one or more techniques of the present disclosure to further utilize top-ranking features to obtain insights into the biological underpinnings of the phenotype of interest.

Consistent with disclosed embodiments, a behavioral platform can assign weights to different samples by quantifying the degree of membership of each sample in a given cohort. These weights can be calculated by averaging the pairwise distances between all samples in a cohort. In some embodiments, illustrative computing devices of present disclosure can be programmed with one or more techniques of the present disclosure to further utilize these weights to compare two experimental groups to assess the fold change of each feature. For example, FIG. 47 below describes the original data for a disease model, the TG4510 mouse model of Alzheimer's disease. Feature fold change values are presented relative to the control and ranked. These relative values are capped between −100% at the lower end and 100% at the higher end. Zero percent in this plot represents no change between disease and control group. A discrimination score can be calculated for each feature, ranging from one, representing most relevant, to zero, representing no relevance. The discrimination score can be used to rank the features. FIG. 47 shows the top fifty or so top features. A threshold can be used to separate relevant versus non relevant features (e.g., at 0.2 of the discrimination score).

FIG. 47 illustrates an exemplary representation of feature values. Example showing features increased and decreased with respect to control for the mouse model of Alzheimer's disease TG4510 at 24 months of age. Overall discrimination was 86% achieved through the contribution of many decorrelated features. Each column represents a feature expressed as percent of the corresponding wild-type control (Feature Percent of Control) ranked by their contribution to the total discrimination.

### Illustrative Non-Limiting Examples of Recovery or Proximity Analysis

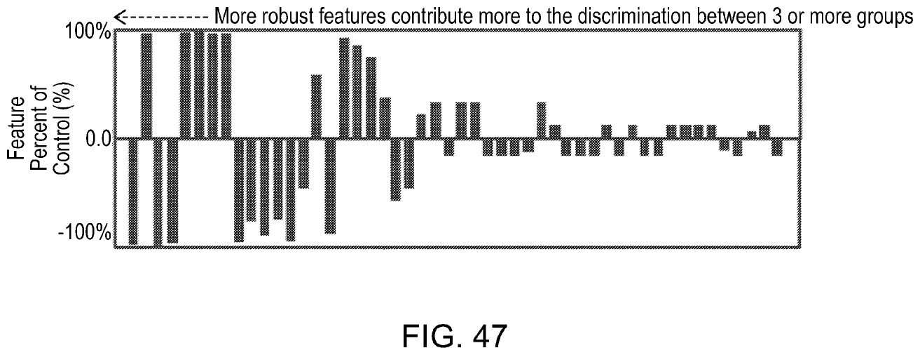

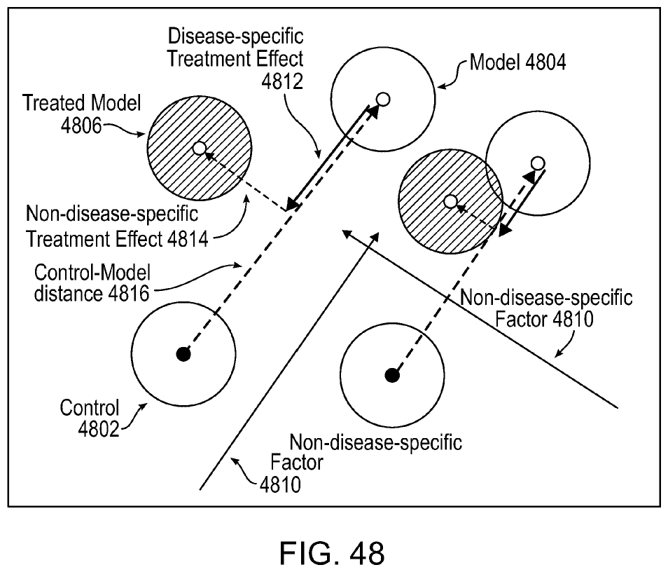

Consistent with disclosed embodiments, a behavioral platform can use, as an extension to the two-group DRFA analysis, a de-correlated feature space coordinate system to evaluate the impact of a rescue experimental group. Typically, the first two groups may be a control (e.g., control cloud 4802 with black center in FIG. 48), and a model group (e.g., model cloud 4804 with white center, FIG. 48). The third group, referred to as treated model group (e.g., treated model 4806 cloud, FIG. 48), represents the effects of a treatment expected to alleviate the symptoms of the disease. The drug treatment effect is represented as a combination of two components: a component along the direction of the disease specific factor axis 4808 (e.g., disease specific treatment effect 4812), and component along the direction of the non-disease-specific factor 4810 (e.g., other effects 4814) (FIG. 48). The relative length of the disease specific treatment effect 4812 with respect to the Control-Model distance 4816 can then be interpreted as the extent to which the drug induced a Model-specific recovery, whereas the relative length of the other effects arrow 4814 represents feature drug effects that are not Model-specific, representing other effects of the drug, or even side effects (“Other Effects”).

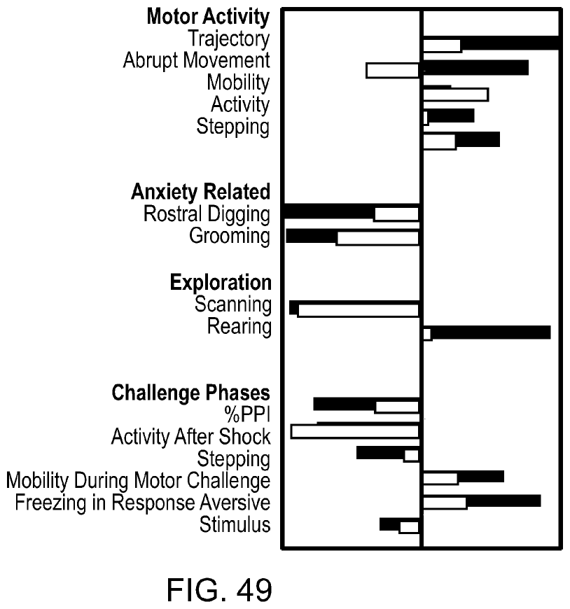

Consistent with disclosed embodiments, a behavioral platform can generate an exemplary plot of FIG. 48 to support assessment of the recovery of different features that separate the Control and the Model groups. The behavioral platform can calculate the fold changes between Control and Model Treated group and overlay them as white bars in the bar graph (FIG. 49). Values close to baseline or zero represent recovery of these features after drug treatment. Values close to the relative changes between control and disease group represent no or minimal effect of the drug. The black bars represent the original feature change profile of the Model. Percent changes are capped at −100% and 100%. FIG. 49 illustrates an overlap of relative fold changes between Control versus Model (black bars) and Control vs Treated Model group (white bars) showing recovery of various features following drug treatment.

### Illustrative Non-Limiting Examples of P Value Analysis: Assessing Statistical Significance

As may be appreciated, discrimination values can be artificially high in over-determined systems. Consistent with disclosed embodiments, a behavioral platform can address this technical problem by calculating the statistical significance of a result. For example, the behavioral platform can randomly split each cohort in a 1:3 ratio and use the larger set to build a distribution of “true” discrimination values for which

$${\int }_{50}^{100} {p}_{t r u e} ( x ) d x = 1$$

Consistent with disclosed embodiments, the behavioral platform can randomize the labels across different cohorts and again randomly split the data in, for example, without limitation, a 1:3 ratio and use to it build a distribution of “random” discrimination values

$${\int }_{50}^{100} {p}_{random} ( x ) d x = 1$$

In some embodiments, after normalization, the equation

$$sum P = \int {\int }_{x}^{100} {p}_{t r u e} ( x ) {p}_{random} ( y ) dx dy$$

represents the probability that the observed discrimination of the labeled set was produced just by chance. For drug studies, without limitation, at least some embodiments apply a similar approach to the projected vectors representing Recovery and Other Effects (e.g., generating multiple recovery indexes by random splitting and then compare with the results using an unlabeled set; the same process is used for side effects. In some embodiments, alpha may be set at the standard 0.05 level).

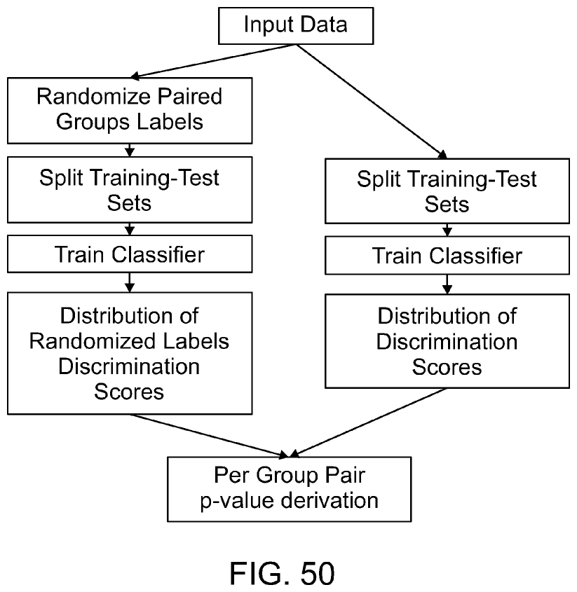

FIG. 50 illustrates assessing statistical significance of variability between control and disease groups. True discrimination and its variability are assessed by randomly splitting the control and disease datasets (represented by black and white dots in the input dataset) and repeatedly calculating discrimination indexes for each item, building a distribution of resulting discrimination indexes. Using sets of randomly labeled data, the process is repeated to build a distribution of discrimination indexes that are due to chance. The overlap area between the two distributions is a measure of the probability that the two correctly labeled groups are different just due to chance.

### Illustrative Non-Limiting Examples of Cube Specific Analysis

Consistent with disclosed embodiments, a behavioral platform can support platform-specific analysis(es). At least some analyses for SmartCube data, for example, could be performed using more than half a million instant features or about 2000 higher order features, or a combination thereof, obtained from one or more SmartCube platforms. Although these features may contain the overall information content underlying the mouse behavior and may be, typically, sufficient for the analysis, direct interpretation of these features, particularly the top features in the feature ranking, may not be feasible. Consistent with disclosed embodiments, to overcome this technical problem, a behavioral platform can use cluster feature analysis to pool multiple features into a single group or cluster in such that a way that they are both data-compatible and interpretable. In some embodiments, selection of features for a given cluster may be performed using at least two approaches. The first exemplary approach, called the legacy approach in the DRFA platform, is a data driven approach where data from a large cohort of vehicle treated samples (e.g., over 10,000; 15,000; 20,000; etc.) may be clustered to identify features with similar profile. The second exemplary approach uses knowledge domain about the 2000 raw features by identifying features with similar underlying interpretability. In both these approaches, once the features for a given cluster are identified, they are independently normalized using the parameters learned from the large cohort of vehicle treated samples and then averaged. Clustered features thus make the top features from the feature analysis more interpretable and constitute a type of dimensionality reduction focused on functional domains.

### Illustrative Non-Limiting Features

Features group analysis: This feature allows us to iterate through various feature combinations, which are based on underlying differing functional domains, and perform all the DRFA analysis independently or select any specific group. These feature groups are divided based on predefined subject feature categories. These features groups are:
Gait (both geometry and dynamics) features
Imaging features
Rhythmicity features
Paw Position features
Body Motion features

Weight decorrelation. In some implementations, feature values can be affected by characteristics of the subject. For example, gait feature values may depend on the weight of the subject. When subjects in a given cohort have very distinct weights, comparing them or combining them may be inappropriate. Weight decorrelation can remove bias introduced in feature values across different subjects due to their different weights. It removes this bias by first building a linear model between weight and a given feature and computing the residuals from this fitting. By definition, these residuals are statistically independent of the weights. The original feature values can then be replaced with these residual values.

### Illustrative Non-Limiting Preprocessing Steps

Imputation of missing data. Analysis within the DRFA platform may require data for all combinations of samples and features. An imputation step in the DRFA pipeline can handle missing data. The disclosed embodiments are not limited to any particular manner of replacing the missing data. In some embodiments, the missing data can be replaced with the average of all non-missing data for a given feature and cohort. In some embodiments, the missing data can be replaced by sampling an estimated underlying distribution of a feature in a given cohort. Each of these methods suffered from drawbacks. Averaging the data might not necessarily preserve the underlying datatype. Accurate estimation of the distribution often requires more samples than are available. Alternatively, values from subjects with non-missing values can be randomly selected and assigned to a subject with corresponding missing values, thus preserving both the datatype and the underlying distribution.

Outlier analysis. The DRFA platform can enable identification of outlier samples and removal of such samples from the analysis as an optional preprocessing step. A distance metric can be computed between all pairs of samples in each cohort. Any sample further than a threshold values from all other samples in the cohort can be labelled as an outlier sample and removed from subsequent analyses.

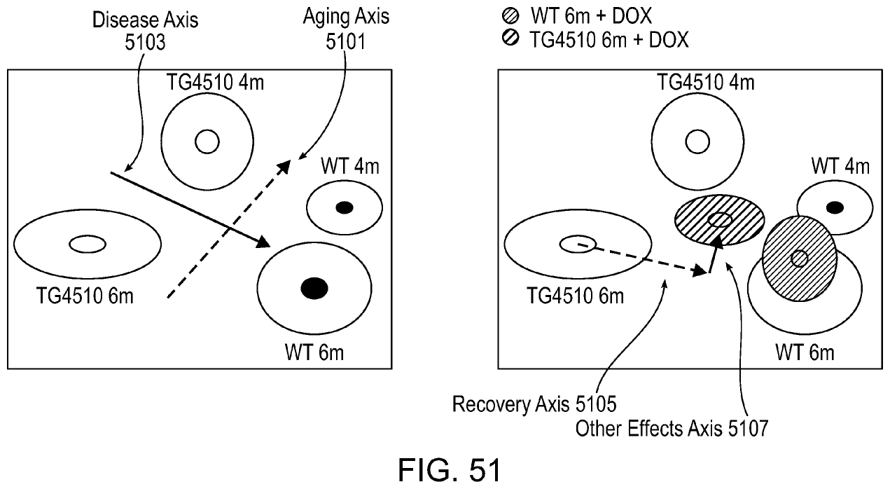

Separating orthogonal components in DRF space corresponding to different experimental factors. Consistent with disclosed embodiments, a behavioral platform can separate different factors driving a phenotype, such as progression of a Disease Model, effects of Treatment, Ageing, Drug response, and/or any other suitable factor(s). For example, it is possible to retest subjects in the platforms, and use DRFA to identify the axis that best explains the variance due to ageing. In the same way, an axis explaining disease progression can be calculated, and separated from ageing. As example, in FIG. 51, a DRFA analysis shows the ageing component of the space created by training two groups of wild type controls at different ages, and the corresponding Model groups at the same ages, where the Model is the TG4510 model of Alzheimer's Disease (FIG. 51, left panel). Treatment with doxycycline, which stops expression of the transgenic gene that drives pathology in this model, rescues the phenotype of the model as shown in the DRFA space (FIG. 51, right panel).

FIG. 51 illustrates exemplary DRFA analysis of the TG4510 model of Alzheimer's Disease showing separation of two, quite independent (orthogonal) axes corresponding to ageing (aging axis 5101 in the left panel) and disease phenotype (disease axis 5103 in the left panel). Note the separation of the Model and its Control (wild type mice or WT) is greater at 6 months (see the separation between the TG4510 6 m and WT 6 m) than at 4 months (TG4510 4 m versus WT 4 m). This Model is a conditional transgenic that can be rescued by treatment with doxycycline. Indeed, the Treated Model (TG4510+DOX) moved toward the WT control (Recovery axis 5105 in the right panel). The Treatment has a minor, non-disease specific effect in both the Model and the Control (Other Effect axis 5107 on the right panel).

Another example, using the TSC1 tissue-specific knockout, which is an animal model of Tuberous Sclerosis Complex, a developmental disorder, shows the ability of the present invention to separate the phenotype of an animal Model of disease, the Model-specific treatment effect, and the Model-non-specific treatment effects (FIG. 52). In this case the treatment is the drug rapamycin, which is very effective against some manifestations of Tuberous Sclerosis Complex.

FIG. 52 illustrates exemplary DRFA analysis of the TSC1 mouse Model of Tuberous Sclerosis Complex. The clouds on the left of the figure with thin borders are the TSC1 Model 5202 (stripped clouds) and the Control 5204 (white cloud), treated with vehicle, showing significant separation. The two thick border clouds correspond to the TSC1 Model (stripped cloud) 5206 and the Control 5208 (uniform grey) treated with 3 mg/kg rapamycin. Mice were dosed intraperitoneally 3 times/week, starting at postnatal day 10, with either vehicle (0.25% PEG200, 0.25% Tween80 in water) or rapamycin at 3 mg/kg. The dose volume was 10 ml/kg and the drug was prepped fresh prior to injection. All groups were tested in SmartCube® on postnatal day 24. The last dose of compound/vehicle was administered 60 minutes prior to testing. The analysis shows separation of two, quite independent (orthogonal) axes corresponding to disease phenotype 5210 (dashed arrow on the left of the graph) and treatment effect 5212 (thick arrows). Note the separation of the Model and its Control is greater at when both were treated with vehicle than when the Model was treated with rapamycin. That is, rapamycin rescued the Model. The Treatment with rapamycin has a minor, non-TSA specific effect in both the Model and the Control. (n=15; mixed gender).

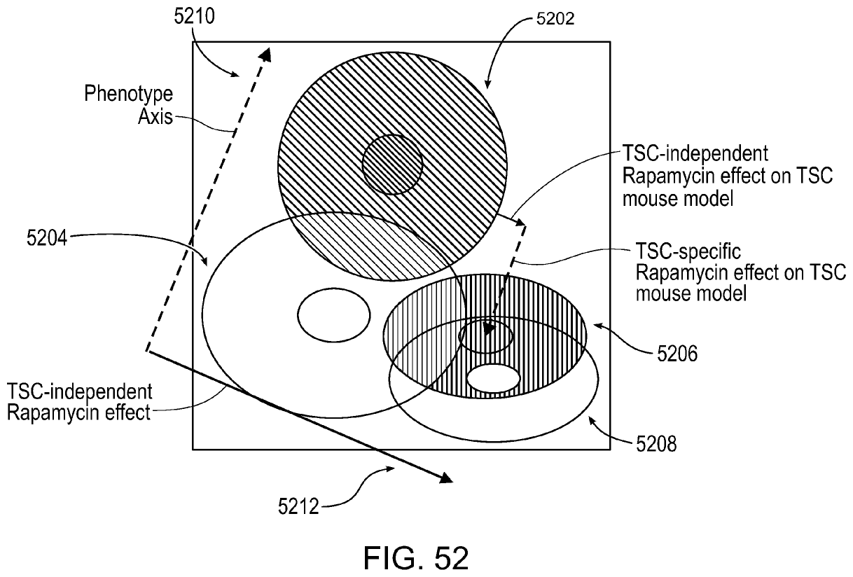

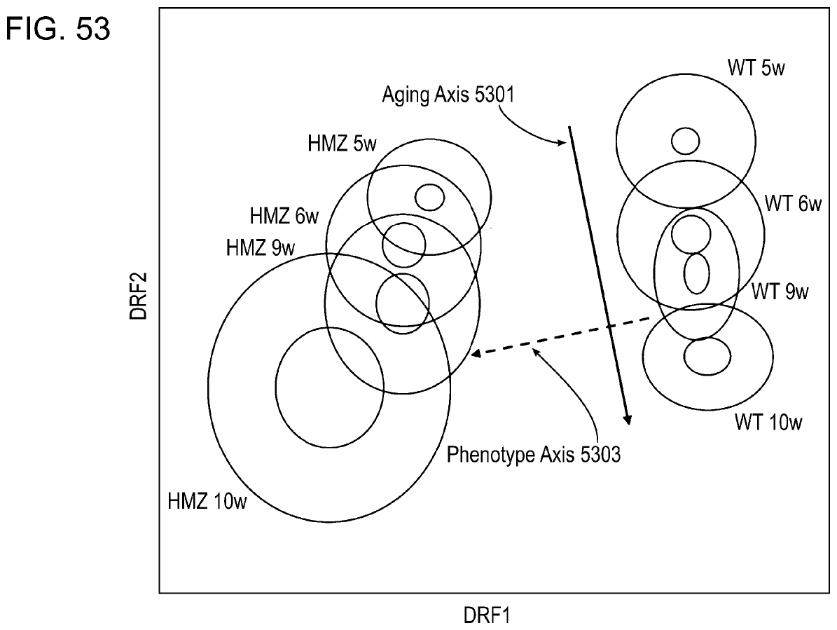

In another example, this multi-cloud DRFA analysis was able to separate disease and ageing axis of a mouse model of Rett syndrome tested in SmartCube at different timepoints between 5 to 10 weeks of age (FIG. 53). Of note, this multi-cloud analysis is supervised only in terms of one of the two factors, the disease factor in these examples, as disease groups and wild type groups are assigned to Reference and Target clouds, respectively, when applying DRFA algorithm. The observed separation of age in the DRF space, and the consistent progression over time of the different timepoints along the ageing axis is completely data-driven.

FIG. 53 illustrates exemplary DRFA analysis of the Mecp2 model of Rett syndrome showing separation of two, quite independent (orthogonal) axes corresponding to ageing (aging axis 5301) and disease phenotype (phenotype axis 5303). Note the separation of the Model and its Control (wild type mice or WT) is greater at 10 weeks (see the separation between the HMZ 10 w and HMZ 10 w) than at 5 weeks (HMZ 5 w versus HMZ 10 w).

In some embodiments, given orthogonal axes corresponding to distinct experimental factors (e.g., disease and ageing factors), a behavioral platform can be configured to extract the most relevant features contributing to the discrimination along each axis. The disease features, for example, can be extracted directly by the Feature Ranking method built in DRFA, as this function assigns weights to each feature based on the discrimination between Target and Reference groups. In some embodiments, to extract the ageing features, we need to run a new DRFA analysis in which the role of Target and Reference are assigned to wildtype groups of an early and a late timepoint respectively. If the experimental design consists of multiple timepoints, we can repeat this analysis for each pairwise comparison of different time points and combine them, or, in another embodiment, we can compare the earliest and the latest timepoint only.

Consistent with disclosed embodiments, a behavioral platform can use a cloud analysis method to quantify discriminations between WT groups and LacQ140I(*) mice. For example, this is an animal model of Huntington Disease, a neurodegenerative disorder. This method is based on the training of a classifier to separate, in a multidimensional space, the DRFA space, one or more “Reference” datasets versus “Target” datasets, and uses the classifier to examine where independent “Test” datasets fall in such space. We applied this method on the combined SmartCube® features data, grouping the samples as follows: the Reference (disease) groups consist of three LacQ140I(*) groups (tested at 6, 9, or 12 month of age). The Target (control) groups consist of the three wild type groups (combination of two different controls, WTI and WTI(*) groups) tested at the same ages. Finally, there are 3 sets of Test groups, which represent attempts to lower the mutant protein content driving neurodegeneration (huntingtin protein). These are the mHTT lowering groups LacQ140I and LacQ140I(2M) tested at three ages, and the LacQ140I(8M) tested at two ages.

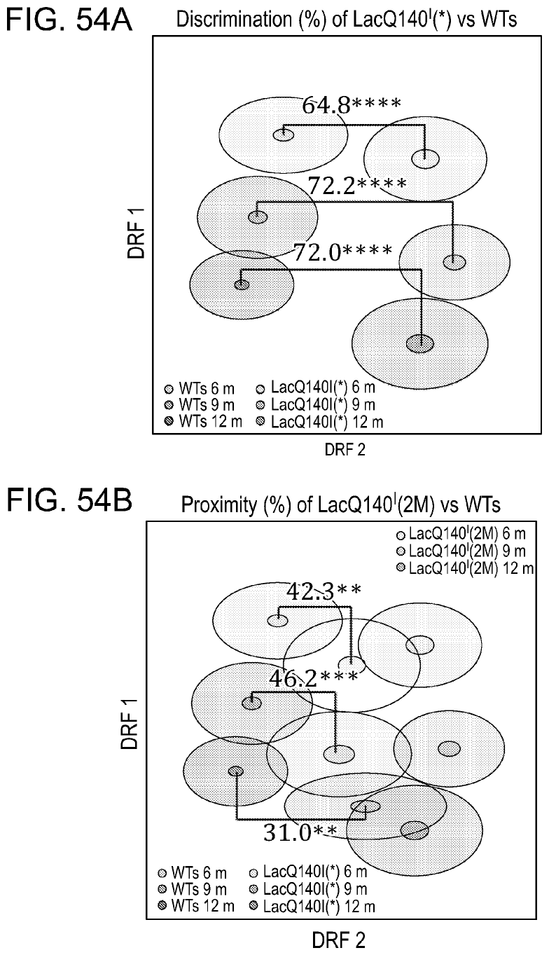

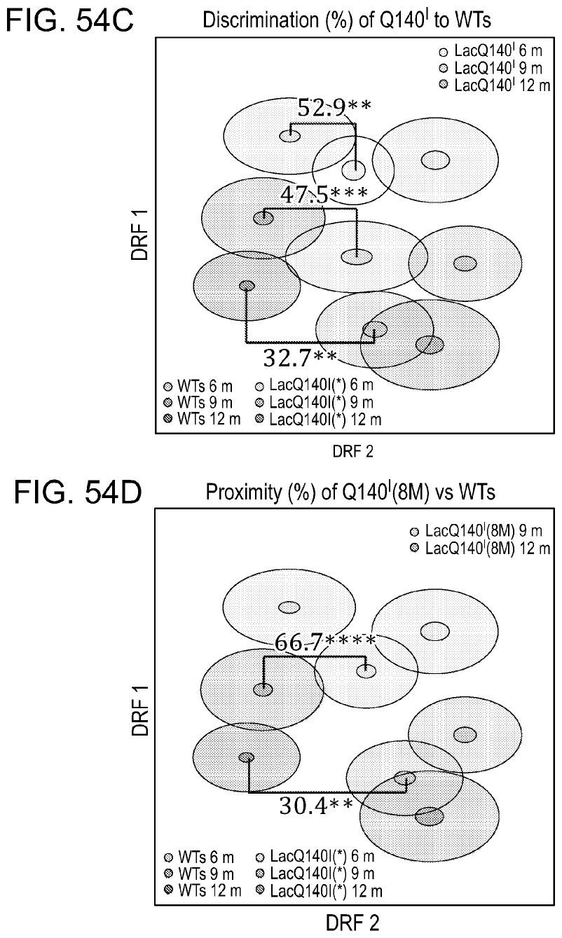

A cloud analysis method was used to infer the degree of discrimination between the Model LacQ140I(*) and the Control WT groups in a unique 6-groups (6G) DRF space. This method enabled separation of what can be interpreted as “aging” and “disease” effects along orthogonal axes (FIG. 54A). Discrimination values are percentages calculated as overlap of Target and Reference cloud, as previously described. Once the 2-dimensional 6G DRF space was defined as described, for each of the mHTT lowering regimens [LacQ140I, LacQ140I(2M) or LacQ140I(8M)], the Test clouds were directly projected into this space (one for each time point, green clouds in FIGS. 54B-54D). Proximity scores were computed as the percent overlap between the Test cloud (mHTT lowering regimens) and the Target cloud (WT), ranging from 0% (Test cloud completely overlaps with Reference; no phenotypic benefit) to 100% (Test cloud completely overlaps with Target; complete reversal of HD phenotypes). For cube analysis, p-values for proximity were estimated based on the group mean and variance of the Reference and Test groups as projected on the segment joining the center of the Reference and Target clouds.

FIGS. 54A-54D illustrate that Cube phenotypic signature in the LacQ140I(*) mouse model is prevented short-term by early mHTT lowering. DRF Space is defined by 6 clouds: 3 WTs Target clouds (blue), and 3 Reference clouds (red). (a) discrimination values between LacQ140I(*), (n=14 males, n=16 females) and WTs (WT: n=8 males, n=16 females, and WT*: n=15 males, n=16 females) defines the phenotypic signature of this HD mouse model. (b-d) illustrate three Test clouds (green), one for each test age (6, 9, 12 months) as they are ‘dropped in’ the DRF space defined in (a) with proximity values determined for each Test cloud in comparison to age-appropriate corresponding Target cloud (WTs). (b): LacQ140I (n=8 males, n=15 females) Test clouds; (c): LacQ140I(2M), (n=16 males, n=16 females) Test clouds; (d): LacQ140I(8M), (n=16 males, n=16 females) Test clouds. Significance level: **P<0.01; ***p<0.001; ****p<0.0001.

The aforementioned examples are illustrative and not restrictive.

### Section H

The present disclosure addresses technological problems arising in the technical field of screening and filtering model data in a database, drug discovery, and other related technical fields. These technological problems include efficiently identifying drugs or compounds having similar or reverse effects to a target model. Typically, such an identification would require computational expensive similarity tests based on a corpus of data for each of the drugs or compounds and the target model.

Disclosed embodiments address these technological problems by using observational data to produce a new data structure, called a target signature, which includes a set of filtered and ranked observational features. A target signature can enable comparison of the effects of a target modification to stored effects of other modifications, thus resulting in a more efficient and reliable technological solution for querying and screening a database storing the effects of modifications such as drugs or compounds.

Disclosed embodiments further address these technological problems by linking a list signature to each modification of the database. The list signature may include a set of behavioral features extracted from one or more test groups of subjects. The list signature may include a rank ordered set of the behavioral features that is precomputed and linked to each drug or compound. Thus, the technological solution of creating a database schema that links a list signature for each modification can enable efficient and accurate screening of modifications upon a query with a target model of behavioral data.

An additional technical problem addressed herein is that, in at least some cases, individual features from the behavioral platform can be analyzed independently such as comparing them across two phenotypes using differential statistics that may not reveal, on their own, the overall underlying phenotype or phenotypic differences, similarities, synergies or other relational properties. To at least address such technical shortcoming, in some embodiments, illustrative computing devices of present disclosure may be programmed with one or more techniques of the present disclosure to provide a technical solution that, without limitation, may provide computational methods for in vivo drug discovery using large-scale datasets based on drug screening with correlative analysis. The correlative analysis can use a wide collection of in subject data (e.g., behavioral and/or physiological data measurements) following treatment with bioactive small molecules together with pattern-matching algorithms to discover functional connections between drugs and diseases.

At least one technical problem being addressed herein is that, in at least some cases, in vivo drug discovery using large-scale datasets may be implemented for genome assays, but no such technique for searching behavioral data for drug discovery exists. To at least address such technical shortcoming, in some embodiments, illustrative computing devices of present disclosure may be programmed with one or more techniques of the present disclosure to provide a technical solution that, without limitation, may provide computational methods for in vivo drug discovery using large-scale datasets based on drug screening with correlative analysis of the behavioral phenotype. The behavioral phenotype represents a specialized structure of measurements of subject data following treatment with bioactive small molecules that can be used with pattern-matching algorithms to discover functional connections between drugs and diseases.

The solutions to the technical shortcoming may operate on the principle that a drug effective in the treatment of a disease (or reduction of side-effects of another compound) has the opposite subject feature profile to that of the disease (or the other compound), whereas drugs with similar therapeutic indications exhibit similar subject feature profiles. Thus, constructing subject feature profiles from observational features (e.g., subject and external features, or the like) extracted from large-scale collections of observational data, and processing such data as phenotypes with correlative analysis, including a Drug Behavioral Signature Analysis (DBSA) method, can enable efficient drug discovery.

In some embodiments, illustrative computing devices of present disclosure may be programmed with one or more techniques of the present disclosure to utilize high-content subject data derived from one or more sensors and/or other suitable measurement devices, such as, e.g., the sensors of a behavioral platform.

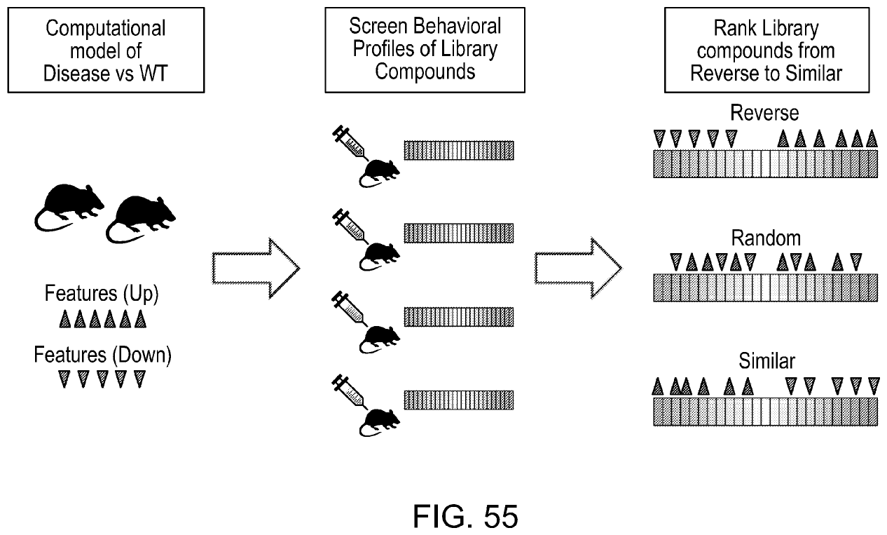

FIG. 55 illustrates an overall process for using subject feature profiles as phenotypes for drug discovery. Consistent with disclosed embodiments, a behavioral platform can perform large scale, drug-induced subject feature profile screening using a library of compounds and a signature analyzer. As illustrated, a comparison of behavioral features for a disease model and a control (“wild type” or “WT”) can result in a set of up-regulated features and down-regulated features. These features can be used to screen subject feature profiles in a library of compounds. A similarity value can be calculated for disease model and compounds in the library based on the features and the subject feature profiles. The disease model and the compounds can be ranked in terms of feature up-regulation and down-regulation from reverse to similar based on the similarity value.

Consistent with disclosed embodiments, one subject feature profile can be compared to other subject feature profiles stored in a library or database of subject feature profiles. For example, the library or database can be queried for compounds (or combinations of compound and dose) exhibiting similar and/or reverse subject feature profiles. As may be appreciated, the more subject feature profiles for different compounds stored in the library or database, the more comprehensive and accurate the identification of compounds having similar and/or reverse subject feature profiles becomes. Additionally, as described herein, the subject feature profile of a target compound can be described in terms of its similarity to the subject feature profiles of a set of reference compounds. Such a description can support characterization of the target compound in terms of the reference compounds (or those reference compounds to which it is most similar). Such an approach can leverage the existing subject feature profiles that are available for numerous reference compounds.

In some embodiments, illustrative computing devices of present disclosure may be programmed with one or more techniques of the present disclosure to query the database of subject feature profiles to identify potential beneficial therapeutics, such as a treatment with a compound. Other datasets can be used for the analysis to the extent that they comprise a content-rich set, and sufficient diverse features being measured for each sample. In an embodiment of this invention, the number of features to have sufficient diversity may be 500 or more. In another preferred embodiment the number of features will be at least 1000. In some embodiments, each computation model being analyzed may be represented by, e.g., at least 2 samples or replicas, at least 3 samples or replicas, at least 4 samples or replicas, at least 5 samples or replicas, at least 6 samples or replicas, at least 7 samples or replicas, at least 8 samples or replicas, at least 9 samples or replicas, at least 10 samples or replicas or more.

In some embodiments, illustrative computing devices of present disclosure may be programmed with one or more techniques of the present disclosure to employ one or more signature analyzers to analyze subject feature profiles relative to the compendia of large-scale profiles to screen for drug discovery. In some embodiments, the signature analyzer(s) may include one or more signature analyzers. There may be several methods for signature identification, including, e.g., enrichment analysis, which may be implemented as alternatives or in any suitable combination. Some such methods may include, e.g., Fisher Exact test (FET), correlation analysis (Pearson/Spearman/Euclidian/Manhattan etc.), or Gene set enrichment analysis (GSEA), among others or any suitable combination thereof. Formulating the subject feature profiles as behavioral phenotypes of features results in a new type of data that enables use of techniques such as FET, GSEA and others to improve signature identification for drug discovery. These techniques can support efficient and accurate identification of similar and/or reverse compounds.

In some embodiments, FET and GSEA can each have particular advantages and disadvantages that can be overcome by the technical solutions described herein. For instance, FET is based on fixed thresholds and uses hypergeometric distribution, which does not preserve correlations between biological features for statistical assessment. Furthermore, correlation methods may be biased by high numbers of non-differentially expressed features. Additionally, GSEA evaluates independent enrichments for up-regulated and down-regulated features.

In some embodiments, illustrative computing devices of present disclosure may be programmed with one or more techniques of the present disclosure to implement a signature analyzer including GSEA. GSEA may analyze up-regulated features and down-regulated features separately and independently. To improve the speed and efficiency of the signature analyzer, GSEA may be modified to form GSEA2. In some embodiments, the signature analyzer can use a modified form of GSEA (“GSEA2”), which can both account for the differential changes for each feature and also perform a single analysis that considers both up-regulated and down-regulated features simultaneously.

In some embodiments, illustrative computing devices of present disclosure may be programmed with one or more techniques of the present disclosure to, for example, form a subject feature profile of a mouse disease model (such as a model of Rett Syndrome, or a model of Parkinson's Disease), where the subject feature profile includes features quantifying a comparison between the mouse disease model with a control group (the “WT”). Alternatively, the subject feature profile can represent a treatment effect by a target compound that mimics the symptoms of a disease. In another example, the subject feature profile can be obtained from a compound whose subject feature profile represent a desired profile, for which raw data is available in the database.

In some embodiments, illustrative computing devices of present disclosure may be programmed with one or more techniques of the present disclosure to use the signature analyzer to receive a model. A model could be a genetically altered animal, a pharmacologically created model, an ontogenetically created model, a model of disease, symptom, dysfunction, neural circuit function, drug action, among other suitable models or any combination thereof. In some embodiments, the model may include a target signature having a set of target features. In some embodiments, the target signature may include a set of measured and/or derived values representing behavioral characteristics of the model. In some embodiments, the target signature may be obtained via a suitable technique for identifying target model features using a model target group (e.g., sample of animal subjects). For example, the target signature may be obtained using a machine learning classifier.

In some embodiments, target signature can include the difference values between the values of a set of observational features for a subject (or group of subjects) under a modification (e.g., subjects administered a drug, knockout genotype subjects, or the like) and the values of the set of observational features of an appropriate control group (e.g., subjects administered a vehicle, or wild-type subjects, or the like). In various embodiments, the difference values can be the standard difference (e.g., b-a), the fold change value (e.g., b/a), or another suitable function of the values of the two sets of observational features. In some embodiments, the set of observational features can be selected using a suitable statistical comparison and/or statistical scoring. In some embodiments, the statistical comparison and/or statistical scoring can be used to identify features which are significantly different between the model and control group (e.g., based on a statistical hypothesis testing using the test statistic, such as, e.g., a p-score, confidence intervals, likelihood ratios, Bayes factors, etc.). In some embodiments, a significant difference may be indicated by, e.g., a p-value less than 0.05 or other suitable p-value threshold.

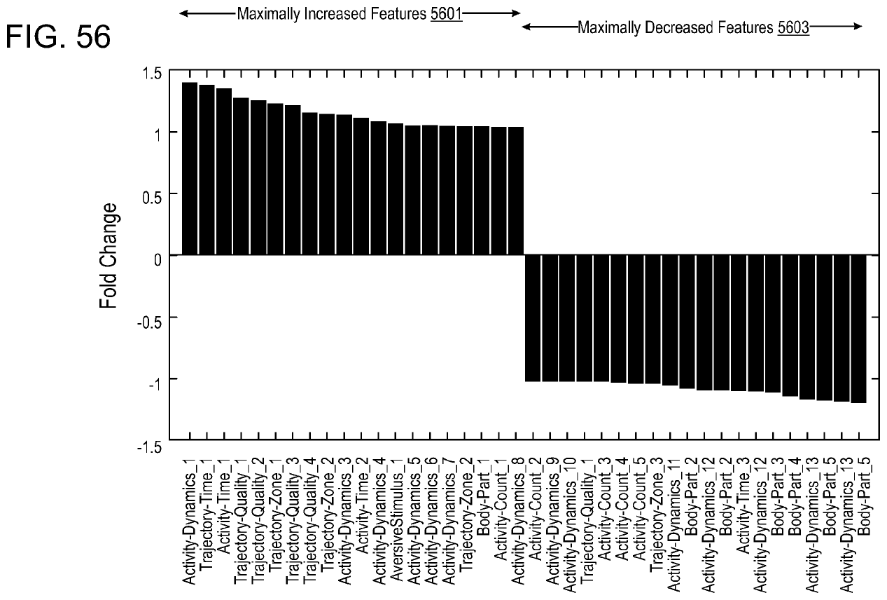

Consistent with disclosed embodiments, the target signature can be divided into increased (S+) and decreased (S−) feature sets. Alternatively, the target signature can be defined using other criteria such as those features relevant for a particular domain of a disease model (gait features, for example) or a domain of importance for a drug discovery project (such as negative symptoms in an animal model of depression). An example of a target signature (e.g., formed from the ranked and divided set of target differential features) for the TG4510 transgenic model of Alzheimer's Disease at 6 months of age is illustrated in FIG. 56. Features that were maximally increased 5601 or decreased 5603 in the TG4510 model as compared to the wild-type control are labeled according to activity type, quantity, temporal dynamics, type of locomotor trajectory, and body parts characteristics.

In some embodiments, a database can be queried for compounds that have a subject feature profile that includes the features in the target signature. In some embodiments, the compounds can be associated with list signatures defined by the differences between a subject feature profile for the compound and a subject feature profile of an appropriate control group. The database can be queried for compounds that have a list signature that includes the features in the target signature. The target signature can then be compared to the list signature of the compound.

In some embodiments, illustrative computing devices of present disclosure can be programmed with one or more techniques of the present disclosure to further apply a feature ranking algorithm to estimate the discrimination power (ability to separate the two groups e.g., control and disease) of each feature to identify relevant target features. In some embodiments, an exemplary feature ranking algorithm can be programmed to weigh each feature change by its relevance. If there is a significant change in some irrelevant feature measured for a particular phenotype, the low rank of this feature may automatically reduce the effect of such change in the analyses. In some embodiments, illustrative computing devices of present disclosure can be programmed with one or more techniques of the present disclosure to further utilize top-ranking features to obtain insights into the biological underpinnings of the phenotype of interest. An example of the feature ranking algorithm may include decorrelated ranked feature analysis (DRFA) as described in Section G, attached herewith.

Consistent with disclosed embodiments, a list signature can be defined for one or more compounds in the library. In some embodiments, the compounds may be selected based on a test condition. In some embodiments, when screening the library of compounds, a list signature can be defined for each compound of the library. As described herein, this list signature can be used to identify compounds with similar or reverse subject feature profiles to a specified subject feature profile, depending on the underlying requirements or questions being asked (the test condition). In some embodiments, these list signatures can be generated similarly to the generation of the target signatures discussed herein. For example, the differences between a subject feature profile for the compound and a subject feature profile of an appropriate control group can be determined. The features in the list signature can be ranked. An appropriate statistic can be used to rank (and/or select) features in the list signature based on these differences. Appropriate statistics may include t-statistic, a z-score, a normal score, an F-statistic, or other suitable test statistic. In some embodiments, the ranking may be determined by a function of the appropriate statistic (e.g., a signed log of a p-value, or the like), by a non-parametric statistic, or by another suitable ranking metric.

Consistent with disclosed embodiments, feature enrichment and enrichment scores can be determined. In some embodiments, once the target signature and list signatures are defined, a suitable statistical test for comparing probability distributions can be used to assess whether the S+ and S− sets are statistically enriched in the two extremes of the ranked list signature, such as, e.g., Kolmogorov-Smirnov test or other suitable test or any combination thereof. In some embodiments, statistical enrichment can be quantified as enrichment score (ES) as described below. For randomly distributed S+ and S−, ES are relatively small, but if S+ and S− are concentrated at the top and bottom, respectively, of the list signature then ES is correspondingly high.

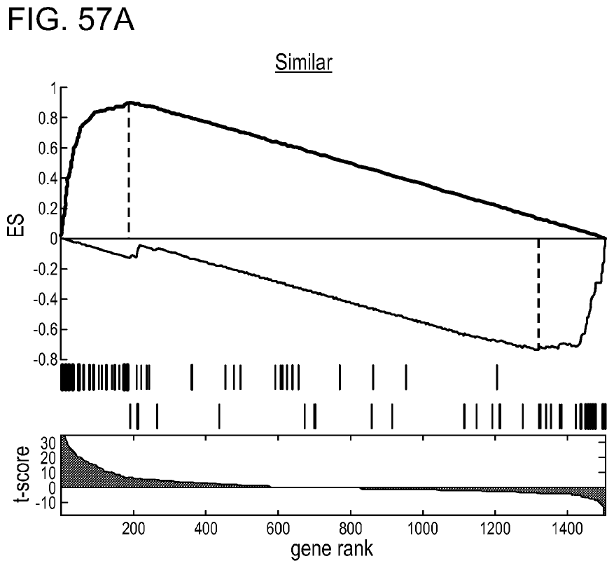

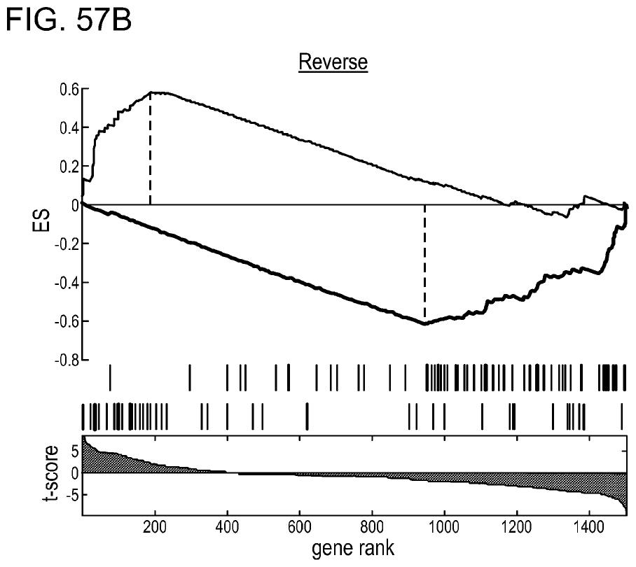

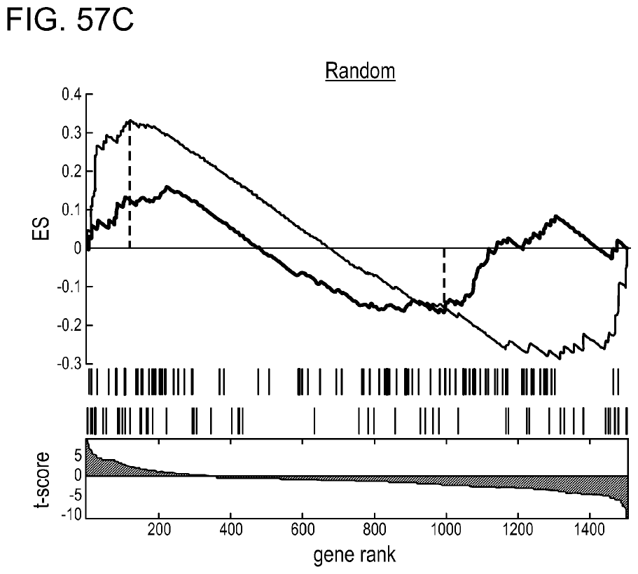

FIGS. 57A-57C illustrate an exemplary determination of an ES score using a target signature and a list signature in three different scenarios, in accordance with disclosed embodiments. The features of the list signature that match the target feature set are highlighted on the x-axis as a “barcode” plot (middle subpanel). Their position on the x-axis is used to generate the enrichment curve, indicated by blue (for increased) and red (decreased) features (upper subpanel). As the statistical testing progresses from the left (increased features) to the right (decreased features), the ES+ score is appended by adding the ranking score of a list signature feature every time it matches a feature in the target feature set (indicated by the bars in FIGS. 57A-57C). If the list signature feature does not match the target feature set, ES+ is appended by subtracting a constant. For the decreased S− a similar procedure is followed.

Consistent with disclosed embodiments, enrichment can be estimated simultaneously, rather than independently, for S+ and S−, using GSEA2. In some embodiments, after S+ matches are assigned to the list signature, a duplicate of the list signature with its values inverted can be used to match S−. The list signature and the inverted list signature can then be combined into one, preserving the S+ and S− matches. Upon combining the list signature and the inverted list signature, the test features of the list signature may be re-sorted. The resulting unified list signature can be used to estimate a single enrichment score.

Consistent with disclosed embodiments, a null distribution can be estimated for an ES. In some embodiments, to do so, sample labels can be used to calculate the list signature (where a label indicates if the sample belongs to the drug and/or compound treatment group underlying the list signature, or to its corresponding vehicle control group). The sample labels can be randomly shuffled to create permutations of the sample labels. Here, a sample refers to a subject feature profile for a single subject. The permutation of sample labels, as opposed to feature label permutation, preserves correlations between different features and thus can generate a biologically relevant null distribution. The statistical significance of the ES can then be computed by comparing the observed ES to the null distribution when there are at least 6 or samples in each group. If there are fewer than 6 samples in each group, the null distribution can be calculated by randomly shuffling sample labels. An embodiment of the present invention may advantageously employ, e.g., 500 permutations, 600 permutations, 700 permutations, 800 permutations, 900 permutations, 1000 or more permutations.

In some embodiments, illustrative computing devices of present disclosure can be programmed with one or more techniques of the present disclosure to normalize the enrichment scores and/or calculate an odds ratio. In some embodiments, the final output can thus include one or both of two statistics that measure the level of enrichment: the normalized enrichment score (NES) and the odds ratio (OR). The NES can be calculated by rescaling the observed ES by an average of randomized enrichment scores (where the average is calculated only for the randomized ES with the same sign as the observed ES. To calculate the OR, a leading edge (LE) may first be defined as the portion of the ranked list signature up to the feature corresponding to the maximum ES (for positive ES) (see FIGS. 57A-57C). The OR may then be defined as the ratio between the odds of the LE features belonging to the target feature set, and the odds of the LE features not belonging to the target feature set.

FIGS. 57A-57C show three representative outcomes of DBSA analysis using observational data, collected with a behavioral platform, in accordance with disclosed embodiments. Each panel includes a lower sub-panel that illustrates a list signature for a compound. The list signature includes features ranked according to a difference between a value of the feature for the compound and a value of the feature for a control. The features are ranked from the most up-regulated features to the most down-regulated features. The list signature is compared to a target signature including target features. The target features were selected based on a value of the feature for the target compound and a value of the feature for a control (which may be the same control as used to generate the list signature). The target features can be selected based on a function of these values (e.g., a difference, or weighted difference, between the value of the feature for the target compound and the value of the feature for a control). The selected features can include the features exhibiting the greatest increase in value (or weighted value) between the target compound and the control. The selected features can include the features exhibiting the greatest decrease in value (or weighted value) between the target compound and the control. In some embodiments other criteria can be used such as most significantly increased, or some other metric, such as features belonging to a particular domain such as social behavior or locomotor activity. Each panel includes a middle subpanel with the top increased and decreased target features highlighted in their ranked position as a ‘barcode’ plot. The position of each target feature in the ranked order in the target feature set (x-axis) is used to generate an enrichment curve, indicated by blue (for increased) and red (decreased) target features (upper subpanel). In this example, the analysis moves through the increased target features set, S+, from the left to the right. As the analysis progresses, the t-score at each match between target feature and list signature updates a running sum of the t-scores of the target features and plots the updated sum at the location of match. Where the analysis encounters a location with no increased target feature, a constant is subtracted from the running sum. In some embodiments, a similar process for the decreased features set (S−) may be followed.

Consistent with disclosed embodiments, one or more outcomes can be generated based on the enrichment score, normalized enrichment score and/or odds ratio. For example, an outcome may include a determination that the target signature is “similar” to the test phenotype (such as that illustrated in FIG. 57A). In another example, an outcome may include a determination that the target signature can be a ‘reverse’ of the test phenotype, where “reverse” indicates that the target feature set and the test signature have opposite feature rankings (such as that illustrated in FIG. 57B), thus suggesting the therapeutic potential on the disease. In another example, an outcome can include a determination that the target signature has a “random” relationship to the test phenotype (such as that illustrated in FIG. 57C) indicating no similarity.

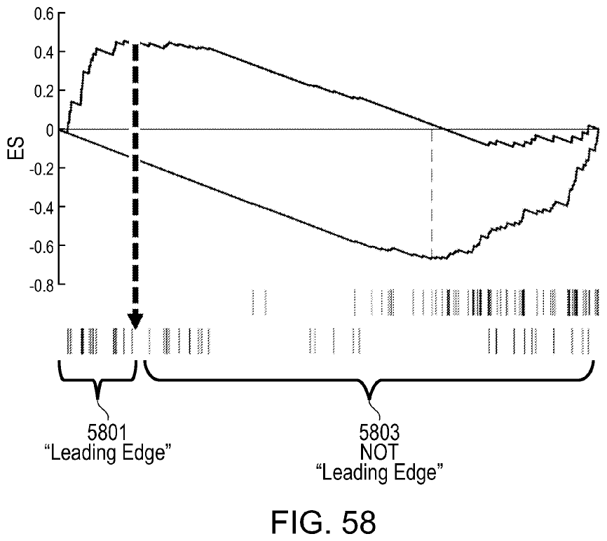

FIG. 58 illustrates an example GSEA plot illustrating the leading edge of the positive enrichment, consistent with embodiments of the present disclosure. In some embodiments, the ranked list signature can be partitioned into leading edge 5801 and not-leading edge 5803 components based on the maximum deviation of the ES from zero. FIG. 58 illustrates the maximum ES value that partitions the list signature into leading edge and not leading-edge components

In some embodiments, illustrative computing devices of present disclosure can be programmed with one or more techniques of the present disclosure to use the DBSA signature analyzer for in silico prediction of the result of combining two or more drugs, compounds or other manipulations. In some embodiments, such comparison can predict the partial or complete cancellation of the feature changes leading to a reduced activity of the combination relative to either manipulation on its own. Alternatively, or in addition, the prediction can indicate additive properties, synergistic or potentiation of the individual manipulation effects, leading to a more potent effects through experimental treatment with the manipulation combination.

In some embodiments, illustrative computing devices of present disclosure can be programmed with one or more techniques of the present disclosure to prioritize certain domains for the combinations of modifications according to DBSA. In one embodiment of the invention, it can be possible to choose a compound that increases certain features hypothesized to reflect an anxiolytic response (such as increased time in the center of an experimental arena) and another compound that increases features related to certain aspects of depression (such as social behavior). Combination of two such compounds can result in a novel therapy that may address more than one symptom or aspect of disease. Combinations can be done for compounds in early stage of development, for drugs in the market, or for combinations of pharmacological and other modifications (optogenetic, genetic, electrophysiological manipulations and the like).

### Illustrative Non-Limiting Examples of DBSA

### Example 1—DBSA for Identification of Novel Cognitive Enhancer

In some embodiments, illustrative computing devices of the present disclosure can be programmed with one or more techniques of the present disclosure to one or more test manipulations based on a known effect on physiology and behavior caused by one or more target manipulations. The one or more target manipulations can have undesirable effects on a subject. In some embodiments, the above-described techniques can be used to identify a list manipulation have a particular improvement in subjects relative to the one or more target manipulations by using the above-described enrichment analysis to identity a reverse list signature based on the target signature and the list signatures.

In some embodiments, illustrative computing devices of the present disclosure can be programmed with one or more techniques of the present disclosure to identify a novel cognitive enhancer compound by querying a large library of compounds profiled in by a behavioral platform. This example is based on a hypothesis that compounds with a drug-induced behavioral data profile that is reversed to that of scopolamine, which impairs cognition, are potentially cognitive enhancers. Other ways to impair cognition, such as other drugs or genetic manipulations, can be used to generate similar hypotheses.

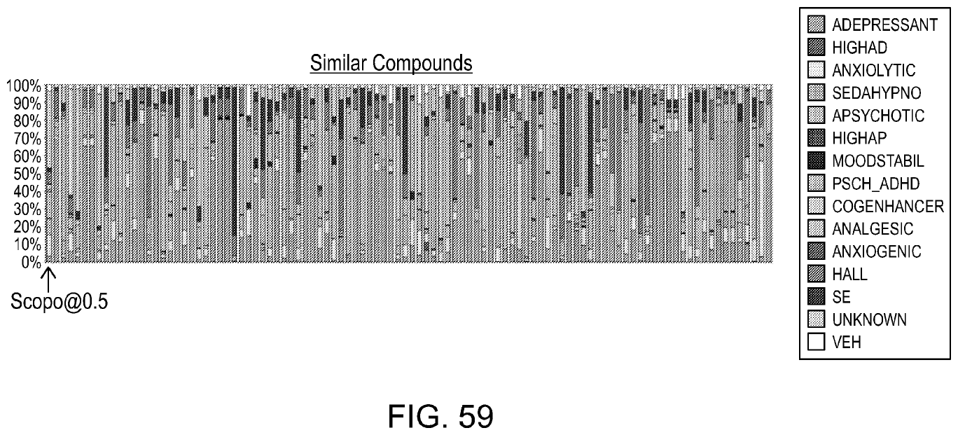

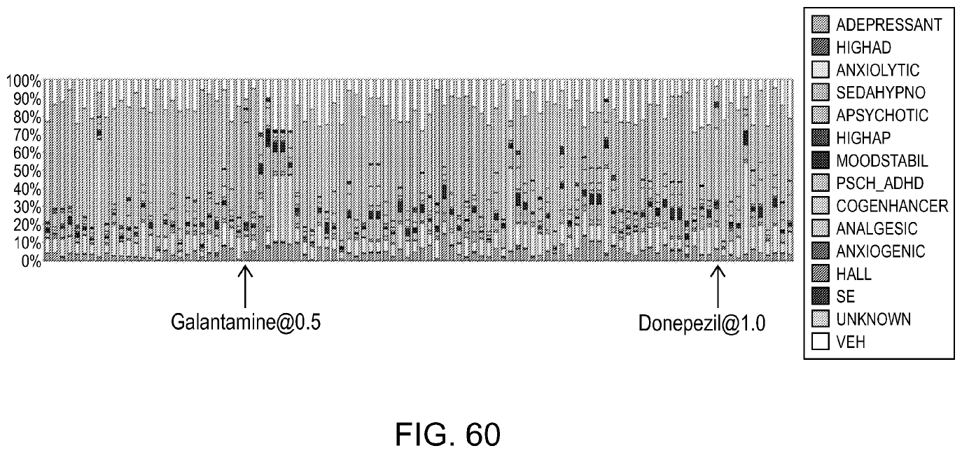

In a particular embodiment, computing devices of present disclosure can be programmed with a specific technique of the present disclosure to use DBSA to measure statistically significant enrichment of behavioral target features differentially induced by scopolamine (the target compound). The target signature was used to query a library of list signatures associated with active library and reference compounds to rank the compounds by their capacity to reverse the behavioral response to scopolamine. As illustrated in FIG. 59, among the most similar compounds, DBSA identified scopolamine itself (at a different dose than the one used for the DBSA model). As illustrated in FIG. 60, among the top reverse list signature predictions, DBSA identified galantamine and donepezil, which are known cognitive enhancers, thus demonstrating the validity of this method.

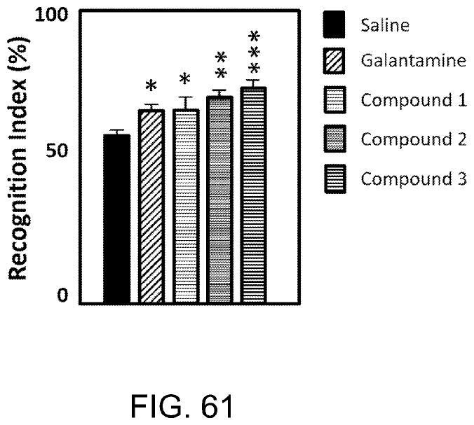

Among the top DBSA predictions, three library compounds were selected for further validation using standard tests of cognitive function, the novel object recognition (NOR) assay. In this test, subjects are first exposed to an inert object in a first experimental session. In a second session, the first object (the familiar one) is presented in conjunction with a new one (the novel one) and an observer records the time spent exploring each object. The results of the NOR test may be expressed as recognition index (RI), which is defined as the ratio of time spent exploring the novel object over the total time spent exploring both objects (RI=Novel/(Familiar+Novel)×100%) during a 5-min test session. The NOR data is analyzed using one-way ANOVA followed by Fisher's LSD post hoc test aggregating the data in three different ways: for the first minute, first three minutes, and whole 5-min session, with significance set at p<0.05. Galantamine was used as a positive control in this assay. Results showed that all these selected compounds showed increased recognition index when compared to saline treatment. In fact, considering just the first 3 minutes of the session, the RI for all three compounds was higher than that of galantamine (FIG. 61).

### Example 2—DBSA for Identification of Novel Therapy for Huntington Disease

In some embodiments, illustrative computing devices of present disclosure can be programmed with one or more techniques of the present disclosure to screen active reference compounds against an animal model of a neurodegenerative disease. As an example, in an application of DBSA, the Q175 heterozygous (HET) mice model of Huntington Disease was used to generate a target signature. Male and female cohorts of the Q175 HET model mice at 6 months were compared against the associated wild type (WT) controls. The DBSA Target signature model for the Q175 HET at 6 months is, for example, illustrated in FIG. 56. The target signature was used to query a database containing list signatures for reference compounds profiled at multiple doses.

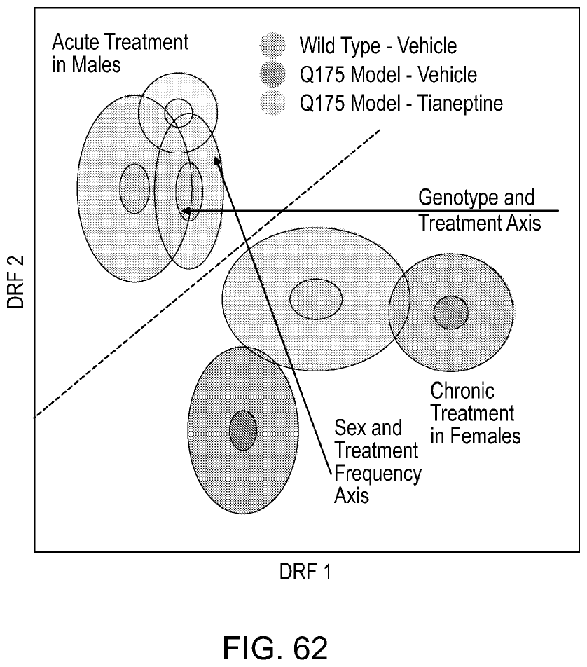

The top predictions confirmed several compounds known to be clinically effective in the symptomatic treatment of HD, including bupropion, modafinil, methylphenidate, and several SSRIs. Of interest was the identification of the atypical antidepressant tianeptine, currently not used for the treatment of HD in the clinic. Follow-up validation experiments for this novel prediction were performed by treating the Q175 HET mice with saline tianeptine at 20 mg/kg, both acutely and chronically (4-week regimen). A control group of WT mice was treated equally with saline. In both experiments tianeptine significantly rescued the behavioral phenotype of the Q175 HET mice (see FIG. 62). The beneficial effect of tianeptine was quantified using DRFA, which transforms the original subject feature profile to a non-redundant, decorrelated ranked 2D features space. The non-redundant decorrelated ranked features space was used to calculate the discrimination between control (WT treated with saline) and disease group (Q175 HET group treated with saline). The disease group (Q175 HET) treated with tianeptine was then projected onto this 2D space and its proximity to the control group was calculated. Both acute and chronic treatments showed recovery of the phenotype.

The disclosed embodiments can be implemented using system 500, described above with regards to FIG. 5. In some embodiments, DBSA can be implemented to perform the query and screening analyses as a local process using local data local to the database, e.g., at a server 506 and/or 507. In some embodiments, DBSA can be implemented in a distributed fashion, e.g., by processing the target behavioral profile to create the target differential feature set remotely from the database, e.g., using devices 502-504 remote from the server 506 and/or 507. In some embodiments, any one or more of devices 502-504 may be used to initiate a request to perform DBSA on a target behavioral phenotype relative to the database of compounds which causes the server 506 and/or 507 to generate the target differential feature set, the list signature and perform the enrichment and odds ration analyses to screen for similar and/or reverse compounds.

In some embodiments, system 500 can be based on a scalable computer and/or network architecture that incorporates varies strategies for assessing the data, caching, searching, and/or database connection pooling. Such a scalable architecture can be capable of operating multiple servers and adding or removing servers in response to demand. In some embodiments, the exemplary inventive computing devices and/or the exemplary inventive computing components of the system 500 may be configured to manage the exemplary dynamic mapping module 118 of the present disclosure, utilizing at least one machine-learning model described herein.

A method to identify a compound using Drug Behavioral Signature Analysis may include:
receiving, by at least one processor, a target signature associated with a target condition;
where the target behavioral phenotype includes a plurality of target features;
determining, by the at least one processor, a target differential feature set including a plurality of target differential features based at least in part on difference between the plurality of target features and a plurality of control features associated with a control phenotype;
determining, by the at least one processor, a target feature set within the target differential feature set, where the target feature set includes at least one target differential feature of the plurality of target differential features having a significant difference from the control behavioral profile based at least in part on a statistical scoring of each target differential feature of the plurality of target differential features;
generating, by the at least one processor, a ranked target feature set including rank ordering of the at least one target differential feature based at least in part on the statistical scoring of each target differential feature;
accessing, by the at least one processor, a test phenotype database including a plurality of test behavioral phenotypes;
where each test behavioral phenotype includes a plurality of test behavioral phenotype features;
identifying, by at least one processor, at least one test behavioral phenotype associated with a test condition;
determining, by the at least one processor, a plurality of test differential features based at least in part on difference between the plurality of test behavioral profile features and a plurality of vehicle features associated with the vehicle behavioral profile;
generating, by the at least one processor, a list signature including rank ordering of the plurality of test differential features based at least in part on the statistical scoring of each test differential feature;
determining, by the at least one processor, an inverted list signature including a plurality of inverted test features based at least in part on an inverse of the statistical scoring of the plurality of test features;
generating, by the at least one processor, a unified list signature including rank order of the plurality of test features and the plurality of inverted test features so as combine the list signature and the inverted list signature;
determining, by the at least one processor, a plurality of enrichment scores utilizing at least one statistical test comparing the target feature set to the unified list signature;
where the at least one statistical test includes an addition of a statistical score of each test differential feature that matches the at least one target differential feature, and a subtraction of a constant for each test differential feature that differs from the at least one target differential feature;
determining, by the at least one processor, a plurality of normalized enrichment scores based on a normalizing of each enrichment sub-score of the plurality of enrichment sub-scores;
determining, by the at least one processor, a maximum normalized enrichment score;
determining, by the at least one processor, a leading-edge portion of the rank order of the plurality of test features, the leading-edge portion including the test features of the plurality of test features higher in the rank order than the maximum normalized enrichment score;
determining, by the at least one processor, a first probability of the test features in the leading-edge portion being within the plurality of target features;
determining, by the at least one processor, a second probability of the test features in the leading-edge portion not being within the plurality of target features;
determining, by the at least one processor, an odds ratio based at least in part on a ration of the first probability and the second probability;
determining, by the at least one processor, a similarity measure between the target behavior phenotype and the test behavior phenotype based at least in part on the odds ratio; and
returning, by the at least one processor, a treatment recommendation based at least in part on the similarity measure.

### Section I

The present disclosure addresses technological problems arising in the technical field of screening and filtering model data in a database, drug discovery, and other related technical fields. These technological problems include efficiently identifying drugs or compounds having similar or reverse effects to a target model. Typically, such an identification would require computational expensive similarity tests based on a corpus of data for each of the drugs or compounds and the target model.

Disclosed embodiments address these technological problems by using observational data to produce a new data structure, called a target signature, which includes a set of filtered and ranked observational features. A target signature can enable comparison of the effects of a target modification to stored effects of other modifications, thus resulting in a more efficient and reliable technological solution for querying and screening a database storing the effects of modifications such as drugs or compounds.

Disclosed embodiments further address these technological problems by linking a list signature to each modification of the database. The list signature may include a set of observational features extracted from one or more test groups of subjects. The list signature may include a rank ordered set of the observational features that is precomputed and linked to each drug or compound. Thus, the technological solution of creating a database schema that links a list signature for each modification can enable efficient and accurate screening of modifications upon a query with a target model of behavioral data.

An additional technical problem addressed herein is that, in at least some cases, individual features from the behavioral platform can be analyzed independently, such as comparing them across two phenotypes using differential statistics that may not reveal, on their own, the overall underlying phenotype or phenotypic differences, similarities, synergies, or other relational properties. To at least address such technical shortcoming, in some embodiments, illustrative computing devices of present disclosure may be programmed with one or more techniques of the present disclosure to provide a technical solution that, without limitation, may provide computational methods for in vivo drug discovery using large-scale datasets based on drug screening with correlative analysis. The correlative analysis can use a wide collection of in subject data (e.g., behavioral and/or physiological data measurements) following treatment with drugs, such as bioactive small molecules, together with pattern-matching algorithms to discover functional connections between drugs and diseases.

At least one technical problem being addressed herein is that, in at least some cases, in vivo drug discovery using large-scale datasets may be implemented for genome assays, but no such technique for searching behavioral data for drug discovery exists. To at least address such technical shortcoming, in some embodiments, illustrative computing devices of present disclosure may be programmed with one or more techniques of the present disclosure to provide a technical solution that, without limitation, may provide computational methods for in vivo drug discovery using large-scale datasets based on drug screening with correlative analysis of the behavioral phenotype. The behavioral phenotype represents a specialized structure of measurements of subject data following treatment with drugs, such as bioactive small molecules, which can be used with pattern-matching algorithms to discover functional connections between drugs and diseases and identify possible synergies between manipulations that may lead to behavioral phenotype recovery. Here, recovery refers to the test signature indicating an offsetting of the effects on behavior of the target manipulation according to the target signature. In other words, it is the reduction of the features of an undesired phenotype by application of a treatment.

The solutions to the technical shortcoming may operate on the principle that a manipulation effective in the treatment of a disease (or reduction of side-effects of a compound) has the opposite behavioral data profile to that of the disease (or the compound), whereas manipulations with similar therapeutic indications exhibit similar behavioral data profiles. Thus, constructing behavioral data profiles from features of large-scale collections of in vivo behavioral data and processing such data as phenotypes with correlative analysis, such as a Drug-induced Behavioral Signature Recovery (DBSR) analysis, enable efficient and accurate drug discovery of manipulation that exhibits a likelihood of recovery of the Target model.

As described above, FIG. 55 illustrates an overall process for using subject feature profiles as phenotypes for drug discovery. While FIG. 55 shows drug discovery, the same process may be used for any manipulation using subject feature profiles. Consistent with disclosed embodiments, a behavioral platform can perform large scale, drug-induced subject feature profile screening using a library of compounds and a signature analyzer. The signature analyzer can perform Drug-induced Behavioral Signature Recovery (DBSR) analysis using the drug-induced subject feature profiles. As illustrated, a comparison of behavioral features for a disease model and a control (“wild type” or “WT”) can result in a set of up-regulated features and down-regulated features. These features can be used to screen subject feature profiles in a library of compounds. A similarity value can be calculated for disease model and compounds in the library based on the features and the subject feature profiles. The disease model and the compounds can be ranked in terms of feature up-regulation and down-regulation from reverse to similar based on the similarity value.

Consistent with disclosed embodiments, one subject feature profile can be compared to other subject feature profiles stored in a library or database of subject feature profiles. For example, the library or database can be queried for compounds (or combinations of compound and dose) exhibiting similar and/or reverse subject feature profiles. As may be appreciated, the more subject feature profiles for different compounds stored in the library or database, the more comprehensive and accurate the identification of compounds having similar and/or reverse subject feature profiles becomes. Additionally, as described herein, the subject feature profile of a target compound can be described in terms of its similarity to the subject feature profiles of a set of reference compounds. Such a description can support characterization of the target compound in terms of the reference compounds (or those reference compounds to which it is most similar). Such an approach can leverage the existing subject feature profiles that are available for numerous reference compounds.

Consistent with disclosed embodiments, a behavioral platform can query the database of behavioral data profiles to identify potential beneficial therapeutics through multi-modification synergies, such as a treatment with a compound to effect phenotypic recovery for the effects of another modification. Other datasets can be used for the analysis to the extent that they comprise a rich-content set, and sufficient diverse features being measured for each sample. In an embodiment of this invention the number of features to have sufficient diversity may be 500 or more. In another preferred embodiment the number of features will be at least 1000. Although a large number of features can be used, one advantage of the DBSR method, as opposed to other correlational enrichment methods, is that the number of features can be restricted to the most meaningful ones, reducing therefore the chance of finding spurious results. Such restriction can limit the features compared to between 10 and 100, between 10 and 200, between 10 and 300, between 10 and 400, between 10 and 500 or other suitable number of the most meaningful or relevant features.

In some embodiments, a subject feature profile of a modification (e.g., administration of a drug or compound, or the like) can be generated using at least two samples or replicates, at least 3 samples or replicates, at least 4 samples or replicates, at least 5 samples or replicates, at least 6 samples or replicates, at least 7 samples or replicates, at least 8 samples or replicates, at least 9 samples or replicates, at least 10 samples or replicates or more. Each sample of replicate can contain observational features corresponding to an experiment in which observational data was acquired for a subject under the modification.

Consistent with disclosed embodiments, a behavioral platform can employ one or more signature analyzers to analyze behavioral data profiles relative to the compendia of large-scale profiles to screen for drug discovery. In some embodiments, the signature analyzer(s) can include one or more signature analyzers. Drug-induced Behavioral Signature Recovery (DBSR) analysis can compare two subject feature profiles (e.g., generated by the behavioral platform, or another system). DBSR analysis can be used to quantify the potential of a treatment to reverse or recover the changes in features observed in a model.

Consistent with disclosed embodiments, a behavioral system can form a subject feature profile of a mouse disease model (such as a model of Rett Syndrome, or a model of Parkinson's Disease). In some embodiments, the subject feature profile can include features quantifying a comparison between the mouse disease model with a control group (the “WT”). Alternatively, the behavioral data profile may represent a treatment effect by a target compound that mimics the symptoms of a disease. In another example, the subject feature profile can be obtained from a compound whose subject feature profile represents a desired profile, for which raw data is available in the database.

Consistent with disclosed embodiments, a behavioral platform can receive a target signature. In some embodiments, target signature can include the difference values between the values of a set of observational features for a subject (or group of subjects) under a modification (e.g., subjects administered a drug, knockout genotype subjects, or the like) and the values of the set of observational features of an appropriate control group (e.g., subjects administered a vehicle, or wild-type subjects, or the like). In various embodiments, the difference values can be the standard difference (e.g., b-a), the fold change value (e.g., b/a), or another suitable function of the values of the two sets of observational features. In some embodiments, the set of observational features can be selected using a suitable statistical comparison and/or statistical scoring. In some embodiments, the statistical comparison and/or statistical scoring can be used to identify features which are significantly different between the model and control group (e.g., based on a statistical hypothesis testing using the test statistic, such as, e.g., a p-score, confidence intervals, likelihood ratios, Bayes factors, etc.). In some embodiments, a significant difference may be indicated by, e.g., a p-value less than 0.05 or other suitable p-value threshold.

Consistent with disclosed embodiments, the target signature can be divided into increased (S+) and decreased (S−) feature sets. Alternatively, the target signature can be defined using other criteria such as those features relevant for a particular domain of a disease model (gait features, for example) or a domain of importance for a drug discovery project (such as negative symptoms in an animal model of depression).

Consistent with disclosed embodiments, a behavioral platform can apply a feature ranking algorithm to estimate the discrimination power (ability to separate the two groups e.g., control and disease) of each feature to identify the target feature relevant for the domain. In some embodiments, an exemplary feature ranking algorithm may be programmed to weigh each feature change by its relevance: if there is a significant change in some irrelevant feature measured for a particular phenotype the low rank of this feature would automatically reduce the effect of such change in the analyses. In another embodiment the ranking score may be determined by the signed log of the p-value, or by a non-parametric statistic or other suitable ranking metric.

Consistent with disclosed embodiments, a behavioral platform can use top-ranking features to obtain insights into the biological underpinnings of the phenotype of interest. An example of the feature ranking algorithm may include decorrelated ranked feature analysis (DRFA) as described in Section G.

Consistent with disclosed embodiments, a behavioral platform can define a list signature for one or more modifications in a library. Similar to a target signature, a list signature can include value differences (e.g., standard differences or fold change values, or another suitable function), for a set of observational features, between a subject feature profile for the modification and a subject feature profile of an appropriate control group. As with the target signature, the set of observational features included in the list signature can be selected using a suitable statistical comparison and/or statistical scoring. In some embodiments, the statistical comparison and/or statistical scoring can be used to identify features which are significantly different between the subject feature profile for the modification and a subject feature profile of an appropriate control group. Alternatively, the set of observational features can be defined using other criteria such as those features relevant for a particular domain of a disease model (gait features, for example) or a domain of importance for a drug discovery project (such as negative symptoms in an animal model of depression).

In some embodiments, when screening the library of modifications, these list signatures can be compared to a target signature to identify reference modifications with profiles that indicate reversal and/or recovery (as desired) of the effects of the target modification.

In some embodiments, illustrative computing devices of present disclosure may be programmed with one or more techniques of the present disclosure to rank the observational features in the list signature according to the ranking of the observational features in the target signature such that the list signature and the target signature have the same order.

Alternatively, consistent with disclosed embodiments, a behavioral platform can apply a feature ranking algorithm to estimate the discrimination power (ability to separate the two groups e.g., vehicle and disease) of each feature to identify the observational features relevant for the domain. In some embodiments, an exemplary feature ranking algorithm may be programmed to weigh each feature change by its relevance: if there is a significant change in some irrelevant feature measured for a particular phenotype the low rank of this feature would automatically reduce the effect of such change in the analyses. In another embodiment the ranking score may be determined by the signed log of the p-value, or by a non-parametric statistic or other suitable ranking metric. Thus, the list signatures of the library of modifications can be initially filtered to those that have the same order as the target signature.

In some embodiments, the ranking of features for both the list signature and the target signature serves several purposes: first, it allows both signatures to be reordered based on the ranking of the target signature; second, it generates weights for each feature proportional to the relevance of each feature in determining the separability of the two experimental groups being compared and/or the relevance of features to target in a disease model; and third, it ensures maximal relevance of the features that should be addressed by treatment in a disease model. In some embodiments, separability can be quantified using any suitable data modelling technique, such as with a machine learning classifier or other statistical approach, or any combination of suitable data modelling techniques.

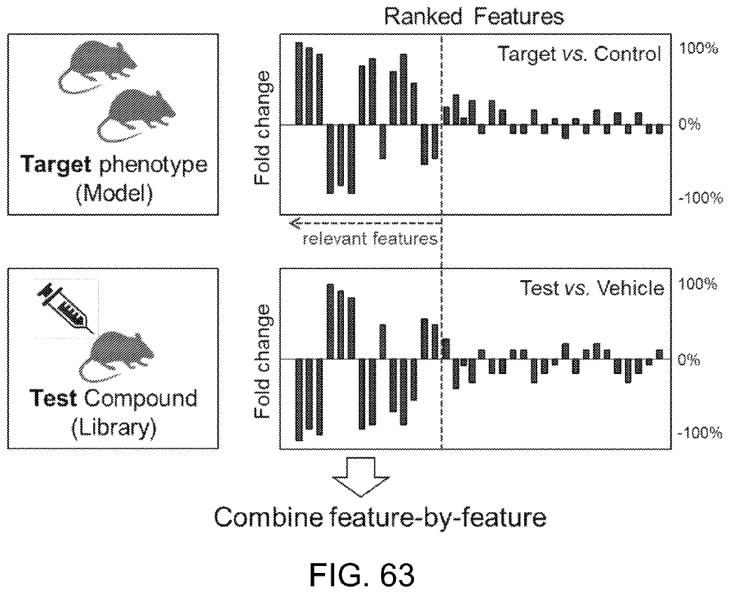

Consistent with disclosed embodiments, a behavioral platform can perform a DBSR analysis using the statistics of both the target signature and the list signatures, thus increasing the specificity of the results. In some embodiments, the DBSR analysis includes combining the sets of features of the target signature and the list signature (as illustrated in FIG. 63), to generate a statistic that quantifies the putative overall recovery (or similarity between Test and Target) of the Target modification by the Test modification. Combining the sets of features can be performed via any suitable feature-by-feature combination, such as, e.g., feature-by-feature sum and/or difference, weighted combination, thresholded sum and/or difference, thresholded weighted combination, or any other suitable feature-by-feature combination or any combination thereof.

Sum and/or Difference

In some embodiments, the target signature and list signature can be combined by adding the fold change, or corresponding statistical score, of each feature in the target signature to the fold change, or corresponding statistical score, of each matching feature in the list signature and taking the absolute value. Alternatively, the difference may be taken instead of the sum. If the sum method is chosen a lower DBSR score for a particular feature corresponds to higher potential of phenotypic recovery. Conversely, if the target signature and list signature are combined using the difference, a higher DBSR score for a particular feature corresponds to higher potential of phenotypic recovery. In some embodiments, the sum/difference across all features may be aggregated to form a final DBSR score. The final DBSR score corresponds to the potential of phenotypic recovery, where a phenotypic recovery is indicated with a score nearer to zero when using sums, and is indicated with a score further from zero when using differences.

In some embodiments, as described above, the observational features of the target signature (and of the list signature) can be associated with weights indicating the relevance of each observational feature. The sum/difference combining may be modified to incorporate the weights of each observational feature such that more relevant features are given greater effect. Accordingly, in some embodiments, illustrative computing devices of present disclosure may be programmed with one or more techniques of the present disclosure to multiply each fold change (or standard difference, or the like) of each feature with the weight of the feature.

In some embodiments, the weights associated with each observational features of the target signature may be applied to the value of each observational features of the target signature prior to taking the sum and/or difference of the features between the target signature and the list signature. The observational features of the list signatures can likewise be modified using the associated weights prior to taking the sum and or difference. As may be appreciated, other mathematically equivalent implementations could also be used, without departing from the envisioned embodiments.

Weighting and/or Thresholding

In some embodiments, illustrative computing devices of present disclosure may be programmed with one or more techniques of the present disclosure to filter the set of features to disregard less significant features. Thus, the Sum/Difference combining and/or weighting may be applied to only the most significant features, thus improving accuracy while decreasing unnecessary computational resource use. In some embodiments, the filter may be based on the weights associated with the target signature and can include any suitable value, such as, e.g., 0.1, 0.2, 0.3 or greater, or corresponding statistical score.

In some embodiments, the sum/difference combining, weighting and thresholding may be applied in any suitable combination to develop a metric (e.g., the DBSR score) to indicate phenotypic recovery based on the list signature cancelling the effects of the target modification based on the phenotype. Below is presented pseudo-code illustrating in mathematical terms twelve possible combinations of the above to compute the DBSR score, where f1=fold change vector of Target; f2=fold change vector of Test; w1=weights vector of Target; w2=weights vector of Test; x=w1>threshold:

$$sum ( abs ( f 1 + f 2 ) ) \qquad ( 1 )$$

$$sum ( abs ( w 1 ⋆ ( f 1 + f 2 ) ) ) \qquad ( 2 )$$

$$sum ( abs ( w 1 ⋆ f 1 + w 2 ⋆ f 2 ) ) \qquad ( 3 )$$

$$sum ( abs ( f 1 ( x ) + f 2 ( x ) ) ) \qquad ( 4 )$$

$$sum ( abs ( w 1 ( x ) ⋆ ( f 1 ( x ) + f 2 ( x ) ) ) ) \qquad ( 5 )$$

$$sum ( abs ( w 1 ( x ) ⋆ f 1 ( x ) + w 2 ( x ) ⋆ f 2 ( x ) ) ) \qquad ( 6 )$$

$$sum ( abs ( f 1 - f 2 ) ) \qquad ( 7 )$$

$$sum ( abs ( w 1 ⋆ ( f 1 - f 2 ) ) ) \qquad ( 8 )$$

$$sum ( abs ( w 1 ⋆ f 1 - w 2 ⋆ f 2 ) ) \qquad ( 9 )$$

$$sum ( abs ( f 1 ( x ) - f 2 ( x ) ) ) \qquad ( 10 )$$

$$sum ( abs ( w 1 ( x ) ⋆ ( f 1 ( x ) - f 2 ( x ) ) ) ) \qquad ( 11 )$$

$$sum ( abs ( w 1 ( x ) ⋆ f 1 ( x ) - w 2 ( x ) ⋆ f 2 ( x ) ) ) \qquad ( 12 )$$

In some embodiments, illustrative computing devices of present disclosure may be programmed with one or more techniques of the present disclosure to use DBSR for in silico drug screening on large libraries of modifications using target signatures obtained from test groups. The target signatures can be obtained using a behavioral platform, as described herein. In some embodiments, the modifications can be screened against an animal model (e.g., a target signature of the animal model), with the goal of prioritizing modifications that can be beneficial for a disease of interest. In some embodiments, DBSR may be employed for in silico screening of drug combinations/drug synergy that can rescue the phenotype of the animal model. In the drug combinations/drug synergy application the signatures of the two drugs that are independently tested in a behavioral platform are added together in a preprocessing step and then treated as a single list signature to be tested against the target phenotype using the DBSR analysis.

Once a library of compounds is assessed against a target, analysis of the binding profile of the top compounds with higher DBSR scores can reveal a common mechanism of action. For example, in an animal model of autism, it is possible that the top compounds showing putative beneficial signatures are all sigma 1 agonists. A new drug may then be developed that binds to the receptor or a drug in the market or in development may be selected that acts through the receptor for an experimental test of the hypothesis.

In some embodiments, illustrative computing devices of present disclosure may be programmed with one or more techniques of the present disclosure to use the DBSR signature analyzer for in silico prediction of the result of combining two or more modifications. In some embodiments, such comparison may predict the partial or complete cancellation of the feature changes leading to a reduced activity of the combination relative to either manipulation on its own. Alternatively, or in addition, the prediction may indicate additive properties, synergistic or potentiation of the individual modification effects, leading to more potent effects through experimental treatment with the manipulation combination.

In some embodiments, illustrative computing devices of present disclosure may be programmed with one or more techniques of the present disclosure to prioritize certain domains for the combinations of modifications according to DBSR. In one embodiment of the invention, it is possible to choose a compound that increases certain features hypothesized to reflect an anxiolytic response (such as increased time in the center of an experimental arena) and another compound that increases features related to certain aspects of depression (such as social behavior). Combination of two such compounds may result in a novel therapy that may address more than one symptom or aspect of disease. Combinations can be done for compounds in early stage of development, for drugs in the market, or for combinations of pharmacological and other modifications (optogenetic, genetic, electrophysiological manipulations and the like).

In some embodiments, instead of looking for complementary signatures between Test and Target, the system and methods described herein can be used to find similarity between Test and Target where similarity is the complement of discrimination when using a machine learning technique. These embodiments can be used to find phenotypic analogs of a signature of interest.

In some embodiments, instead of looking for complementary signatures between Test and Target, the system and methods described herein can be used to find signatures of Test and Target predictive of a desired signature when combined. These embodiments can be used to find combinations of drugs of particular interest.

The aforementioned examples are illustrative and not restrictive.

As described above, FIG. 5 illustrates a block diagram of an exemplary system 500 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the exemplary inventive computing devices and/or the exemplary inventive computing components of the system 500 may be configured to dynamically query and screen a database schema to create a mapping of at least one Target behavioral profile to at least one Test behavior profile of a plurality of Test behavioral profiles associated with a plurality of chemical compounds, as detailed herein.

In some embodiments, DBSR may be implemented to perform the query and screening analyses as a local process using local data local to the database, e.g., at a server 506 and/or 507. In some embodiments, DBSR may be implemented in a distributed fashion, e.g., by processing the Target behavioral profile to create the Target differential feature set remotely from the database, e.g., at devices 502-504 remote from the server 506 and/or 507. In some embodiments, any one or more of devices 502-504 may be used to initiate a request to perform DBSR on a Target behavioral phenotype relative to the database of compounds which causes the server 506 and/or 507 to generate the Target differential feature set, the Test Signature and perform the enrichment and odds ration analyses to screen for similar and/or reverse compounds.

In some embodiments, the system 500 may be based on a scalable computer and/or network architecture that incorporates varies strategies for assessing the data, caching, searching, and/or database connection pooling. Such a scalable architecture can be capable of operating multiple servers and adding or removing servers in response to demand. In some embodiments, the exemplary inventive computing devices and/or the exemplary inventive computing components of the system 500 may be configured to manage the exemplary dynamic mapping module 118 of the present disclosure, utilizing at least one machine-learning model described herein.

In some embodiments, referring to FIG. 5, the devices 502-504 of the system 500 may include virtually any computing device capable of simultaneously launching a plurality of software applications via a network (e.g., cloud network), such as network 505, to and from another computing device, such as servers 506 and 507, each other, and the like. In some embodiments, the devices 502-504 may be personal computers, multiprocessor systems, microprocessor-based or programmable consumer electronics, network PCs, and the like. In some embodiments, one or more client devices within devices 502-504 may be devices that are capable of connecting using a wired or wireless communication medium such as a PDA, POCKET PC, wearable computer, a laptop, tablet, desktop computer, a netbook, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.).

In some embodiments, the devices 502-504 may configured to present the results of DBSR for the Target behavioral profile by employing virtually any web-based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a client device within devices 502-504 may be specifically programmed by either Java, .Net, QT, C, C++ and/or other suitable programming language.

In some embodiments, the exemplary network 505 may provide network access, data transport and/or other services to any computing device coupled to it in order to communicate requests for screen the library of compounds in the database for a particular Target behavioral profile and/or to communicate the results of the DBSR processes. In some embodiments, the exemplary network 505 may include and implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 505 may include and implement, as an alternative or in conjunction with one or more of the above, a WiMAX architecture defined by the WiMAX forum. In some embodiments and, optionally, in combination of any embodiment described above or below, the exemplary network 505 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 505 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 505 may also include mass storage, such as network attached storage (NAS), a storage area network (SAN), a content delivery network (CDN) or other forms of computer or machine-readable media.

In some embodiments, the server 506 or the server 507 may be a web server (or a series of servers) running a network operating system, examples of which may include but are not limited to Microsoft Windows Server, Novell NetWare, or Linux. In some embodiments, server 506 or server 507 may be used for and/or provide cloud and/or network computing. Any of the features of server 506 may be implemented in server 507 and vice versa.

In some embodiments and, optionally, in combination of any embodiment described above or below, for example, one or more of devices 502-504, the server 506, and/or the server 507 may include a specifically programmed module that may be configured to dynamically access and query the database to screen the Test behavioral profiles therein based on a particular Target behavioral profile.

In some embodiments, illustrative computing devices of present disclosure may be programmed with one or more techniques of the present disclosure to use one or more computer engines or engines to perform the techniques of DBSR.

The aforementioned examples are illustrative and not restrictive.

At least some aspects of the present disclosure will now be described with reference to the following numbered clauses:

1. A method to identify a compound using Drug Behavioral Signature Analysis, comprising: receiving, by at least one processor, a target behavioral phenotype associated with a target condition; wherein the target behavioral phenotype comprises a plurality of target behavioral phenotype features; determining, by the at least one processor, a target differential feature set comprising a plurality of target differential features based at least in part on difference between the plurality of target behavioral profile features and a plurality of control features associated with a control behavioral profile; determining, by the at least one processor, a target feature set within the target differential feature set, wherein the target feature set comprises at least one target differential feature of the plurality of target differential features having a significant difference from the control behavioral profile based at least in part on a target statistical scoring of each target differential feature of the plurality of target differential features; generating, by the at least one processor, a ranked target feature set comprising rank ordering of the at least one target differential feature based at least in part on the target statistical scoring of each target differential feature; accessing, by the at least one processor, a test phenotype databased comprising a plurality of test behavioral phenotypes; wherein each test behavioral phenotype comprises a plurality of test behavioral phenotype features; identifying, by at least one processor, at least one test behavioral phenotype associated with a test condition; determining, by the at least one processor, a test differential feature set comprising a plurality of test differential features based at least in part on difference between the plurality of test behavioral profile features and the plurality of vehicle features associated with the vehicle behavioral profile; determining, by the at least one processor, a test feature set within the test differential feature set, wherein the test feature set comprises at least one test differential feature of the plurality of test differential features having a significant difference from the vehicle behavioral profile based at least in part on a test statistical scoring of each test differential feature of the plurality of test differential features; generating, by the at least one processor, a list signature comprising rank ordering of the at least one test differential feature based at least in part on the test statistical scoring of each test differential feature; determining, by the at least one processor, a combined signature comprising a combined signature rank ordering of combined signature statistical values, wherein each combined signature statistical value at each position in the combined signature rank ordering comprises a combination of: the statistical scoring of each target differential feature of each position in the target rank ordering, and the statistical scoring of each test differential feature of each position in the test rank ordering; determining, by the at least one processor, a score of the combined signature based at least in part on an aggregation of the combined signature statistical values; determining, by the at least one processor, a likelihood of phenotypic recovery based at least in part on the score; and returning, by the at least one processor, a treatment recommendation based at least in part on the likelihood of phenotypic recovery.

### Section J

Consistent with disclosed embodiments, a behavioral platform can evaluate a plurality of datasets, including, without limitations, descriptive information about the context, quality, condition, and/or characteristics of the data, based at least in part on at least one similarity score (e.g., a distance metric) to determine similarity or difference(s) between two or more datasets. Some embodiments described herein may include datasets that may represent the effects of modifications. In some embodiments described herein may utilize one or more techniques described herein that may quantify activity of a plurality of compounds, identify compounds with pharmacological profiles similar to a target, desired compound, and/or or drug. In some embodiments, one or more techniques described herein may rank a plurality of library compounds according to the at least one similarity score to one or more reference drugs. In some embodiments, one or more techniques described herein may compare dose responses for two or more drugs. In some embodiments, one or more techniques described herein may generate a granular profile of pharmacological similarity using a panel of desired reference drugs and/or controls. In some embodiments, one or more techniques described herein may input data from in vivo rich-content behavioral assays into one or more trained machine learning models. In yet some embodiments, data from in vitro assays, omics, and structural descriptors can be added to the datasets being compared with the methods described herein.

Consistent with disclosed embodiments, a behavioral platform can use one or more techniques to estimate at least one similarity score associated with at least two or more rich-content datasets obtained from groups of subjects treated with different modifications, or a desired control group. In some embodiments, one or more techniques described herein may utilize observational data obtained with a behavioral platform. For example, one or more techniques described herein may quantify the overall behavioral activity of a treatment group that would be given an active compound as compared to a vehicle group that would be given a composition in which the active compound would be typically given.

In some embodiments, the data flow pipeline 100 shown in FIG. 1 may be executed, for example, by at least one processor of the control computer 50 (FIG. 3A) to generate instant features, state features, domains, motions, and behaviors. Control computer 50 may include an experiment controller 70 that may receive commands from experiment time protocol data 105 to control the plurality of actuators as shown in FIG. 1. The plurality of sensors (e.g., 3D cameras and/or thermal cameras) may send output data to the image device interface circuitry 370 (FIG. 3A) configured to perform time synchronized data collection. Additional sensors may output data to the sensor interface circuitry 368 (FIG. 3A), also referred to as instrumented measurers.

In some embodiments, the plurality of sensors may include sensors associated with some actuators to capture specific responses to the actuators. In some embodiments, the plurality of sensors may be combined with multiple actuators to challenge the test subject to react to various events which are recorded and analyzed by the system.

In some embodiments, the information from the output data of the sensors may be inputted to at least one features extractor machine learning model. These may include at least one instant feature extractor 121, at least one state feature extractor 115, at least one motif feature extractor 110, and/or at least one domain feature extractor 117. Sensor calibration data 123 may be used by the algorithms and/or machine learning models to extract the instant features, state features and/or motif features.

In some embodiments, the extracted instant features, state features, motif features, and/or domain features may be applied to a data collection module 125 that may be used to query a database 130 of instant, states, motif, domain features to identify a target signature associated with a target condition where the subject may exhibit a plurality of target features representative of the target condition. The outputted target signature may be inputted to a first machine learning model 140 whose output may be used to query a drug class signature database 145. In parallel, the at least one processor may extract novel compound features 135 from the outputted target signature from database 130. The output of the drug class signature database 145 and the extracted novel compound features 135 may be inputted to a second machine learning model 155 to output a drug classification 160 for novel drug class.

In some embodiments, the data flow pipeline 100 shown in FIG. 1 utilizes a segmentation model to identify an object (e.g., mouse) and its parts. In some embodiments, the at least one processor can perform operations to segment body parts (e.g., the head, body, tail, or the like) of an animal, in addition to identifying the entire animal as a single entity.

In some embodiments, one or more techniques described herein may quantify at least one activity score that may be quantified based on a discriminability score between at least two datasets, where the discriminability score may refer to an accuracy of a classifier, or a quantity derived therefrom. At least one dataset is a control group. In some embodiments, one or more techniques described herein may quantify at least one similarity value, which is the complement of the at least one activity value, where at least two groups are compared.

In some embodiments, one or more techniques described herein may calculate the activity score between the two groups using, for example, without limitation, a binary machine learning classifier. In some embodiments, a discriminability score of the at least two groups, as assessed by the classifier, may be used as the at least one activity score, representing how phenotypically different the two groups are. For example, the at least two groups may refer to a treatment group and a vehicle group associated with the at least two datasets.

In some embodiments, one or more techniques described herein may utilize a machine learning algorithm to perform the following steps: obtaining a plurality of subsamples, where a number, k, of iterations of the plurality of subsamples of a larger group match sample size n of a smaller group. In some embodiments, when the two groups are of equal size, this step is still repeated k times, using all the data (without subsampling). In some instances, a default value for k may be 100 for activity. In some instances, a default value for k may be 20 for the similarity screening. In some instances, a default value for k may be selected between 20 and 100. In some embodiments, one or more techniques described herein may normalize the subsampled dataset. In some instances, one or more techniques described herein may normalize the subsampled dataset by, without limitation, utilizing a z-normalization. In some embodiments, one or more techniques described herein may dimensionally reduce the dataset by utilizing a Principal Component Analysis (PCA). In some instances, one or more techniques described herein may dimensionally reduce the dataset by selecting the top components (e.g., eigenvalues) covering at least 50% of the variance. In some instances, one or more techniques described herein may dimensionally reduce the dataset by selecting the top components covering at least 30% of the variance. In some instances, one or more techniques described herein may dimensionally reduce the dataset by selecting the top components covering at least 30% of the variance and not more than 80% of the variance to avoid overfitting. Overfitting leads to a reduction of the training accuracy during validation of an algorithm. In some embodiments, where the majority of the data is categorical, PCA can be replaced by Multiple Correspondence Analysis (MCA). In some embodiments, where the data is a mix of continuous and categorical data, PCA can be replaced by Factor Analysis of Mixed Data (FAMD).

In some embodiments, for each k iteration, the success rate of an Linear Discriminant Analysis (LDA) binary classifier may be computed as follows: iterate n*40 times a random partitioning of each group, where ⅓ of the data is left out, and ⅔ are used for training, and compute classification accuracy as (TP+TN)/total where TP represents the true positive and TN the true negatives and total represents all samples; compute average classification accuracy across random partitioning iterations, where a minimum accuracy may be capped at 0.5; and, compute the final activity score as the average classification accuracy across the iterations. In some embodiments, the activity score may range between 0.5 (treatment group is undistinguishable from vehicle group, when the corresponding sample sizes are the same or similar) and 1 (complete separability) can be rescaled as desired.

In some embodiments, the statistical significance of the similarity scores can be calculated using at least one technique under the null hypothesis (H0=there is no difference between the two samples). In some embodiments, the average value of a group activity may be compared against the no-activity threshold (0.5, in a scale from 0.5 to 1) using a one sample t-test (using the calculated standard deviation of the group activity scores obtained from the multiple iterations through the subsets). In some embodiments, the collection of vehicle samples in a dataset may be partitioned in appropriately sized subsets pairs, and the activity score may be calculated multiple times (e.g., more than 500; more than 1,000; more than 1,500, more than 2,000, etc.). For example, the distribution of activity scores that results from this iteration may refer to a threshold, representing the maximal activity score that can be obtained by chance with a probability of 0.05 or less. In some embodiments, the threshold may be calculated for different subset sizes (e.g., for a size of n=and n=8 samples of replicas per group, or any other suitable group size). In some embodiments, the labels of the actual samples being compared may be randomized, and the resulting average and standard deviation may be used to compare against the non-randomized activity average score and standard deviation. For example, p-values may be further processed using a correction to set a given experiment- or family-wise alpha, using a false discovery rate approach or other correction.

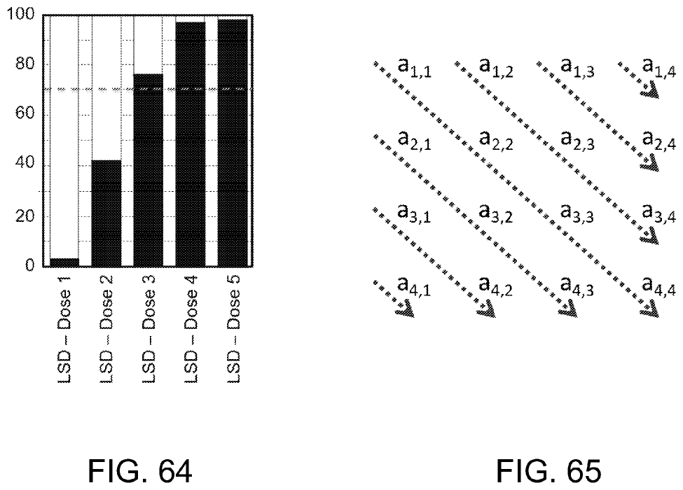

FIG. 64 illustrates a graph illustrating a total activity as a function of dose for the psychedelic drug lysergic acid diethylamide (LSD). In FIG. 64, activity was rescaled from 0% to 100%, and the threshold found using one or more techniques described herein was marked at 70%. In this example, the activity dose-response for LSD was very smooth, with only the 3 mg/kg and 5 mg/kg being different from vehicle in a statistically significant manner. Here, the total activity was calculated against the vehicle control and the type of activity was analyzed using different types of classifiers.

In some embodiments, it is possible to measure the complement of the activity score between two groups, termed the “similarity score.” In a behavioral platform, such a similarity score may be used to quantify the degree of similarity between two or more phenotypes, typically generated by application of two different drug treatments. The similarity score, representing the phenotype closeness, may be calculated as 1—activity score. The data used to calculate activity and the similarity could represent overt behavior, and/or physiology, and/or EEG, and/or biomarkers, and/or structural descriptors of the compounds involved, or any other such rich-content dataset, or any combinations thereof. In some embodiments, the similarity score may range from 0.5 to 1 and can be rescaled as desired. In some embodiments, the similarity score may be applied to a plurality of screen library compounds against a panel of reference compounds tailored toward a specific therapeutic indication of interest. In some embodiments, the similarity score may be applied to a plurality of screen library compounds against a panel of all reference compounds available.

TABLE VIII

| | Ref- | Ref- | Ref- | Ref- | Ref- | Ref- | Ref- | Ref- |
|---|---|---|---|---|---|---|---|---|
| | erence | erence | erence | erence | erence | erence | erence | erence |
| | 1 | 1 | 1 | 2 | 2 | 2 | 3 | 3 |
| | Dose | Dose | Dose | Dose | Dose | Dose | Dose | Dose |
| | 1 | 2 | 3 | 1 | 2 | 3 | 1 | 2 |
| Drug A- | 0.32 | 0.42 | 0.37 | 0.05 | 0.06 | 0.15 | 0.03 | 0.05 |
| Dose 1 | | | | | | | | |
| Drug A- | 0.22 | 0.46 | 0.3 [symbol] | 0.04 | 0.00 | 0.13 | 0.03 | 0.03 |
| Dose 2 | | | | | | | | |
| | | Ref- | Ref- | Ref- | Ref- | Ref- | Ref- | Ref- |
| | | erence | erence | erence | erence | erence | erence | erence |
| | | 3 | 4 | 4 | 4 | 6 | 6 | 6 |
| | | Dose | Dose | Dose | Dose | Dose | Dose | Dose |
| | | 3 | 1 | 2 | 3 | 1 | 2 | 3 |
| | Drug A- | 0.01 | 0.24 | 0.29 | 0.18 | 0.00 | 0.02 | 0.03 |
| | Dose 1 | | | | | | | |
| | Drug A- | 0.01 | 0.07 | 0.15 | 0.15 | 0.01 | 0.04 | 0.04 |
| | Dose 2 | | | | | | | |

[symbol]

indicates data missing or illegible when filed

Table VIII illustrates a plurality of similarity scores calculated using real in vivo data from the SmartCube® system, between a drug development candidate and a number of reference compounds. For example, drug A shows high similarity to the Reference drug #1, with scores of 0.22 up to 0.46, and low similarity to reference drug #2, with scores of 0.00 to 0.15.

In some embodiments, a single score called a diagonal index (DD) may summarize all pair-wise similarity scores calculated for all the possible dose-dose combination of a plurality of compounds assessed at multiple doses, using one or more techniques described herein. In some embodiments, the rationale behind the diagonal index may be that if two compounds are similar, then they would be expected to have maximal similarity across all or most of their doses (with low doses being similar and higher doses also being similar) resulting in a diagonal pattern in the pairwise similarity matrix, as seen in Table IX. In some embodiments, one or more techniques described herein may summarize the totality of the similarity table into a diagonal index. For example, one or more techniques described herein may identify two compounds A and B, with n and m doses each, resulting table has dimensions n×m. Table IX shows an exemplary 4×4 table with k=7 diagonals (FIG. 64) going from top left to bottom right, built with in silico simulated similarity scores.

TABLE IX

| | | Compound B | | | |
|---|---|---|---|---|---|
| | | dose 1 | dose 2 | dose 3 | dose 4 |
| Compound | dose 1 | 0.50 | 0.31 | 0.21 | 0.05 |
| A | dose 2 | 0.27 | 0.50 | 0.29 | 0.20 |
| | dose 3 | 0.22 | 0.30 | 0.50 | 0.33 |
| | dose 4 | 0.09 | 0.18 | 0.32 | 0.50 |

In some embodiments, one or more techniques described herein may compute the diagonal index where the n×m similarity data matrix is considered in terms of all possible k diagonals as seen in FIG. 65, with k=n+m−1, where the central diagonal may be assigned a weight of 1, and the two extreme diagonals (each of 1 element) may be assigned a weight of 0. In some embodiments, the diagonal matrix may have an even number of columns, where the two central diagonals may be assigned a weight of one. In some embodiments, the weights (Wᵢ) for the remaining diagonals within the diagonal matrix may be interpolated linearly between 0 and 1. For example, the weights of a 4×4 matrix would be equal to: W={0 0.33 0.66 1 0.66 0.33 0}, from the left most diagonal to the right most, whereas the weights for a 4×5 matrix would be equal to: W={0 0.33 0.66 1 1 0.66 0.33 0}.

In some embodiments, one or more techniques described herein may compute the diagonal index for each diagonal (D) as the average activity score across the diagonal from which the expected value for the diagonal index is subtracted, where the expected value of the diagonal index is equal to the overall average activity of the whole matrix. In some embodiments, the expected value for the diagonal index may be derived as the average activity across the diagonal in which all elements are assigned the overall average of the activity matrix. In some instances, any negative value after subtraction of the expected value is set to 0. For example, the diagonal index may be computed as:

$$D D = \sum D i W i / \sum W i .$$

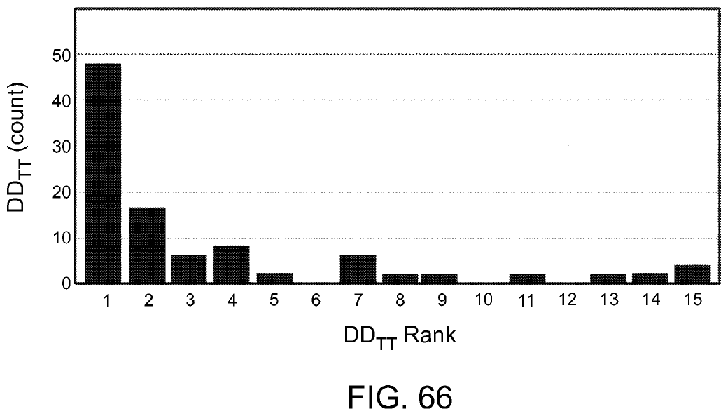

To validate the diagonal index, a large dataset was selected comprising in vivo data obtained with the SmartCube® system covering the phenotypic signature of 90 reference drugs with a combined total of 279 active doses (termed “R” for “Reference” drugs), and separate runs of the same drugs (termed “T” for “Test” drugs), run at a different time, with different subjects, but the same standard SmartCube® procedure. The similarity score for each of the dose-dose pairs of the T and R drugs was computed and used to calculate the diagonal index for each T-R drug pair or DD_{TiRj}. For each Tᵢ drug, the Rⱼ drugs were then ranked by the corresponding DD_{TiRj}. As each T drug was tested twice, it was possible to identify the rank at which similarity to itself was found (DDTTi=DD_{TiRj}, for which Tᵢ is the same drug as Rⱼ). FIG. 66 shows the relative frequency of the ranks for DD_{TTi}, for all Tᵢ drugs. A T drug that is perfectly replicated should be maximally similar to itself, and therefore its DD_{TTi} rank should be very high. The vast majority of correctly predicted compounds ranked first or second, thus validating the method. For example, a second run of the drug LSD had a high DD_{TTi} against a previous run of the LSD, and much lower DD_{TR} for the rest of the R drugs, and therefore its rank was very high. Due to some variability, a newer run of diazepam showed a slightly higher DD_{TR} against chlordiazepoxide and lorazepam (compounds that could have similar phenotypic profiles) than its own DD_{TT} against an older run of diazepam, thus ranking third out of 90. FIG. 66 shows a histogram of all DD_{TTi} as a function of their ranks, indicating that the majority of the drugs can be recognized using this method, having ranks tenth of better, out of 90. The probability to obtain such a distribution by chance is 10\hat{ }(−11), calculated using Kolmogorov-Smirnoff test.

In some embodiments, a compound run in a system producing rich-content datasets can be used to identify other compounds with similar signatures out of a library of compounds using the techniques described herein.

In some embodiments, drugs belonging in the same class of the correct drug may often be among the top-ranking predictions. For instance, the top two predictions for citalopram, in the previously described validation analysis, are sertraline and escitalopram, all SSRI antidepressants.

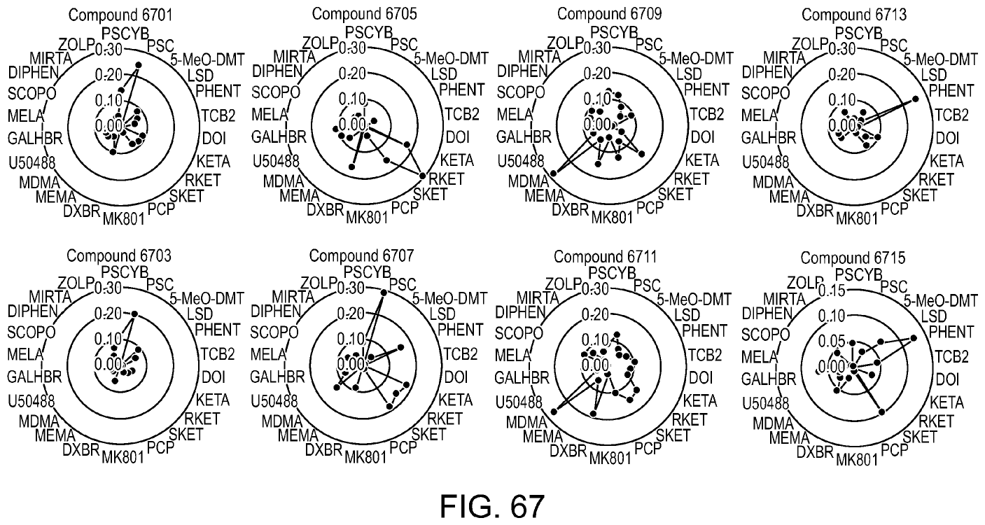

In some embodiments, a plurality of subsets of reference drugs may be created for special purposes. For example, a panel of psychedelic drugs and dissociative reference controls can be used to find drugs with a psychedelic signature, but novel chemistry. FIG. 67 illustrates eight different compounds from a library of novel compounds, and their “psychedelic” profile using spider graphs to represent all the diagonal indexes DDR obtained comparing the test drug (T) against the psychedelic drugs (R), using data obtained from the SmartCube® system. For example, psychedelics can include, as in this example, psilocybin, psilocin, LSD, and other references of interest. For example, dissociative references may include scopolamine and the z-drugs, as examples. For example, compounds (see example compounds 6701 and 6703 in FIG. 67) may show similarity to psilocin, psilocybin, LSD, mescaline, DMT, 5-MeO-DMT, and or derivatives and enantiomers are therefore called Psilomimics. In some examples, the Psilomimic class can be further divided into Triptamimics (similar to Triptamines) and Lisermimics (similar to Lisergamides). In some instances, compounds (see example compounds 6705 and 6707 in FIG. 67) may show similarity to ketamine, R-ketamine, and or S-ketamine, and are called Ketamimics; compounds (see example compounds 6709 and 6711 in FIG. 67) may be similar to MDMA and are called Mollymimics. In some instances, Phenemimics may be similar to phenethylamine (example compounds 6713 and 6715 in FIG. 67). In some instances, compounds may show similarity to mushroom extracts, ayahuasca, psilocin, psilocybin, LSD, DMT, ketamine, MDMA, phenethylamine, ibogaine, other psychedelic and respective enantiomers and all derivatives of therapeutic potential and are called Psychemimics. For example, in FIG. 67, each dot is the DDR of each compound (6701-6715) against each of the

### Reference Drugs

PSCYB: psilocybin;
PS: psilocin;
5-MeO-DMT;
LSD: Lysergic Acid Diethylamide;
PHENT: 2-phenethylamine;
TCB2;
DOI: 1-(4-iodo-2,5-dimethoxyphenyl)propan-2-amine hydrochloride;
KETA: (±) Ketamine hydrochloride;
RKET: R-ketamine;
SKET: S-Ketamine;
PCP: Phencyclidine hydrochloride;
MK801: (1S,9R)-1-methyl-16-azatetracyclo[7.6.1.02,7.010,15]hexadeca-2,4,6,10,12,14-hexaene;
DXBR: Dextromethorphan hydrobromide;
MEMA: [3,5-bis(trideuteriomethyl)-1-adamantyl]amine;
MDMA: 3,4-Methylenedioxymethamphetamine;
U50488: 2-(3,4-dichlorophenyl)-N-methyl-N-[(1R,2R)-2-pyrrolidinocyclohexyl]acetamide;
GALHBR: galantamine hydrobromide;
MELA: melatonin;
SCOPO: scopolamine;
DIPHEN: diphenhydramine;
MIRTA: mirtazapine; and
ZOLPI: zolpidem.

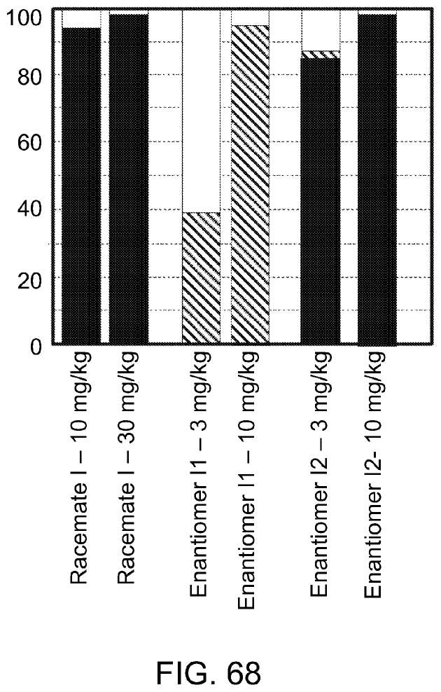

As a non-limiting example according to the present disclosure, one of the top ranked Psilomimic compounds (“Racemate I”) was chosen for analysis. As the first step, the separation of the enantiomers (“Enantiomer I1” and “Enantiomer I2”) was performed. The enantiomers were used to re-run experiments in SmartCube® to test their total activity and type of signature. FIG. 68 illustrates the Class signature of the Racemate Compound I and its two enantiomers according to the SmartCube® system. Whereas the total activity of the compounds, as compared to vehicle, is represented by the height of the filled bars (FIG. 68), the color and texture of the bars represent the Class signature according to a deep learning classifier, trained on a large set of reference drugs. The original Racemate I showed high activity at 10 and 30 mg/kg, and an antidepressant class signature (FIG. 68, black fill). Its Enantiomer I1 showed high activity at the 10 mg/kg dose, with a different Class type (most of its signal corresponded to an antipsychotic profile; FIG. 68, striped fill). Enantiomer I2 exhibited the expected antidepressant signature (black fill in FIG. 68) with putative higher potency than Enantiomer I1 (see higher activity at 3 mg/kg in FIG. 68).

Analysis of binding activity of both enantiomers revealed a significant activity at a number of serotonin receptors for both enantiomers, with a much higher potency for Enantiomer I2 (CEREP panel) based on a binding assay, as provided in Table X. This result may underly the antidepressant signature of both the original Racemate I and its Enantiomer I2.

TABLE X

| | Enantiomer 11 | Enantiomer 12 |
|---|---|---|
| Receptors | % Inhibition @ 10 mM | % inhibition @ 10 mM |
| 5-HT1A | 80% | 95% |
| 5-HT18 | 61% | 94% |
| 5-HTZA | 75% | 95% |
| 5-HT2B | 97% | 100% |
| 5-HT2C | 81% | 97% |
| 5-HTSa | 31% | 91% |
| 5-HT6 | 63% | 93% |
| 5-HT7 | 89% | 98% |
| Alpha 1 adrenergic | 73% | 53% |
| Alpha 2 adrenergic | 51% | 84% |
| D1 | 32% | 71% |
| D2 | 29% | 58% |
| D3 | 53% | 74% |
| D5 | 17% | 63% |
| H1 | 87% | 95% |
| M1 | 33% | 58% |
| M3 | 34% | 56% |
| M4 | 54% | 78% |
| M5 | 32% | 51% |
| Sigma | 83% | 65% |

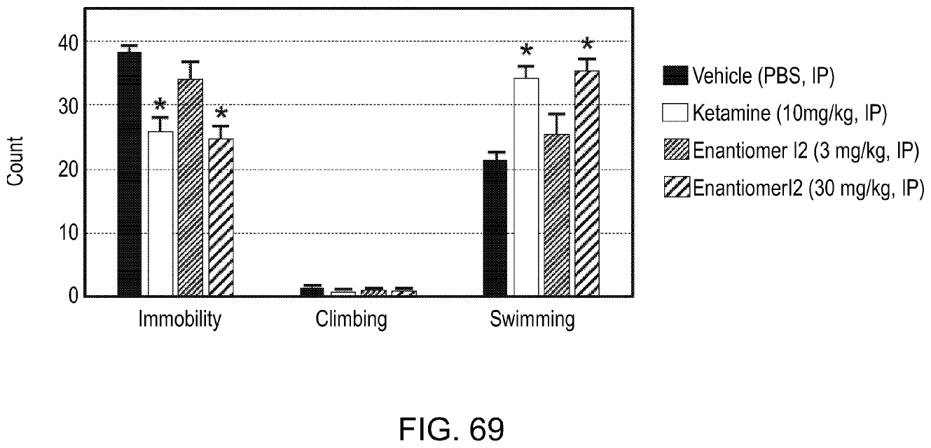

To further explain the results illustrated in FIG. 68 and Table X, a functional assay for Enantiomer I2 revealed weak 5-HT1B agonism, 5-HT2B antagonism, weak 5-HT2A antagonism, 5-HT3 antagonism, weak to no dopaminergic activity, and weak H1 antagonism, revealing putative mechanisms for the antidepressant activity. A further confirmatory efficacy assay, based on ketamine's ability to produce long lasting antidepressant-like effects in the rat forced swim test, showed significant effects “ala-ketamine” of Enantiomer I2 at 30 mg/kg (FIG. 69). Frequency of observations of the three behaviors in the forced swim showed that ketamine and Enantiomer I2 reduced immobility and increased swimming as compared to the vehicle group (p<0.05). Male SD rats from Envigo (IN) were used in the study. Ketamine (10 mg/kg) was dissolved in saline and administered IP at a dose volume of 1 ml/kg 24 H prior testing. Enantiomer I2 was dissolved in PBS and administered IP at a dose volume of 1 ml/kg 24 H prior to testing. Videos were scored by an observer blinded to the treatment and frequency of immobility, climbing and swimming were measured. Data were analyzed by ANOVA followed by post hoc analysis where appropriate.

As detailed herein, active compounds (i.e., the Racemate I), may be identified based on the similarity to a reference of interest such as psychedelic antidepressant, and that such identification can be corroborated with follow up assay(s), confirming the long-lasting antidepressant action, similar to ketamine. Consequently, various embodiments detailed in the present disclosure show the ability to navigate the complexities of racemate mixture and the individual enantiomers, enabling to study compounds with one or more chiral centers. In at least some embodiments resulting candidates for drug development (e.g., exemplary output) may have pharmacology that would be dissimilar to that of reference compound(s) of interest (i.e., not “me-too” drug candidate), as illustrated by the resulting candidate for drug development (Enantiomer I2) that shows complex polypharmacology, dissimilar to that of the reference compound of interest.

In some embodiments, the present disclosure provides, without limitation, a computer-implemented method that may include steps of: identifying an overall set of features associated with a particular manipulation; identifying a first compound and a second compound based on the overall set of features; identifying a plurality of reference compounds associated with the first compound and the second compound; calculating an activity score for each compound utilizing a pre-generated diagonal matrix; determining a similarity value for the first compound and the second compound based on the activity score for each compound and the identified plurality of reference compounds; dynamically synthetize the first compound and the second compound based on the similarity value for each compound; and dynamically perturb a test subject to generate a plurality of new manipulations based on the synthetization of the first compound and the second compound.

The aforementioned examples are illustrative and not restrictive.

### Section K

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

Embodiments of the present disclosure herein describe systems and methods to generate a network visualization of feature transitions between two cohorts. A behavioral platform such as SmartCube® may employ a combination of computer vision, thermal data, electric and mechanical actuators to detect spontaneous responses in a novel environment and responses elicited through anxiogenic and startling stimuli, resulting in measurement of over half a million instant features per subject per session. Readouts include instant features such as estimators of body shape, posture, body parts coordinates, movement, trajectory, and other, and identifiable states such as locomotion, trajectory complexity, body posture and shape, simple behaviors, and behavioral motifs. Although many of the features measured are continuous, it is convenient to focus on traditional behavioral states to provide an interpretable profile for animal models of disease and recovery thereof.

In some embodiments, the processing and storage resources list in this description may include various interconnected computers and/or storage devices that support at least some operations related to the capture, processing and/or archival of data as related to various operations associated with system 310. In some embodiments, at least some resources described herein, including, without limitation, some the equipment directly attached to the animal enclosures, may be connected through network connections on a local area network, and/or using cloud services, as detailed herein.

In some embodiments, the data flow pipeline 100 shown in FIG. 1 may be executed, for example, by the at least one processor 360 of the control computer 50 (FIGS. 3A-3B). A more complete description of system 310 may be found in Section A. System 310 may use computer vision using deep learning for generating instant features, state features, domains, motions, and behaviors as shown in the exemplary data flow pipeline 100 of FIG. 2. Control computer 50 may include an experiment controller 70 (e.g., the control circuitry 364 of FIGS. 3A-3B) that may receive commands from experiment time protocol data 105 to control the plurality of actuators as shown in FIG. 3A. The plurality of sensors (e.g., 2D cameras, 3D cameras, and/or thermal cameras) may send output data to the image device interface circuitry 370 configured to perform time synchronized data collection. Additional sensors may output data to the sensor interface circuitry 368 also referred to instrumented measurers.

In some embodiments, the information from the output data of the sensors may be inputted to at least one feature extractors machine learning model. These may include at least one instant feature extractor 121, at least one state feature extractor 115, at least one motif feature extractor 110, and/or at least one domain feature extractor 117. Sensor calibration data 123 may be used by the algorithm and/or machine learning models to extract instant features, state features, motif features and/or domain features.

In some embodiments, the extracted instant features, state features, motif features and/or domain features may be applied to a data collection module 125 that may be used to query a database 130 of instant, states, motif, domain features to identify a target signature associated with a target condition where the subject may exhibit a plurality of target features representative of the target condition. The outputted target signature may be inputted to a first machine learning model 140 whose output may be used to query a drug class signature database 145. In parallel, the at least one processor 360 may extract novel compound features 135 from the outputted target signature from the database 130. The output of the drug class signature database 145 and the extracted novel compound features 135 may be inputted to a second machine learning model 155 to output a drug classification 160 for novel drug class.

In some embodiments, the data flow pipeline 100 shown in FIG. 2 may include a segmentation model to identify the object 315 and its parts. In some embodiments, the object 315 may be an animal. In some embodiments, object 315 may be a rodent such as a mouse or rat. In some embodiments, the at least one processor 360 can perform operations to segment body parts (e.g., the head, body, tail, or the like) of an animal, in addition to identifying the entire animal as a single entity.

In some embodiments, within an experimental session for data acquisition and analysis performed over a predefined duration, analyses of videos may be done frame by frame, or of other time series done at the highest resolution possible, so as to be used in the extraction of “Instant Features” as represented by sets of values taken by the measured variables. For example, the instant feature set of a mouse in a given point of time within an experimental session may be the set of x, y, z coordinates for its head, body center, paw positions, heart rate and eye temperature.

In some embodiments, an instant feature may be analyzed “as is,” or may be summarized and/or integrated into higher order features, or modified in any number of other ways. These higher order features may include a number of Instant Features be combined into “States”, and states further combined into motifs, and other methods to build complex phenotypic models directly from the instant features, which may further provide a more machine learning based investigation into advanced aspects of function and complex drug effects.

In some embodiments, moving one step higher in the hierarchy of behavioral or other states in a time series, a motif may be a particular set or sequence of values (e.g., at least two time samples) in a time series stream that occurs with a probability higher than chance. Values may represent instant features and/or states. Transitions between every pair of discrete states. Transitions may be from one instant feature to another, or also to the same feature. This is a “first order” motif. It is possible that a set or sequence of several states occurs with a certain probability, and these are n-order motifs, given n states.

In some embodiments, a domain may be a particular scenario, area, or type of collected data that may be related to behavioral manifestation, physiological manifestation, or both. A domain is the highest feature hierarchy. For example, a collection of features representing physiology such as temperature represents a domain (i.e., physiology). Other domains may be measured by neurological behavioral systems configured to assess gait geometry, motor coordination, paw position and paw pressure. Other domains may be exploratory behavior versus consummatory behavior. Domains can be defined using features from the same or across modality such as optical information for overt behavior or thermal information for temperature.

Consistent with disclosed embodiments, complex signals can be summarized in probability maps using, for example, hidden Markov models (fMMs) to assign transition probabilities between different combinations of the many variables collected. In some embodiments, low-probability states are retained in the model, as these low-probability states can help define the more subtle characteristics of the drug or gene signature. These probability maps will be differential, a result of the comparison between a control group and the experimental group. Multiple-dimensional modeling of the test subject based on the systems interpretation of the data allows pattern recognition of the drug signature, predictive drug analysis, and interpretation of the phenotype of a genetically engineered animal.

In some embodiments, motifs may include transitions between every pair of discrete states. Transitions may be from one instant feature to another, or also to the same feature. Thus, auto-transitions may measure the duration of a state. In this way both states and events may be treated with the same model. For example, an instant feature that may be interpreted to represent “grooming” may be analyzed to establish grooming to grooming transitions (e.g., capturing the duration of grooming as a state), or to another instant feature such as that interpreted as “mobility” (e.g., capturing the cessation of grooming and the beginning of mobility). Transition may also be measured as an instant feature representing an event, such as “jumping.” Transitions may be calculated, for example, between two consecutive frames in the video output, thus resulting in same number of transitions (1 less) as number of frames in the videos.

In some embodiments, the at least one processor 360 may use a statistical method to quantify the significant changes in transitions between two cohorts, such as an animal model of disease and its wild-type control, and to visualize them in a network representation. This network visualization may help in understanding the overall feature changes specific to a given cohort relative to a control group. Comparison of these networks in an animal model between treatment and vehicle may also help in the understanding of the features rectified by the drug treatment.

In some embodiments, the at least one processor 360 may apply the Poisson test on the average counts (averaged over all samples within each cohort) of a specific transition in each period between two cohorts may be used to identify statistically significant changes. The null hypothesis for this test is that the ratio of behavior transitions between two cohorts (for example, the ratio of mutant to wild type) is 1. The p-values are calculated by comparing the test statistic to a Poisson distribution. Since hypothesis for multiple transitions are evaluated simultaneously, we adjust for multiple comparisons. The p-values are adjusted using false-discovery rate (FDR) with a cutoff of FDR <0.05 to identify significant transitions. In another embodiment, a collection of control samples (e.g., subjects injected with a vehicle) can be used to determine suitable “normative’ thresholds for consideration of transitions between n=2 (first order) or n>2 states (n-order) that can be considered to have occurred not due to chance. For instance, a collection of 10K control samples can be used to determine such normative thresholds. In another embodiment, normative thresholds can be determined from a collection of between 10K and 60K control samples.

In some embodiments, for each significant transition, the at least one processor 360 may calculate the log of ratio (denoted as “logratio”) of the average counts in each group, i.e., (Average Counts in Mutant)/(Average Counts in WT). The at least one processor 360 may use the sign of the “logratio” to determine the direction of increase or decrease of a given feature in two groups. The at least one processor 360 may calculate the difference of the average counts (denoted as “averagecounts”) and report the difference between the average counts between the two groups, and the product of the log ratio and the average counts (denoted as “logratioxcounts”). Logratioxcounts may be used to estimate the strength or degree of change of the transition while accounting for both fold change and the number of transitions. For all these estimates, any transition behavior with low average count (<2) may be excluded.

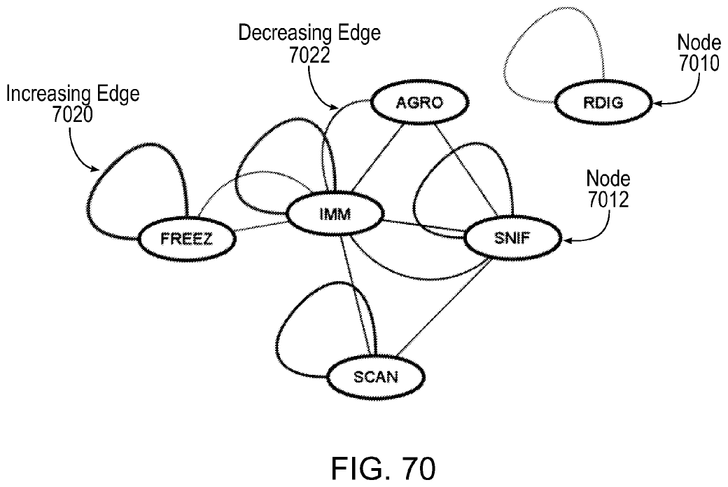

FIG. 70 illustrates an exemplary network visualization in accordance with one or more embodiments of the present disclosure. Finally, the at least one processor 360 may represent these estimates in a network visualization using Cytoscape, an open-source bioinformatics software platform for visualizing interaction networks, for example. The at least one processor 360 may display the visual representation on the GUI 388 of the display 386 as shown in FIG. 3A. In this visualization, the at least one processor 360 may report all significant transition behaviors (FDR<0.05), where each behavior is a node (e.g., nodes 7010, 7012) and transitions between behaviors are edges (e.g., increasing edge 7020, decreasing edge 7022). The at least one processor 360 may use the sign of logratio to determine whether the transition is increased or decreased transition, where increased transitions may be represented as red and decreased transitions as blue edges. The thickness of each interaction may be proportional to the strength or degree of change, as defined by Logratioxcounts.

In some embodiments, a system may include a memory, a display, and at least one processor. The at least one processor may be configured to execute computer code stored in the memory that causes the at least one processor to receive a plurality of features associated with a plurality of subjects, where the plurality of features may include instant features, state features, and motif features, to determine at least one feature transition metric for a plurality of feature transitions in the plurality of features associated with two cohorts from the plurality of subjects, to identify at least one statistically significant feature transition for the plurality of feature transitions in the plurality of features based on the at least one feature transition metric, to determine, based on the at least one statistically significant feature transition associated with the two cohorts, a direction of increase or decrease of at least one feature of interest from the plurality of features, to generate a visualization for all of the at least one feature of interest from the plurality of features for the at least one statistically significant feature transition associated with the two cohorts based on the direction of increase or decrease of at least one feature of interest from the plurality of features and the at least one statistically significant feature transition associated with the two cohorts, where each node in the visualization may be the at least one feature of interest for the at least one statistically significant feature transition, where each edge between each node may be representative of the direction of the increase or the decrease of the at least one statistically significant feature transition, and to display the visualization in a graphic user interface displayed on the display.

In some embodiments, the two cohorts may be a control group and an experimental group.

In some embodiments, the at least one processor may be configured to determine the at least one feature transition metric by applying a Poisson test on average counts on the plurality of feature transitions in the plurality of features associated with the two cohorts from the plurality of subjects.

In some embodiments, the at least one processor may be configured to identify the at least one statistically significant feature transition from the plurality of feature transitions associated with the two cohorts when a false-discovery rate (FDR) based on a p-value of the Poisson test is less than a predefined value.

The aforementioned examples are illustrative and not restrictive.

### Section L

In some embodiments, the at least one processor 360 of system 310 may execute a machine learning model configured to automatically detect HTRs and other psychedelic-induced symptoms to integrate with other analytical tools. In some embodiments, the frequency of HTR can be visualized against the dose-response signature of the compounds being analyzed considering therapeutic and side effects.

FIG. 71 illustrates a response curve for R-(−)-4-iodo-2,5-dimethoxyamphetamine (DOI), showing anxiolytic, another therapeutic signal, and the head twitch response (HTR) in accordance with one or more embodiments of the present disclosure. FIG. 71 represents a signature of DOI in a behavioral platform, where multiple classes can be collapsed into a few classes of interest. For example, multiple classes can be collapsed into five classes of interest for simplicity. Using this particular classifier, DOI shows a predominant anxiolytic therapeutic signal. The therapeutic signature does not separate from the HTR dose-response which is high at both doses (Line in FIG. 71).

In some embodiments, the response curve may be processed in a behavioral platform. The class signals can be obtained with a machine learning classifier utilizing a large number of drugs and doses. The HTR may be obtained from analysis of the data generated for the vehicle and compound only.

In some embodiments, the first step may be to generate a collection of ground-truth examples by manually identifying features corresponding to behaviors of interest (e.g., using a labelling software or platform to add labels identifying such features). Each experimental session can be recorded in terms of precise start and end frame which can set the predefined duration of the experimental session. For manual identification purposes, the system may use a top camera view, at a 50% slower viewing speed, although other angles from the other cameras and other sensors such as piezoelectric and thermal sensors may also be used, and the resulting data integrated. Class analysis (e.g., FIG. 71), on the other hand, may include data acquired from all sensors of a behavioral platform.

The disclosed embodiments can extract behavioral features corresponding to exemplary behaviors. The disclosed embodiments are not limited to any particular implementation of these features. In some embodiments, a behavioral feature can be implemented as a time-stamped indication that a behavior occurred at that time, or a Boolean value in a time series indicating the occurrence of a behavior. In some embodiments, a training dataset including observational data and corresponding, manually identified features can be used to train the machine learning classifier. The exemplary behaviors can include: HTR: Vigorous Head Twitch Response (HTR), with visible blurring of ears in medium to low resolution cameras as seen with treatment with DOI; the system may be trained to exclude blurring due to locomotion, tremor, seizures, or video glitches.
ESH: Unilateral Ear Scratch with hind limb.
ESF: Ear Scratch with both forelimbs consisting of a single-stroke grooming of the head starting behind ears; the system excludes it if part of grooming sequence.
ELO: Body Elongation consisting of a quick elongation forward and return to original posture; the system excludes movements due to rearing, grooming or stretch attend postures, the latter being slower and of longer duration.
SHS: Subtle Head Shake without visible blurring of ears; less intense than the DOI-caused HTRs.

In some embodiments, the extraction of behavioral features can be performed on pre-processed image data. The pre-processing can assist in the detection of behaviors of interest. In some embodiments, the pre-processing can include application of motion enhancement techniques to the image data. Such techniques can amplify or exaggerate motions such as head twitches, ear scratches, head shakes, or body elongations, improving the ability of a machine learning classifier to accurately detect these motions. In some embodiments, the motion enhancement technique can be Eulerian Video Magnification.

In some embodiments, the main behavioral feature of interest may be HTR. In some embodiments, a HTR can be identified as a paroxysmal side-to-side head rotation, along the sagittal axis, which is mediated via 5HT2A receptor activation. Although the 3D cameras may be used to detect the HTR, the system 310 may additionally and/or optionally include a head-mounted magnet and magnetometer coil attached to the head of the mouse. Head-mounted magnetometer measurements may detect HTR as sinusoidal wavelets with a frequency of 80-100 Hz, with a duration of less than 0.15. Control circuitry 364 of the system 310 can be configured to control the head-mounted magnet and magnetometer coil. The sensor interface circuitry 368 can be used to receive the signal data from the head-mounted magnet and magnetometer coil and to process the head-mounted magnetometer measurements.

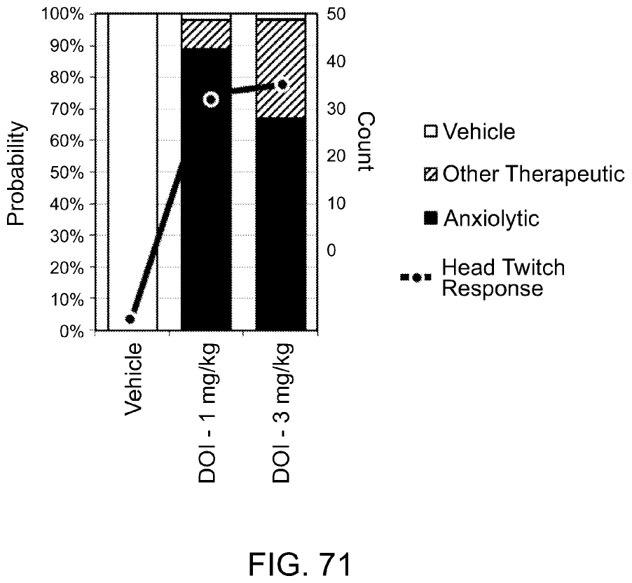

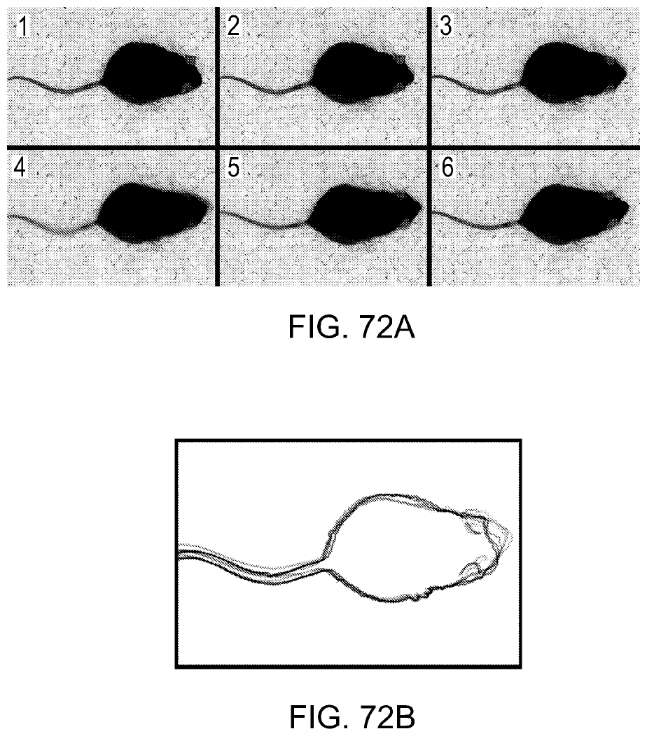

FIG. 72A illustrates frames of a video clip showing a DOI-induced HTR in accordance with one or more embodiments of the present disclosure.

FIG. 72B illustrates combined mouse silhouettes from the FIG. 72A image frames in accordance with one or more embodiments of the present disclosure.

FIGS. 72A-72B illustrate exemplary characteristics that enable the at least one processor 360 to execute a segmentation model so as to capture DOI-induced HTR from the collected images. The head twitch responses may be reliably detected by a human observer in videos of a behavioral platform. The twitch itself can cause a rapid movement of the ears which appear blurred, in medium to low resolution videos, across the span of ˜3 consecutive frames for sampling rate of 7.5 frames/s. Another indirect effect of the HTR observable in videos is the rapid elongation and contraction of the body shape along the sagittal axis. False positives may arise from scenarios that may induce the blurring of the mouse's ears, such as during rapid locomotion. To avoid these false positives the search for these behavioral features can be limited to segments of the videos in which the mouse is not in active locomotion.

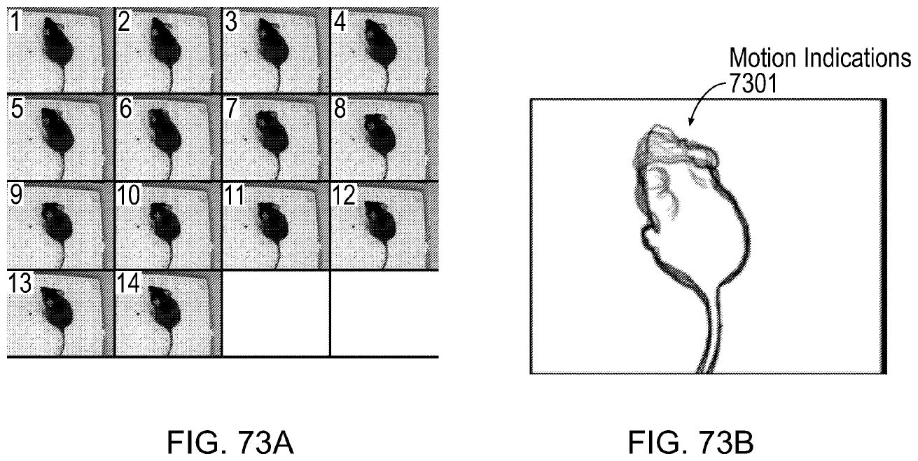

FIG. 73A shows an image frame sequence from a captured video illustrating ear scratch behavior with forepaw (ESF) in accordance with one or more embodiments of the present disclosure.

FIG. 73B shows combined silhouettes from the image frame sequence depicted in FIG. 73A. The combined silhouettes depict aspects of interest (e.g., motion indications 7301) for a segmentation model that may capture DOI-induced ESF in accordance with one or more embodiments of the present disclosure.

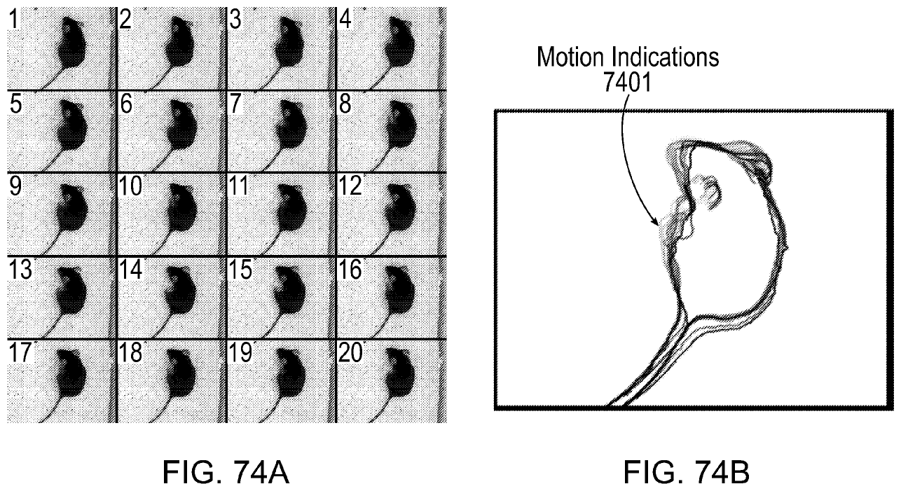

FIG. 74A shows an image frame sequence from a captured video illustrating ear scratch behavior with hindpaw (ESH) in accordance with one or more embodiments of the present disclosure.

FIG. 74B shows combined silhouettes from the image frame sequence depicted in FIG. 74A. The combined silhouettes depict aspects of interest (e.g., motion indications 7401) for a segmentation model that may capture DOI-induced ESH in accordance with one or more embodiments of the present disclosure.

In some embodiments, ear scratches may be commonly associated with psychedelic treatments in rodents. Ear Scratch with Forepaw (ESF) and Hind paw (ESH) may be easily distinguished and detected. They are illustrated in the exemplary embodiments shown in FIGS. 73A-73B and 74A-74B respectively. Grooming (GRO) behavior may be potentially confused with ESF behavior. However, the difference is that GRO may occur as stereotyped set or sequence of behaviors which may include the grooming of the head using the forepaws. True ESF behavior may include an isolated single stroke of the head. Annotated examples of GRO behaviors and other psychedelic-induced behaviors may be used as negative controls during training of a machine learning classifier.

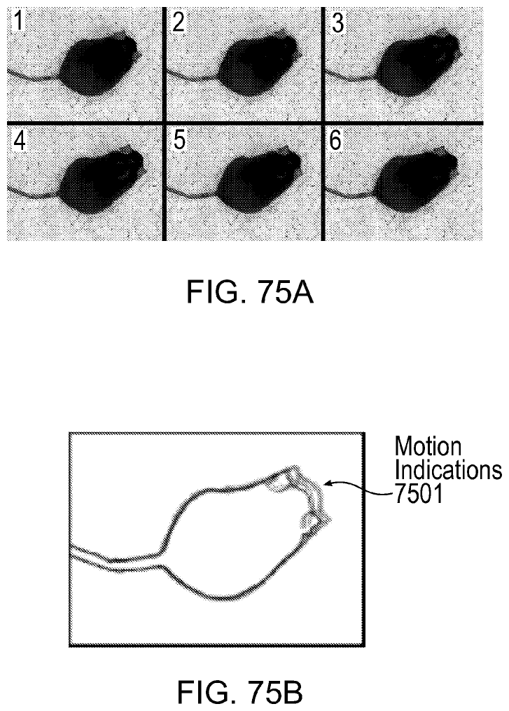

FIG. 75A shows an image frame sequence from a captured video illustrating rapid elongation-contraction of body (ELO) behavior associated with lysergic acid diethylamide (LSD) treatment in accordance with one or more embodiments of the present disclosure.

FIG. 75B shows combined silhouettes from the image frame sequence depicted in FIG. 75A. The combined silhouettes depict aspects of interest (e.g., motion indications 7501) for a segmentation model that can capture DOI-induced ELO in accordance with one or more embodiments of the present disclosure.

As shown in FIGS. 75A-75B, another behavior of interest is a rapid body elongation-contraction (ELO) across the span of about 3 frames. This specific behavior may be associated with psychedelics, as empirical data indicates increased ELO in LSD treated mice.

In some embodiments, separate behavioral features can be associated with HTR and subtle head shakes (SHS), which may appear similar to HTR but are distinct from the HTR sequences that may be observed in DOI-treated mice. The apparent similarity may be due to the fact that the expected head blurring is not observed, or that the head shaking may exhibit a much lower frequency that may be detectable at a 30 fps videos, or that the behavior may be a side-to-side movement as opposed to sagittal. These cases may be potentially confused by the machine learning model, so the subtle head shake features can be manually identified in observational data and used as negative controls in training a machine learning model.

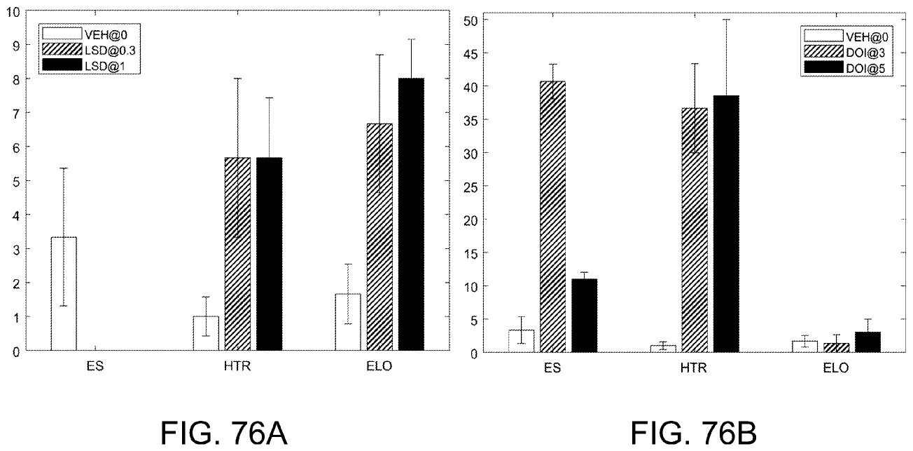

FIG. 76 illustrates graphs illustrating a quantification of psychedelic-related behaviors detected in behavioral platform videos: ear scratch with hind paw (ESH), head twitch response (HTR), and elongation-contraction (ELO) for LSD and DOI at two doses in accordance with one or more embodiments of the present disclosure. FIG. 76 indicates a number of occurrences of HTRs, ESH, and ELOs, in behavioral platform videos of mice treated with DOI, LSD or vehicle, in triplicates, were recorded during the periods of rest for a total of 12 minutes for each subject. The drug DOI induced the most robust increase of HTRs and ear scratches. The use of LSD did not result in occurrences of ear scratches but showed a significant increase in the number of ELOs. These results confirmed the feasibility of detecting and quantifying the psychedelic-associated behaviors in a behavioral platform to predict the hallucinogenic potential of novel compounds and to help find compounds that show therapeutic potential at doses that do not produce such dissociative effects.

In some embodiments, suitable machine learning methods can be used to track segmented objects and silhouettes across consecutive frames to detect the psychedelic associated behaviors of interest. For this purpose, a Deep Learning (DL) framework using a RNN architecture or any other suitable architecture, can be trained on collected ground truth labelled frame sequences for each behavior, as well as the labelled negative controls as described above.

This DL framework based on the RNN MLM architecture may be used to identify and quantify the psychedelic-induced behaviors. Data from sensors and actuators can be used as inputs to the network as described the system 310 of FIG. 3A. Force sensors, which may sample at a higher rate than cameras, can pick up subtle movements and provide additional information that may be combined through the Sensor Fusion algorithms. In addition, these novel features may be added as part of a classifier. The classifier can be used to train to predict dissociative versus non dissociative drug doses, using known psychedelic and other drug data as input.

In some embodiments, a system may include a memory, a plurality of actuators, an enclosure, a plurality of imaging devices, at least one thermal imaging sensor, at least one floor force sensor coupled to the floor of the enclosure, at least one piezoelectric sensor, and at least one processor. The plurality of actuators may include any combination of an aversive stimulus probe, a motor challenge, a controllable light source, a tactile stimulator, or waterers and feeders. The enclosure may include the plurality of actuators coupled to at least one side, a floor, or any combination thereof of the enclosure. The plurality of imaging devices may be configured to capture image data of a plurality of images of a predefined region of the enclosure, including a subject, after the subject has been administered with at least one psychedelic inducing drug. The subject may exhibit at least one psychedelic-induced behavioral feature related to the at least one psychedelic inducing drug. The at least one thermal imaging sensor may be configured to capture thermal image data of the subject of the predefined region of the enclosure. The at least one floor force sensor may be coupled to the floor of the enclosure. The at least one piezoelectric sensor may be coupled to at least one side of the enclosure. The at least one processor may be configured to execute computer code stored in the memory that causes the at least one processor to continuously receive the image data of the plurality of images of the predefined region including the subject over a predefined time interval, to continuously receive thermal imaging data of the subject from the at least one thermal imaging sensor over the predefined time interval, to continuously receive movement data of the least one side of the enclosure, from the floor of the enclosure, or both respectively from the at least one piezoelectric sensor or the floor force sensor over the predefined time interval, and to input the image data of the plurality of images, the thermal imaging data, the movement data of the least one side of the enclosure, the movement data from the floor of the enclosure, or any combination thereof into at least one machine learning model to identify at least one psychedelic-induced behavioral feature related to the administration of the at least one psychedelic inducing drug to the subject.

In some embodiments, the system may further include a head-mounted magnet attached to the head of the subject and a magnetometer.

In some embodiments, the at least one behavioral feature is a head twitch, and the at least one processor is further configured to continuously receive the magnetometer data, and to input the magnetometer data into the at least one machine learning model to identify the head twitch related to the administration of the at least one psychedelic inducing drug to the subject.

In some embodiments, the at least one behavioral feature may be selected from the group consisting of a head twitch, an ear scratch with hindpaws, an ear scratch with forepaws, a body elongation, and a subtle head shake.

As may be appreciated, the embodiments discussed in this section can be implemented using system 500.

The embodiments may further be described as follows:
1. A computer-implemented method of classifying a drug, comprising:
   obtaining observational data concerning an animal subject to which the drug is administered, the observational data acquired using an enclosure for the animal subject, the enclosure instrumented with at least one sensing device;
   extracting features by applying the observational data to a machine-learning feature-extraction component;
   predicting a class label of the drug by applying the features to a machine-learning classifier component, the machine-learning classifier component trained to predict the class label of the drug from, at least in part, the features; and
   providing an indication of the class label.
2. A computer-implemented method of classifying a drug, comprising:
   obtaining observational data concerning an animal subject to which the drug is administered, the observational data comprising at least one of thermal data or respirational data, the observation data acquired using an enclosure for the animal subject, the enclosure instrumented with at least one sensing device;
   extracting features by applying the observational data to a machine-learning feature-extraction component;
   predicting a class label of the drug by applying the features to a machine-learning classifier component trained to predict the class label of the drug from, at least in part, the features; and
   providing an indication of the class label.
3. A computer-implemented method of classifying a psychedelic drug, comprising:
   obtaining observational data concerning an animal subject to which a predetermined dose of the psychedelic drug is administered, the observation data acquired using an enclosure for the animal subject, the enclosure instrumented with at least one sensing device;
   extracting features by applying the observational data to a machine-learning feature-extraction component, the features comprising at least one of head twitch, nose scratch, ear scratch, head shake, body elongation, or elongation-contraction;
   predicting a class label of the psychedelic drug at the predetermined dose by applying the features to a machine-learning classifier component trained to predict the class label of the psychedelic drug from, at least in part, the features; and
   providing an indication of the class label.
4. The computer-implemented method of any one of embodiments 1-3, wherein a machine-learning component is a layer or branch of a machine-learning model.
5. The computer-implemented method of embodiment 4, wherein the machine-learning model is one of an ensemble of machine-learning models.
6. The computer-implemented method of any one of embodiments 1-5, wherein the features comprise instant behavioral features corresponding to sequences of data points indexed in time order of a first predetermined time scale.
7. The computer-implemented method of any one of embodiments 6, further comprising extracting the instant behavioral features using hard-coded definitions contained within the machine-learning feature-extraction component.
8. The computer-implemented method of any one of embodiments 1-7, further comprising:
   deriving higher-order features based on the instant behavioral features using a machine-learning higher-order-extraction component; and
   predicting the class label of the drug by applying the higher-order features to the machine-learning classifier component, the machine-learning classifier component trained to predict the class label of the drug from, at least in part, the higher-order features.
9. The computer-implemented method of embodiment 8, wherein the higher-order features correspond to sets or sequences of instant behavioral features indexed in time order of a second predetermined time scale, the second predetermined time scale being greater than the first predetermined time scale.
10. The computer-implemented method of any one of embodiments 1-9, further comprising obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.
11. The computer-implemented method of any one of embodiments 1-10, wherein the at least one sensing device comprises at least one imaging sensor configured to obtain image data.
12. The computer-implemented method of any one of embodiments 1-11, wherein the at least one imaging sensor comprises a thermal imaging sensor configured to obtain thermal image data and/or a video recording device configured to obtain video data.
13. The computer-implemented method of embodiment 11 or 12, wherein the at least one imaging sensor comprises a camera having a frame rate of at least 30 frames-per-second (fps).
14. The computer-implemented method of any one of embodiments 11-13, wherein the at least one imaging sensor comprises a high-speed camera having a frame rate of at least 70 fps.
15. The computer-implemented method of any one of embodiments 11-13, wherein the at least one imaging sensor comprises a high-speed camera having a frame rate that is equal or superior to:
   a predetermined sampling rate for a behavior or action of the animal subject, or
   the maximum of the predetermined sampling rates for a collection of behaviors or actions extracted from a single data source.
16. The computer-implemented method of any one of embodiments 11-15, wherein the at least one imaging sensor comprises an event imaging sensor configured to obtain dynamic image data.
17. The computer-implemented method of embodiment 16, wherein the event imaging sensor is configured to have a dynamic range of at least 100 dB or an equivalent frame rate of at least 500,000 fps.
18. The computer-implemented method of any one of embodiments 11-17, further comprising using the at least one imaging sensor with at least one mirror to obtain 3D image data.
19. The computer-implemented method of any one of embodiments 11-18, wherein the at least one imaging sensor comprises a plurality of imaging sensors configured to obtain 3D image data.
20. The computer-implemented method of any one of embodiments 11-19, wherein the observational data comprises a video of the animal subject obtained using the at least one imaging sensor, and the method further comprises segmenting image frames of the video using a machine-learning segmentation model.
21. The computer-implemented method of embodiment 20, further comprising:
   segmenting image frames of the video using a machine-learning segmentation model; and
   extracting the features by tracking at least one segmented object in the image frames using a trained deep learning component.
22. The computer-implemented method of any one of embodiments 1-21, wherein the observational data comprises external data, the external data comprising data concerning one or more environmental designs of the enclosure, data concerning one or more stimuli given to the animal subject, or one or more rewards given to the animal subject.
23. The computer-implemented method of any one of embodiments 1-22, wherein the observational data comprises physiological data of the animal subject.
24. The computer-implemented method of any one of embodiments 23, wherein the at least one sensing device comprises a thermal sensor and the physiological data comprises temperature data obtained using the thermal sensor.
25. The computer-implemented method of embodiment 24, wherein the temperature data comprises temperature measurements of at least one body part of the animal subject, the at least one body part comprising at least one or more eyes, paws, tail, or limbs.
26. The computer-implemented method of any one of embodiments 23-25, wherein the at least one sensing device comprises at least one electroencephalogram (EEG) electrode and the physiological data comprises EEG data obtained using the least one EEG electrode.
27. The computer-implemented method of any one of embodiments 2-26, wherein the respirational data comprises a respiration rate during a period when the animal subject is not in active locomotion.
28. The computer-implemented method of any one of embodiments 11-27, further comprising deriving the respirational data, using a machine-learning respiration component, from image data obtained from at least one imaging sensor.
29. The computer-implemented method of any one of embodiments 1-28, wherein the machine-learning feature-extraction component comprises a supervised machine-learning component, an unsupervised machine-learning component, or both.
30. The computer-implemented method of any one of embodiments 8-29, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.
31. The computer-implemented method of any one of embodiments 8-30, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.
32. The computer-implemented method of any one of embodiments 8-31, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.
33. The computer-implemented method of any one of embodiments 1-32, further comprising:
   creating a treatment signature from the features;
   generating a signature difference between the treatment signature and a baseline signature concerning a control animal, the baseline signature comprising the features; and
   identifying a reference drug based on the signature difference and the treatment signature; and
   providing the indication of the class label based on the identified reference drug.
34. The computer-implemented method of embodiment 33, further comprising ranking the features of the treatment signature based on the signature difference using a support vector machine-learning component.
35. The computer-implemented method of embodiment 33 or 34, further comprising weighting one or more of feature difference values between the treatment signature and the baseline signature prior to identifying the reference drug.
36. The computer-implemented method of embodiment 35, wherein the weights are generated using decorrelated ranked feature analysis.
37. The computer-implemented method of any one of embodiments 33-36, wherein the identification of the reference drug comprises generating a similarity value for the reference drug using the treatment signature and a reference signature corresponding to the reference drug, the reference signature comprising the features.
38. The computer-implemented method of any one of embodiments 33-37, wherein the drug is administered to the animal subject at a first dose and the reference drug is administered at a second dose.
39. The computer-implemented method of any one of embodiments 33-37, further comprising generating a plurality of similarity values corresponding to the administration of the reference drug at different doses.
40. The computer-implemented method of any one of embodiments 37-39, wherein the generation of the similarity value comprises generating an upregulation enrichment score and a downregulation enrichment score for the reference drug using the treatment signature and reference signature.
41. The computer-implemented method of any one of embodiments 37-40, wherein the generation of the similarity value comprises generating a combined enrichment score for the reference drug using the treatment signature and the reference signature.
42. The computer-implemented method of any one of embodiments 33-41, further comprising deriving a recovery value using a function of the treatment signature and a target signature concerning the animal subject prior to administration of the drug, the target signature comprising the features.
43. The computer-implemented method of any one of embodiments 1-42, further comprising deriving a treatment Markov model concerning the animal subject using a machine-learning Markov component, the treatment Markov model comprising a plurality of Markov states representing a selection of the features, each Markov state being associated with one or more Markov states by one or more transition probabilities.
44. The computer-implemented method of embodiment 43, wherein the selection of the higher-order features comprise a selection of state features, and the plurality of Markov states represent the selection of state features, and the method further comprises deriving at least one motif feature representing a sequence of transitions of one or more of the selected state features.
45. The computer-implemented method of embodiment 43 or 44, wherein the treatment Markov model is a hidden Markov model comprising at least one hidden state.
46. The computer-implemented method of any one of embodiments 43-45, further comprising generating a visual representation of the treatment Markov model concerning the animal subject; and displaying the visual representation on a display.
47. The computer-implemented method of any one of embodiments 43-46, further comprising obtaining, using the machine-learning Markov component, a control Markov model concerning a control animal to which a vehicle is administered, the control Markov model comprising the plurality of Markov states representing the selection of the features.
48. The computer-implemented method of any one of embodiments 43-47, further comprising generating transition probability differences between the transition probabilities of the treatment Markov model and the transition probabilities of the control Markov model; and generating a visual representation of the transition probability differences associated with the plurality of Markov states.
49. The computer-implemented method of any one of embodiments 8-48, wherein the higher-order features comprise at least one of head twitch, nose scratch, ear scratch, head shake, body elongation, or elongation-contraction, and the method further comprises predicting the class label to be associated with psychedelics.
50. The computer-implemented method of any one of embodiments 1-49, further comprising predicting the class label to be associated with one or more subclasses of psychedelics, entheogens, or psychoplastogens.
51. The computer-implemented method of any one of embodiments 1-50, wherein the animal subject is a rodent.
52. The computer-implemented method of any one of embodiments 1-51, wherein the animal subject is a mouse or a rat.
53. The computer-implemented method of any one of embodiments 1-52, wherein the drug is administered before the data is acquired or during acquisition of the data.
54. The computer-implemented method of any one of embodiments 1-53, further comprising obtaining the observational data concerning the animal subject while the animal subject is not in active locomotion.
55. The computer-implemented method of any one of embodiments 1-54, wherein the at least one sensing device comprises a headset comprising at least one of an accelerometer, gyroscope, or magnetometer, the headset configured to detect at least one type of motion of the head of the animal subject.
56. The computer-implemented method of any of embodiments 3-55, further comprising training the machine-learning classifier component to predict the class label of the psychedelic drug representing a treatment effect at the predetermined dose, the predetermined dose being a non-dissociative drug dose.
57. The computer-implemented method of any one of embodiments 3-56, further comprising training the machine-learning classifier component to predict the class label of the psychedelic drug representing a non-specific treatment effect at the predetermined dose, the predetermined dose being a dissociative drug dose.
58. A computer-implemented drug screening method, comprising:
   obtaining observational data concerning an animal subject, the observational data acquired using an enclosure for the animal subject, the enclosure instrumented with at least one sensing device;
   extracting instant behavioral features from the observational data;
   creating a treatment signature, the treatment signature including higher-order features derived from the instant behavioral features using a first machine-learning component, the higher-order features including at least one of a state feature, a motif feature, or a domain feature;
   generating a target signature difference between the treatment signature and a baseline signature;
   identifying at least one reference drug or condition based on the target signature difference, identification comprising:
      generating an upregulation enrichment score and a downregulation enrichment score for the at least one reference drug or condition using the target signature difference and a reference signature difference corresponding to the one of the at least one reference drug or condition;
      generating a combined enrichment score for the at least one reference drug or condition using the target signature difference and a reference signature difference corresponding to the one of the at least one reference drug or condition; or
      generating a similarity value for the at least one reference drug or condition using the target signature difference and a reference signature difference corresponding to the one of the at least one reference drug or condition; and
   providing an indication of the similarity value, combined enrichment score, or upregulation and downregulation enrichment scores for the at least one reference drug or condition.
59. The computer-implemented drug screening method of embodiment 58, wherein identifying the at least one reference drug or condition based on the target signature difference comprises generating the upregulation enrichment score and the downregulation enrichment score for the at least one reference drug or condition.
60. The computer-implemented drug screening method of embodiment 58 or 59, wherein the upregulation enrichment score and the downregulation enrichment score comprise gene set enrichment analysis scores.
61. The computer-implemented drug screening method of any one of embodiments 58-60, wherein identifying the at least one reference drug or condition based on the target signature difference comprises generating the combined enrichment score for the at least one reference drug or condition.
62. The computer-implemented drug screening method of any one of embodiments 58-61, wherein generating the combined enrichment score for the at least one reference drug or condition comprises:
   generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
   generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.
63. The computer-implemented drug screening method of any one of embodiments 58-62, wherein identifying the at least one reference drug or condition based on the target signature difference comprises generating the similarity value for the at least one reference drug or condition.
64. The computer-implemented drug screening method of any one of embodiments 58-63, wherein the animal subject is an animal raised or modified to serve as a model of a human disease.
65. The computer-implemented drug screening method of embodiment 64, wherein the human disease is Rett syndrome, Parkinson's disease, Alzheimer's disease, Huntington disease, Tuberous Sclerosis Complex, or Autism Spectrum Disorder.
66. The computer-implemented drug screening method of any one of embodiments 58-65, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.
67. The computer-implemented drug screening method of any one of embodiments 58-66, further comprising weighting one or more of behavioral feature difference values of the reference signature difference or the target signature difference prior to identifying the at least one reference drug or condition.
68. The computer-implemented drug screening method of embodiment 67, wherein the weights are generated using decorrelated ranked feature analysis.
69. A computer-implemented method drug screening method, comprising:
   in a training phase:
   obtaining, for each first animal subject in three or more sets of first animal subjects, each of the first sets corresponding to a combination of values of two or more characteristics of the first animals, a first value for each behavioral feature in a set of features, the features including:
      instant behavioral features extracted from observational data acquired for the first animal subjects using an enclosure instrumented with at least one sensing device; and
      higher-order features derived from the instant behavioral features using a first machine-learning component;
   determining, using a second machine-learning component and the first values, a mapping between at least two dimensions and corresponding functions of the features, the at least two dimensions including a treatment dimension and a secondary dimension; and
   in a screening phase:
   obtaining a second value for each behavioral feature in the set of the features for a second animal subject to which a compound is administered;
   determining, using the mapping and the second values, a treatment effect of the compound; and
   providing an indication of the treatment effect.
70. The computer-implemented method of embodiment 69, wherein the corresponding function of the features represents a weighted combination of the features, and the method further comprises determining weights of the function of the features based on a discrimination power of each behavioral feature derived using a third machine-learning component.
71. The computer-implemented method of embodiment 70, wherein the third machine-learning component is a support vector machine-learning component trained to determine the weights based on features of a test animal group and a control animal group.
72. The computer-implemented method of any of embodiments 69-72, wherein the secondary dimension comprises a dimension orthogonal to the treatment dimension.
73. The computer-implemented method of embodiment 72, further comprising determining, using the mapping and the second values, a secondary effect along the secondary dimension.
74. The computer-implemented method of embodiment 73, wherein the secondary effect comprises a dissociative effect of the compound.
75. The computer-implemented method of embodiment 73 or 74, wherein the secondary effect comprises a side effect of the compound.
76. The computer-implemented method of any one of embodiments 73-75, wherein the secondary effect comprises a physiological condition.
77. The computer-implemented method of embodiment 76, wherein the physiological condition is aging.
78. The computer-implemented method of embodiment 76, wherein the physiological condition is a neurological disease, disorder, or dysfunction.
79. A computer-implemented method of classifying a drug comprising:
   obtaining EEG data from a plurality of electrodes positioned on an animal subject to which the drug is administered at a first dose;
   obtaining acceleration data from one or more accelerometers positioned on the animal subject to which the drug is administered;
   predicting a class label for the drug by applying the EEG data and the acceleration data to a machine-learning classifier component trained to predict the class label using the EEG data and the acceleration data; and
   providing an indication of the class label.
80. The computer-implemented method of embodiment 79, further comprising obtaining observational data concerning the animal subject, the observation data acquired using an enclosure for the animal subject, the enclosure instrumented with at least one sensing device.
81. The computer-implemented method of embodiment 80, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.
82. The computer-implemented method of embodiment 80 or 81, further comprising extracting features by applying the observational data to a machine-learning feature-extraction component, the observational data comprising the EEG data and the acceleration data.
83. The computer-implemented method of embodiment 82, wherein the features comprise instant behavioral features.
84. The computer-implemented method of embodiment 83, wherein the features comprise higher-order features derived from the instant behavioral features using a machine-learning higher-order feature-extraction component.
85. The computer-implemented method of embodiment 84, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.
86. The computer-implemented method of embodiment 85, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.
87. The computer-implemented method of embodiment 86, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.
88. The computer-implemented method of any one of embodiments 79-87, wherein the EEG data comprises wake EEG and sleep EEG, and the method further comprises automatically separating the wake EEG from the sleep EEG based on the EEG data and the acceleration data.
89. The computer-implemented method of any one of embodiments 79-88, further comprising:
   obtaining reference EEG data from the plurality of electrodes positioned on the animal subject to which a reference drug is administered at a second dose; and
   obtaining reference acceleration data from the one or more accelerometers positioned on the animal subject to which the reference drug is administered at the second dose.
90. The computer-implemented method of embodiment 89, further comprising generating a similarity value for the reference drug using the EEG data, the acceleration data, the reference EEG data, and the reference acceleration data.
91. The computer-implemented method of any one of embodiments 79-90, wherein the machine-learning classifier component comprises a Recurrent Neural Network (RNN).
92. The computer-implemented method of any one of embodiments 79-91, wherein machine-learning classifier component is a layer or branch of a machine-learning model.
93. The computer-implemented method of embodiment 92, wherein the machine-learning model is one of an ensemble of machine-learning models.
94. The computer-implemented method of embodiment 93, wherein the ensemble of machine-learning models comprises an ensemble of neural network models.
95. The computer-implemented method of any one of embodiments 79-94, further comprising:
   obtaining low temporal resolution and low frequency resolution power spectra data from the EEG data; and
   predicting the class label for the drug further comprising applying the low temporal resolution and low frequency resolution power spectra data to a low-resolution machine-learning classifier component trained to predict the class label using the low temporal resolution and low frequency resolution power spectra data.
96. The computer-implemented method of any one of embodiments 79-95, further comprising:
   obtaining high temporal resolution and high frequency resolution power spectra data from the EEG data; and
   predicting the class label for the drug further comprising applying the high temporal resolution and high frequency resolution power spectra data to a high-resolution machine-learning classifier component trained to predict the class label using the high temporal resolution and high frequency resolution power spectra data.
97. The computer-implemented method of any one of embodiments 79-96, further comprising:
   obtaining covariance data of EEG data obtained from at least two of the plurality of electrodes; and
   predicting the class label for the drug further comprising applying the covariance data to a covariance machine-learning classifier component trained to predict the class label using the covariance data.
98. The computer-implemented method of any one of embodiments 79-94, further comprising at least one of:
   obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
   obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
   obtaining covariance data of EEG data obtained from at least two of the plurality of electrodes; and predicting the class label for the drug further comprising applying the covariance data to a covariance machine-learning classifier component trained to predict the class label using the covariance data.
99. The computer-implemented method of any one of embodiments 79-98, wherein the animal subject is a rodent.
100. A computer-implemented method of extracting gait features of a rodent comprising:
   obtaining video data concerning a rodent, to which a drug is administered, over a predetermined period, the video data acquired using an enclosure for the rodent, the enclosure instrumented with an illuminated track for the rodent and at least one imaging device positioned to image an underside of the illuminated track;
   annotating frames in the video data with labels using two machine-learning components, the labels including:
      a first one of the two machine-learning components configured to divide a frame in video data into segments corresponding to first object classes, the first object classes comprising a paw class; and
      a second one of the two machine-learning components configured to detect bounding boxes corresponding to second object classes, the second object classes including hind left, hind right, front left, and front right paws;
   generating segmented images using the annotating frames, the segmented images divided into segments corresponding to third object classes including hind left, hind right, front left, and front right paws; and extracting gait features of the rodent from the segmented images.
101. The computer-implemented method of embodiment 100, wherein the first object classes further comprise a background class and a body class.
102. The computer-implemented method of embodiment 100 or 101
103. wherein the second object class further comprise a background class, a first body class indicating the rodent moving from left to right or clockwise, and a second body class indicating the rodent moving from right to left or counterclockwise.
104. The computer-implemented method of any one of embodiments 100-102, wherein the first one of the two machine-learning components comprises a U-net convolutional neural network (CNN).
105. The computer-implemented method of any one of embodiments 100-103, wherein the second one of the two machine-learning components comprises a region-based CNN (R-CNN).
106. The computer-implemented method of any one of embodiments 100-104, further comprising automatically correcting the division of the frame into segments and/or identification of the third object classes using a plurality of heuristic rules based on positional relationship of the third object classes.
107. The computer-implemented method of any one of embodiments 100-105, further comprising extracting positional data of the body center and one or more paws over a sequence of frames in the video data.
108. The computer-implemented method of embodiment 106, further comprising extracting the gait features from the positional data over the sequence of frames in the video data.
109. The computer-implemented method of any one of embodiments 100-107, further comprising extracting the gait features over a plurality of cycles; and deriving a gait pattern of the rodent from the gait features.
110. The computer-implemented method of any one of embodiments 100-108, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.
111. The computer-implemented method of any one of embodiments 100-108, further comprising extracting features of the rodent from the gait features using a machine-learning feature-extraction component.
112. The computer-implemented method of embodiment 110, wherein the features comprise at least one of forward walk, immobile, turn around, or backward walk.
113. The computer-implemented method of embodiment 110, wherein the features comprise instant behavioral features.
114. The computer-implemented method of embodiment 112, wherein the features comprise higher-order features derived from the instant behavioral features using a machine-learning higher-order feature-extraction component.
115. The computer-implemented method of embodiment 113, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.
116. The computer-implemented method of embodiment 114, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.
117. The computer-implemented method of embodiment 115, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.
118. A system for classifying a drug, comprising:
   at least one processor; and
   a non-transitory storage medium storing instructions that, when executed by the at least one processor, cause the system to perform operations comprising:
      obtaining observational data concerning an animal subject to which the drug is administered, the observational data acquired using an enclosure for the animal subject, the enclosure instrumented with at least one sensing device;
      extracting features by applying the observational data to a machine-learning feature-extraction component;
      predicting a class label of the drug by applying the features to a machine-learning classifier component, the machine-learning classifier component trained to predict the class label of the drug from, at least in part, the features; and
      providing an indication of the class label.
119. A system for classifying a drug, comprising:
   at least one processor; and
   a non-transitory storage medium storing instructions that, when executed by the at least one processor, cause the system to perform operations comprising:
      obtaining observational data concerning an animal subject to which the drug is administered, the observational data comprising at least one of thermal data or respirational data, the observation data acquired using an enclosure for the animal subject, the enclosure instrumented with at least one sensing device;
      extracting features by applying the observational data to a machine-learning feature-extraction component;
      predicting a class label of the drug by applying the features to a machine-learning classifier component trained to predict the class label of the drug from, at least in part, the features; and
      providing an indication of the class label.
120. A system for classifying a psychedelic drug, comprising:
   at least one processor; and
   a non-transitory storage medium storing instructions that, when executed by the at least one processor, cause the system to perform operations comprising:
      obtaining observational data concerning an animal subject to which a predetermined dose of the psychedelic drug is administered, the observation data acquired using an enclosure for the animal subject, the enclosure instrumented with at least one sensing device;
      extracting features by applying the observational data to a machine-learning feature-extraction component, the features comprising at least one of head twitch, nose scratch, ear scratch, head shake, body elongation, or elongation-contraction;
      predicting a class label of the psychedelic drug at the predetermined dose by applying the features to a machine-learning classifier component trained to predict the class label of the psychedelic drug from, at least in part, the features; and
      providing an indication of the class label.
121. The system of any one of embodiments 117-119, wherein a machine-learning component is a layer or branch of a machine-learning model.
122. The system of embodiment 120, wherein the machine-learning model is one of an ensemble of machine-learning models.
123. The system of any one of embodiments 117-121, wherein the features comprise instant behavioral features corresponding to sets or sequences of data points indexed in time order of a first predetermined time scale.
124. The system of any one of embodiments 122, wherein the operations further comprise extracting the instant behavioral features using hard-coded definitions contained within the machine-learning feature-extraction component.
125. The system of any one of embodiments 117-123, wherein the operations further comprise:
   deriving higher-order features based on the instant behavioral features using a machine-learning higher-order-extraction component; and
   predicting the class label of the drug by applying the higher-order features to the machine-learning classifier component, the machine-learning classifier component trained to predict the class label of the drug from, at least in part, the higher-order features.
126. The system of embodiment 124, wherein the higher-order features correspond to sets or sequences of instant behavioral features indexed in time order of a second predetermined time scale, the second predetermined time scale being greater than the first predetermined time scale.
127. The system of any one of embodiments 117-125, wherein the operations further comprise obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.
128. The system of any one of embodiments 117-126, wherein the at least one sensing device comprises at least one imaging sensor configured to obtain image data.
129. The system of any one of embodiments 117-127, wherein the at least one imaging sensor comprises a thermal imaging sensor configured to obtain thermal image data.
130. The system of embodiment 127 or 128, wherein the at least one imaging sensor comprises a camera having a frame rate of at least 30 frames-per-second (fps).
131. The system of any one of embodiments 127-129, wherein the at least one imaging sensor comprises a high-speed camera having a frame rate of at least 70 fps.
132. The system of any one of embodiments 127-129, wherein the at least one imaging sensor comprises a high-speed camera having a frame rate that is equal or superior to:
   a predetermined sampling rate for a behavior or action of the animal subject, or
   the maximum of the predetermined sampling rates for a collection of behaviors or actions extracted from a single data source.
133. The system of any one of embodiments 127-131, wherein the at least one imaging sensor comprises an event imaging sensor configured to obtain dynamic image data.
134. The system of embodiment 132, wherein the event imaging sensor is configured to have a dynamic range of at least 100 dB or an equivalent frame rate of at least 500,000 fps.
135. The system of any one of embodiments 127-133, wherein the operations further comprise using the at least one imaging sensor with at least one mirror to obtain 3D image data.
136. The system of any one of embodiments 127-134, wherein the at least one imaging sensor comprises a plurality of imaging sensors configured to obtain 3D image data.
137. The system of any one of embodiments 127-135, wherein the observational data comprises a video of the animal subject obtained using the at least one imaging sensor, and the operations further comprise segmenting image frames of the video using a machine-learning segmentation model.
138. The system of embodiment 136, wherein the operations further comprise:
   segmenting image frames of the video using a machine-learning segmentation model; and
   extracting the features by tracking at least one segmented object in the image frames using a trained deep learning component.
139. The system of any one of embodiments 117-137, wherein the observational data comprises external data, the external data comprising data concerning one or more environmental designs of the enclosure, data concerning one or more stimuli given to the animal subject, or one or more rewards given to the animal subject.
140. The system of any one of embodiments 117-138, wherein the observational data comprises physiological data of the animal subject.
141. The system of any one of embodiments 139, wherein the at least one sensing device comprises a thermal sensor and the physiological data comprises temperature data obtained using the thermal sensor.
142. The system of embodiment 140, wherein the temperature data comprises temperature measurements of at least one body part of the animal subject, the at least one body part comprising at least one or more eyes, paws, tail, or limbs.
143. The system of any one of embodiments 139-141, wherein the at least one sensing device comprises at least one electroencephalogram (EEG) electrode and the physiological data comprises EEG data obtained using the least one EEG electrode.
144. The system of any one of embodiments 118-142, wherein the respirational data comprises a respiration rate during a period when the animal subject is not in active locomotion.
145. The system of any one of embodiments 127-143, wherein the operations further comprise deriving the respirational data, using a machine-learning respiration component, from image data obtained from at least one imaging sensor.
146. The system of any one of embodiments 117-144, wherein the machine-learning feature-extraction component comprises a supervised machine-learning component, an unsupervised machine-learning component, or both.
147. The system of any one of embodiments 124-145, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
148. The system of any one of embodiments 124-146, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
149. The system of any one of embodiments 124-147, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
150. The system of any one of embodiments 117-148, wherein the operations further comprise:
   creating a treatment signature from the features;
   generating a signature difference between the treatment signature and a baseline signature concerning a control animal, the baseline signature comprising the features; and
   identifying a reference drug based on the signature difference and the treatment signature; and
   providing the indication of the class label based on the identified reference drug.
151. The system of embodiment 149, wherein the operations further comprise ranking the features of the treatment signature based on the signature difference using a support vector machine-learning component.
152. The system of embodiment 149 or 150, wherein the operations further comprise weighting one or more of feature difference values between the treatment signature and the baseline signature prior to identifying the reference drug.
153. The system of embodiment 151, wherein the weights are generated using decorrelated ranked feature analysis.
154. The system of any one of embodiments 149-152, wherein the identification of the reference drug comprises generating a similarity value for the reference drug using the treatment signature and a reference signature corresponding to the reference drug, the reference signature comprising the features.
155. The system of any one of embodiments 149-153, wherein the drug is administered to the animal subject at a first dose and the reference drug is administered at a second dose.
156. The system of any one of embodiments 149-153, wherein the operations further comprise generating a plurality of similarity values corresponding to the administration of the reference drug at different doses.
157. The system of any one of embodiments 153-155, wherein the generation of the similarity value comprises generating an upregulation enrichment score and a downregulation enrichment score for the reference drug using the treatment signature and reference signature.
158. The system of any one of embodiments 153-156, wherein the generation of the similarity value comprises generating a combined enrichment score for the reference drug using the treatment signature and the reference signature.
159. The system of any one of embodiments 149-157, wherein the operations further comprise deriving a recovery value using a function of the treatment signature and a target signature concerning the animal subject prior to administration of the drug, the target signature comprising the features.
160. The system of any one of embodiments 117-158, wherein the operations further comprise deriving a treatment Markov model concerning the animal subject using a machine-learning Markov component, the treatment Markov model comprising a plurality of Markov states representing a selection of the features, each Markov state being associated with one or more Markov states by one or more transition probabilities.
161. The system of embodiment 159, wherein the selection of the higher-order features comprise a selection of state features, and the plurality of Markov states represent the selection of state features, and the operations further comprise deriving at least one motif feature representing a sequence of transitions of one or more of the selected state features.
162. The system of embodiment 159 or 160, wherein the treatment Markov model is a hidden Markov model comprising at least one hidden state.
163. The system of any one of embodiments 159-161, wherein the operations further comprise generating a visual representation of the treatment Markov model concerning the animal subject; and displaying the visual representation on a display.
164. The system of any one of embodiments 159-162, wherein the operations further comprise obtaining, using the machine-learning Markov component, a control Markov model concerning a control animal to which a vehicle is administered, the control Markov model comprising the plurality of Markov states representing the selection of the features.
165. The system of any one of embodiments 159-163, wherein the operations further comprise generating transition probability differences between the transition probabilities of the treatment Markov model and the transition probabilities of the control Markov model; and generating a visual representation of the transition probability differences associated with the plurality of Markov states.
166. The system of any one of embodiments 124-164, wherein the higher-order features comprise at least one of head twitch, nose scratch, ear scratch, head shake, body elongation, or elongation-contraction, and the operations further comprise predicting the class label to be associated with psychedelics.
167. The system of any one of embodiments 117-165, wherein the operations further comprise predicting the class label to be associated with one or more subclasses of psychedelics, entheogens, or psychoplastogens.
168. The system of any one of embodiments 117-166, wherein the animal subject is a rodent.
169. The system of any one of embodiments 117-167, wherein the animal subject is a mouse or a rat.
170. The system of any one of embodiments 117-168, wherein the drug is administered before the data is acquired or during acquisition of the data.
171. The system of any one of embodiments 117-169, wherein the operations further comprise obtaining the observational data concerning the animal subject while the animal subject is not in active locomotion.
172. The system of any one of embodiments 117-170, wherein the at least one sensing device comprises a headset comprising at least one of an accelerometer, gyroscope, or magnetometer, the headset configured to detect at least one type of motion of the head of the animal subject.
173. The system of any of embodiments 119-171, wherein the operations further comprise training the machine-learning classifier component to predict the class label of the psychedelic drug representing a treatment effect at the predetermined dose, the predetermined dose being a non-dissociative drug dose.
174. The system of any one of embodiments 119-172, wherein the operations further comprise training the machine-learning classifier component to predict the class label of the psychedelic drug representing a non-specific treatment effect at the predetermined dose, the predetermined dose being a dissociative drug dose.
175. A system for drug screening, comprising:
   at least one processor; and
   a non-transitory storage medium storing instructions that, when executed by the at least one processor, cause the system to perform operations comprising:
      obtaining observational data concerning an animal subject, the observational data acquired using an enclosure for the animal subject, the enclosure instrumented with at least one sensing device;
      extracting instant behavioral features from the observational data;
      creating a treatment signature, the treatment signature including higher-order features derived from the instant behavioral features using a first machine-learning component, the higher-order features including at least one of a state feature, a motif feature, or a domain feature;
      generating a target signature difference between the treatment signature and a baseline signature;
      identifying at least one reference drug or condition based on the target signature difference, identification comprising:
         generating an upregulation enrichment score and a downregulation enrichment score for the at least one reference drug or condition using the target signature difference and a reference signature difference corresponding to the one of the at least one reference drug or condition;
         generating a combined enrichment score for the at least one reference drug or condition using the target signature difference and a reference signature difference corresponding to the one of the at least one reference drug or condition; or
         generating a similarity value for the at least one reference drug or condition using the target signature difference and a reference signature difference corresponding to the one of the at least one reference drug or condition; and
      providing an indication of the similarity value, combined enrichment score, or upregulation and downregulation enrichment scores for the at least one reference drug or condition.
176. The system of embodiment 174, wherein identifying the at least one reference drug or condition based on the target signature difference comprises generating the upregulation enrichment score and the downregulation enrichment score for the at least one reference drug or condition.
177. The system of embodiment 174 or 175, wherein the upregulation enrichment score and the downregulation enrichment score comprise gene set enrichment analysis scores.
178. The system of any one of embodiments 174-176, wherein identifying the at least one reference drug or condition based on the target signature difference comprises generating the combined enrichment score for the at least one reference drug or condition.
179. The system of any one of embodiments 174-177, wherein generating the combined enrichment score for the at least one reference drug or condition comprises:
   generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
   generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.
180. The system of any one of embodiments 174-178, wherein identifying the at least one reference drug or condition based on the target signature difference comprises generating the similarity value for the at least one reference drug or condition.
181. The system of any one of embodiments 174-179, wherein the animal subject is an animal raised or modified to serve as a model of a human disease.
182. The system of embodiment 180, wherein the human disease is Rett syndrome, Parkinson's disease, Alzheimer's disease, Huntington disease, Tuberous Sclerosis Complex, or Autism Spectrum Disorder.
183. The system of any one of embodiments 174-181, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.
184. The system of any one of embodiments 174-182, wherein the operations further comprise weighting one or more of behavioral feature difference values of the reference signature difference or the target signature difference prior to identifying the at least one reference drug or condition.
185. The system of embodiment 183, wherein the weights are generated using decorrelated ranked feature analysis.
186. A system for drug screening, comprising:
   at least one processor; and
   a non-transitory storage medium storing instructions that, when executed by the at least one processor, cause the system to perform operations comprising:
      in a training phase:
      obtaining, for each first animal subject in three of more sets of first animal subjects, each of the first sets corresponding to a combination of values of two or more characteristics of the first animals, a first value for each behavioral feature in a set of features, the features including:
         instant behavioral features extracted from observational data acquired for the first animal subjects using an enclosure instrumented with at least one sensing device; and
         higher-order features derived from the instant behavioral features using a first machine-learning component;
      determining, using a second machine-learning component and the first values, a mapping between at least two dimensions and a corresponding function of the features, the at least two dimensions including a treatment dimension and a secondary dimension; and
      in a screening phase:
      obtaining a second value for each behavioral feature in the set of the features for a second animal subject to which a compound is administered;
      determining, using the mapping and the second values, a treatment effect of the compound; and
      providing an indication of the treatment effect.
187. The system of embodiment 185, wherein the corresponding function of the features represents a weighted combination of the features, and the operations further comprise determining weights of the function of the features based on a discrimination power of each behavioral feature derived using a third machine-learning component.
188. The system of embodiment 186, wherein the third machine-learning component is a support vector machine-learning component trained to determine the weights based on features of a test animal group and a control animal group.
189. The system of any of embodiments 185-188, wherein the secondary dimension comprises a dimension orthogonal to the treatment dimension.
190. The system of embodiment 188, wherein the operations further comprise determining, using the mapping and the second values, a secondary effect along the a secondary dimension.
191. The system of embodiment 189, wherein the secondary effect comprises a dissociative effect of the compound.
192. The system of embodiment 189 or 190, wherein secondary effect comprises a side effect of the compound.
193. The system of any one of embodiments 189-191, wherein the secondary effect comprises a physiological condition.
194. The system of embodiment 192, wherein the physiological condition is aging.
195. The system of embodiment 192, wherein the physiological condition is a neurological disease, disorder, or dysfunction.
196. A system of classifying a drug comprising:
   at least one processor; and
   a non-transitory storage medium storing instructions that, when executed by the at least one processor, cause the system to perform operations comprising:
      obtaining EEG data from a plurality of electrodes positioned on an animal subject to which the drug is administered at a first dose;
      obtaining acceleration data from one or more accelerometers positioned on the animal subject to which the drug is administered;
      predicting a class label for the drug by applying the EEG data and the acceleration data to a machine-learning classifier component trained to predict the class label using the EEG data and the acceleration data; and
      providing an indication of the class label.
197. The system of embodiment 195, wherein the operations further comprise obtaining observational data concerning the animal subject, the observation data acquired using an enclosure for the animal subject, the enclosure instrumented with at least one sensing device.
198. The system of embodiment 196, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.
199. The system of embodiment 196 or 197, wherein the operations further comprise extracting features by applying the observational data to a machine-learning feature-extraction component, the observational data comprising the EEG data and the acceleration data.
200. The system of embodiment 198, wherein the features comprise instant behavioral features.
201. The system of embodiment 199, wherein the features comprise higher-order features derived from the instant behavioral features using a machine-learning higher-order feature-extraction component.
202. The system of embodiment 200, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
203. The system of embodiment 201, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
204. The system of embodiment 202, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
205. The system of any one of embodiments 195-203, wherein the EEG data comprises wake EEG and sleep EEG, and the operations further comprise automatically separating the wake EEG from the sleep EEG based on the EEG data and the acceleration data.
206. The system of any one of embodiments 195-204, wherein the operations further comprise:
   obtaining reference EEG data from the plurality of electrodes positioned on the animal subject to which a reference drug is administered at a second dose; and
   obtaining reference acceleration data from the one or more accelerometers positioned on the animal subject to which the reference drug is administered at the second dose.
207. The system of embodiment 205, wherein the operations further comprise generating a similarity value for the reference drug using the EEG data, the acceleration data, the reference EEG data, and the reference acceleration data.
208. The system of any one of embodiments 195-206, wherein the machine-learning classifier component comprises a Recurrent Neural Network (RNN).
209. The system of any one of embodiments 195-207, wherein machine-learning classifier component is a layer or branch of a machine-learning model.
210. The system of embodiment 208, wherein the machine-learning model is one of an ensemble of machine-learning models.
211. The system of embodiment 209, wherein the ensemble of machine-learning models comprises an ensemble of neural network models.
212. The system of any one of embodiments 195-210, wherein the operations further comprise:
   obtaining low temporal resolution and low frequency resolution power spectra data from the EEG data; and
   predicting the class label for the drug further comprising applying the low temporal resolution and low frequency resolution power spectra data to a low-resolution machine-learning classifier component trained to predict the class label using the low temporal resolution and low frequency resolution power spectra data.
213. The system of any one of embodiments 195-211, wherein the operations further comprise:
obtaining high temporal resolution and high frequency resolution power spectra data from the EEG data; and
   predicting the class label for the drug further comprising applying the high temporal resolution and high frequency resolution power spectra data to a high-resolution machine-learning classifier component trained to predict the class label using the high temporal resolution and high frequency resolution power spectra data.
214. The system of any one of embodiments 195-212, wherein the operations further comprise:
obtaining covariance data of EEG data obtained from at least two of the plurality of electrodes; and
   predicting the class label for the drug further comprising applying the covariance data to a covariance machine-learning classifier component trained to predict the class label using the covariance data.
215. The system of any one of embodiments 195-210, wherein the operations further comprise at least one of:
   obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
   obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
   obtaining covariance data of EEG data obtained from at least two of the plurality of electrodes; and predicting the class label for the drug further comprising applying the covariance data to a covariance machine-learning classifier component trained to predict the class label using the covariance data.
216. The system of any one of embodiments 195-214, wherein the animal subject is a rodent.
217. A system of extracting gait features of a rodent comprising:
   at least one processor; and
   a non-transitory storage medium storing instructions that, when executed by the at least one processor, cause the system to perform operations comprising:
      obtaining video data concerning a rodent, to which a drug is administered, over a predetermined period, the video data acquired using an enclosure for the rodent, the enclosure instrumented with an illuminated track for the rodent and at least one imaging device positioned to image an underside of the illuminated track;
   annotating frames in the video data with labels using two machine-learning components, the labels including:
      a first one of the two machine-learning components configured to divide a frame in video data into segments corresponding to first object classes, the first object classes comprising a paw class; and
      a second one of the two machine-learning components configured to detect bounding boxes corresponding to second object classes, the second object classes including hind left, hind right, front left, and front right paws;
   generating segmented images using the annotating frames, the segmented images divided into segments corresponding to third object classes including hind left, hind right, front left, and front right paws; and extracting gait features of the rodent from the segmented images.
218. The system of embodiment 216, wherein the first object classes further comprise a background class and a body class.
219. The system of embodiment 216 or 217, wherein the second object class further comprise a background class, a first body class indicating the rodent moving from left to right or clockwise, and a second body class indicating the rodent moving from right to left or counterclockwise.
220. The system of any one of embodiments 216-218, wherein the first one of the two machine-learning components comprises a U-net convolutional neural network (CNN).
221. The system of any one of embodiments 216-219, wherein the second one of the two machine-learning components comprises a region-based CNN (R-CNN).
222. The system of any one of embodiments 216-220, wherein the operations further comprise automatically correcting the division of the frame into segments and/or identification of the third object classes using a plurality of heuristic rules based on positional relationship of the third object classes.
223. The system of any one of embodiments 216-221, wherein the operations further comprise extracting positional data of the body center and one or more paws over a sequence of frames in the video data.
224. The system of embodiment 222, wherein the operations further comprise extracting the gait features from the positional data over the sequence of frames in the video data.
225. The system of any one of embodiments 216-223, wherein the operations further comprise extracting the gait features over a plurality of cycles; and deriving a gait pattern of the rodent from the gait features.
226. The system of any one of embodiments 216-224, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.
227. The system of any one of embodiments 216-224, wherein the operations further comprise extracting features of the rodent from the gait features using a machine-learning feature-extraction component.
228. The system of embodiment 226, wherein the features comprise at least one of forward walk, immobile, turn around, or backward walk.
229. The system of embodiment 226, wherein the features comprise instant behavioral features.
230. The system of embodiment 228, wherein the features comprise higher-order features derived from the instant behavioral features using a machine-learning higher-order feature-extraction component.
231. The system of embodiment 229, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
232. The system of embodiment 230, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
233. The system of embodiment 231, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
234. A non-transitory computer-readable medium containing instructions that, when executed by at least one processor of a system, cause the system to perform operations for classifying a drug, the operations comprising:
   obtaining observational data concerning an animal subject to which the drug is administered, the observational data acquired using an enclosure for the animal subject, the enclosure instrumented with at least one sensing device;
   extracting features by applying the observational data to a machine-learning feature-extraction component;
   predicting a class label of the drug by applying the features to a machine-learning classifier component, the machine-learning classifier component trained to predict the class label of the drug from, at least in part, the features; and
   providing an indication of the class label.
235. A non-transitory computer-readable medium containing instructions that, when executed by at least one processor of a system, cause the system to perform operations for classifying a drug, the operations comprising:
   obtaining observational data concerning an animal subject to which the drug is administered, the observational data comprising at least one of thermal data or respirational data, the observation data acquired using an enclosure for the animal subject, the enclosure instrumented with at least one sensing device;
   extracting features by applying the observational data to a machine-learning feature-extraction component;
   predicting a class label of the drug by applying the features to a machine-learning classifier component trained to predict the class label of the drug from, at least in part, the features; and
   providing an indication of the class label.
236. A non-transitory computer-readable medium containing instructions that, when executed by at least one processor of a system, cause the system to perform operations for classifying a psychedelic drug comprising:
   obtaining observational data concerning an animal subject to which a predetermined dose of the psychedelic drug is administered, the observation data acquired using an enclosure for the animal subject, the enclosure instrumented with at least one sensing device;
   extracting features by applying the observational data to a machine-learning feature-extraction component, the features comprising at least one of head twitch, nose scratch, ear scratch, head shake, body elongation, or elongation-contraction;
   predicting a class label of the psychedelic drug at the predetermined dose by applying the features to a machine-learning classifier component trained to predict the class label of the psychedelic drug from, at least in part, the features; and
   providing an indication of the class label.
237. The non-transitory computer-readable medium of any one of embodiments 233-235, wherein a machine-learning component is a layer or branch of a machine-learning model.
238. The non-transitory computer-readable medium of embodiment 236, wherein the machine-learning model is one of an ensemble of machine-learning models.
239. The non-transitory computer-readable medium of any one of embodiments 233-237, wherein the features comprise instant behavioral features corresponding to sets or sequences of data points indexed in time order of a first predetermined time scale.
240. The non-transitory computer-readable medium of any one of embodiments 238, wherein the operations further comprise extracting the instant behavioral features using hard-coded definitions contained within the machine-learning feature-extraction component.
241. The non-transitory computer-readable medium of any one of embodiments 233-239, wherein the operations further comprise:
   deriving higher-order features based on the instant behavioral features using a machine-learning higher-order-extraction component; and
   predicting the class label of the drug by applying the higher-order features to the machine-learning classifier component, the machine-learning classifier component trained to predict the class label of the drug from, at least in part, the higher-order features.
242. The non-transitory computer-readable medium of embodiment 240, wherein the higher-order features correspond to sets or sequences of instant behavioral features indexed in time order of a second predetermined time scale, the second predetermined time scale being greater than the first predetermined time scale.
243. The non-transitory computer-readable medium of any one of embodiments 233-241, wherein the operations further comprise obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.
244. The non-transitory computer-readable medium of any one of embodiments 233-242, wherein the at least one sensing device comprises at least one imaging sensor configured to obtain image data.
245. The non-transitory computer-readable medium of any one of embodiments 233-243, wherein the at least one imaging sensor comprises a thermal imaging sensor configured to obtain thermal image data.
246. The non-transitory computer-readable medium of embodiment 243 or 244, wherein the at least one imaging sensor comprises a camera having a frame rate of at least 30 frames-per-second (fps).
247. The non-transitory computer-readable medium of any one of embodiments 243-245, wherein the at least one imaging sensor comprises a high-speed camera having a frame rate of at least 70 fps.
248. The non-transitory computer-readable medium of any one of embodiments 243-245, wherein the at least one imaging sensor comprises a high-speed camera having a frame rate that is equal or superior to:
   a predetermined sampling rate for a behavior or action of the animal subject, or
   the maximum of the predetermined sampling rates for a collection of behaviors or actions extracted from a single data source.
249. The non-transitory computer-readable medium of any one of embodiments 243-247, wherein the at least one imaging sensor comprises an event imaging sensor configured to obtain dynamic image data.
250. The non-transitory computer-readable medium of embodiment 248, wherein the event imaging sensor is configured to have a dynamic range of at least 100 dB or an equivalent frame rate of at least 500,000 fps.
251. The non-transitory computer-readable medium of any one of embodiments 243-249, wherein the operations further comprise using the at least one imaging sensor with at least one mirror to obtain 3D image data.
252. The non-transitory computer-readable medium of any one of embodiments 243-250, wherein the at least one imaging sensor comprises a plurality of imaging sensors configured to obtain 3D image data.
253. The non-transitory computer-readable medium of any one of embodiments 243-251, wherein the observational data comprises a video of the animal subject obtained using the at least one imaging sensor, and the operations further comprise segmenting image frames of the video using a machine-learning segmentation model.
254. The non-transitory computer-readable medium of embodiment 252, wherein the operations further comprise:
   segmenting image frames of the video using a machine-learning segmentation model; and
   extracting the features by tracking at least one segmented object in the image frames using a trained deep learning component.
255. The non-transitory computer-readable medium of any one of embodiments 233-253, wherein the observational data comprises external data, the external data comprising data concerning one or more environmental designs of the enclosure, data concerning one or more stimuli given to the animal subject, or one or more rewards given to the animal subject.
256. The non-transitory computer-readable medium of any one of embodiments 233-254, wherein the observational data comprises physiological data of the animal subject.
257. The non-transitory computer-readable medium of any one of embodiments 255, wherein the at least one sensing device comprises a thermal sensor and the physiological data comprises temperature data obtained using the thermal sensor.
258. The non-transitory computer-readable medium of embodiment 256, wherein the temperature data comprises temperature measurements of at least one body part of the animal subject, the at least one body part comprising at least one or more eyes, paws, tail, or limbs.
259. The non-transitory computer-readable medium of any one of embodiments 255-257, wherein the at least one sensing device comprises at least one electroencephalogram (EEG) electrode and the physiological data comprises EEG data obtained using the least one EEG electrode.
260. The non-transitory computer-readable medium of any one of embodiments 234-258, wherein the respirational data comprises a respiration rate during a period when the animal subject is not in active locomotion.
261. The non-transitory computer-readable medium of any one of embodiments 243-259, wherein the operations further comprise deriving the respirational data, using a machine-learning respiration component, from image data obtained from at least one imaging sensor.
262. The non-transitory computer-readable medium of any one of embodiments 233-260, wherein the machine-learning feature-extraction component comprises a supervised machine-learning component, an unsupervised machine-learning component, or both.
263. The non-transitory computer-readable medium of any one of embodiments 240-261, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
264. The non-transitory computer-readable medium of any one of embodiments 240-262, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
265. The non-transitory computer-readable medium of any one of embodiments 240-263, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
266. The non-transitory computer-readable medium of any one of embodiments 233-264, wherein the operations further comprise:
   creating a treatment signature from the features;
   generating a signature difference between the treatment signature and a baseline signature concerning a control animal, the baseline signature comprising the features; and
   identifying a reference drug based on the signature difference and the treatment signature; and
   providing the indication of the class label based on the identified reference drug.
267. The non-transitory computer-readable medium of embodiment 265, wherein the operations further comprise ranking the features of the treatment signature based on the signature difference using a support vector machine-learning component.
268. The non-transitory computer-readable medium of embodiment 265 or 266, wherein the operations further comprise weighting one or more of feature difference values between the treatment signature and the baseline signature prior to identifying the reference drug.
269. The non-transitory computer-readable medium of embodiment 267, wherein the weights are generated using decorrelated ranked feature analysis.
270. The non-transitory computer-readable medium of any one of embodiments 265-268, wherein the identification of the reference drug comprises generating a similarity value for the reference drug using the treatment signature and a reference signature corresponding to the reference drug, the reference signature comprising the features.
271. The non-transitory computer-readable medium of any one of embodiments 265-269, wherein the drug is administered to the animal subject at a first dose and the reference drug is administered at a second dose.
272. The non-transitory computer-readable medium of any one of embodiments 265-269, wherein the operations further comprise generating a plurality of similarity values corresponding to the administration of the reference drug at different doses.
273. The non-transitory computer-readable medium of any one of embodiments 269-271, wherein the generation of the similarity value comprises generating an upregulation enrichment score and a downregulation enrichment score for the reference drug using the treatment signature and reference signature.
274. The non-transitory computer-readable medium of any one of embodiments 269-272, wherein the generation of the similarity value comprises generating a combined enrichment score for the reference drug using the treatment signature and the reference signature.
275. The non-transitory computer-readable medium of any one of embodiments 265-273, wherein the operations further comprise deriving a recovery value using a function of the treatment signature and a target signature concerning the animal subject prior to administration of the drug, the target signature comprising the features.
276. The non-transitory computer-readable medium of any one of embodiments 233-274, wherein the operations further comprise deriving a treatment Markov model concerning the animal subject using a machine-learning Markov component, the treatment Markov model comprising a plurality of Markov states representing a selection of the features, each Markov state being associated with one or more Markov states by one or more transition probabilities.
277. The non-transitory computer-readable medium of embodiment 275, wherein the selection of the higher-order features comprise a selection of state features, and the plurality of Markov states represent the selection of state features, and the operations further comprise deriving at least one motif feature representing a sequence of transitions of one or more of the selected state features.
278. The non-transitory computer-readable medium of embodiment 275 or 276, wherein the treatment Markov model is a hidden Markov model comprising at least one hidden state.
279. The non-transitory computer-readable medium of any one of embodiments 275-277, wherein the operations further comprise generating a visual representation of the treatment Markov model concerning the animal subject; and displaying the visual representation on a display.
280. The non-transitory computer-readable medium of any one of embodiments 275-278, wherein the operations further comprise obtaining, using the machine-learning Markov component, a control Markov model concerning a control animal to which a vehicle is administered, the control Markov model comprising the plurality of Markov states representing the selection of the features.
281. The non-transitory computer-readable medium of any one of embodiments 275-279, wherein the operations further comprise generating transition probability differences between the transition probabilities of the treatment Markov model and the transition probabilities of the control Markov model; and generating a visual representation of the transition probability differences associated with the plurality of Markov states.
282. The non-transitory computer-readable medium of any one of embodiments 240-280, wherein the higher-order features comprise at least one of head twitch, nose scratch, ear scratch, head shake, body elongation, or elongation-contraction, and the operations further comprise predicting the class label to be associated with psychedelics.
283. The non-transitory computer-readable medium of any one of embodiments 233-281, wherein the operations further comprise predicting the class label to be associated with one or more subclasses of psychedelics, entheogens, or psychoplastogens.
284. The non-transitory computer-readable medium of any one of embodiments 233-282, wherein the animal subject is a rodent.
285. The non-transitory computer-readable medium of any one of embodiments 233-283, wherein the animal subject is a mouse or a rat.
286. The non-transitory computer-readable medium of any one of embodiments 233-284, wherein the drug is administered before the data is acquired or during acquisition of the data.
287. The non-transitory computer-readable medium of any one of embodiments 233-285, wherein the operations further comprise obtaining the observational data concerning the animal subject while the animal subject is not in active locomotion.
288. The non-transitory computer-readable medium of any one of embodiments 233-286, wherein the at least one sensing device comprises a headset comprising at least one of an accelerometer, gyroscope, or magnetometer, the headset configured to detect at least one type of motion of the head of the animal subject.
289. The non-transitory computer-readable medium of any of embodiments 235-287, wherein the operations further comprise training the machine-learning classifier component to predict the class label of the psychedelic drug representing a treatment effect at the predetermined dose, the predetermined dose being a non-dissociative drug dose.
290. The non-transitory computer-readable medium of any one of embodiments 235-288, wherein the operations further comprise training the machine-learning classifier component to predict the class label of the psychedelic drug representing a non-specific treatment effect at the predetermined dose, the predetermined dose being a dissociative drug dose.
291. A non-transitory computer-readable medium containing instructions that, when executed by at least one processor of a system, cause the system to perform operations for drug screening, the operations comprising:
   obtaining observational data concerning an animal subject, the observational data acquired using an enclosure for the animal subject, the enclosure instrumented with at least one sensing device;
   extracting instant behavioral features from the observational data;
   creating a treatment signature, the treatment signature including higher-order features derived from the instant behavioral features using a first machine-learning component, the higher-order features including at least one of a state feature, a motif feature, or a domain feature;
   generating a target signature difference between the treatment signature and a baseline signature;
   identifying at least one reference drug or condition based on the target signature difference, identification comprising:
      generating an upregulation enrichment score and a downregulation enrichment score for the at least one reference drug or condition using the target signature difference and a reference signature difference corresponding to the one of the at least one reference drug or condition;
      generating a combined enrichment score for the at least one reference drug or condition using the target signature difference and a reference signature difference corresponding to the one of the at least one reference drug or condition; or
      generating a similarity value for the at least one reference drug or condition using the target signature difference and a reference signature difference corresponding to the one of the at least one reference drug or condition; and
   providing an indication of the similarity value, combined enrichment score, or upregulation and downregulation enrichment scores for the at least one reference drug or condition.
292. The computer-implemented drug screening method of embodiment 290, wherein identifying the at least one reference drug or condition based on the target signature difference comprises generating the upregulation enrichment score and the downregulation enrichment score for the at least one reference drug or condition.
293. The computer-implemented drug screening method of embodiment 290 or 291, wherein the upregulation enrichment score and the downregulation enrichment score comprise gene set enrichment analysis scores.
294. The computer-implemented drug screening method of any one of embodiments 290-292, wherein identifying the at least one reference drug or condition based on the target signature difference comprises generating the combined enrichment score for the at least one reference drug or condition.
295. The computer-implemented drug screening method of any one of embodiments 290-293, wherein generating the combined enrichment score for the at least one reference drug or condition comprises:
   generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
   generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.
296. The computer-implemented drug screening method of any one of embodiments 290-294, wherein identifying the at least one reference drug or condition based on the target signature difference comprises generating the similarity value for the at least one reference drug or condition.
297. The computer-implemented drug screening method of any one of embodiments 290-295, wherein the animal subject is an animal raised or modified to serve as a model of a human disease.
298. The computer-implemented drug screening method of embodiment 296, wherein the human disease is Rett syndrome, Parkinson's disease, Alzheimer's disease, Huntington disease, Tuberous Sclerosis Complex, or Autism Spectrum Disorder.
299. The computer-implemented drug screening method of any one of embodiments 290-297, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.
300. The computer-implemented drug screening method of any one of embodiments 290-298, wherein the operations further comprise weighting one or more of behavioral feature difference values of the reference signature difference or the target signature difference prior to identifying the at least one reference drug or condition.
301. The computer-implemented drug screening method of embodiment 299, wherein the weights are generated using decorrelated ranked feature analysis.
302. A non-transitory computer-readable medium containing instructions that, when executed by at least one processor of a system, cause the system to perform operations for drug screening, the operations comprising:
   in a training phase:
   obtaining, for each first animal subject in three or more sets of first animal subjects, each of the first sets corresponding to a combination of values of two or more characteristics of the first animals, a first value for each behavioral feature in a set of features, the features including:
      instant behavioral features extracted from observational data acquired for the first animal subjects using an enclosure instrumented with at least one sensing device; and
      higher-order features derived from the instant behavioral features using a first machine-learning component;
   determining, using a second machine-learning component and the first values, a mapping between at least two dimensions and corresponding functions of the features, the at least two dimensions including a treatment dimension and a secondary dimension; and
   in a screening phase:
   obtaining a second value for each behavioral feature in the set of the features for a second animal subject to which a compound is administered;
   determining, using the mapping and the second values, a treatment effect of the compound; and
   providing an indication of the treatment effect.
303. The non-transitory computer-readable medium of embodiment 301, wherein the corresponding function of the features represents a weighted combination of the features, and the operations further comprise determining weights of the function of the features based on a discrimination power of each behavioral feature derived using a third machine-learning component.
304. The non-transitory computer-readable medium of embodiment 302, wherein the third machine-learning component is a support vector machine-learning component trained to determine the weights based on features of a test animal group and a control animal group.
305. The non-transitory computer-readable medium of any of embodiments 301-304, wherein the secondary dimension comprises a dimension orthogonal to the treatment dimension.
306. The non-transitory computer-readable medium of embodiment 304, wherein the operations further comprise determining, using the mapping and the second values, a secondary effect along secondary dimension.
307. The non-transitory computer-readable medium of embodiment 305, wherein the secondary effect comprises a dissociative effect of the compound.
308. The non-transitory computer-readable medium of embodiment 305 or 306, wherein the secondary effect comprises a side effect of the compound.
309. The non-transitory computer-readable medium of any one of embodiments 305-307, wherein the secondary effect comprises a physiological condition.
310. The non-transitory computer-readable medium of embodiment 308, wherein the physiological condition is aging.
311. The non-transitory computer-readable medium of embodiment 308, wherein the physiological condition is a neurological disease, disorder, or dysfunction.
312. A non-transitory computer-readable medium containing instructions that, when executed by at least one processor of a system, cause the system to perform operations for classifying a drug, the operations comprising:
   obtaining EEG data from a plurality of electrodes positioned on an animal subject to which the drug is administered at a first dose;
   obtaining acceleration data from one or more accelerometers positioned on the animal subject to which the drug is administered;
   predicting a class label for the drug by applying the EEG data and the acceleration data to a machine-learning classifier component trained to predict the class label using the EEG data and the acceleration data; and
   providing an indication of the class label.
313. The non-transitory computer-readable medium of embodiment 311, wherein the operations further comprise obtaining observational data concerning the animal subject, the observation data acquired using an enclosure for the animal subject, the enclosure instrumented with at least one sensing device.
314. The non-transitory computer-readable medium of embodiment 312, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.
315. The non-transitory computer-readable medium of embodiment 312 or 313, wherein the operations further comprise extracting features by applying the observational data to a machine-learning feature-extraction component, the observational data comprising the EEG data and the acceleration data.
316. The non-transitory computer-readable medium of embodiment 314, wherein the features comprise instant behavioral features.
317. The non-transitory computer-readable medium of embodiment 315, wherein the features comprise higher-order features derived from the instant behavioral features using a machine-learning higher-order feature-extraction component.
318. The non-transitory computer-readable medium of embodiment 316, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
319. The non-transitory computer-readable medium of embodiment 317, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
320. The non-transitory computer-readable medium of embodiment 318, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
321. The non-transitory computer-readable medium of any one of embodiments 311-319, wherein the EEG data comprises wake EEG and sleep EEG, and the operations further comprise automatically separating the wake EEG from the sleep EEG based on the EEG data and the acceleration data.
322. The non-transitory computer-readable medium of any one of embodiments 311-320, wherein the operations further comprise:
   obtaining reference EEG data from the plurality of electrodes positioned on the animal subject to which a reference drug is administered at a second dose; and
   obtaining reference acceleration data from the one or more accelerometers positioned on the animal subject to which the reference drug is administered at the second dose.
323. The non-transitory computer-readable medium of embodiment 321, wherein the operations further comprise generating a similarity value for the reference drug using the EEG data, the acceleration data, the reference EEG data, and the reference acceleration data.
324. The non-transitory computer-readable medium of any one of embodiments 311-322, wherein the machine-learning classifier component comprises a Recurrent Neural Network (RNN).
325. The non-transitory computer-readable medium of any one of embodiments 311-323, wherein machine-learning classifier component is a layer or branch of a machine-learning model.
326. The non-transitory computer-readable medium of embodiment 324, wherein the machine-learning model is one of an ensemble of machine-learning models.
327. The non-transitory computer-readable medium of embodiment 325, wherein the ensemble of machine-learning models comprises an ensemble of neural network models.
328. The non-transitory computer-readable medium of any one of embodiments 311-326, wherein the operations further comprise:
   obtaining low temporal resolution and low frequency resolution power spectra data from the EEG data; and
   predicting the class label for the drug further comprising applying the low temporal resolution and low frequency resolution power spectra data to a low-resolution machine-learning classifier component trained to predict the class label using the low temporal resolution and low frequency resolution power spectra data.
329. The non-transitory computer-readable medium of any one of embodiments 311-327, wherein the operations further comprise:
   obtaining high temporal resolution and high frequency resolution power spectra data from the EEG data; and
   predicting the class label for the drug further comprising applying the high temporal resolution and high frequency resolution power spectra data to a high-resolution machine-learning classifier component trained to predict the class label using the high temporal resolution and high frequency resolution power spectra data.
330. The non-transitory computer-readable medium of any one of embodiments 311-328, wherein the operations further comprise:
   obtaining covariance data of EEG data obtained from at least two of the plurality of electrodes; and predicting the class label for the drug further comprising applying the covariance data to a covariance machine-learning classifier component trained to predict the class label using the covariance data.
331. The non-transitory computer-readable medium of any one of embodiments 311-326, wherein the operations further comprise at least one of:
   obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
   obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
   obtaining covariance data of EEG data obtained from at least two of the plurality of electrodes; and predicting the class label for the drug further comprising applying the covariance data to a covariance machine-learning classifier component trained to predict the class label using the covariance data.
332. The non-transitory computer-readable medium of any one of embodiments 311-330, wherein the animal subject is a rodent.
333. A non-transitory computer-readable medium containing instructions that, when executed by at least one processor of a system, cause the system to perform operations for extracting gait features of a rodent, the operations comprising:
   obtaining video data concerning a rodent, to which a drug is administered, over a predetermined period, the video data acquired using an enclosure for the rodent, the enclosure instrumented with an illuminated track for the rodent and at least one imaging device positioned to image an underside of the illuminated track;
   annotating frames in the video data with labels using two machine-learning components, the labels including:
      a first one of the two machine-learning components configured to divide a frame in video data into segments corresponding to first object classes, the first object classes comprising a paw class; and
      a second one of the two machine-learning components configured to detect bounding boxes corresponding to second object classes, the second object classes including hind left, hind right, front left, and front right paws;
   generating segmented images using the annotating frames, the segmented images divided into segments corresponding to third object classes including hind left, hind right, front left, and front right paws; and
   extracting gait features of the rodent from the segmented images.
334. The non-transitory computer-readable medium of embodiment 332, wherein the first object classes further comprise a background class and a body class.
335. The non-transitory computer-readable medium of embodiment 332 or 333, wherein the second object class further comprise a background class, a first body class indicating the rodent moving from left to right or clockwise, and a second body class indicating the rodent moving from right to left or counterclockwise.
336. The non-transitory computer-readable medium of any one of embodiments 332-334, wherein the first one of the two machine-learning components comprises a U-net convolutional neural network (CNN).
337. The non-transitory computer-readable medium of any one of embodiments 332-335, wherein the second one of the two machine-learning components comprises a region-based CNN (R-CNN).
338. The non-transitory computer-readable medium of any one of embodiments 332-336, wherein the operations further comprise automatically correcting the division of the frame into segments and/or identification of the third object classes using a plurality of heuristic rules based on positional relationship of the third object classes.
339. The non-transitory computer-readable medium of any one of embodiments 332-337, wherein the operations further comprise extracting positional data of the body center and one or more paws over a sequence of frames in the video data.
340. The non-transitory computer-readable medium of embodiment 338, wherein the operations further comprise extracting the gait features from the positional data over the sequence of frames in the video data.
341. The non-transitory computer-readable medium of any one of embodiments 332-339, wherein the operations further comprise extracting the gait features over a plurality of cycles; and deriving a gait pattern of the rodent from the gait features.
342. The non-transitory computer-readable medium of any one of embodiments 332-340, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.
343. The non-transitory computer-readable medium of any one of embodiments 332-340, wherein the operations further comprise extracting features of the rodent from the gait features using a machine-learning feature-extraction component.
344. The non-transitory computer-readable medium of embodiment 342, wherein the features comprise at least one of forward walk, immobile, turn around, or backward walk.
345. The non-transitory computer-readable medium of embodiment 342, wherein the features comprise instant behavioral features.
346. The non-transitory computer-readable medium of embodiment 344, wherein the features comprise higher-order features derived from the instant behavioral features using a machine-learning higher-order feature-extraction component.
347. The non-transitory computer-readable medium of embodiment 345, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.
348. The non-transitory computer-readable medium of embodiment 346, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.
349. The non-transitory computer-readable medium of embodiment 347, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some embodiments” as used herein do not necessarily refer to a different embodiment, although it may. Thus, as described below, various embodiments may be readily combined, without departing from the scope or spirit of the present disclosure.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical, or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor such as central processing unit (CPU) and/or graphical processing unit (GPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In some embodiments, exemplary inventive computer-based systems/platforms, exemplary inventive computer-based devices, and/or exemplary inventive computer-based components of the present disclosure may be: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

All patents, patent applications, publications of patent applications, and other material, such as articles, books, specifications, publications, documents, things, and/or the like, referenced herein are hereby incorporated herein by this reference in their entirety for at least specific purposes identified herein, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

The aforementioned examples are, of course, illustrative and not restrictive. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Furthermore, the various steps may be carried out in any desired order (and any desired steps may be added and/or any desired steps may be eliminated).

What is claimed is:

1. A computer-implemented method of classifying a drug, comprising:
obtaining observational data concerning an animal subject to which the drug is administered, the observational data acquired using an enclosure for the animal subject, the enclosure instrumented with at least one sensing device;
extracting features by applying the observational data to a machine-learning feature-extraction component;
predicting a class label of the drug by applying the features to a machine-learning classifier component, the machine-learning classifier component trained to predict the class label of the drug from, at least in part, the features; and
providing an indication of the class label.

2. A computer-implemented method of classifying a drug, comprising:
obtaining observational data concerning an animal subject to which the drug is administered, the observational data comprising at least one of thermal data or respirational data, the observation data acquired using an enclosure for the animal subject, the enclosure instrumented with at least one sensing device;
extracting features by applying the observational data to a machine-learning feature-extraction component;
predicting a class label of the drug by applying the features to a machine-learning classifier component trained to predict the class label of the drug from, at least in part, the features; and
providing an indication of the class label.

3. A computer-implemented method of classifying a psychedelic drug, comprising:
obtaining observational data concerning an animal subject to which a predetermined dose of the psychedelic drug is administered, the observation data acquired using an enclosure for the animal subject, the enclosure instrumented with at least one sensing device;
extracting features by applying the observational data to a machine-learning feature-extraction component, the features comprising at least one of head twitch, nose scratch, ear scratch, head shake, body elongation, or elongation-contraction;
predicting a class label of the psychedelic drug at the predetermined dose by applying the features to a machine-learning classifier component trained to predict the class label of the psychedelic drug from, at least in part, the features; and
providing an indication of the class label.

4. The computer-implemented method of any one of claims 1-3, wherein a machine-learning component is a layer or branch of a machine-learning model.

5. The computer-implemented method of claim 4, wherein the machine-learning model is one of an ensemble of machine-learning models.

6. The computer-implemented method of any one of claims 1-5, wherein the features comprise instant behavioral features corresponding to sets or sequences of data points indexed in time order of a first predetermined time scale.

7. The computer-implemented method of any one of claim 6, further comprising extracting the instant behavioral features using hard-coded definitions contained within the machine-learning feature-extraction component.

8. The computer-implemented method of any one of claims 1-7, further comprising:
deriving higher-order features based on the instant behavioral features using a machine-learning higher-order-extraction component; and
predicting the class label of the drug by applying the higher-order features to the machine-learning classifier component, the machine-learning classifier component trained to predict the class label of the drug from, at least in part, the higher-order features.

9. The computer-implemented method of claim 8, wherein the higher-order features correspond to sets or sequences of instant behavioral features indexed in time order of a second predetermined time scale, the second predetermined time scale being greater than the first predetermined time scale.

10. The computer-implemented method of any one of claims 1-9, further comprising obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.

11. The computer-implemented method of any one of claims 1-10, wherein the at least one sensing device comprises at least one imaging sensor configured to obtain image data.

12. The computer-implemented method of any one of claims 1-11, wherein the at least one imaging sensor comprises a thermal imaging sensor configured to obtain thermal image data.

13. The computer-implemented method of claim 11 or 12, wherein the at least one imaging sensor comprises a camera having a frame rate of at least 30 frames-per-second (fps).

14. The computer-implemented method of any one of claims 11-13, wherein the at least one imaging sensor comprises a high-speed camera having a frame rate of at least 70 fps.

15. The computer-implemented method of any one of claims 11-13, wherein the at least one imaging sensor comprises a high-speed camera having a frame rate that is equal or superior to:
a predetermined sampling rate for a behavior or action of the animal subject, or
the maximum of the predetermined sampling rates for a collection of behaviors or actions extracted from a single data source.

16. The computer-implemented method of any one of claims 11-15, wherein the at least one imaging sensor comprises an event imaging sensor configured to obtain dynamic image data.

17. The computer-implemented method of claim 16, wherein the event imaging sensor is configured to have a dynamic range of at least 100 dB or an equivalent frame rate of at least 500,000 fps.

18. The computer-implemented method of any one of claims 11-17, further comprising using the at least one imaging sensor with at least one mirror to obtain 3D image data.

19. The computer-implemented method of any one of claims 11-18, wherein the at least one imaging sensor comprises a plurality of imaging sensors configured to obtain 3D image data.

20. The computer-implemented method of any one of claims 11-19, wherein the observational data comprises a video of the animal subject obtained using the at least one imaging sensor, and the method further comprises segmenting image frames of the video using a machine-learning segmentation model.

21. The computer-implemented method of claim 20, further comprising:
segmenting image frames of the video using a machine-learning segmentation model; and
extracting the features by tracking at least one segmented object in the image frames using a trained deep learning component.

22. The computer-implemented method of any one of claims 1-21, wherein the observational data comprises external data, the external data comprising data concerning one or more environmental designs of the enclosure, data concerning one or more stimuli given to the animal subject, or one or more rewards given to the animal subject.

23. The computer-implemented method of any one of claims 1-22, wherein the observational data comprises physiological data of the animal subject.

24. The computer-implemented method of any one of claim 23, wherein the at least one sensing device comprises a thermal sensor and the physiological data comprises temperature data obtained using the thermal sensor.

25. The computer-implemented method of claim 24, wherein the temperature data comprises temperature measurements of at least one body part of the animal subject, the at least one body part comprising at least one or more eyes, paws, tail, or limbs.

26. The computer-implemented method of any one of claims 23-25, wherein the at least one sensing device comprises at least one electroencephalogram (EEG) electrode and the physiological data comprises EEG data obtained using the least one EEG electrode.

27. The computer-implemented method of any one of claims 2-26, wherein the respirational data comprises a respiration rate during a period when the animal subject is not in active locomotion.

28. The computer-implemented method of any one of claims 11-27, further comprising deriving the respirational data, using a machine-learning respiration component, from image data obtained from at least one imaging sensor.

29. The computer-implemented method of any one of claims 1-28, wherein the machine-learning feature-extraction component comprises a supervised machine-learning component, an unsupervised machine-learning component, or both.

30. The computer-implemented method of any one of claims 8-29, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.

31. The computer-implemented method of any one of claims 8-30, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.

32. The computer-implemented method of any one of claim 8-31, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.

33. The computer-implemented method of any one of claims 1-32, further comprising:
creating a treatment signature from the features;
generating a signature difference between the treatment signature and a baseline signature concerning a control animal, the baseline signature comprising the features; and
identifying a reference drug based on the signature difference and the treatment signature; and
providing the indication of the class label based on the identified reference drug.

34. The computer-implemented method of claim 33, further comprising ranking the features of the treatment signature based on the signature difference using a support vector machine-learning component.

35. The computer-implemented method of claim 33 or 34, further comprising weighting one or more of feature difference values between the treatment signature and the baseline signature prior to identifying the reference drug.

36. The computer-implemented method of claim 35, wherein the weights are generated using decorrelated ranked feature analysis.

37. The computer-implemented method of any one of claims 33-36, wherein the identification of the reference drug comprises generating a similarity value for the reference drug using the treatment signature and a reference signature corresponding to the reference drug, the reference signature comprising the features.

38. The computer-implemented method of any one of claims 33-37, wherein the drug is administered to the animal subject at a first dose and the reference drug is administered at a second dose.

39. The computer-implemented method of any one of claims 33-37, further comprising generating a plurality of similarity values corresponding to the administration of the reference drug at different doses.

40. The computer-implemented method of any one of claims 37-39, wherein the generation of the similarity value comprises generating an upregulation enrichment score and a downregulation enrichment score for the reference drug using the treatment signature and reference signature.

41. The computer-implemented method of any one of claims 37-40, wherein the generation of the similarity value comprises generating a combined enrichment score for the reference drug using the treatment signature and the reference signature.

42. The computer-implemented method of any one of claims 33-41, further comprising deriving a recovery value using a function of the treatment signature and a target signature concerning the animal subject prior to administration of the drug, the target signature comprising the features.

43. The computer-implemented method of any one of claims 1-42, further comprising deriving a treatment Markov model concerning the animal subject using a machine-learning Markov component, the treatment Markov model comprising a plurality of Markov states representing a selection of the features, each Markov state being associated with one or more Markov states by one or more transition probabilities.

44. The computer-implemented method of claim 43, wherein the selection of the higher-order features comprise a selection of state features, and the plurality of Markov states represent the selection of state features, and the method further comprises deriving at least one motif feature representing a sequence of transitions of one or more of the selected state features.

45. The computer-implemented method of claim 43 or 44, wherein the treatment Markov model is a hidden Markov model comprising at least one hidden state.

46. The computer-implemented method of any one of claims 43-45, further comprising generating a visual representation of the treatment Markov model concerning the animal subject; and displaying the visual representation on a display.

47. The computer-implemented method of any one of claims 43-46, further comprising obtaining, using the machine-learning Markov component, a control Markov model concerning a control animal to which a vehicle is administered, the control Markov model comprising the plurality of Markov states representing the selection of the features.

48. The computer-implemented method of any one of claims 43-47, further comprising generating transition probability differences between the transition probabilities of the treatment Markov model and the transition probabilities of the control Markov model; and generating a visual representation of the transition probability differences associated with the plurality of Markov states.

49. The computer-implemented method of any one of claims 8-48, wherein the higher-order features comprise at least one of head twitch, nose scratch, ear scratch, head shake, body elongation, or elongation-contraction, and the method further comprises predicting the class label to be associated with psychedelics.

50. The computer-implemented method of any one of claims 1-49, further comprising predicting the class label to be associated with one or more subclasses of psychedelics, entheogens, or psychoplastogens.

51. The computer-implemented method of any one of claims 1-50, wherein the animal subject is a rodent.

52. The computer-implemented method of any one of claims 1-51, wherein the animal subject is a mouse or a rat.

53. The computer-implemented method of any one of claims 1-52, wherein the drug is administered before the data is acquired or during acquisition of the data.

54. The computer-implemented method of any one of claims 1-53, further comprising obtaining the observational data concerning the animal subject while the animal subject is not in active locomotion.

55. The computer-implemented method of any one of claims 1-54, wherein the at least one sensing device comprises a headset comprising at least one of an accelerometer, gyroscope, or magnetometer, the headset configured to detect at least one type of motion of the head of the animal subject.

56. The computer-implemented method of any of claims 3-55, further comprising training the machine-learning classifier component to predict the class label of the psychedelic drug representing a treatment effect at the predetermined dose, the predetermined dose being a non-dissociative drug dose.

57. The computer-implemented method of any one of claims 3-56, further comprising training the machine-learning classifier component to predict the class label of the psychedelic drug representing a non-specific treatment effect at the predetermined dose, the predetermined dose being a dissociative drug dose.

58. A computer-implemented drug screening method, comprising:
obtaining observational data concerning an animal subject, the observational data acquired using an enclosure for the animal subject, the enclosure instrumented with at least one sensing device;
extracting instant behavioral features from the observational data;
creating a treatment signature, the treatment signature including higher-order features derived from the instant behavioral features using a first machine-learning component, the higher-order features including at least one of a state feature, a motif feature, or a domain feature;
generating a target signature difference between the treatment signature and a baseline signature;
identifying at least one reference drug or condition based on the target signature difference, identification comprising:
generating an upregulation enrichment score and a downregulation enrichment score for the at least one reference drug or condition using the target signature difference and a reference signature difference corresponding to the one of the at least one reference drug or condition;
generating a combined enrichment score for the at least one reference drug or condition using the target signature difference and a reference signature difference corresponding to the one of the at least one reference drug or condition; or
generating a similarity value for the at least one reference drug or condition using the target signature difference and a reference signature difference corresponding to the one of the at least one reference drug or condition; and
providing an indication of the similarity value, combined enrichment score, or upregulation and downregulation enrichment scores for the at least one reference drug or condition.

59. The computer-implemented drug screening method of claim 58, wherein identifying the at least one reference drug or condition based on the target signature difference comprises generating the upregulation enrichment score and the downregulation enrichment score for the at least one reference drug or condition.

60. The computer-implemented drug screening method of claim 58 or 59, wherein the upregulation enrichment score and the downregulation enrichment score comprise gene set enrichment analysis scores.

61. The computer-implemented drug screening method of any one of claims 58-60, wherein identifying the at least one reference drug or condition based on the target signature difference comprises generating the combined enrichment score for the at least one reference drug or condition.

62. The computer-implemented drug screening method of any one of claims 58-61, wherein generating the combined enrichment score for the at least one reference drug or condition comprises:
generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.

63. The computer-implemented drug screening method of any one of claims 58-62, wherein identifying the at least one reference drug or condition based on the target signature difference comprises generating the similarity value for the at least one reference drug or condition.

64. The computer-implemented drug screening method of any one of claims 58-63, wherein the animal subject is an animal raised or modified to serve as a model of a human disease.

65. The computer-implemented drug screening method of claim 64, wherein the human disease is Rett syndrome, Parkinson's disease, Alzheimer's disease, Huntington disease, Tuberous Sclerosis Complex, or Autism Spectrum Disorder.

66. The computer-implemented drug screening method of any one of claims 58-65, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.

67. The computer-implemented drug screening method of any one of claims 58-66, further comprising weighting one or more of behavioral feature difference values of the reference signature difference or the target signature difference prior to identifying the at least one reference drug or condition.

68. The computer-implemented drug screening method of claim 67, wherein the weights are generated using decorrelated ranked feature analysis.

69. A computer-implemented method drug screening method, comprising:
in a training phase:
obtaining, for each first animal subject in three or more first sets of first animal subjects, each of the first sets corresponding to a combination of values of two or more characteristics of the first animals, a first value for each behavioral feature in a set of features, the features including:
instant behavioral features extracted from observational data acquired for the first animal subjects using an enclosure instrumented with at least one sensing device; and
higher-order features derived from the instant behavioral features using a first machine-learning component;
determining, using a second machine-learning component and the first values, a mapping between at least two dimensions and corresponding functions of the features, the at least two dimensions including a treatment dimension and a secondary dimension; and
in a screening phase:
obtaining a second value for each behavioral feature in the set of the features for a second animal subject to which a compound is administered;
determining, using the mapping and the second values, a treatment effect of the compound; and
providing an indication of the treatment effect.

70. The computer-implemented method of claim 69, wherein the corresponding function of the features represents a weighted combination of the features, and the method further comprises determining weights of the function of the features based on a discrimination power of each behavioral feature derived using a third machine-learning component.

71. The computer-implemented method of claim 70, wherein the third machine-learning component is a support vector machine-learning component trained to determine the weights based on features of a test animal group and a control animal group.

72. The computer-implemented method of any of claims 69-72, wherein the secondary dimension comprises a dimension orthogonal to the treatment dimension.

73. The computer-implemented method of claim 72, further comprising determining, using the mapping and the second values, a secondary effect along the secondary dimension.

74. The computer-implemented method of claim 73, wherein the secondary effect comprises a dissociative effect of the compound.

75. The computer-implemented method of claim 73 or 74, wherein the secondary effect comprises a side effect of the compound.

76. The computer-implemented method of any one of claims 73-75, wherein the secondary effect comprises a physiological condition.

77. The computer-implemented method of claim 76, wherein the physiological condition is aging.

78. The computer-implemented method of claim 76, wherein the physiological condition is a neurological disease, disorder, or dysfunction.

79. A computer-implemented method of classifying a drug comprising:
obtaining EEG data from a plurality of electrodes positioned on an animal subject to which the drug is administered at a first dose;
obtaining acceleration data from one or more accelerometers positioned on the animal subject to which the drug is administered;
predicting a class label for the drug by applying the EEG data and the acceleration data to a machine-learning classifier component trained to predict the class label using the EEG data and the acceleration data; and
providing an indication of the class label.

80. The computer-implemented method of claim 79, further comprising obtaining observational data concerning the animal subject, the observation data acquired using an enclosure for the animal subject, the enclosure instrumented with at least one sensing device.

81. The computer-implemented method of claim 80, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.

82. The computer-implemented method of claim 80 or 81, further comprising extracting features by applying the observational data to a machine-learning feature-extraction component, the observational data comprising the EEG data and the acceleration data.

83. The computer-implemented method of claim 82, wherein the features comprise instant behavioral features.

84. The computer-implemented method of claim 83, wherein the features comprise higher-order features derived from the instant behavioral features using a machine-learning higher-order feature-extraction component.

85. The computer-implemented method of claim 84, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.

86. The computer-implemented method of claim 85, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.

87. The computer-implemented method of claim 86, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.

88. The computer-implemented method of any one of claims 79-87, wherein the EEG data comprises wake EEG and sleep EEG, and the method further comprises automatically separating the wake EEG from the sleep EEG based on the EEG data and the acceleration data.

89. The computer-implemented method of any one of claims 79-88, further comprising:
obtaining reference EEG data from the plurality of electrodes positioned on the animal subject to which a reference drug is administered at a second dose; and
obtaining reference acceleration data from the one or more accelerometers positioned on the animal subject to which the reference drug is administered at the second dose.

90. The computer-implemented method of claim 89, further comprising generating a similarity value for the reference drug using the EEG data, the acceleration data, the reference EEG data, and the reference acceleration data.

91. The computer-implemented method of any one of claims 79-90, wherein the machine-learning classifier component comprises a Recurrent Neural Network (RNN).

92. The computer-implemented method of any one of claims 79-91, wherein machine-learning classifier component is a layer or branch of a machine-learning model.

93. The computer-implemented method of claim 92, wherein the machine-learning model is one of an ensemble of machine-learning models.

94. The computer-implemented method of claim 93, wherein the ensemble of machine-learning models comprises an ensemble of neural network models.

95. The computer-implemented method of any one of claims 79-94, further comprising:
obtaining low temporal resolution and low frequency resolution power spectra data from the EEG data; and
predicting the class label for the drug further comprising applying the low temporal resolution and low frequency resolution power spectra data to a low-resolution machine-learning classifier component trained to predict the class label using the low temporal resolution and low frequency resolution power spectra data.

96. The computer-implemented method of any one of claims 79-95, further comprising:
obtaining high temporal resolution and high frequency resolution power spectra data from the EEG data; and
predicting the class label for the drug further comprising applying the high temporal resolution and high frequency resolution power spectra data to a high-resolution machine-learning classifier component trained to predict the class label using the high temporal resolution and high frequency resolution power spectra data.

97. The computer-implemented method of any one of claims 79-96, further comprising:
obtaining covariance data of EEG data obtained from at least two of the plurality of electrodes; and
predicting the class label for the drug further comprising applying the covariance data to a covariance machine-learning classifier component trained to predict the class label using the covariance data.

98. The computer-implemented method of any one of claims 79-94, further comprising at least one of:
obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
obtaining covariance data of EEG data obtained from at least two of the plurality of electrodes; and predicting the class label for the drug further comprising applying the covariance data to a covariance machine-learning classifier component trained to predict the class label using the covariance data.

99. The computer-implemented method of any one of claims 79-98, wherein the animal subject is a rodent.

100. A computer-implemented method of extracting gait features of a rodent comprising:
obtaining video data illustrating a rodent, to which a drug is administered, over a predetermined period, the video data acquired using an enclosure for the rodent, the enclosure instrumented with an illuminated track for the rodent and at least one imaging device positioned to image an underside of the illuminated track;
annotating frames in the video data with labels using two machine-learning components, the labels including:
a first one of the two machine-learning components configured to divide a frame in video data into segments corresponding to first object classes, the first object classes comprising a paw class; and
a second one of the two machine-learning components configured to detect bounding boxes corresponding to second object classes, the second object classes including hind left, hind right, front left, and front right paws;
generating segmented images using the annotating frames, the segmented images divided into segments corresponding to third object classes including hind left, hind right, front left, and front right paws; and
extracting gait features of the rodent from the segmented images.

101. The computer-implemented method of claim 100, wherein the first object classes further comprise a background class and a body class.

102. The computer-implemented method of claim 100 or 101, wherein the second object class further comprise a background class, a first body class indicating the rodent moving from left to right or clockwise, and a second body class indicating the rodent moving from right to left or counterclockwise.

103. The computer-implemented method of any one of claims 100-102, wherein the first one of the two machine-learning components comprises a U-net convolutional neural network (CNN).

104. The computer-implemented method of any one of claims 100-103, wherein the second one of the two machine-learning components comprises a region-based CNN (R-CNN).

105. The computer-implemented method of any one of claims 100-104, further comprising automatically correcting the division of the frame into segments and/or identification of the third object classes using a plurality of heuristic rules based on positional relationship of the third object classes.

106. The computer-implemented method of any one of claims 100-105, further comprising extracting positional data of the body center and one or more paws over a sequence of frames in the video data.

107. The computer-implemented method of claim 106, further comprising extracting the gait features from the positional data over the sequence of frames in the video data.

108. The computer-implemented method of any one of claims 100-107, further comprising extracting the gait features over a plurality of cycles; and deriving a gait pattern of the rodent from the gait features.

109. The computer-implemented method of any one of claims 100-108, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.

110. The computer-implemented method of any one of claims 100-108, further comprising extracting features of the rodent from the gait features using a machine-learning feature-extraction component.

111. The computer-implemented method of claim 110, wherein the features comprise at least one of forward walk, immobile, turn around, or backward walk.

112. The computer-implemented method of claim 110, wherein the features comprise instant behavioral features.

113. The computer-implemented method of claim 112, wherein the features comprise higher-order features derived from the instant behavioral features using a machine-learning higher-order feature-extraction component.

114. The computer-implemented method of claim 113, wherein the higher-order features comprise one or more state features, and the method further comprises extracting the state features from the instant behavioral features using a machine-learning state-extraction component, wherein the machine-learning state-extraction component comprises a supervised machine-learning component, an unsupervised machine-learning component, or both.

115. The computer-implemented method of claim 114, wherein the higher-order features comprise one or more motif features, and the method further comprises extracting the motif features from the state features using a machine-learning motif-extraction component, wherein the machine-learning motif-extraction component comprises a supervised machine-learning component, an unsupervised machine-learning component, or both.

116. The computer-implemented method of claim 115, wherein the higher-order features comprise one or more domain features, and the method further comprises extracting the domain features from the motif features using a machine-learning higher-order-extraction component, wherein the machine-learning higher-order-extraction component comprises a supervised machine-learning component, an unsupervised machine-learning component, or both.

117. A system for classifying a drug, comprising:
at least one processor; and
a non-transitory storage medium storing instructions that, when executed by the at least one processor, cause the system to perform operations comprising:
obtaining observational data concerning an animal subject to which the drug is administered, the observational data acquired using an enclosure for the animal subject, the enclosure instrumented with at least one sensing device;
extracting features by applying the observational data to a machine-learning feature-extraction component;
predicting a class label of the drug by applying the features to a machine-learning classifier component, the machine-learning classifier component trained to predict the class label of the drug from, at least in part, the features; and
providing an indication of the class label.

118. A system for classifying a drug, comprising:
at least one processor; and
a non-transitory storage medium storing instructions that, when executed by the at least one processor, cause the system to perform operations comprising:
obtaining observational data concerning an animal subject to which the drug is administered, the observational data comprising at least one of thermal data or respirational data, the observation data acquired using an enclosure for the animal subject, the enclosure instrumented with at least one sensing device;
extracting features by applying the observational data to a machine-learning feature-extraction component;
predicting a class label of the drug by applying the features to a machine-learning classifier component trained to predict the class label of the drug from, at least in part, the features; and
providing an indication of the class label.

119. A system for classifying a psychedelic drug, comprising:
at least one processor; and
a non-transitory storage medium storing instructions that, when executed by the at least one processor, cause the system to perform operations comprising:
obtaining observational data concerning an animal subject to which a predetermined dose of the psychedelic drug is administered, the observation data acquired using an enclosure for the animal subject, the enclosure instrumented with at least one sensing device;
extracting features by applying the observational data to a machine-learning feature-extraction component, the features comprising at least one of head twitch, nose scratch, ear scratch, head shake, body elongation, or elongation-contraction;
predicting a class label of the psychedelic drug at the predetermined dose by applying the features to a machine-learning classifier component trained to predict the class label of the psychedelic drug from, at least in part, the features; and
providing an indication of the class label.

120. The system of any one of claims 117-119, wherein a machine-learning component is a layer or branch of a machine-learning model.

121. The system of claim 120, wherein the machine-learning model is one of an ensemble of machine-learning models.

122. The system of any one of claims 117-121, wherein the features comprise instant behavioral features corresponding to sets or sequences of data points indexed in time order of a first predetermined time scale.

123. The system of any one of claim 122, wherein the operations further comprise extracting the instant behavioral features using hard-coded definitions contained within the machine-learning feature-extraction component.

124. The system of any one of claims 117-123, wherein the operations further comprise:
deriving higher-order features based on the instant behavioral features using a machine-learning higher-order-extraction component; and
predicting the class label of the drug by applying the higher-order features to the machine-learning classifier component, the machine-learning classifier component trained to predict the class label of the drug from, at least in part, the higher-order features.

125. The system of claim 124, wherein the higher-order features correspond to sets or sequences of instant behavioral features indexed in time order of a second predetermined time scale, the second predetermined time scale being greater than the first predetermined time scale.

126. The system of any one of claims 117-125, wherein the operations further comprise obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.

127. The system of any one of claims 117-126, wherein the at least one sensing device comprises at least one imaging sensor configured to obtain image data.

128. The system of any one of claims 117-127, wherein the at least one imaging sensor comprises a thermal imaging sensor configured to obtain thermal image data.

129. The system of claim 127 or 128, wherein the at least one imaging sensor comprises a camera having a frame rate of at least 30 frames-per-second (fps).

130. The system of any one of claims 127-129, wherein the at least one imaging sensor comprises a high-speed camera having a frame rate of at least 70 fps.

131. The system of any one of claims 127-129, wherein the at least one imaging sensor comprises a high-speed camera having a frame rate that is equal or superior to:
a predetermined sampling rate for a behavior or action of the animal subject, or
the maximum of the predetermined sampling rates for a collection of behaviors or actions extracted from a single data source.

132. The system of any one of claims 127-131, wherein the at least one imaging sensor comprises an event imaging sensor configured to obtain dynamic image data.

133. The system of claim 132, wherein the event imaging sensor is configured to have a dynamic range of at least 100 dB or an equivalent frame rate of at least 500,000 fps.

134. The system of any one of claims 127-133, wherein the operations further comprise using the at least one imaging sensor with at least one mirror to obtain 3D image data.

135. The system of any one of claims 127-134, wherein the at least one imaging sensor comprises a plurality of imaging sensors configured to obtain 3D image data.

136. The system of any one of claims 127-135, wherein the observational data comprises a video of the animal subject obtained using the at least one imaging sensor, and the operations further comprise segmenting image frames of the video using a machine-learning segmentation model.

137. The system of claim 136, wherein the operations further comprise:
segmenting image frames of the video using a machine-learning segmentation model; and
extracting the features by tracking at least one segmented object in the image frames using a trained deep learning component.

138. The system of any one of claims 117-137, wherein the observational data comprises external data, the external data comprising data concerning one or more environmental designs of the enclosure, data concerning one or more stimuli given to the animal subject, or one or more rewards given to the animal subject.

139. The system of any one of claims 117-138, wherein the observational data comprises physiological data of the animal subject.

140. The system of any one of claim 139, wherein the at least one sensing device comprises a thermal sensor and the physiological data comprises temperature data obtained using the thermal sensor.

141. The system of claim 140, wherein the temperature data comprises temperature measurements of at least one body part of the animal subject, the at least one body part comprising at least one or more eyes, paws, tail, or limbs.

142. The system of any one of claims 139-141, wherein the at least one sensing device comprises at least one electroencephalogram (EEG) electrode and the physiological data comprises EEG data obtained using the least one EEG electrode.

143. The system of any one of claims 118-142, wherein the respirational data comprises a respiration rate during a period when the animal subject is not in active locomotion.

144. The system of any one of claims 127-143, wherein the operations further comprise deriving the respirational data, using a machine-learning respiration component, from image data obtained from at least one imaging sensor.

145. The system of any one of claims 117-144, wherein the machine-learning feature-extraction component comprises a supervised machine-learning component, an unsupervised machine-learning component, or both.

146. The system of any one of claims 124-145, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

147. The system of any one of claims 124-146, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

148. The system of any one of claims 124-147, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

149. The system of any one of claims 117-148, wherein the operations further comprise:
creating a treatment signature from the features;
generating a signature difference between the treatment signature and a baseline signature concerning a control animal, the baseline signature comprising the features; and
identifying a reference drug based on the signature difference and the treatment signature; and
providing the indication of the class label based on the identified reference drug.

150. The system of claim 149, wherein the operations further comprise ranking the features of the treatment signature based on the signature difference using a support vector machine-learning component.

151. The system of claim 149 or 150, wherein the operations further comprise weighting one or more of feature difference values between the treatment signature and the baseline signature prior to identifying the reference drug.

152. The system of claim 151, wherein the weights are generated using decorrelated ranked feature analysis.

153. The system of any one of claims 149-152, wherein the identification of the reference drug comprises generating a similarity value for the reference drug using the treatment signature and a reference signature corresponding to the reference drug, the reference signature comprising the features.

154. The system of any one of claims 149-153, wherein the drug is administered to the animal subject at a first dose and the reference drug is administered at a second dose.

155. The system of any one of claims 149-153, wherein the operations further comprise generating a plurality of similarity values corresponding to the administration of the reference drug at different doses.

156. The system of any one of claims 153-155, wherein the generation of the similarity value comprises generating an upregulation enrichment score and a downregulation enrichment score for the reference drug using the treatment signature and reference signature.

157. The system of any one of claims 153-156, wherein the generation of the similarity value comprises generating a combined enrichment score for the reference drug using the treatment signature and the reference signature.

158. The system of any one of claims 149-157, wherein the operations further comprise deriving a recovery value using a function of the treatment signature and a target signature concerning the animal subject prior to administration of the drug, the target signature comprising the features.

159. The system of any one of claims 117-158, wherein the operations further comprise deriving a treatment Markov model concerning the animal subject using a machine-learning Markov component, the treatment Markov model comprising a plurality of Markov states representing a selection of the features, each Markov state being associated with one or more Markov states by one or more transition probabilities.

160. The system of claim 159, wherein the selection of the higher-order features comprise a selection of state features, and the plurality of Markov states represent the selection of state features, and the operations further comprise deriving at least one motif feature representing a sequence of transitions of one or more of the selected state features.

161. The system of claim 159 or 160, wherein the treatment Markov model is a hidden Markov model comprising at least one hidden state.

162. The system of any one of claims 159-161, wherein the operations further comprise generating a visual representation of the treatment Markov model concerning the animal subject; and displaying the visual representation on a display.

163. The system of any one of claims 159-162, wherein the operations further comprise obtaining, using the machine-learning Markov component, a control Markov model concerning a control animal to which a vehicle is administered, the control Markov model comprising the plurality of Markov states representing the selection of the features.

164. The system of any one of claims 159-163, wherein the operations further comprise generating transition probability differences between the transition probabilities of the treatment Markov model and the transition probabilities of the control Markov model; and generating a visual representation of the transition probability differences associated with the plurality of Markov states.

165. The system of any one of claims 124-164, wherein the higher-order features comprise at least one of head twitch, nose scratch, ear scratch, head shake, body elongation, or elongation-contraction, and the operations further comprise predicting the class label to be associated with psychedelics.

166. The system of any one of claims 117-165, wherein the operations further comprise predicting the class label to be associated with one or more subclasses of psychedelics, entheogens, or psychoplastogens.

167. The system of any one of claims 117-166, wherein the animal subject is a rodent.

168. The system of any one of claims 117-167, wherein the animal subject is a mouse or a rat.

169. The system of any one of claims 117-168, wherein the drug is administered before the data is acquired or during acquisition of the data.

170. The system of any one of claims 117-169, wherein the operations further comprise obtaining the observational data concerning the animal subject while the animal subject is not in active locomotion.

171. The system of any one of claims 117-170, wherein the at least one sensing device comprises a headset comprising at least one of an accelerometer, gyroscope, or magnetometer, the headset configured to detect at least one type of motion of the head of the animal subject.

172. The system of any of claims 119-171, wherein the operations further comprise training the machine-learning classifier component to predict the class label of the psychedelic drug representing a treatment effect at the predetermined dose, the predetermined dose being a non-dissociative drug dose.

173. The system of any one of claims 119-172, wherein the operations further comprise training the machine-learning classifier component to predict the class label of the psychedelic drug representing a non-specific treatment effect at the predetermined dose, the predetermined dose being a dissociative drug dose.

174. A system for drug screening, comprising:
at least one processor; and
a non-transitory storage medium storing instructions that, when executed by the at least one processor, cause the system to perform operations comprising:
obtaining observational data concerning an animal subject, the observational data acquired using an enclosure for the animal subject, the enclosure instrumented with at least one sensing device;
extracting instant behavioral features from the observational data;
creating a treatment signature, the treatment signature including higher-order features derived from the instant behavioral features using a first machine-learning component, the higher-order features including at least one of a state feature, a motif feature, or a domain feature;
generating a target signature difference between the treatment signature and a baseline signature;
identifying at least one reference drug or condition based on the target signature difference, identification comprising:
generating an upregulation enrichment score and a downregulation enrichment score for the at least one reference drug or condition using the target signature difference and a reference signature difference corresponding to the one of the at least one reference drug or condition;
generating a combined enrichment score for the at least one reference drug or condition using the target signature difference and a reference signature difference corresponding to the one of the at least one reference drug or condition; or
generating a similarity value for the at least one reference drug or condition using the target signature difference and a reference signature difference corresponding to the one of the at least one reference drug or condition; and
providing an indication of the similarity value, combined enrichment score, or upregulation and downregulation enrichment scores for the at least one reference drug or condition.

175. The system of claim 174, wherein identifying the at least one reference drug or condition based on the target signature difference comprises generating the upregulation enrichment score and the downregulation enrichment score for the at least one reference drug or condition.

176. The system of claim 174 or 175, wherein the upregulation enrichment score and the downregulation enrichment score comprise gene set enrichment analysis scores.

177. The system of any one of claims 174-176, wherein identifying the at least one reference drug or condition based on the target signature difference comprises generating the combined enrichment score for the at least one reference drug or condition.

178. The system of any one of claims 174-177, wherein generating the combined enrichment score for the at least one reference drug or condition comprises:
generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.

179. The system of any one of claims 174-178, wherein identifying the at least one reference drug or condition based on the target signature difference comprises generating the similarity value for the at least one reference drug or condition.

180. The system of any one of claims 174-179, wherein the animal subject is an animal raised or modified to serve as a model of a human disease.

181. The system of claim 180, wherein the human disease is Rett syndrome, Parkinson's disease, Alzheimer's disease, Huntington disease, Tuberous Sclerosis Complex, or Autism Spectrum Disorder.

182. The system of any one of claims 174-181, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.

183. The system of any one of claims 174-182, wherein the operations further comprise weighting one or more of behavioral feature difference values of the reference signature difference or the target signature difference prior to identifying the at least one reference drug or condition.

184. The system of claim 183, wherein the weights are generated using decorrelated ranked feature analysis.

185. A system for drug screening, comprising:
at least one processor; and
a non-transitory storage medium storing instructions that, when executed by the at least one processor, cause the system to perform operations comprising:
in a training phase:
obtaining, for each first animal subject in three or more sets of first animal subjects, each of the first sets corresponding to a combination of values of two or more characteristics of the first animals, a first value for each behavioral feature in a set of features, the features including:
instant behavioral features extracted from observational data acquired for the first animal subjects using an enclosure instrumented with at least one sensing device; and
higher-order features derived from the instant behavioral features using a first machine-learning component;
determining, using a second machine-learning component and the first values, a mapping between at least two dimensions and corresponding functions of the features, the at least two dimensions including a treatment dimension and a secondary dimension; and
in a screening phase:
obtaining a second value for each behavioral feature in the set of the features for a second animal subject to which a compound is administered;
determining, using the mapping and the second values, a treatment effect of the compound; and
providing an indication of the treatment effect.

186. The system of claim 185, wherein the corresponding function of the features represents a weighted combination of the features, and the operations further comprise determining weights of the function of the features based on a discrimination power of each behavioral feature derived using a third machine-learning component.

187. The system of claim 186, wherein the third machine-learning component is a support vector machine-learning component trained to determine the weights based on features of a test animal group and a control animal group.

188. The system of any of claims 185-188, wherein the secondary dimension comprises a dimension orthogonal to the treatment dimension.

189. The system of claim 188, wherein the operations further comprise determining, using the mapping and the second values, a secondary effect along the secondary dimension.

190. The system of claim 189, wherein the secondary effect comprises a dissociative effect of the compound.

191. The system of claim 189 or 190, wherein the secondary effect comprises a side effect of the compound.

192. The system of any one of claims 189-191, wherein the secondary effect comprises a physiological condition.

193. The system of claim 192, wherein the physiological condition is aging.

194. The system of claim 192, wherein the physiological condition is a neurological disease, disorder, or dysfunction.

195. A system of classifying a drug comprising:
at least one processor; and
a non-transitory storage medium storing instructions that, when executed by the at least one processor, cause the system to perform operations comprising:
obtaining EEG data from a plurality of electrodes positioned on an animal subject to which the drug is administered at a first dose;
obtaining acceleration data from one or more accelerometers positioned on the animal subject to which the drug is administered;
predicting a class label for the drug by applying the EEG data and the acceleration data to a machine-learning classifier component trained to predict the class label using the EEG data and the acceleration data; and
providing an indication of the class label.

196. The system of claim 195, wherein the operations further comprise obtaining observational data concerning the animal subject, the observation data acquired using an enclosure for the animal subject, the enclosure instrumented with at least one sensing device.

197. The system of claim 196, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.

198. The system of claim 196 or 197, wherein the operations further comprise extracting features by applying the observational data to a machine-learning feature-extraction component, the observational data comprising the EEG data and the acceleration data.

199. The system of claim 198, wherein the features comprise instant behavioral features.

200. The system of claim 199, wherein the features comprise higher-order features derived from the instant behavioral features using a machine-learning higher-order feature-extraction component.

201. The system of claim 200, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

202. The system of claim 201, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

203. The system of claim 202, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

204. The system of any one of claims 195-203, wherein the EEG data comprises wake EEG and sleep EEG, and the operations further comprise automatically separating the wake EEG from the sleep EEG based on the EEG data and the acceleration data.

205. The system of any one of claims 195-204, wherein the operations further comprise:
obtaining reference EEG data from the plurality of electrodes positioned on the animal subject to which a reference drug is administered at a second dose; and
obtaining reference acceleration data from the one or more accelerometers positioned on the animal subject to which the reference drug is administered at the second dose.

206. The system of claim 205, wherein the operations further comprise generating a similarity value for the reference drug using the EEG data, the acceleration data, the reference EEG data, and the reference acceleration data.

207. The system of any one of claims 195-206, wherein the machine-learning classifier component comprises a Recurrent Neural Network (RNN).

208. The system of any one of claims 195-207, wherein machine-learning classifier component is a layer or branch of a machine-learning model.

209. The system of claim 208, wherein the machine-learning model is one of an ensemble of machine-learning models.

210. The system of claim 209, wherein the ensemble of machine-learning models comprises an ensemble of neural network models.

211. The system of any one of claims 195-210, wherein the operations further comprise:
obtaining low temporal resolution and low frequency resolution power spectra data from the EEG data; and
predicting the class label for the drug further comprising applying the low temporal resolution and low frequency resolution power spectra data to a low-resolution machine-learning classifier component trained to predict the class label using the low temporal resolution and low frequency resolution power spectra data.

212. The system of any one of claims 195-211, wherein the operations further comprise:
obtaining high temporal resolution and high frequency resolution power spectra data from the EEG data; and
predicting the class label for the drug further comprising applying the high temporal resolution and high frequency resolution power spectra data to a high-resolution machine-learning classifier component trained to predict the class label using the high temporal resolution and high frequency resolution power spectra data.

213. The system of any one of claims 195-212, wherein the operations further comprise:
obtaining covariance data of EEG data obtained from at least two of the plurality of electrodes; and
predicting the class label for the drug further comprising applying the covariance data to a covariance machine-learning classifier component trained to predict the class label using the covariance data.

214. The system of any one of claims 195-210, wherein the operations further comprise at least one of:
obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
obtaining covariance data of EEG data obtained from at least two of the plurality of electrodes; and predicting the class label for the drug further comprising applying the covariance data to a covariance machine-learning classifier component trained to predict the class label using the covariance data.

215. The system of any one of claims 195-214, wherein the animal subject is a rodent.

216. A system of extracting gait features of a rodent comprising:
at least one processor; and
a non-transitory storage medium storing instructions that, when executed by the at least one processor, cause the system to perform operations comprising:
obtaining video data illustrating a rodent, to which a drug is administered, over a predetermined period, the video data acquired using an enclosure for the rodent, the enclosure instrumented with an illuminated track for the rodent and at least one imaging device positioned to image an underside of the illuminated track;
annotating frames in the video data with labels using two machine-learning components, the labels including:
a first one of the two machine-learning components configured to divide a frame in video data into segments corresponding to first object classes, the first object classes comprising a paw class; and
a second one of the two machine-learning components configured to detect bounding boxes corresponding to second object classes, the second object classes including hind left, hind right, front left, and front right paws;
generating segmented images using the annotating frames, the segmented images divided into segments corresponding to third object classes including hind left, hind right, front left, and front right paws; and
extracting gait features of the rodent from the segmented images.

217. The system of claim 216, wherein the first object classes further comprise a background class and a body class.

218. The system of claim 216 or 217, wherein the second object class further comprise a background class, a first body class indicating the rodent moving from left to right or clockwise, and a second body class indicating the rodent moving from right to left or counterclockwise.

219. The system of any one of claims 216-218, wherein the first one of the two machine-learning components comprises a U-net convolutional neural network (CNN).

220. The system of any one of claims 216-219, wherein the second one of the two machine-learning components comprises a region-based CNN (R-CNN).

221. The system of any one of claims 216-220, wherein the operations further comprise automatically correcting the division of the frame into segments and/or identification of the third object classes using a plurality of heuristic rules based on positional relationship of the third object classes.

222. The system of any one of claims 216-221, wherein the operations further comprise extracting positional data of the body center and one or more paws over a sequence of frames in the video data.

223. The system of claim 222, wherein the operations further comprise extracting the gait features from the positional data over the sequence of frames in the video data.

224. The system of any one of claims 216-223, wherein the operations further comprise extracting the gait features over a plurality of cycles; and deriving a gait pattern of the rodent from the gait features.

225. The system of any one of claims 216-224, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.

226. The system of any one of claims 216-224, wherein the operations further comprise extracting features of the rodent from the gait features using a machine-learning feature-extraction component.

227. The system of claim 226, wherein the features comprise at least one of forward walk, immobile, turn around, or backward walk.

228. The system of claim 226, wherein the features comprise instant behavioral features.

229. The system of claim 228, wherein the features comprise higher-order features derived from the instant behavioral features using a machine-learning higher-order feature-extraction component.

230. The system of claim 229, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

231. The system of claim 230, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

232. The system of claim 231, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

233. A non-transitory computer-readable medium containing instructions that, when executed by at least one processor of a system, cause the system to perform operations for classifying a drug, the operations comprising:
obtaining observational data concerning an animal subject to which the drug is administered, the observational data acquired using an enclosure for the animal subject, the enclosure instrumented with at least one sensing device;
extracting features by applying the observational data to a machine-learning feature-extraction component;
predicting a class label of the drug by applying the features to a machine-learning classifier component, the machine-learning classifier component trained to predict the class label of the drug from, at least in part, the features; and
providing an indication of the class label.

234. A non-transitory computer-readable medium containing instructions that, when executed by at least one processor of a system, cause the system to perform operations for classifying a drug, the operations comprising:
obtaining observational data concerning an animal subject to which the drug is administered, the observational data comprising at least one of thermal data or respirational data, the observation data acquired using an enclosure for the animal subject, the enclosure instrumented with at least one sensing device;
extracting features by applying the observational data to a machine-learning feature-extraction component;
predicting a class label of the drug by applying the features to a machine-learning classifier component trained to predict the class label of the drug from, at least in part, the features; and
providing an indication of the class label.

235. A non-transitory computer-readable medium containing instructions that, when executed by at least one processor of a system, cause the system to perform operations for classifying a psychedelic drug comprising:
obtaining observational data concerning an animal subject to which a predetermined dose of the psychedelic drug is administered, the observation data acquired using an enclosure for the animal subject, the enclosure instrumented with at least one sensing device;
extracting features by applying the observational data to a machine-learning feature-extraction component, the features comprising at least one of head twitch, nose scratch, ear scratch, head shake, body elongation, or elongation-contraction;
predicting a class label of the psychedelic drug at the predetermined dose by applying the features to a machine-learning classifier component trained to predict the class label of the psychedelic drug from, at least in part, the features; and
providing an indication of the class label.

236. The non-transitory computer-readable medium of any one of claims 233-235, wherein a machine-learning component is a layer or branch of a machine-learning model.

237. The non-transitory computer-readable medium of claim 236, wherein the machine-learning model is one of an ensemble of machine-learning models.

238. The non-transitory computer-readable medium of any one of claims 233-237, wherein the features comprise instant behavioral features corresponding to sets or sequences of data points indexed in time order of a first predetermined time scale.

239. The non-transitory computer-readable medium of any one of claim 238, wherein the operations further comprise extracting the instant behavioral features using hard-coded definitions contained within the machine-learning feature-extraction component.

240. The non-transitory computer-readable medium of any one of claims 233-239, wherein the operations further comprise:
deriving higher-order features based on the instant behavioral features using a machine-learning higher-order-extraction component; and
predicting the class label of the drug by applying the higher-order features to the machine-learning classifier component, the machine-learning classifier component trained to predict the class label of the drug from, at least in part, the higher-order features.

241. The non-transitory computer-readable medium of claim 240, wherein the higher-order features correspond to sets or sequences of instant behavioral features indexed in time order of a second predetermined time scale, the second predetermined time scale being greater than the first predetermined time scale.

242. The non-transitory computer-readable medium of any one of claims 233-241, wherein the operations further comprise obtaining the observational data from the at least one sensing device, wherein the at least one sensing device comprises at least one of an imaging sensor, a force sensor, a pressure sensor, a piezoelectric sensor, a pseudo piezoelectric sensor, an accelerometer, a stimulus sensor associated with a stimulus actuator, or a thermal sensor.

243. The non-transitory computer-readable medium of any one of claims 233-242, wherein the at least one sensing device comprises at least one imaging sensor configured to obtain image data.

244. The non-transitory computer-readable medium of any one of claims 233-243, wherein the at least one imaging sensor comprises a thermal imaging sensor configured to obtain thermal image data.

245. The non-transitory computer-readable medium of claim 243 or 244, wherein the at least one imaging sensor comprises a camera having a frame rate of at least 30 frames-per-second (fps).

246. The non-transitory computer-readable medium of any one of claims 243-245, wherein the at least one imaging sensor comprises a high-speed camera having a frame rate of at least 70 fps.

247. The non-transitory computer-readable medium of any one of claims 243-245, wherein the at least one imaging sensor comprises a high-speed camera having a frame rate that is equal or superior to:
a predetermined sampling rate for a behavior or action of the animal subject, or
the maximum of the predetermined sampling rates for a collection of behaviors or actions extracted from a single data source.

248. The non-transitory computer-readable medium of any one of claims 243-247, wherein the at least one imaging sensor comprises an event imaging sensor configured to obtain dynamic image data.

249. The non-transitory computer-readable medium of claim 248, wherein the event imaging sensor is configured to have a dynamic range of at least 100 dB or an equivalent frame rate of at least 500,000 fps.

250. The non-transitory computer-readable medium of any one of claims 243-249, wherein the operations further comprise using the at least one imaging sensor with at least one mirror to obtain 3D image data.

251. The non-transitory computer-readable medium of any one of claims 243-250, wherein the at least one imaging sensor comprises a plurality of imaging sensors configured to obtain 3D image data.

252. The non-transitory computer-readable medium of any one of claims 243-251, wherein the observational data comprises a video of the animal subject obtained using the at least one imaging sensor, and the operations further comprise segmenting image frames of the video using a machine-learning segmentation model.

253. The non-transitory computer-readable medium of claim 252, wherein the operations further comprise:
segmenting image frames of the video using a machine-learning segmentation model; and
extracting the features by tracking at least one segmented object in the image frames using a trained deep learning component.

254. The non-transitory computer-readable medium of any one of claims 233-253, wherein the observational data comprises external data, the external data comprising data concerning one or more environmental designs of the enclosure, data concerning one or more stimuli given to the animal subject, or one or more rewards given to the animal subject.

255. The non-transitory computer-readable medium of any one of claims 233-254, wherein the observational data comprises physiological data of the animal subject.

256. The non-transitory computer-readable medium of any one of claim 255, wherein the at least one sensing device comprises a thermal sensor and the physiological data comprises temperature data obtained using the thermal sensor.

257. The non-transitory computer-readable medium of claim 256, wherein the temperature data comprises temperature measurements of at least one body part of the animal subject, the at least one body part comprising at least one or more eyes, paws, tail, or limbs.

258. The non-transitory computer-readable medium of any one of claims 255-257, wherein the at least one sensing device comprises at least one electroencephalogram (EEG) electrode and the physiological data comprises EEG data obtained using the least one EEG electrode.

259. The non-transitory computer-readable medium of any one of claims 234-258, wherein the respirational data comprises a respiration rate during a period when the animal subject is not in active locomotion.

260. The non-transitory computer-readable medium of any one of claims 243-259, wherein the operations further comprise deriving the respirational data, using a machine-learning respiration component, from image data obtained from at least one imaging sensor.

261. The non-transitory computer-readable medium of any one of claims 233-260, wherein the machine-learning feature-extraction component comprises a supervised machine-learning component, an unsupervised machine-learning component, or both.

262. The non-transitory computer-readable medium of any one of claims 240-261, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

263. The non-transitory computer-readable medium of any one of claims 240-262, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

264. The non-transitory computer-readable medium of any one of claims 240-263, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

265. The non-transitory computer-readable medium of any one of claims 233-264, wherein the operations further comprise:
creating a treatment signature from the features;
generating a signature difference between the treatment signature and a baseline signature concerning a control animal, the baseline signature comprising the features; and
identifying a reference drug based on the signature difference and the treatment signature; and
providing the indication of the class label based on the identified reference drug.

266. The non-transitory computer-readable medium of claim 265, wherein the operations further comprise ranking the features of the treatment signature based on the signature difference using a support vector machine-learning component.

267. The non-transitory computer-readable medium of claim 265 or 266, wherein the operations further comprise weighting one or more of feature difference values between the treatment signature and the baseline signature prior to identifying the reference drug.

268. The non-transitory computer-readable medium of claim 267, wherein the weights are generated using decorrelated ranked feature analysis.

269. The non-transitory computer-readable medium of any one of claims 265-268, wherein the identification of the reference drug comprises generating a similarity value for the reference drug using the treatment signature and a reference signature corresponding to the reference drug, the reference signature comprising the features.

270. The non-transitory computer-readable medium of any one of claims 265-269, wherein the drug is administered to the animal subject at a first dose and the reference drug is administered at a second dose.

271. The non-transitory computer-readable medium of any one of claims 265-269, wherein the operations further comprise generating a plurality of similarity values corresponding to the administration of the reference drug at different doses.

272. The non-transitory computer-readable medium of any one of claims 269-271, wherein the generation of the similarity value comprises generating an upregulation enrichment score and a downregulation enrichment score for the reference drug using the treatment signature and reference signature.

273. The non-transitory computer-readable medium of any one of claims 269-272, wherein the generation of the similarity value comprises generating a combined enrichment score for the reference drug using the treatment signature and the reference signature.

274. The non-transitory computer-readable medium of any one of claims 265-273, wherein the operations further comprise deriving a recovery value using a function of the treatment signature and a target signature concerning the animal subject prior to administration of the drug, the target signature comprising the features.

275. The non-transitory computer-readable medium of any one of claims 233-274, wherein the operations further comprise deriving a treatment Markov model concerning the animal subject using a machine-learning Markov component, the treatment Markov model comprising a plurality of Markov states representing a selection of the features, each Markov state being associated with one or more Markov states by one or more transition probabilities.

276. The non-transitory computer-readable medium of claim 275, wherein the selection of the higher-order features comprise a selection of state features, and the plurality of Markov states represent the selection of state features, and the operations further comprise deriving at least one motif feature representing a sequence of transitions of one or more of the selected state features.

277. The non-transitory computer-readable medium of claim 275 or 276, wherein the treatment Markov model is a hidden Markov model comprising at least one hidden state.

278. The non-transitory computer-readable medium of any one of claims 275-277, wherein the operations further comprise generating a visual representation of the treatment Markov model concerning the animal subject; and displaying the visual representation on a display.

279. The non-transitory computer-readable medium of any one of claims 275-278, wherein the operations further comprise obtaining, using the machine-learning Markov component, a control Markov model concerning a control animal to which a vehicle is administered, the control Markov model comprising the plurality of Markov states representing the selection of the features.

280. The non-transitory computer-readable medium of any one of claims 275-279, wherein the operations further comprise generating transition probability differences between the transition probabilities of the treatment Markov model and the transition probabilities of the control Markov model; and generating a visual representation of the transition probability differences associated with the plurality of Markov states.

281. The non-transitory computer-readable medium of any one of claims 240-280, wherein the higher-order features comprise at least one of head twitch, nose scratch, ear scratch, head shake, body elongation, or elongation-contraction, and the operations further comprise predicting the class label to be associated with psychedelics.

282. The non-transitory computer-readable medium of any one of claims 233-281, wherein the operations further comprise predicting the class label to be associated with one or more subclasses of psychedelics, entheogens, or psychoplastogens.

283. The non-transitory computer-readable medium of any one of claims 233-282, wherein the animal subject is a rodent.

284. The non-transitory computer-readable medium of any one of claims 233-283, wherein the animal subject is a mouse or a rat.

285. The non-transitory computer-readable medium of any one of claims 233-284, wherein the drug is administered before the data is acquired or during acquisition of the data.

286. The non-transitory computer-readable medium of any one of claims 233-285, wherein the operations further comprise obtaining the observational data concerning the animal subject while the animal subject is not in active locomotion.

287. The non-transitory computer-readable medium of any one of claims 233-286, wherein the at least one sensing device comprises a headset comprising at least one of an accelerometer, gyroscope, or magnetometer, the headset configured to detect at least one type of motion of the head of the animal subject.

288. The non-transitory computer-readable medium of any of claims 235-287, wherein the operations further comprise training the machine-learning classifier component to predict the class label of the psychedelic drug representing a treatment effect at the predetermined dose, the predetermined dose being a non-dissociative drug dose.

289. The non-transitory computer-readable medium of any one of claims 235-288, wherein the operations further comprise training the machine-learning classifier component to predict the class label of the psychedelic drug representing a non-specific treatment effect at the predetermined dose, the predetermined dose being a dissociative drug dose.

290. A non-transitory computer-readable medium containing instructions that, when executed by at least one processor of a system, cause the system to perform operations for drug screening, the operations comprising:
obtaining observational data concerning an animal subject, the observational data acquired using an enclosure for the animal subject, the enclosure instrumented with at least one sensing device;
extracting instant behavioral features from the observational data;
creating a treatment signature, the treatment signature including higher-order features derived from the instant behavioral features using a first machine-learning component, the higher-order features including at least one of a state feature, a motif feature, or a domain feature;
generating a target signature difference between the treatment signature and a baseline signature;
identifying at least one reference drug or condition based on the target signature difference, identification comprising:
generating an upregulation enrichment score and a downregulation enrichment score for the at least one reference drug or condition using the target signature difference and a reference signature difference corresponding to the one of the at least one reference drug or condition;
generating a combined enrichment score for the at least one reference drug or condition using the target signature difference and a reference signature difference corresponding to the one of the at least one reference drug or condition; or
generating a similarity value for the at least one reference drug or condition using the target signature difference and a reference signature difference corresponding to the one of the at least one reference drug or condition; and
providing an indication of the similarity value, combined enrichment score, or upregulation and downregulation enrichment scores for the at least one reference drug or condition.

291. The computer-implemented drug screening method of claim 290, wherein identifying the at least one reference drug or condition based on the target signature difference comprises generating the upregulation enrichment score and the downregulation enrichment score for the at least one reference drug or condition.

292. The computer-implemented drug screening method of claim 290 or 291, wherein the upregulation enrichment score and the downregulation enrichment score comprise gene set enrichment analysis scores.

293. The computer-implemented drug screening method of any one of claims 290-292, wherein identifying the at least one reference drug or condition based on the target signature difference comprises generating the combined enrichment score for the at least one reference drug or condition.

294. The computer-implemented drug screening method of any one of claims 290-293, wherein generating the combined enrichment score for the at least one reference drug or condition comprises:
generating a re-sorted magnitude version of the reference signature difference; identifying, in the target signature difference, a set of increased features and a set of decreased features; creating a combined feature set using the set of increased features and the set of decreased features; and generating the combined enrichment score using the combined feature set and the re-sorted magnitude version of the reference signature difference; or
generating a re-sorted magnitude version of the target signature difference; identifying, in the reference signature difference, a set of increased features and a set of decreased features; creating a combined feature set using the set of increased features and the set of decreased features; and generating the combined enrichment score using the combined feature set and the re-sorted magnitude version of the target signature difference.

295. The computer-implemented drug screening method of any one of claims 290-294, wherein identifying the at least one reference drug or condition based on the target signature difference comprises generating the similarity value for the at least one reference drug or condition.

296. The computer-implemented drug screening method of any one of claims 290-295, wherein the animal subject is an animal raised or modified to serve as a model of a human disease.

297. The computer-implemented drug screening method of claim 296, wherein the human disease is Rett syndrome, Parkinson's disease, Alzheimer's disease, Huntington disease, Tuberous Sclerosis Complex, or Autism Spectrum Disorder.

298. The computer-implemented drug screening method of any one of claims 290-297, wherein the animal subject is administered a compound having a known effect in humans, and the at least one reference drug is identified based on the similarity value, combined enrichment score, or upregulation and downregulation enrichment scores as having a similar drug-induced behavioral data profile or a reversed drug-induced behavioral data profile as the administered compound.

299. The computer-implemented drug screening method of any one of claims 290-298, wherein the operations further comprise weighting one or more of behavioral feature difference values of the reference signature difference or the target signature difference prior to identifying the at least one reference drug or condition.

300. The computer-implemented drug screening method of claim 299, wherein the weights are generated using decorrelated ranked feature analysis.

301. A non-transitory computer-readable medium containing instructions that, when executed by at least one processor of a system, cause the system to perform operations for drug screening, the operations comprising:
in a training phase:
obtaining, for each first animal subject in three or more sets of first animal subjects, each of the first sets corresponding to a combination of values of two or more characteristics of the first animals, a first value for each behavioral feature in a set of features, the features including:
instant behavioral features extracted from observational data acquired for the first animal subjects using an enclosure instrumented with at least one sensing device; and
higher-order features derived from the instant behavioral features using a first machine-learning component;
determining, using a second machine-learning component and the first values, a mapping between at least two dimensions and corresponding functions of the features, the at least two dimensions including a treatment dimension and a secondary dimension; and
in a screening phase:
obtaining a second value for each behavioral feature in the set of the features for a second animal subject to which a compound is administered;
determining, using the mapping and the second values, a treatment effect of the compound; and
providing an indication of the treatment effect.

302. The non-transitory computer-readable medium of claim 301, wherein the corresponding function of the features represents a weighted combination of the features, and the operations further comprise determining weights of the function of the features based on a discrimination power of each behavioral feature derived using a third machine-learning component.

303. The non-transitory computer-readable medium of claim 302, wherein the third machine-learning component is a support vector machine-learning component trained to determine the weights based on features of a test animal group and a control animal group.

304. The non-transitory computer-readable medium of any of claims 301-304, wherein the secondary dimension comprises a dimension orthogonal to the treatment dimension.

305. The non-transitory computer-readable medium of claim 304, wherein the operations further comprise determining, using the mapping and the second values, a secondary effect along the secondary dimension.

306. The non-transitory computer-readable medium of claim 305, wherein the secondary effect comprises a dissociative effect of the compound.

307. The non-transitory computer-readable medium of claim 305 or 306, wherein the secondary effect comprises a side effect of the compound.

308. The non-transitory computer-readable medium of any one of claims 305-307, wherein the secondary effect comprises a physiological condition.

309. The non-transitory computer-readable medium of claim 308, wherein the physiological condition is aging.

310. The non-transitory computer-readable medium of claim 308, wherein the physiological condition is a neurological disease, disorder, or dysfunction.

311. A non-transitory computer-readable medium containing instructions that, when executed by at least one processor of a system, cause the system to perform operations for classifying a drug, the operations comprising:
obtaining EEG data from a plurality of electrodes positioned on an animal subject to which the drug is administered at a first dose;
obtaining acceleration data from one or more accelerometers positioned on the animal subject to which the drug is administered;
predicting a class label for the drug by applying the EEG data and the acceleration data to a machine-learning classifier component trained to predict the class label using the EEG data and the acceleration data; and
providing an indication of the class label.

312. The non-transitory computer-readable medium of claim 311, wherein the operations further comprise obtaining observational data concerning the animal subject, the observation data acquired using an enclosure for the animal subject, the enclosure instrumented with at least one sensing device.

313. The non-transitory computer-readable medium of claim 312, wherein the at least one sensing device comprises at least one of an imaging sensor, a force sensor, a pressure sensor, a piezoelectric sensor, a pseudo piezoelectric sensor, a stimulus sensor associated with a stimulus actuator, or a thermal sensor.

314. The non-transitory computer-readable medium of claim 312 or 313, wherein the operations further comprise extracting features by applying the observational data to a machine-learning feature-extraction component, the observational data comprising the EEG data and the acceleration data.

315. The non-transitory computer-readable medium of claim 314, wherein the features comprise instant behavioral features.

316. The non-transitory computer-readable medium of claim 315, wherein the features comprise higher-order features derived from the instant behavioral features using a machine-learning higher-order feature-extraction component.

317. The non-transitory computer-readable medium of claim 316, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

318. The non-transitory computer-readable medium of claim 317, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

319. The non-transitory computer-readable medium of claim 318, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.

320. The non-transitory computer-readable medium of any one of claims 311-319, wherein the EEG data comprises wake EEG and sleep EEG, and the operations further comprise automatically separating the wake EEG from the sleep EEG based on the EEG data and the acceleration data.

321. The non-transitory computer-readable medium of any one of claims 311-320, wherein the operations further comprise:
obtaining reference EEG data from the plurality of electrodes positioned on the animal subject to which a reference drug is administered at a second dose; and
obtaining reference acceleration data from the one or more accelerometers positioned on the animal subject to which the reference drug is administered at the second dose.

322. The non-transitory computer-readable medium of claim 321, wherein the operations further comprise generating a similarity value for the reference drug using the EEG data, the acceleration data, the reference EEG data, and the reference acceleration data.

323. The non-transitory computer-readable medium of any one of claims 311-322, wherein the machine-learning classifier component comprises a Recurrent Neural Network (RNN).

324. The non-transitory computer-readable medium of any one of claims 311-323, wherein machine-learning classifier component is a layer or branch of a machine-learning model.

325. The non-transitory computer-readable medium of claim 324, wherein the machine-learning model is one of an ensemble of machine-learning models.

326. The non-transitory computer-readable medium of claim 325, wherein the ensemble of machine-learning models comprises an ensemble of neural network models.

327. The non-transitory computer-readable medium of any one of claims 311-326, wherein the operations further comprise:
obtaining low temporal resolution and low frequency resolution power spectra data from the EEG data; and
predicting the class label for the drug further comprising applying the low temporal resolution and low frequency resolution power spectra data to a low-resolution machine-learning classifier component trained to predict the class label using the low temporal resolution and low frequency resolution power spectra data.

328. The non-transitory computer-readable medium of any one of claims 311-327, wherein the operations further comprise:
obtaining high temporal resolution and high frequency resolution power spectra data from the EEG data; and
predicting the class label for the drug further comprising applying the high temporal resolution and high frequency resolution power spectra data to a high-resolution machine-learning classifier component trained to predict the class label using the high temporal resolution and high frequency resolution power spectra data.

329. The non-transitory computer-readable medium of any one of claims 311-328, wherein the operations further comprise:
obtaining covariance data of EEG data obtained from at least two of the plurality of electrodes; and
predicting the class label for the drug further comprising applying the covariance data to a covariance machine-learning classifier component trained to predict the class label using the covariance data.

330. The non-transitory computer-readable medium of any one of claims 311-326, wherein the operations further comprise at least one of:
obtaining low temporal resolution and low frequency resolution power spectra data from the EEG data; and predicting the class label for the drug further comprising applying the low temporal resolution and low frequency resolution power spectra data to a low-resolution machine-learning classifier component trained to predict the class label using the low temporal resolution and low frequency resolution power spectra data;
obtaining high temporal resolution and high frequency resolution power spectra data from the EEG data; and predicting the class label for the drug further comprising applying the high temporal resolution and high frequency resolution power spectra data to a high-resolution machine-learning classifier component trained to predict the class label using the high temporal resolution and high frequency resolution power spectra data; or
obtaining covariance data of EEG data obtained from at least two of the plurality of electrodes; and predicting the class label for the drug further comprising applying the covariance data to a covariance machine-learning classifier component trained to predict the class label using the covariance data.

331. The non-transitory computer-readable medium of any one of claims 311-330, wherein the animal subject is a rodent.

332. A non-transitory computer-readable medium containing instructions that, when executed by at least one processor of a system, cause the system to perform operations for extracting gait features of a rodent, the operations comprising:
obtaining video data concerning a rodent, to which a drug is administered, over a predetermined period, the video data acquired using an enclosure for the rodent, the enclosure instrumented with an illuminated track for the rodent and at least one imaging device positioned to image an underside of the illuminated track;
annotating frames in the video data with labels using two machine-learning components, the labels including:
a first one of the two machine-learning components configured to divide a frame in video data into segments corresponding to first object classes, the first object classes comprising a paw class; and
a second one of the two machine-learning components configured to detect bounding boxes corresponding to second object classes, the second object classes including hind left, hind right, front left, and front right paws;
generating segmented images using the annotating frames, the segmented images divided into segments corresponding to third object classes including hind left, hind right, front left, and front right paws; and
extracting gait features of the rodent from the segmented images.

333. The non-transitory computer-readable medium of claim 332, wherein the first object classes further comprise a background class and a body class.

334. The non-transitory computer-readable medium of claim 332 or 333, wherein the second object class further comprise a background class, a first body class indicating the rodent moving from left to right or clockwise, and a second body class indicating the rodent moving from right to left or counterclockwise.

335. The non-transitory computer-readable medium of any one of claims 332-334, wherein the first one of the two machine-learning components comprises a U-net convolutional neural network (CNN).

336. The non-transitory computer-readable medium of any one of claims 332-335, wherein the second one of the two machine-learning components comprises a region-based CNN (R-CNN).

337. The non-transitory computer-readable medium of any one of claims 332-336, wherein the operations further comprise automatically correcting the division of the frame into segments and/or identification of the third object classes using a plurality of heuristic rules based on positional relationship of the third object classes.

338. The non-transitory computer-readable medium of any one of claims 332-337, wherein the operations further comprise extracting positional data of the body center and one or more paws over a sequence of frames in the video data.

339. The non-transitory computer-readable medium of claim 338, wherein the operations further comprise extracting the gait features from the positional data over the sequence of frames in the video data.

340. The non-transitory computer-readable medium of any one of claims 332-339, wherein the operations further comprise extracting the gait features over a plurality of cycles; and deriving a gait pattern of the rodent from the gait features.

341. The non-transitory computer-readable medium of any one of claims 332-340, wherein the gait features comprise at least one of cycle type duration, cycle sequence type, total distance moved, average speed, movement direction, body parameters, paw position, paw parameters, number of paws, stride length, stride duration, step length, step duration, splay length, swing duration, stand duration, base width, or asymmetry.

342. The non-transitory computer-readable medium of any one of claims 332-340, wherein the operations further comprise extracting features of the rodent from the gait features using a machine-learning feature-extraction component.

343. The non-transitory computer-readable medium of claim 342, wherein the features comprise at least one of forward walk, immobile, turn around, or backward walk.

344. The non-transitory computer-readable medium of claim 342, wherein the features comprise instant behavioral features.

345. The non-transitory computer-readable medium of claim 344, wherein the features comprise higher-order features derived from the instant behavioral features using a machine-learning higher-order feature-extraction component.

346. The non-transitory computer-readable medium of claim 345, wherein the higher-order features comprise one or more state features, and the operations further comprise extracting the state features from the instant behavioral features using a machine-learning state-extraction component, wherein the machine-learning state-extraction component comprises a supervised machine-learning component, an unsupervised machine-learning component, or both.

347. The non-transitory computer-readable medium of claim 346, wherein the higher-order features comprise one or more motif features, and the operations further comprise extracting the motif features from the state features using a machine-learning motif-extraction component, wherein the machine-learning motif-extraction component comprises a supervised machine-learning component, an unsupervised machine-learning component, or both.

348. The non-transitory computer-readable medium of claim 347, wherein the higher-order features comprise one or more domain features, and the operations further comprise extracting the domain features from the motif features using a machine-learning higher-order-extraction component, wherein the machine-learning higher-order-extraction component comprises a supervised machine-learning component, an unsupervised machine-learning component, or both.
